(19) United States
(12) Patent Application Publication
(10) Pub. No.: US 2021/0253710 A1
(43) Pub. Date: Aug. 19, 2021

(54) DIAGNOSTIC AND THERAPEUTIC METHODS FOR KIDNEY CANCER

(71) Applicant: Genentech, Inc., South San Francisco, CA (US)

(72) Inventors: Christina SCHIFF, South San Francisco, CA (US); Marjorie GREEN, South San Francisco, CA (US); Mahrukh HUSENI, South San Francisco, CA (US)

(21) Appl. No.: 17/232,263

(22) Filed: Apr. 16, 2021

Related U.S. Application Data
(63) Continuation of application No. PCT/US2019/056665, filed on Oct. 17, 2019.
(60) Provisional application No. 62/855,517, filed on May 31, 2019.
(60) Provisional application No. 62/747,559, filed on Oct. 18, 2018.

(51) Int. Cl. C07K 16/28; C07K 16/22; A61K 45/06; G01N 33/574; A61P 35/00

(52) U.S. Cl. CPC C07K 16/2827; C07K 16/22; A61K 45/06; A61K 2039/507; A61P 35/00; G01N 2800/52; G01N 2496/00; G01N 33/57438

(57) ABSTRACT

The present invention provides diagnostic methods, therapeutic methods, and compositions for the treatment of cancer (e.g., kidney cancer (e.g., renal cell carcinoma (RCC)). The invention is based, at least in part, on the discovery that expression levels of one or more biomarkers described herein in a sample from an individual having cancer can be used in methods of predicting the therapeutic efficacy of treatment with a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)), or with an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))).

### SEQUENCE LISTING

The instant application contains a Sequence Listing which has been submitted electronically in ASCII format and is hereby incorporated by reference in its entirety. Said ASCII copy, created on Apr. 14, 2021, is named 50474-191004_Sequence_Listing_4_14_21_ST25 and is 235,626 bytes in size.

### FIELD OF THE INVENTION

The present invention is directed to diagnostic and therapeutic methods for the treatment of cancer (e.g., kidney cancer).

### BACKGROUND OF THE INVENTION

Cancer remains one of the most deadly threats to human health. In the U.S., cancer affects nearly 1.3 million new patients each year and is the second leading cause of death after heart disease, accounting for approximately 1 in 4 deaths. It is also predicted that cancer may surpass cardiovascular diseases as the number one cause of death within 5 years. Solid tumors are responsible for most of those deaths. Although there have been significant advances in the medical treatment of certain cancers, the overall 5-year survival rate for all cancers has improved only by about 10% in the past 20 years. Malignant solid tumors, in particular, metastasize and grow rapidly in an uncontrolled manner, making their timely detection and treatment extremely difficult. Renal cell carcinoma (RCC) is the most common type of kidney cancer and has multiple histological subtypes. Sarcomatoid RCC, which can occur in all histological subtypes, is characterized in part by features similar to sarcomas, including spindle-like cells, high cellularity, and cellular atypia. Sarcomatoid RCC is associated with a poor prognosis, including a median survival of about 6 months, and a higher percentage of sarcomatoid components is associated with a worse outcome. Sarcomatoid RCC is typically considered to be a poorly treatable and aggressive form of RCC.

Studies in humans with immune checkpoint inhibitors have demonstrated the promise of harnessing the immune system to control and eradicate tumor growth. The programmed death 1 (PD-1) receptor and its ligand programmed death-ligand 1 (PD-L1) are immune checkpoint proteins that have been implicated in the suppression of immune system responses during chronic infections, pregnancy, tissue allografts, autoimmune diseases, and cancer. PD-L1 regulates the immune response by binding to the inhibitory receptor PD-1, which is expressed on the surface of T-cells, B-cells, and monocytes. PD-L1 negatively regulates T-cell function also through interaction with another receptor, B7-1. Formation of the PD-L1/PD-1 and PD-L1/B7-1 complexes negatively regulates T-cell receptor signaling, resulting in the subsequent downregulation of T-cell activation and suppression of anti-tumor immune activity.

Despite the significant advancement in the treatment of cancer (e.g., kidney cancer), improved diagnostic methods and cancer therapies and are still being sought.

### SUMMARY OF THE INVENTION

The present invention provides diagnostic and therapeutic methods and compositions for treating an individual having a cancer (e.g., a kidney cancer (e.g., a renal cell carcinoma (RCC)), including a sarcomatoid cancer (e.g., a sarcomatoid kidney cancer (e.g., a sarcomatoid RCC, including locally advanced or metastatic sarcomatoid RCC)).

In one aspect, the invention features a method of treating an individual having a sarcomatoid cancer (e.g., a sarcomatoid kidney cancer (e.g., a sarcomatoid RCC, including locally advanced or metastatic sarcomatoid RCC)), the method comprising administering to the individual an effective amount of an anti-cancer therapy comprising a VEGF antagonist and a PD-L1 axis binding antagonist. In some embodiments, the individual is previously untreated for the sarcomatoid cancer.

In another aspect, the invention features a method of treating an individual having a cancer (e.g., a kidney cancer (e.g., an RCC, including locally advanced or metastatic RCC)) with a poor or intermediate Memorial Sloan Kettering Cancer Center (MSKCC) risk score, the method comprising administering to the individual an effective amount of an anti-cancer therapy comprising a VEGF antagonist and a PD-L1 axis binding antagonist. In some embodiments, the individual is previously untreated for the cancer.

In another aspect, the invention features a method of treating an individual having a kidney cancer, the method comprising administering to the individual an effective amount of an anti-cancer therapy comprising a VEGF antagonist and a PD-L1 axis binding antagonist, wherein the individual has been identified as likely to benefit from the anti-cancer therapy based on having a sarcomatoid kidney cancer.

In another aspect, the invention features a method of treating an individual having a kidney cancer, the method comprising: (a) determining whether the individual has a sarcomatoid kidney cancer, wherein the presence of a sarcomatoid kidney cancer indicates that the individual is likely to benefit from an anti-cancer therapy comprising a VEGF antagonist and a PD-L1 axis binding antagonist; and (b) administering an effective amount of an anti-cancer therapy comprising a VEGF antagonist and a PD-L1 axis binding antagonist to the individual based on the presence of a sarcomatoid kidney cancer.

In another aspect, the invention features a method of identifying an individual having a kidney cancer who may benefit from treatment with an anti-cancer therapy comprising a VEGF antagonist and a PD-L1 axis binding antagonist, the method comprising determining whether the individual has a sarcomatoid kidney cancer, wherein the presence of a sarcomatoid kidney cancer identifies the individual as one who may benefit from treatment with an anti-cancer therapy comprising a VEGF antagonist and a PD-L1 axis binding antagonist. In some embodiments, the method further comprises administering an effective amount of an anti-cancer therapy comprising a VEGF antagonist and a PD-L1 axis binding antagonist to the individual.

In another aspect, the invention features a method for selecting a therapy for an individual having a kidney cancer, the method comprising (a) determining whether the individual has a sarcomatoid kidney cancer, wherein the presence of a sarcomatoid kidney cancer identifies the individual as one who may benefit from treatment with an anti-cancer therapy comprising a VEGF antagonist and a PD-L1 axis binding antagonist; and (b) selecting an anti-cancer therapy comprising a VEGF antagonist and a PD-L1 axis binding antagonist based on the presence of a sarcomatoid kidney cancer. In some embodiments, the method further comprises administering an effective amount of an anti-cancer therapy comprising a VEGF antagonist and a PD-L1 axis binding antagonist to the individual.

In another aspect, the invention features a pharmaceutical composition comprising a PD-L1 axis binding antagonist for use in treatment of an individual having a sarcomatoid cancer (e.g., a sarcomatoid kidney cancer (e.g., a sarcomatoid RCC, including locally advanced or metastatic sarcomatoid RCC)), wherein the treatment comprises administration of the PD-L1 axis binding antagonist in combination with a VEGF antagonist. In some embodiments, the individual is previously untreated for the sarcomatoid cancer.

In another aspect, the invention provides for the use of a PD-L1 axis binding antagonist in the manufacture of a medicament for treatment of an individual having a sarcomatoid cancer (e.g., a sarcomatoid kidney cancer (e.g., a sarcomatoid RCC, including locally advanced or metastatic sarcomatoid RCC)), wherein the treatment comprises administration of the PD-L1 axis binding antagonist in combination with a VEGF antagonist. In some embodiments, the individual is previously untreated for the sarcomatoid cancer.

In another aspect, the invention features a pharmaceutical composition comprising a PD-L1 axis binding antagonist for use in treatment of an individual having a kidney cancer, wherein the treatment comprises administration of the PD-L1 axis binding antagonist in combination with a VEGF antagonist, wherein the individual is identified as likely to benefit from the anti-cancer therapy based on having a sarcomatoid kidney cancer.

In another aspect, the invention provides for the use of a PD-L1 axis binding antagonist in the manufacture of a medicament for treatment of an individual having a kidney cancer, wherein the treatment comprises administration of the PD-L1 axis binding antagonist in combination with a VEGF antagonist, wherein the individual is identified as likely to benefit from the anti-cancer therapy based on having a sarcomatoid kidney cancer.

In some embodiments of any of the preceding aspects, the presence of a sarcomatoid kidney cancer is assessed by histological analysis of a sample obtained from the individual. In some embodiments, the kidney cancer is sarcomatoid if a tumor sample from the individual contains a focus or foci of high-grade malignant spindle cells of any component relative to the entire tumor area. In some embodiments, the spindle cells show moderate to marked atypia and/or resemble any form of sarcoma.

In some embodiments, the spindle cells show evidence of epithelial differentiation as assessed by immunohistological positivity for keratin or epithelial membrane antigen (EMA). In some embodiments, the kidney cancer is renal cell carcinoma, and the tumor sample has epithelial differentiation with concurrent areas of renal cell carcinoma.

In some embodiments of any of the preceding aspects, the benefit is in terms of improved progression-free survival (PFS), overall survival (OS), overall response rate (ORR), complete response (CR) rate, or deterioration-free rate (DFR). In some embodiments, the benefit is in terms of improved PFS. In some embodiments, the benefit is in terms of improved OS. In some embodiments, the benefit is in terms of improved ORR. In some embodiments, the benefit is in terms of improved CR rate. In some embodiments, the benefit is in terms of improved DFR. In some embodiments, DFR is determined in terms of the time from onset of treatment to the individual's first increase of greater than or equal to 2 points above baseline on the MD Anderson Symptom Inventory (MDASI) interference scale.

In some embodiments of any of the preceding aspects, the individual has a poor or intermediate Memorial Sloan Kettering Cancer Center (MSKCC) risk score.

In another aspect, the invention features a method of treating an individual having a kidney cancer, the method comprising administering to the individual an effective amount of an anti-cancer therapy comprising a VEGF antagonist and a PD-L1 axis binding antagonist, wherein the individual has been identified as likely to benefit from the anti-cancer therapy based on the individual having a poor or intermediate MSKCC risk score.

In another aspect, the invention features a method of treating an individual having a kidney cancer, the method comprising: (a) determining the individual's MSKCC risk score, wherein a poor or intermediate MSKCC risk score indicates that the individual is likely to benefit from an anti-cancer therapy comprising a VEGF antagonist and a PD-L1 axis binding antagonist; and (b) administering an effective amount of an anti-cancer therapy comprising a VEGF antagonist and a PD-L1 axis binding antagonist to the individual based on the individual having a poor or intermediate MSKCC risk score.

In another aspect, the invention features a method of identifying an individual having a kidney cancer who may benefit from treatment with an anti-cancer therapy comprising a VEGF antagonist and a PD-L1 axis binding antagonist, the method comprising determining the individual's MSKCC risk score, wherein a poor or intermediate MSKCC risk score identifies the individual as likely to benefit from an anti-cancer therapy comprising a VEGF antagonist and a PD-L1 axis binding antagonist.

In another aspect, the invention features a method for selecting a therapy for an individual having a kidney cancer, the method comprising (a) determining the individual's MSKCC risk score, wherein a poor or intermediate MSKCC risk score identifies the individual as likely to benefit from an anti-cancer therapy comprising a VEGF antagonist and a PD-L1 axis binding antagonist; and (b) selecting an anti-cancer therapy comprising a VEGF antagonist and a PD-L1 axis binding antagonist based on the individual having a poor or intermediate MSKCC risk score.

In another aspect, the invention features a pharmaceutical composition comprising a PD-L1 axis binding antagonist for use in treatment of an individual having a kidney cancer, wherein the treatment comprises administration of the PD-L1 axis binding antagonist in combination with a VEGF antagonist, wherein the individual is identified as likely to benefit from the anti-cancer therapy based on having a poor or intermediate MSKCC risk score.

In another aspect, the invention provides for the use of a PD-L1 axis binding antagonist in the manufacture of a medicament for treatment of an individual having a kidney cancer, wherein the treatment comprises administration of the PD-L1 axis binding antagonist in combination with a VEGF antagonist, wherein the individual is identified as likely to benefit from the anti-cancer therapy based on having a poor or intermediate MSKCC risk score.

In another aspect, the invention features a pharmaceutical composition comprising a PD-L1 axis binding antagonist for use in treatment of an individual having a cancer (e.g., a kidney cancer (e.g., an RCC, including locally advanced or metastatic RCC)) with a poor or intermediate MSKCC risk score, wherein the treatment comprises administration of the PD-L1 axis binding antagonist in combination with a VEGF antagonist. In some embodiments, the individual is previously untreated for the cancer.

In another aspect, the invention provides for the use of a PD-L1 axis binding antagonist in the manufacture of a medicament for treatment of an individual having a cancer (e.g., a kidney cancer (e.g., an RCC, including locally advanced or metastatic RCC)) with a poor or intermediate MSKCC risk score, wherein the treatment comprises administration of the PD-L1 axis binding antagonist in combination with a VEGF antagonist. In some embodiments, the individual is previously untreated for the cancer.

In some embodiments of any of the preceding aspects, the individual has a poor MSKCC risk score if the individual has three or more of the following characteristics: (i) a time from nephrectomy to systemic treatment of less than one year, a lack of a nephrectomy, or an initial diagnosis with metastatic disease; (ii) a hemoglobin level less than the lower limit of normal (LLN), optionally wherein the normal range for hemoglobin is between 13.5 and 17.5 g/dL for men and between 12 and 15.5 g/dL for women; (iii) a serum corrected calcium level greater than 10 mg/dL, optionally wherein the serum corrected calcium level is the serum calcium level (mg/dL)+0.8(4−serum albumin (g/dL)); (iv) a serum lactate dehydrogenase (LDH) level greater than 1.5 times the upper limit of normal (ULN), optionally wherein the ULN is 140 U/L; and/or (v) a Karnofsky Performance Status (KPS) score of <80.

In some embodiments of any of the preceding aspects, the individual has an intermediate MSKCC risk score if the individual has one or two of the following characteristics: (i) a time from nephrectomy to systemic treatment of less than one year, a lack of a nephrectomy, or an initial diagnosis with metastatic disease; (ii) a hemoglobin level less than the LLN, optionally wherein the normal range for hemoglobin is between 13.5 and 17.5 g/dL for men and between 12 and 15.5 g/dL for women; (iii) a serum corrected calcium level greater than 10 mg/dL, optionally wherein the serum corrected calcium level is the serum calcium level (mg/dL)+0.8(4−serum albumin (g/dL)); (iv) a serum LDH level greater than 1.5 times the ULN, optionally wherein the ULN is 140 U/L; and/or (v) a KPS score of <80. In some embodiments of any of the preceding aspects, the benefit is in terms of improved progression-free survival (PFS), overall survival (OS), overall response rate (ORR), complete response (CR) rate, or deterioration-free rate (DFR). In some embodiments, the benefit is in terms of improved PFS. In some embodiments, the benefit is in terms of improved OS. In some embodiments, the benefit is in terms of improved ORR. In some embodiments, the benefit is in terms of improved CR rate. In some embodiments, the benefit is in terms of improved DFR. In some embodiments, DFR is determined in terms of the time from onset of treatment to the individual's first increase of greater than or equal to 2 points above baseline on the MD Anderson Symptom Inventory (MDASI) interference scale.

In some embodiments of any of the preceding aspects, the individual has a sarcomatoid kidney cancer.

In some embodiments of any of the preceding aspects, the method further comprises determining the expression level of one or more of the following genes in a sample from the individual: CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2; VEGFA, KDR, ESM1, PECAM1, FLT1, ANGPTL4, or CD34; or IL6, CXCL1, CXCL2, CXCL3, CXCL8, or PTGS2.

In some embodiments of any of the preceding aspects: (i) an expression level of one or more of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample that is at or above a reference expression level of the one or more genes; or (ii) an expression level of one or more of VEGFA, KDR, ESM1, PECAM1, FLT1, ANGPTL4, or CD34; or IL6, CXCL1, CXCL2, CXCL3, CXCL8, or PTGS2 in the sample that is below a reference expression level of the one or more genes identifies the individual as one who may benefit from treatment with an anti-cancer therapy comprising a VEGF antagonist and a PD-L1 axis binding antagonist.

In some embodiments of any of the preceding aspects, the expression level of one or more of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample is determined to be at or above a reference level of the one or more genes. In some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample is determined to be at or above a reference level of the one or more genes. In some embodiments, the expression level of one or more of CD8A, EOMES, PRF1, IFNG, or PD-L1 in the sample is determined to be at or above a reference level of the one or more genes. In some embodiments, the expression level of CD8A, EOMES, PRF1, IFNG, and PD-L1 in the sample is determined to be at or above a reference level of CD8A, EOMES, PRF1, IFNG, and PD-L1.

In some embodiments of any of the preceding aspects, the expression level of one or more of IL6, CXCL1, CXCL2, CXCL3, CXCL8, or PTGS2 in the sample is determined to be at or above a reference level of the one or more genes. In some embodiments, the expression level of at least one, at least two, at least three, at least four, at least five, or all six of IL6, CXCL1, CXCL2, CXCL3, CXCL8, or PTGS2 in the sample is determined to be at or above a reference level of the one or more genes. In some embodiments, the expression level of IL6, CXCL1, CXCL2, CXCL3, CXCL8, and PTGS2 in the sample is determined to be at or above a reference level of IL6, CXCL1, CXCL2, CXCL3, CXCL8, and PTGS2. In some embodiments, the expression level of PD-L1 in the sample is determined to be at or above a reference expression level of PD-L1, and the expression level of one or more additional genes selected from the group consisting of CD8A, EOMES, GZMA, GZMB, PRF1, IFNG, CXCL9, CXCL10, CXCL11, CD27, FOXP3, PD-1, CTLA4, TIGIT, IDO1, PSMB8, PSMB9, TAP1, or TAP2 in the sample is determined to be at or above a reference expression level of the one or more additional genes.

In some embodiments of any of the preceding aspects, the expression level of one or more of VEGFA, KDR, ESM1, PECAM1, FLT1, ANGPTL4, or CD34 in the sample is determined to be below a reference level of the one or more genes. In some embodiments, the expression level of at least one, at least two, at least three, at least four, at least five, at least six, or all seven of VEGFA, KDR, ESM1, PECAM1, FLT1, ANGPTL4, or CD34 in the sample is determined to be below a reference level of the one or more genes. In some embodiments, the expression level of one or more of VEGFA, KDR, ESM1, PECAM1, ANGPTL4, or CD34 in the sample is determined to be below a reference level of the one or more genes. In some embodiments, the expression level of VEGFA, KDR, ESM1, PECAM1, ANGPTL4, and CD34 in the sample is determined to be below a reference level of VEGFA, KDR, ESM1, PECAM1, ANGPTL4, and CD34.

In some embodiments of any of the preceding aspects, the expression level of one or more of IL6, CXCL1, CXCL2, CXCL3, CXCL8, or PTGS2 in the sample is determined to be below a reference level of the one or more genes. In some embodiments, the expression level of at least one, at least two, at least three, at least four, at least five, or all six of IL6, CXCL1, CXCL2, CXCL3, CXCL8, or PTGS2 in the sample is determined to be below a reference level of the one or more genes. In some embodiments, the expression level of IL6, CXCL1, CXCL2, CXCL3, CXCL8, and PTGS2 in the sample is determined to be below a reference level of IL6, CXCL1, CXCL2, CXCL3, CXCL8, and PTGS2.

In some embodiments of any of the preceding aspects, the reference level of the one or more genes is determined from a population of individuals having a kidney cancer. In some embodiments, the reference level of the one or more genes is a median expression level determined in a population of patients having a kidney cancer. In some embodiments, the reference level is a median of a Z-score of the normalized expression level of the one or more genes.

In some embodiments of any of the preceding aspects, the expression level is a nucleic acid expression level. In some embodiments, the nucleic acid expression level is an mRNA expression level. In some embodiments, the mRNA expression level is determined by RNA-seq, RT-qPCR, qPCR, multiplex qPCR or RT-qPCR, microarray analysis, SAGE, MassARRAY technique, ISH, or a combination thereof.

In other embodiments of any of the preceding aspects, the expression level is a protein expression level. In some embodiments, the protein expression level is determined by immunohistochemistry (IHC), Western blot, enzyme-linked immunosorbent assay (ELISA), immunoprecipitation, immunofluorescence, radioimmunoassay, or mass spectrometry.

In some embodiments of any of the preceding aspects, the sample is a tissue sample, a cell sample, a whole blood sample, a plasma sample, a serum sample, or a combination thereof. In some embodiments, the tissue sample is a tumor tissue sample. In some embodiments, the tumor tissue sample comprises tumor cells, tumor-infiltrating immune cells, stromal cells, or a combination thereof. In some embodiments, the tumor tissue sample is a formalin-fixed and paraffin-embedded (FFPE) sample, an archival sample, a fresh sample, or a frozen sample.

In some embodiments of any of the preceding aspects, the individual has not been previously treated for the kidney cancer.

In some embodiments of any of the preceding aspects, the kidney cancer is renal cell carcinoma (RCC). In some embodiments, the RCC is clear cell RCC. In some embodiments, the RCC is locally advanced or metastatic RCC (mRCC).

In some embodiments of any of the preceding aspects, a tumor sample obtained from the patient has been determined to have a detectable expression level of PD-L1 in tumor-infiltrating immune cells that comprise about 1% or more of the tumor sample. In some embodiments, the tumor sample has been determined to have a detectable expression level of PD-L1 in tumor-infiltrating immune cells that comprise about 1% or more to less than 5% of the tumor sample. In some embodiments, the tumor sample has been determined to have a detectable expression level of PD-L1 in tumor-infiltrating immune cells that comprise about 5% or more of the tumor sample. In some embodiments, the tumor sample has been determined to have a detectable expression level of PD-L1 in tumor-infiltrating immune cells that comprise about 5% or more to less than 10% of the tumor sample. In some embodiments, the tumor sample obtained from the patient has been determined to have a detectable expression level of PD-L1 in tumor-infiltrating immune cells that comprise about 10% or more of the tumor sample.

In other embodiments of any of the preceding aspects, a tumor sample obtained from the patient has been determined to have a detectable expression level of PD-L1 in tumor-infiltrating immune cells that comprise less than 1% of the tumor sample.

In some embodiments of any of the preceding aspects, the VEGF antagonist is an anti-VEGF antibody or a VEGF receptor (VEGFR) inhibitor. In some embodiments, the VEGF antagonist is an anti-VEGF antibody. In some embodiments, the anti-VEGF antibody is bevacizumab. In some embodiments, the VEGF antagonist is a VEGFR inhibitor. In some embodiments, the VEGFR inhibitor is a multi-targeted tyrosine kinase inhibitor. In some embodiments, the multi-targeted tyrosine kinase inhibitor is sunitinib, axitinib, pazopanib, or cabozantinib. In some embodiments, the multi-targeted tyrosine kinase inhibitor is sunitinib.

In some embodiments of any of the preceding aspects, the PD-L1 axis binding antagonist is selected from the group consisting of a PD-L1 binding antagonist, a PD-1 binding antagonist, and a PD-L2 binding antagonist. In some embodiments, the PD-L1 axis binding antagonist is a PD-L1 binding antagonist. In some embodiments, the PD-L1 binding antagonist inhibits the binding of PD-L1 to one or more of its ligand binding partners. In some embodiments, the PD-L1 binding antagonist inhibits the binding of PD-L1 to PD-1. In some embodiments, the PD-L1 binding antagonist inhibits the binding of PD-L1 to B7-1. In some embodiments, the PD-L1 binding antagonist inhibits the binding of PD-L1 to both PD-1 and B7-1. In some embodiments, the PD-L1 binding antagonist is an anti-PD-L1 antibody. In some embodiments, the anti-PD-L1 antibody is selected from the group consisting of: MPDL3280A (atezolizumab), YW243.55.S70, MDX-1105, MED14736 (durvalumab), and MSB0010718C (avelumab). In some embodiments, the anti-PD-L1 antibody comprises the following hypervariable regions (HVRs): (a) an HVR-H1 sequence of GFTFSDSWIH (SEQ ID NO: 63); (b) an HVR-H2 sequence of AWISPYGGSTYYADSVKG (SEQ ID NO: 64); (c) an HVR-H3 sequence of RHWPGGFDY (SEQ ID NO: 65); (d) an HVR-L1 sequence of RASQDVSTAVA (SEQ ID NO: 66); (e) an HVR-L2 sequence of SASFLYS (SEQ ID NO: 67); and (f) an HVR-L3 sequence of QQYLYHPAT (SEQ ID NO: 68). In some embodiments, the anti-PD-L1 antibody comprises: (a) a heavy chain variable (VH) domain comprising an amino acid sequence having at least 90% sequence identity to the amino acid sequence of EVQLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO: 69); (b) a light chain variable (VL) domain comprising an amino acid sequence having at least 90% sequence identity to the amino acid sequence of DIQMTQSPSSLSASVGDRVTITCRASQDVSTAVAWYQQKPGKAPKLLIYSASFLYSGVPSRFSGSGSGTD FTLTISSLQPEDFATYYCQQYLYHPATFGQGTKVEIKR (SEQ ID NO: 70); or (c) a VH domain as in (a) and a VL domain as in (b). In some embodiments, the anti-PD-L1 antibody comprises: (a) a VH domain comprising an amino acid sequence having at least 95% sequence identity to the amino acid sequence of SEQ ID NO: 69; (b) a VL domain comprising an amino acid sequence having at least 95% sequence identity to the amino acid sequence of SEQ ID NO: 70; or (c) a VH domain as in (a) and a VL domain as in (b). In some embodiments, the anti-PD-L1 antibody comprises: (a) a VH domain comprising an amino acid sequence having at least 96% sequence identity to the amino acid sequence of SEQ ID NO: 69; (b) a VL domain comprising an amino acid sequence having at least 96% sequence identity to the amino acid sequence of SEQ ID NO: 70; or (c) a VH domain as in (a) and a VL domain as in (b). In some embodiments, the anti-PD-L1 antibody comprises: (a) a VH domain comprising an amino acid sequence having at least 97% sequence identity to the amino acid sequence of SEQ ID NO: 69; (b) a VL domain comprising an amino acid sequence having at least 97% sequence identity to the amino acid sequence of SEQ ID NO: 70; or (c) a VH domain as in (a) and a VL domain as in (b). In some embodiments, the anti-PD-L1 antibody comprises: (a) a VH domain comprising an amino acid sequence having at least 98% sequence identity to the amino acid sequence of SEQ ID NO: 69; (b) a VL domain comprising an amino acid sequence having at least 98% sequence identity to the amino acid sequence of SEQ ID NO: 70; or (c) a VH domain as in (a) and a VL domain as in (b). In some embodiments, the anti-PD-L1 antibody comprises: (a) a VH domain comprising an amino acid sequence having at least 99% sequence identity to the amino acid sequence of SEQ ID NO: 69; (b) a VL domain comprising an amino acid sequence having at least 99% sequence identity to the amino acid sequence of SEQ ID NO: 70; or (c) a VH domain as in (a) and a VL domain as in (b). In some embodiments, the anti-PD-L1 antibody comprises: (a) a VH domain comprising the amino acid sequence of SEQ ID NO: 69; (b) a VL domain comprising the amino acid sequence of SEQ ID NO: 70; or (c) a VH domain as in (a) and a VL domain as in (b). In some embodiments, the anti-PD-L1 antibody comprises: (a) a VH domain comprising the amino acid sequence of SEQ ID NO: 69; and (b) a VL domain comprising the amino acid sequence of SEQ ID NO: 70. In some embodiments, the anti-PD-L1 antibody is atezolizumab.

In some embodiments of any of the preceding aspects, the PD-L1 axis binding antagonist is atezolizumab and the VEGF antagonist is bevacizumab. In some embodiments, the atezolizumab is administered intravenously every three weeks at a dose of about 1200 mg. In some embodiments, the bevacizumab is administered intravenously every three weeks at a dose of about 15 mg/kg.

In some embodiments of any of the preceding aspects, the method further comprises administering an additional therapeutic agent to the individual. In some embodiments, the additional therapeutic agent is selected from the group consisting of an immunotherapy agent, a cytotoxic agent, a growth inhibitory agent, a radiation therapy agent, an anti-angiogenic agent, and combinations thereof. In some embodiments, the individual is a human.

### BRIEF DESCRIPTION OF THE DRAWINGS

FIG. 1 is a schematic diagram showing the IMmotion151 study design. The co-primary endpoints were progression-free survival (PFS) (investigator-assessed PFS per RECIST v1.1) in the PD-L1+ subgroup and overall survival (OS) in the intent-to-treat (ITT) population. Exploratory endpoints included validation of gene signatures from the IMmotion150 study and their association with PFS, as well as biomarker characterization in Memorial Sloan Kettering Cancer Center (MSKCC) risk subgroups and sarcomatoid tumors. ^{a}≥1% IC: 40% prevalence using the SP142 immunohistochemistry (IHC) assay; ^{b} No dose reduction for atezolizumab or bevacizumab.

FIG. 2 is a series of graphs showing Kaplan-Meier curves showing probability of PFS in the PD-L1+ subgroup (left panel) and in the ITT population (right panel) for patients treated with atezolizumab and bevacizumab (“Atezo+Bev”) or sunitinib. The table shows median PFS (months) as well as the 95% confidence intervals (95% CI). PFS was assessed by investigators. Minimum follow-up, 12 months. Median follow-up, 16 months (PD-L1+) and 15 months (ITT). ^{a} The PFS analysis passed the pre-specified P value boundary of α=0.04.

FIG. 3 is a schematic diagram showing the gene signature analysis scheme for the IMmotion151 study.

FIG. 4 is a heatmap showing that the IMmotion151 transcriptome map confirmed biological subgroups identified in the IMmotion150 study.

FIG. 5 is a series of graphs showing Kaplan-Meier curves showing probability of PFS in the Angiogenesis (Angio)^{Low} (left panel) or Angio^{High} (right panel) subgroups for patients treated with Atezo+Bev or sunitinib. Atezo+Bev improved PFS versus sunitinib in the Angio^{Low} subgroup. The table shows the hazard ratios (HRs) and 95% Cl.

FIG. 6 is a series of graphs showing Kaplan-Meier curves showing probability of PFS for patients treated with sunitinib (left panel) or Atezo+Bev (right panel). Sunitinib demonstrated improved PFS in the Angio^{High} subgroup versus the Angio^{Low} subgroup. The table shows the hazard ratios (HRs) and 95% CI.

FIG. 7 is a series of graphs showing Kaplan-Meier curves showing probability of PFS in the T-effector (Teff)^{Low} (left panel) or Teff^{High} (right panel) subgroups for patients treated with Atezo+Bev or sunitinib. Atezo+Bev improved PFS versus sunitinib in the Teff^{High} subgroup. The table shows the hazard ratios (HRs) and 95% CI.

FIG. 8A is a graph showing the results of subgroup PFS analysis in PD-L1+ and all evaluable patients (in the biomarker evaluable population).

FIG. 8B is a graph showing Kaplan-Meier curves showing probability of PFS for patients treated with sunitinib or Atezo+Bev. Atezo+Bev treatment demonstrated improved PFS in sarcomatoid tumors. The table shows the HR and 95% CI.

FIGS. 9A-9C are a series of graphs showing expression of the Angio gene signature (FIG. 9A), the Teff gene signature (FIG. 9B), and PD-L1 (FIG. 9C) in the sarcomatoid and non-sarcomatoid subgroups. Expression of the Angio gene signature was lower and PD-L1 expression was higher in sarcomatoid tumors.

FIGS. 10A-10C are a series of graphs showing expression of the Angio signature (FIG. 10A), the Teff signature (FIG. 10B), and PD-L1 (FIG. 100) in the favorable or intermediate/poor MSKCC risk subgroups. Expression of the Angio gene signature was higher in the favorable MSKCC risk group.

FIGS. 11A and 11B are a series of graphs showing Kaplan-Meier curves showing probability of PFS for patients treated with Atezo+Bev or sunitinib for all patients with sarcomatoid tumors (“All Sarc”) (FIG. 11A) or PD-L1+ tumors (“PD-L1+ Sarc”) (FIG. 11B). Patients with sarcomatoid histology in the Atezo+Bev arm had a longer median PFS than those in the sunitinib arm, regardless of PD-L1+ status.

FIGS. 12A and 12B are a series of graphs showing Kaplan-Meier curves showing probability of overall survival (OS) for patients treated with Atezo+Bev or sunitinib for all patients with sarcomatoid tumors (“All Sarc”) (FIG. 12A) or PD-L1+ tumors (“PD-L1+ Sarc”) (FIG. 12B). OS was increased in patients with sarcomatoid histology treated with Atezo+Bev versus those treated with sunitinib, regardless of PD-L1+ status.

FIG. 13 is a graph showing time to deteriorations: symptom interference with daily function^{b} in all patients with sarcomatoid tumors. DFR, deterioration-free rate. ^{a}Time to clinically meaningful deterioration pre-specified as the time from randomization to a patient's first ≥2-point increase above baseline on the MD Anderson Symptom Inventory (MDASI) interference scale (range, 0 to 10) (see, e.g., Mendoza et al. Clin. Breast Cancer 13:325-334, 2013; Jones et al. Clin. Genitourin. Cancer 12:41-49, 2014; and Shi et al. Pain 158:1108-1112, 2017). ^{b}Dimensions of daily function include work, general activity, walking, relations with others, enjoyment of life, and mood.

### DETAILED DESCRIPTION OF THE INVENTION

The present invention provides diagnostic methods, therapeutic methods and uses, and compositions for the treatment of cancer (e.g., a kidney cancer (e.g., a renal cell carcinoma (RCC)), including sarcomatoid cancer. The invention is based, at least in part, on the discovery that the presence of a sarcomatoid cancer (e.g., a sarcomatoid kidney cancer such as sarcomatoid RCC) and/or an individual's Memorial Sloan Kettering Cancer Center (MSKCC) risk score can be used as a biomarker (e.g., a predictive biomarker) in methods of identifying whether the individual is likely to respond to treatment including a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A)) or a PD-1 binding antagonist (e.g., an anti-PD-1 antibody)); selecting a therapy for treating the individual; optimizing therapeutic efficacy of a treatment that includes a VEGF antagonist and a PD-L1 axis binding antagonist; and/or monitoring the response of the individual to a treatment that includes a VEGF antagonist and a PD-L1 axis binding antagonist. The invention also provides methods for treating an individual having a cancer (e.g., a kidney cancer (e.g., a renal cell carcinoma (RCC)) by administering an anti-cancer therapy that includes a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A)) or a PD-1 binding antagonist (e.g., an anti-PD-1 antibody)).

### I. DEFINITIONS

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se.

As used herein, the terms “individual,” “patient,” or “subject” are used interchangeably and refer to any single animal, more preferably a mammal (including such non-human animals as, for example, cats, dogs, horses, rabbits, zoo animals, cows, pigs, sheep, and non-human primates) for which treatment is desired. In particular embodiments, the patient herein is a human. The patient may be a “cancer patient,” i.e., one who is suffering from cancer (e.g., kidney cancer (e.g., RCC)), or at risk for suffering from cancer, or suffering from one or more symptoms of cancer.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include, but are not limited to, kidney or renal cancer (e.g., renal cell carcinoma (RCC)); lung cancer, including small-cell lung cancer, non-small cell lung cancer, adenocarcinoma of the lung, and squamous carcinoma of the lung; bladder cancer (e.g., urothelial bladder cancer (UBC), muscle invasive bladder cancer (MIBC), and BCG-refractory non-muscle invasive bladder cancer (NMIBC)); cancer of the urinary tract; breast cancer (e.g., HER2+ breast cancer and triple-negative breast cancer (TNBC), which are estrogen receptors (ER−), progesterone receptors (PR−), and HER2 (HER2−) negative); prostate cancer, such as castration-resistant prostate cancer (CRPC); cancer of the peritoneum; hepatocellular cancer; gastric or stomach cancer, including gastrointestinal cancer and gastrointestinal stromal cancer; pancreatic cancer; glioblastoma; cervical cancer; ovarian cancer; liver cancer (e.g., hepatocellular carcinoma (HCC)); hepatoma; colon cancer; rectal cancer; colorectal cancer; endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases. In some embodiments, the cancer is kidney cancer. In particular embodiments, the kidney cancer is RCC (e.g., advanced RCC or metastatic RCC (mRCC), including previously untreated RCC). In some embodiments, the kidney cancer is sarcomatoid kidney cancer (e.g., sarcomatoid RCC (e.g., sarcomatoid advanced or mRCC)).

By “early stage cancer” or “early stage tumor” is meant a cancer that is not invasive or metastatic or is classified as a Stage 0, I, or II cancer.

An “advanced” cancer is one which has spread outside the site or organ of origin, either by local invasion or metastasis.

A “refractory” cancer is one which progresses even though an anti-tumor agent, such as a chemotherapeutic agent, is being administered to the cancer patient. An example of a refractory cancer is one which is platinum refractory.

A “recurrent” cancer is one which has regrown, either at the initial site or at a distant site, after a response to initial therapy.

The terms “cell proliferative disorder” and “proliferative disorder” refer to disorders that are associated with some degree of abnormal cell proliferation. In one embodiment, the cell proliferative disorder is cancer.

The term “tumor,” as used herein, refers to all neoplastic cell growth and proliferation, whether malignant or benign, and all pre-cancerous and cancerous cells and tissues.

The terms “cancer,” “cancerous,” “cell proliferative disorder,” “proliferative disorder,” and “tumor” are not mutually exclusive as referred to herein.

A “disorder” is any condition that would benefit from treatment including, but not limited to, chronic and acute disorders or diseases including those pathological conditions which predispose the mammal to the disorder in question.

The term “sarcomatoid” refers to a cancer (e.g., a kidney cancer (e.g., an RCC)) that is characterized by sarcomatoid morphology, for example, as assessed by histology. Sarcomatoid kidney cancer (e.g., sarcomatoid RCC) is associated with aggressive behavior and poor prognosis. In some embodiments, a sarcomatoid kidney cancer includes or consists of atypical spindle-shaped cells and/or resembles any form of sarcoma. See, e.g., El Mouallem et al. Urol. Oncol. 36:265-271, 2018, which is incorporated herein by reference in its entirety. Sarcomatoid RCC can occur in any subtype of RCC, including clear cell RCC, chromophobe RCC, collecting duct carcinoma, renal medullary carcinoma, fumarate hydratase (FH)-deficient RCC, and succinate dehydrogenase (SDH)-deficient RCC. The incidence of sarcomatoid RCC varies among subtypes, but is typically higher in clear cell RCC (approximately 5-8%) and chromophobe RCC (approximately 8-10%). The histology of the sarcomatoid component can be variable, and may include a fibrosarcoma-like pattern, a pleomorphic undifferentiated sarcoma-like pattern, or other heterologous sarcomatoid patterns (e.g., osteosarcoma-, chondrosarcoma-, or rhabdomyosarcoma-like patterns). Necrosis is typically present in a large majority (about 90%) of cases. In some embodiments, there is no minimum amount or percentage of sarcomatoid differentiation for an individual's kidney cancer to be classified as sarcomatoid. Sarcomatoid RCC may be assessed as described in Example 1. In other embodiments, sarcomatoid RCC may be characterized as described by the 2012 International Society of Urological Pathology (ISUP) Vancouver consensus (see Srigley et al. Am. J. Surg. Pathol. 37:1469-89, 2013, which is incorporated herein by reference in its entirety).

The term “Memorial Sloan Kettering Cancer Center (MSKCC) risk score” refers to a scoring system based on set of prognostic factors associated with survival in kidney cancer (e.g., RCC, e.g., mRCC) patients. See, e.g., Motzer et al. J. Clin. Oncol. 17(8):2530-2540, 1999 and Motzer et al. J. Clin. Oncol. 20(1):289-296, 2002, which are incorporated herein by reference in their entirety. In some embodiments, a MSKCC risk score can be calculated based on the following factors, as described in Example 1: (i) a time from nephrectomy to treatment (e.g., systemic treatment) of less than one year, a lack of a nephrectomy, or an initial diagnosis with metastatic disease; (ii) a hemoglobin level less than the lower limit of normal (LLN), optionally wherein the normal range for hemoglobin is between 13.5 and 17.5 g/dL for men and between 12 and 15.5 g/dL for women; (iii) a serum corrected calcium level greater than 10 mg/dL, optionally wherein the serum corrected calcium level is the serum calcium level (mg/dL)+0.8(4−serum albumin (g/dL)); (iv) a serum lactate dehydrogenase (LDH) level greater than 1.5 times the upper limit of normal (ULN), optionally wherein the ULN is 140 U/L; and/or (v) a Karnofsky Performance Status (KPS) score of <80. In some embodiments, an individual has a favorable MSKCC risk score if the individual has zero of the preceding characteristics. In some embodiments, an individual has an intermediate MSKCC risk score if the individual has one or two of the preceding characteristics. In some embodiments, an individual has a poor MSKCC risk score if the individual has three or more of the preceding characteristics. In some embodiments, an individual's MSKCC risk score may be used to identify whether the individual may benefit from an anti-cancer therapy, e.g., an anti-cancer therapy that includes a VEGF antagonist (e.g., an anti-VEGF antibody such as bevacizumab) and a PD-L1 axis binding antagonist (e.g., an anti-PD-L1 antibody such as atezolizumab).

The term “detection” includes any means of detecting, including direct and indirect detection.

The term “sample,” as used herein, refers to a composition that is obtained or derived from a patient and/or individual of interest that contains a cellular and/or other molecular entity that is to be characterized and/or identified, for example, based on physical, biochemical, chemical, and/or physiological characteristics. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The terms “biomarker” and “marker” are used interchangeably herein to refer to a DNA, RNA, protein, carbohydrate, glycolipid, cell-based molecular marker, histological or morphological marker (e.g., sarcomatoid morphology), or risk score (e.g., an MSKCC risk score), the expression, presence, and/or level of which in a patient's sample can be detected by standard methods (or methods disclosed herein).

Such markers include the presence of sarcomatoid kidney cancer (e.g., sarcomatoid RCC) and/or the individual's MSKCC risk score (e.g., a poor or intermediate MSKCC risk score). Such biomarkers also include, but are not limited to, CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and/or S100A9. The presence, expression, and/or level of such a biomarker may be determined to be higher or lower in a sample obtained from a patient sensitive or responsive to a treatment (e.g., treatment with an anti-cancer therapy that includes a VEGF antagonist and a PD-L1 axis binding antagonist, or treatment with a multi-targeted tyrosine kinase inhibitor) than a reference level (including, e.g., the median expression level of the biomarker in a sample from a group/population of patients, e.g., patients having cancer, and being tested for responsiveness to a treatment; the median expression level of the biomarker in a sample from a group/population of patients, e.g., patients having cancer, and identified as not responding to a treatment; the level in a sample previously obtained from the individual at a prior time; or the level in a sample from a patient who received prior treatment (e.g., with an anti-cancer therapy that includes a VEGF antagonist and a PD-L1 axis binding antagonist, or treatment with a multi-targeted tyrosine kinase inhibitor) in a primary tumor setting, and who now may be experiencing metastasis).

The term “CD8A” as used herein, refers to any native CD8A from any vertebrate source, including mammals such as primates (e.g., humans) and rodents (e.g., mice and rats), unless otherwise indicated. The term encompasses “full-length,” unprocessed CD8A as well as any form of CD8A that results from processing in the cell. The term also encompasses naturally occurring variants of CD8A, e.g., splice variants or allelic variants. The nucleic acid sequence of an exemplary human CD8A is set forth in SEQ ID NO: 1. The amino acid sequence of an exemplary protein encoded by human CD8A is shown in SEQ ID NO: 2.

The term “EOMES” as used herein, refers to any native EOMES (Eomesodermin) from any vertebrate source, including mammals such as primates (e.g., humans) and rodents (e.g., mice and rats), unless otherwise indicated. The term encompasses “full-length,” unprocessed EOMES as well as any form of EOMES that results from processing in the cell. The term also encompasses naturally occurring variants of EOMES, e.g., splice variants or allelic variants. The nucleic acid sequence of an exemplary human EOMES is set forth in SEQ ID NO: 3. The amino acid sequence of an exemplary protein encoded by human EOMES is shown in SEQ ID NO: 4.

The term “GZMA” as used herein, refers to any native GZMA (Granzyme A) from any vertebrate source, including mammals such as primates (e.g., humans) and rodents (e.g., mice and rats), unless otherwise indicated. The term encompasses “full-length,” unprocessed GZMA as well as any form of GZMA that results from processing in the cell. The term also encompasses naturally occurring variants of GZMA, e.g., splice variants or allelic variants. The nucleic acid sequence of an exemplary human GZMA is set forth in SEQ ID NO: 51. The amino acid sequence of an exemplary protein encoded by human GZMA is shown in SEQ ID NO: 52.

The term “GZMB” as used herein, refers to any native GZMB (Granzyme B) from any vertebrate source, including mammals such as primates (e.g., humans) and rodents (e.g., mice and rats), unless otherwise indicated. The term encompasses “full-length,” unprocessed GZMB as well as any form of GZMB that results from processing in the cell. The term also encompasses naturally occurring variants of GZMB, e.g., splice variants or allelic variants. The nucleic acid sequence of an exemplary human GZMB is set forth in SEQ ID NO: 53. The amino acid sequence of an exemplary protein encoded by human GZMB is shown in SEQ ID NO: 54.

The term “PRF1” as used herein, refers to any native PRF1 (Perforin 1; also known as Pore Forming Protein) from any vertebrate source, including mammals such as primates (e.g., humans) and rodents (e.g., mice and rats), unless otherwise indicated. The term encompasses “full-length,” unprocessed PRF1 as well as any form of PRF1 that results from processing in the cell. The term also encompasses naturally occurring variants of PRF1, e.g., splice variants or allelic variants. The nucleic acid sequence of an exemplary human PRF1 is set forth in SEQ ID NO: 5. The amino acid sequence of an exemplary protein encoded by human PRF1 is shown in SEQ ID NO: 6.

The term “IFNG” as used herein, refers to any native IFNG (Interferon, Gamma) from any vertebrate source, including mammals such as primates (e.g., humans) and rodents (e.g., mice and rats), unless otherwise indicated. The term encompasses “full-length,” unprocessed IFNG as well as any form of IFNG that results from processing in the cell. The term also encompasses naturally occurring variants of IFNG, e.g., splice variants or allelic variants. The nucleic acid sequence of an exemplary human IFNG is set forth in SEQ ID NO: 7. The amino acid sequence of an exemplary protein encoded by human IFNG is shown in SEQ ID NO: 8.

The terms “Programmed Death Ligand 1” and “PD-L1” refer herein to a native sequence PD-L1 polypeptide, polypeptide variants, and fragments of a native sequence polypeptide and polypeptide variants (which are further defined herein). The PD-L1 polypeptide described herein may be that which is isolated from a variety of sources, such as from human tissue types or from another source, or prepared by recombinant or synthetic methods.

A “native sequence PD-L1 polypeptide” comprises a polypeptide having the same amino acid sequence as the corresponding PD-L1 polypeptide derived from nature. The term encompasses “full-length,” unprocessed PD-L1 as well as any form of IFNG that results from processing in the cell. The term also encompasses naturally occurring variants of IFNG, e.g., splice variants or allelic variants.

A “PD-L1 polypeptide variant,” or variations thereof, means a PD-L1 polypeptide, generally an active PD-L1 polypeptide, as defined herein having at least about 80% amino acid sequence identity with any of the native sequence PD-L1 polypeptide sequences as disclosed herein. Such PD-L1 polypeptide variants include, for instance, PD-L1 polypeptides wherein one or more amino acid residues are added, or deleted, at the N- or C-terminus of a native amino acid sequence. Ordinarily, a PD-L1 polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a native sequence PD-L1 polypeptide sequence as disclosed herein. Ordinarily, PD-L1 variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 281, 282, 283, 284, 285, 286, 287, 288, or 289 amino acids in length, or more. Optionally, PD-L1 variant polypeptides will have no more than one conservative amino acid substitution as compared to a native PD-L1 polypeptide sequence, alternatively no more than 2, 3, 4, 5, 6, 7, 8, 9, or 10 conservative amino acid substitutions as compared to the native PD-L1 polypeptide sequence.

The term “vascular endothelial growth factor” or “VEGF” refers to vascular endothelial growth factor protein A (VEGFA), as exemplified by Swiss Prot Accession Number P15692, Gene ID (NCBI): 7422. The term “VEGF” encompasses the protein having the amino acid sequence of Swiss Prot Accession Number P15692, Gene ID (NCBI): 7422 as well as homologues and isoforms thereof. The term “VEGF” also encompasses the known isoforms, e.g., splice isoforms, of VEGF, e.g., VEGF₁₁₁, VEGF₁₂₁, VEGF₁₄₅, VEGF₁₆₅, VEGF₁₈₉, and VEGF₂₀₆, together with the naturally-occurring allelic and processed forms thereof, including the 110 amino acid human vascular endothelial cell growth factor generated by plasmin cleavage of VEGF₁₆₅ as described in Ferrara Mol. Biol. Cell. 21:687, 2010; Leung et al., Science, 246:1306. 1989; and Houck et al., Mol. Endocrin., 5:1806, 1991. The term “VEGF” also refers to VEGFs from non-human species such as mouse, rat or primate. Sometimes the VEGF from a specific species are indicated by terms such as hVEGF for human VEGF, mVEGF for murine VEGF, and the like. The term “VEGF” is also used to refer to truncated forms of the polypeptide comprising amino acids 8 to 109 or 1 to 109 of the 165-amino acid human vascular endothelial cell growth factor. Reference to any such forms of VEGF may be identified in the present application, e.g., by “VEGFios,” “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF. The term “VEGF variant” as used herein refers to a VEGF polypeptide which includes one or more amino acid mutations in the native VEGF sequence. Optionally, the one or more amino acid mutations include amino acid substitution(s). For purposes of shorthand designation of VEGF variants described herein, it is noted that numbers refer to the amino acid residue position along the amino acid sequence of the putative native VEGF (provided in Leung et al., supra and Houck et al., supra). Unless specified otherwise, the term “VEGF” as used herein indicates VEGF-A.

The term “Kinase Insert Domain Receptor” or “KDR” as used herein, refers to any native KDR (also known in the art as Fetal Liver Kinase 1 (FLK1) or Vascular Endothelial Growth Factor Receptor 2 (VEGFR2)) from any vertebrate source, including mammals such as primates (e.g., humans) and rodents (e.g., mice and rats), unless otherwise indicated. The term encompasses “full-length,” unprocessed KDR as well as any form of KDR that results from processing in the cell. The term also encompasses naturally occurring variants of KDR, e.g., splice variants or allelic variants. The nucleic acid sequence of an exemplary human KDR is set forth in SEQ ID NO: 9. The amino acid sequence of an exemplary protein encoded by human KDR is shown in SEQ ID NO: 10.

The term “Endothelial Cell Specific Molecule 1” or “ESM1” as used herein, refers to any native ESM1 (also known in the art as endocan) from any vertebrate source, including mammals such as primates (e.g., humans) and rodents (e.g., mice and rats), unless otherwise indicated. The term encompasses “full-length,” unprocessed ESM1 as well as any form of ESM1 that results from processing in the cell. The term also encompasses naturally occurring variants of ESM1, e.g., splice variants or allelic variants. The nucleic acid sequence of an exemplary human ESM1 is set forth in SEQ ID NO: 11. The amino acid sequence of an exemplary protein encoded by human ESM1 is shown in SEQ ID NO: 12.

The term “Platelet And Endothelial Cell Adhesion Molecule 1” or “PECAM1” as used herein, refers to any native PECAM1 (also known in the art as CD31, endoCAM, GPIIA, or PECA1) from any vertebrate source, including mammals such as primates (e.g., humans) and rodents (e.g., mice and rats), unless otherwise indicated. The term encompasses “full-length,” unprocessed PECAM1 as well as any form of PECAM1 that results from processing in the cell. The term also encompasses naturally occurring variants of PECAM1, e.g., splice variants or allelic variants. The nucleic acid sequence of an exemplary human PECAM1 is set forth in SEQ ID NO: 13. The amino acid sequence of an exemplary protein encoded by human PECAM1 is shown in SEQ ID NO: 14.

The term “FLT1” as used herein, refers to any native FLT1 (also known in the art as Vascular Endothelial Growth Factor Receptor 1 (VEGFR1) or fms related tyrosine kinase 1) from any vertebrate source, including mammals such as primates (e.g., humans) and rodents (e.g., mice and rats), unless otherwise indicated. The term encompasses “full-length,” unprocessed FLT1 as well as any form of FLT1 that results from processing in the cell. The term also encompasses naturally occurring variants of FLT1, e.g., splice variants or allelic variants. The nucleic acid sequence of an exemplary human FLT1 is set forth in SEQ ID NO: 55. The amino acid sequence of an exemplary protein encoded by human FLT1 is shown in SEQ ID NO: 56.

The term “Angiopoietin Like 4” or “ANGPTL4” as used herein, refers to any native ANGPTL4 (also known in the art as Hepatic Fibrinogen/Angiopoietin-Related Protein (HFARP), Peroxisome Proliferator-Activated Receptor (PPAR) Gamma, Hepatic Angiopoietin-Related Protein (HARP), Angiopoietin-Related Protein 4 (Arp4), or Fasting-Induced Adipose Factor (FIAF)) from any vertebrate source, including mammals such as primates (e.g., humans) and rodents (e.g., mice and rats), unless otherwise indicated. The term encompasses “full-length,” unprocessed ANGPTL4 as well as any form of ANGPTL4 that results from processing in the cell. The term also encompasses naturally occurring variants of ANGPTL4, e.g., splice variants or allelic variants. The nucleic acid sequence of an exemplary human ANGPTL4 is set forth in SEQ ID NO: 15. The amino acid sequence of an exemplary protein encoded by human ANGPTL4 is shown in SEQ ID NO: 16.

The term “CD34” as used herein, refers to any native CD34 (also known in the art as CD34 molecule or CD34 antigen) from any vertebrate source, including mammals such as primates (e.g., humans) and rodents (e.g., mice and rats), unless otherwise indicated. The term encompasses “full-length,” unprocessed CD34 as well as any form of CD34 that results from processing in the cell. The term also encompasses naturally occurring variants of CD34, e.g., splice variants or allelic variants. The nucleic acid sequence of an exemplary human CD34 is set forth in SEQ ID NO: 17. The amino acid sequence of an exemplary protein encoded by human CD34 is shown in SEQ ID NO: 18.

The term “interleukin 6” or “IL6” as used herein, refers to any native IL6 from any vertebrate source, including mammals such as primates (e.g., humans) and rodents (e.g., mice and rats), unless otherwise indicated. The term encompasses “full-length,” unprocessed IL6 as well as any form of IL6 that results from processing in the cell. The term also encompasses naturally occurring variants of IL6, e.g., splice variants or allelic variants. The nucleic acid sequence of an exemplary human IL6 is set forth in SEQ ID NO: 19. The amino acid sequence of an exemplary protein encoded by human IL6 is shown in SEQ ID NO: 20.

The term “CXCL1” as used herein, refers to any native CXCL1 (chemokine (C-X-C motif) ligand 1; also known as GRO1 or neutrophil-activating protein 3 (NAP-3)) from any vertebrate source, including mammals such as primates (e.g., humans) and rodents (e.g., mice and rats), unless otherwise indicated. The term encompasses “full-length,” unprocessed CXCL1 as well as any form of CXCL1 that results from processing in the cell. The term also encompasses naturally occurring variants of CXCL1, e.g., splice variants or allelic variants. The nucleic acid sequence of an exemplary human CXCL1 is set forth in SEQ ID NO: 21. The amino acid sequence of an exemplary protein encoded by human CXCL1 is shown in SEQ ID NO: 22.

The term “CXCL2” as used herein, refers to any native CXCL2 (chemokine (C-X-C motif) ligand 2; also known as macrophage inflammatory protein 2-alpha (MIP2-alpha)) from any vertebrate source, including mammals such as primates (e.g., humans) and rodents (e.g., mice and rats), unless otherwise indicated. The term encompasses “full-length,” unprocessed CXCL2 as well as any form of CXCL2 that results from processing in the cell. The term also encompasses naturally occurring variants of CXCL2, e.g., splice variants or allelic variants. The nucleic acid sequence of an exemplary human CXCL2 is set forth in SEQ ID NO: 23. The amino acid sequence of an exemplary protein encoded by human CXCL2 is shown in SEQ ID NO: 24.

The term “CXCL3” as used herein, refers to any native CXCL3 (chemokine (C-X-C motif) ligand 3; also known as macrophage inflammatory protein 2-beta (MIP2-beta)) from any vertebrate source, including mammals such as primates (e.g., humans) and rodents (e.g., mice and rats), unless otherwise indicated. The term encompasses “full-length,” unprocessed CXCL3 as well as any form of CXCL3 that results from processing in the cell. The term also encompasses naturally occurring variants of CXCL3, e.g., splice variants or allelic variants. The nucleic acid sequence of an exemplary human CXCL3 is set forth in SEQ ID NO: 25. The amino acid sequence of an exemplary protein encoded by human CXCL3 is shown in SEQ ID NO: 26.

The term “CXCL8” as used herein, refers to any native CXCL8 (chemokine (C-X-C motif) ligand 8; also known as interleukin 8 (IL8)) from any vertebrate source, including mammals such as primates (e.g., humans) and rodents (e.g., mice and rats), unless otherwise indicated. The term encompasses “full-length,” unprocessed CXCL8 as well as any form of CXCL8 that results from processing in the cell. The term also encompasses naturally occurring variants of CXCL8, e.g., splice variants or allelic variants. The nucleic acid sequence of an exemplary human CXCL8 is set forth in SEQ ID NO: 27. The amino acid sequence of an exemplary protein encoded by human CXCL8 is shown in SEQ ID NO: 28.

The term “PTGS2” as used herein, refers to any native PTGS2 (prostaglandin-endoperoxide synthase 2; also known as cyclooxygenase-2 (COX-2)) from any vertebrate source, including mammals such as primates (e.g., humans) and rodents (e.g., mice and rats), unless otherwise indicated. The term encompasses “full-length,” unprocessed PTGS2 as well as any form of PTGS2 that results from processing in the cell. The term also encompasses naturally occurring variants of PTGS2, e.g., splice variants or allelic variants. The nucleic acid sequence of an exemplary human PTGS2 is set forth in SEQ ID NO: 29. The amino acid sequence of an exemplary protein encoded by human PTGS2 is shown in SEQ ID NO: 30.

The term “CXCR1” as used herein, refers to any native CXCR1 (C-X-C motif chemokine receptor 1; also known as interleukin 8 receptor, alpha, IL8RA, and CD181) from any vertebrate source, including mammals such as primates (e.g., humans) and rodents (e.g., mice and rats), unless otherwise indicated. The term encompasses “full-length,” unprocessed CXCR1 as well as any form of CXCR1 that results from processing in the cell. The term also encompasses naturally occurring variants of CXCR1, e.g., splice variants or allelic variants. The nucleic acid sequence of an exemplary human CXCR1 is set forth in SEQ ID NO: 75. The amino acid sequence of an exemplary protein encoded by human CXCR1 is shown in SEQ ID NO: 76.

The term “CXCR2” as used herein, refers to any native CXCR2 (C-X-C motif chemokine receptor 2; also known as interleukin 8 receptor, beta, IL8RB, and CD182) from any vertebrate source, including mammals such as primates (e.g., humans) and rodents (e.g., mice and rats), unless otherwise indicated. The term encompasses “full-length,” unprocessed CXCR2 as well as any form of CXCR2 that results from processing in the cell. The term also encompasses naturally occurring variants of CXCR2, e.g., splice variants or allelic variants. The nucleic acid sequence of an exemplary human CXCR2 is set forth in SEQ ID NO: 77. The amino acid sequence of an exemplary protein encoded by human CXCR2 is shown in SEQ ID NO: 78.

The term “S100A8” as used herein, refers to any native S100A8 (S100 calcium-binding protein A8; also known as calgranulin A) from any vertebrate source, including mammals such as primates (e.g., humans) and rodents (e.g., mice and rats), unless otherwise indicated. S100A8 can form a heterodimer with S100A9 called calprotectin. The term encompasses “full-length,” unprocessed S100A8 as well as any form of S100A8 that results from processing in the cell. The term also encompasses naturally occurring variants of S100A8, e.g., splice variants or allelic variants. The nucleic acid sequence of an exemplary human S100A8 is set forth in SEQ ID NO: 79. The amino acid sequence of an exemplary protein encoded by human S100A8 is shown in SEQ ID NO: 80.

The term “S100A9” as used herein, refers to any native S100A9 (S100 calcium-binding protein A9; also known as calgranulin B and migration inhibitory factor-related protein 14 (MRP14)) from any vertebrate source, including mammals such as primates (e.g., humans) and rodents (e.g., mice and rats), unless otherwise indicated. The term encompasses “full-length,” unprocessed S100A9 as well as any form of S100A9 that results from processing in the cell. The term also encompasses naturally occurring variants of S100A9, e.g., splice variants or allelic variants. The nucleic acid sequence of an exemplary human S100A9 is set forth in SEQ ID NO: 81. The amino acid sequence of an exemplary protein encoded by human S100A9 is shown in SEQ ID NO: 82.

The term “CXCL9” as used herein, refers to any native CXCL9 (Chemokine (C-X-C Motif) Ligand 9) from any vertebrate source, including mammals such as primates (e.g., humans) and rodents (e.g., mice and rats), unless otherwise indicated. The term encompasses “full-length,” unprocessed CXCL9 as well as any form of CXCL9 that results from processing in the cell. The term also encompasses naturally occurring variants of CXCL9, e.g., splice variants or allelic variants. The nucleic acid sequence of an exemplary human CXCL9 is set forth in SEQ ID NO: 57. The amino acid sequence of an exemplary protein encoded by human CXCL9 is shown in SEQ ID NO: 58.

The term “CXCL10” as used herein, refers to any native CXCL10 (Chemokine (C-X-C Motif) Ligand 10) from any vertebrate source, including mammals such as primates (e.g., humans) and rodents (e.g., mice and rats), unless otherwise indicated. The term encompasses “full-length,” unprocessed CXCL10 as well as any form of CXCL10 that results from processing in the cell. The term also encompasses naturally occurring variants of CXCL10, e.g., splice variants or allelic variants. The nucleic acid sequence of an exemplary human CXCL10 is set forth in SEQ ID NO: 59. The amino acid sequence of an exemplary protein encoded by human CXCL10 is shown in SEQ ID NO: 60.

The term “CXCL11” as used herein, refers to any native CXCL11 (Chemokine (C-X-C Motif) Ligand 11) from any vertebrate source, including mammals such as primates (e.g., humans) and rodents (e.g., mice and rats), unless otherwise indicated. The term encompasses “full-length,” unprocessed CXCL11 as well as any form of CXCL11 that results from processing in the cell. The term also encompasses naturally occurring variants of CXCL11, e.g., splice variants or allelic variants. The nucleic acid sequence of an exemplary human CXCL11 is set forth in SEQ ID NO: 61. The amino acid sequence of an exemplary protein encoded by human CXCL11 is shown in SEQ ID NO: 62.

The term “CD27” as used herein, refers to any native CD27 (also known in the art as CD27L receptor or TNFRSF7) from any vertebrate source, including mammals such as primates (e.g., humans) and rodents (e.g., mice and rats), unless otherwise indicated. The term encompasses “full-length,” unprocessed CD27 as well as any form of CD27 that results from processing in the cell. The term also encompasses naturally occurring variants of CD27, e.g., splice variants or allelic variants. The nucleic acid sequence of an exemplary human CD27 is listed in SEQ ID NO: 31. The amino acid sequence of an exemplary protein encoded by human CD27 is shown in SEQ ID NO: 32.

The term “FOXP3” as used herein, refers to any native FOXP3 (Forkhead Box P3, also known in the art as scurfin) from any vertebrate source, including mammals such as primates (e.g., humans) and rodents (e.g., mice and rats), unless otherwise indicated. The term encompasses “full-length,” unprocessed FOXP3 as well as any form of FOXP3 that results from processing in the cell. The term also encompasses naturally occurring variants of FOXP3, e.g., splice variants or allelic variants. The nucleic acid sequence of an exemplary human FOXP3 is listed in SEQ ID NO: 33. The amino acid sequence of an exemplary protein encoded by human FOXP3 is shown in SEQ ID NO: 34.

The term “PD-1” as used herein, refers to any native PD-1 (also known as PDCD1, programmed cell death protein 1, or CD279) from any vertebrate source, including mammals such as primates (e.g., humans) and rodents (e.g., mice and rats), unless otherwise indicated. The term encompasses “full-length,” unprocessed PD-1 as well as any form of PD-1 that results from processing in the cell. The term also encompasses naturally occurring variants of PD-1, e.g., splice variants or allelic variants. The nucleic acid sequence of an exemplary human PD-1 is listed in SEQ ID NO: 35. The amino acid sequence of an exemplary protein encoded by human PD-1 is shown in SEQ ID NO: 36.

The term “CTLA4” as used herein, refers to any native CTLA4 (Cytotoxic T-lymphocyte-associated protein 4, also known in the art as CD152) from any vertebrate source, including mammals such as primates (e.g., humans) and rodents (e.g., mice and rats), unless otherwise indicated. The term encompasses “full-length,” unprocessed CTLA4 as well as any form of CTLA4 that results from processing in the cell. The term also encompasses naturally occurring variants of CTLA4, e.g., splice variants or allelic variants. The nucleic acid sequence of an exemplary human CTLA4 is listed in SEQ ID NO: 37. The amino acid sequence of an exemplary protein encoded by human CTLA4 is shown in SEQ ID NO: 38.

The term “TIGIT” as used herein, refers to any native TIGIT (T cell immunoreceptor with Ig and ITIM domains) from any vertebrate source, including mammals such as primates (e.g., humans) and rodents (e.g., mice and rats), unless otherwise indicated. The term encompasses “full-length,” unprocessed TIGIT as well as any form of TIGIT that results from processing in the cell. The term also encompasses naturally occurring variants of TIGIT, e.g., splice variants or allelic variants. The nucleic acid sequence of an exemplary human TIGIT is listed in SEQ ID NO: 39. The amino acid sequence of an exemplary protein encoded by human TIGIT is shown in SEQ ID NO: 40.

The term “IDO1” as used herein, refers to any native IDO1 (indoleamine 2,3-dioxygenase 1) from any vertebrate source, including mammals such as primates (e.g., humans) and rodents (e.g., mice and rats), unless otherwise indicated. The term encompasses “full-length,” unprocessed IDO1 as well as any form of IDO1 that results from processing in the cell. The term also encompasses naturally occurring variants of IDO1, e.g., splice variants or allelic variants. The nucleic acid sequence of an exemplary human IDO1 is listed in SEQ ID NO: 41. The amino acid sequence of an exemplary protein encoded by human IDO1 is shown in SEQ ID NO: 42.

The term “PSMB8” as used herein, refers to any native PSMB8 (Proteasome Subunit Beta Type-8) from any vertebrate source, including mammals such as primates (e.g., humans) and rodents (e.g., mice and rats), unless otherwise indicated. The term encompasses “full-length,” unprocessed PSMB8 as well as any form of PSMB8 that results from processing in the cell. The term also encompasses naturally occurring variants of PSMB8, e.g., splice variants or allelic variants. The nucleic acid sequence of an exemplary human PSMB8 is listed in SEQ ID NO: 43. The amino acid sequence of an exemplary protein encoded by human PSMB8 is shown in SEQ ID NO: 44.

The term “PSMB9” as used herein, refers to any native PSMB9 (Proteasome Subunit Beta Type-9) from any vertebrate source, including mammals such as primates (e.g., humans) and rodents (e.g., mice and rats), unless otherwise indicated. The term encompasses “full-length,” unprocessed PSMB9 as well as any form of PSMB9 that results from processing in the cell. The term also encompasses naturally occurring variants of PSMB9, e.g., splice variants or allelic variants. The nucleic acid sequence of an exemplary human PSMB9 is listed in SEQ ID NO: 45. The amino acid sequence of an exemplary protein encoded by human PSMB9 is shown in SEQ ID NO: 46.

The term “TAP1” as used herein, refers to any native TAP1 (Transporter Associated with Antigen Processing 1; also known in the art as antigen peptide transporter 1) from any vertebrate source, including mammals such as primates (e.g., humans) and rodents (e.g., mice and rats), unless otherwise indicated. The term encompasses “full-length,” unprocessed TAP1 as well as any form of TAP1 that results from processing in the cell. The term also encompasses naturally occurring variants of TAP1, e.g., splice variants or allelic variants. The nucleic acid sequence of an exemplary human TAP1 is listed in SEQ ID NO: 47. The amino acid sequence of an exemplary protein encoded by human TAP1 is shown in SEQ ID NO: 48.

The term “TAP2” as used herein, refers to any native TAP2 (antigen peptide transporter 2) from any vertebrate source, including mammals such as primates (e.g., humans) and rodents (e.g., mice and rats), unless otherwise indicated. The term encompasses “full-length,” unprocessed TAP2 as well as any form of TAP2 that results from processing in the cell. The term also encompasses naturally occurring variants of TAP2, e.g., splice variants or allelic variants. The nucleic acid sequence of an exemplary human TAP2 is listed in SEQ ID NO: 49. The amino acid sequence of an exemplary protein encoded by human TAP2 is shown in SEQ ID NO: 50.

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). An expression level for more than one gene of interest may be determined by aggregation methods known to one skilled in the art and also disclosed herein, including, for example, by calculating the median or mean of all the expression levels of the genes of interest. Before aggregation, the expression level of each gene of interest may be normalized by using statistical methods known to one skilled in the art and also disclosed herein, including, for example, normalized to the expression level of one or more housekeeping genes, or normalized to a total library size, or normalized to the median or mean expression level value across all genes measured. In some instances, before aggregation across multiple genes of interest, the normalized expression level of each gene of interest may be standardized by using statistical methods known to one skilled in the art and also disclosed herein, including, for example, by calculating the Z-score of the normalized expression level of each gene of interest.

A sample or cell that “expresses” a protein of interest is one in which mRNA encoding the protein, or the protein, including fragments thereof, is determined to be present in the sample or cell.

As used herein, the term “reference expression level” refers to an expression level against which another expression level, e.g., the expression level of one or more genes described herein (e.g., any gene set forth in Table 1 or any combination thereof (e.g., any combination set forth in any one of Tables 2-12) in a sample from an individual is compared, e.g., to make a predictive, diagnostic, prognostic, and/or therapeutic determination. For example, the reference expression level may be derived from expression levels in a reference population (e.g., the median expression level in a reference population, e.g., a population of patients having a cancer), a reference sample, and/or a pre-assigned value (e.g., a cut-off value which was previously determined to significantly (e.g., statistically significantly) separate a first subset of individuals who have been treated with an anti-cancer therapy (e.g., an anti-cancer therapy including a VEGF antagonist and a PD-L1 axis binding antagonist (e.g., a PD-L1 binding antagonist (e.g., anti-PD-L1 antibody, e.g., atezolizumab (MPDL3280A)) or a PD-1 binding antagonist (e.g., anti-PD-1 antibody)), or an anti-cancer therapy including a multi-targeted tyrosine kinase inhibitor) in a reference population and a second subset of individuals who have been treated with a different anti-cancer therapy (or who have not been treated with the anti-cancer therapy) in the same reference population based on a significant difference between an individual's responsiveness to treatment with the anti-cancer therapy and an individual's responsiveness to treatment with the different anti-cancer therapy above the cut-off value and/or below the cut-off value). In some embodiments, the cut-off value may be the median or mean expression level in the reference population. In other embodiments, the reference level may be the top 40%, the top 30%, the top 20%, the top 10%, the top 5%, or the top 1% of the expression level in the reference population. In particular embodiments, the cut-off value may be the median expression level in the reference population. It will be appreciated by one skilled in the art that the numerical value for the reference expression level may vary depending on the indication (e.g., a cancer (e.g., a kidney cancer, a breast cancer, a lung cancer, or a bladder cancer), the methodology used to detect expression levels (e.g., RNA-seq or RT-qPCR), and/or the specific combinations of genes examined (e.g., any combination of the genes set forth in Table 1; or any one of the combinations of genes listed in Tables 2-12).

Expression “above” a level (e.g., above a reference level), “increased expression,” “increased expression level,” “increased levels,” “elevated expression,” “elevated expression levels,” or “elevated levels” refers to an increased expression or increased levels of a biomarker in an individual relative to the expression level of the biomarker in a control (e.g., an individual or individuals who are not suffering from the disease or disorder (e.g., cancer), an internal control (e.g., a housekeeping biomarker), or the level of a biomarker in a sample obtained prior to administration of a therapy (e.g., an anti-cancer therapy that includes a VEGF antagonist and a PD-L1 antagonist)), or relative to a reference level (e.g., the median expression level of the biomarker in samples from a group/population of patients, e.g., patients having cancer who are being tested for responsiveness to a VEGF antagonist and a PD-L1 axis binding antagonist; the median expression level of the biomarker in samples from a group/population of patients, e.g., patients having cancer who have been identified as not responding to a VEGF antagonist and a PD-L1 axis binding antagonist; or the level in a sample previously obtained from the individual at a prior time).

Expression “below” a level (e.g., below a reference level), “decreased expression,” “decreased expression level,” “decreased levels,” “reduced expression,” “reduced expression levels,” or “reduced levels” refers to a decrease expression or decreased levels of a biomarker in an individual relative to the expression level of the biomarker in a control (e.g., an individual or individuals who are not suffering from the disease or disorder (e.g., cancer), an internal control (e.g., a housekeeping biomarker), or the level of a biomarker in a sample obtained prior to administration of a therapy (e.g., an anti-cancer therapy that includes a VEGF antagonist and a PD-L1 antagonist)), or relative to a reference level (e.g., the median expression level of the biomarker in samples from a group/population of patients, e.g., patients having cancer who are being tested for responsiveness to a VEGF antagonist and a PD-L1 axis binding antagonist; the median expression level of the biomarker in samples from a group/population of patients, e.g., patients having cancer who have been identified as not responding to a VEGF antagonist and a PD-L1 axis binding antagonist; or the level in a sample previously obtained from the individual at a prior time). In some embodiments, reduced expression is little or no expression.

A “reference sample,” “reference cell,” “reference tissue,” “control sample,” “control cell,” or “control tissue,” as used herein, refers to a sample, cell, tissue, or standard that is used for comparison purposes. In one embodiment, a reference sample, reference cell, reference tissue, control sample, control cell, or control tissue is obtained from a healthy and/or non-diseased part of the body (e.g., tissue or cells) of the same patient or individual. For example, a reference sample, reference cell, reference tissue, control sample, control cell, or control tissue may be healthy and/or non-diseased cells or tissue adjacent to the diseased cells or tissue (e.g., cells or tissue adjacent to a tumor). In another embodiment, a reference sample is obtained from an untreated tissue and/or cell of the body of the same patient or individual. In yet another embodiment, a reference sample, reference cell, reference tissue, control sample, control cell, or control tissue is obtained from a healthy and/or non-diseased part of the body (e.g., tissues or cells) of an individual who is not the patient or individual. In even another embodiment, a reference sample, reference cell, reference tissue, control sample, control cell, or control tissue is obtained from an untreated tissue and/or cell of the body of an individual who is not the patient or individual. In another embodiment, a reference sample, reference cell, reference tissue, control sample, control cell, or control tissue is obtained from a patient prior to administration of a therapy (e.g., an anti-cancer therapy that includes a VEGF antagonist and/or a PD-L1 axis binding antagonist).

The phrase “based on” when used herein means that the information about one or more biomarkers is used to inform a treatment decision, information provided on a package insert, or marketing/promotional guidance, and the like.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies (e.g., anti-VEGF antibodies and anti-PD-L1 antibodies or anti-PD-1 antibodies) are used to delay development of a disease or to slow the progression of a disease or disorder.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The term “multiplex-PCR” refers to a single PCR reaction carried out on nucleic acid obtained from a single source (e.g., an individual) using more than one primer set for the purpose of amplifying two or more DNA sequences in a single reaction.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology, (Stockton Press, N Y, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Quantitative real-time polymerase chain reaction” or “qRT-PCR” refers to a form of PCR wherein the amount of PCR product is measured at each step in a PCR reaction. This technique has been described in various publications including, for example, Cronin et al., Am. J. Pathol. 164(1):35-42 (2004) and Ma et al., Cancer Cell 5:607-616 (2004).

The term “microarray” refers to an ordered arrangement of hybridizable array elements, preferably polynucleotide probes, on a substrate.

The term “RNA-seq,” also called “Whole Transcriptome Shotgun Sequencing (WTSS),” refers to the use of high-throughput sequencing technologies to sequence and/or quantify cDNA to obtain information about a sample's RNA content. Publications describing RNA-seq include: Wang et al. Nature Reviews Genetics 10(1):57-63, 2009; Ryan et al. Bio Techniques 45(1):81-94, 2008; and Maher et al. Nature 458(7234):97-101, 2009.

The term “diagnosis” is used herein to refer to the identification or classification of a molecular or pathological state, disease or condition (e.g., cancer (e.g., kidney cancer)). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)). In some embodiments, the diagnosis is of a sarcomatoid cancer (e.g., a sarcomatoid kidney cancer (e.g., sarcomatoid RCC)).

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8⁺ T lymphocytes and/or CD4⁺ T lymphocytes), B lymphocytes, or other bone marrow-lineage cells, including granulocytes (e.g., neutrophils, eosinophils, and basophils), monocytes, macrophages (e.g., CD68⁺/CD163⁺ macrophages), dendritic cells (e.g., interdigitating dendritic cells), histiocytes, and natural killer (NK) cells.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

As used herein, “administering” is meant a method of giving a dosage of a compound (e.g., a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))), a PD-L1 axis binding antagonist (e.g., an anti-PD-L1 antibody, e.g., atezolizumab), and/or an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib))))) or a composition (e.g., a pharmaceutical composition, e.g., a pharmaceutical composition including a VEGF antagonist, a PD-L1 axis binding antagonist, and/or an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib))))) to a patient. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subcutaneously, subconjunctivally, intravesicularly, mucosally, intrapericardially, intraumbilically, intraocularly, intraorbitally, intravitreally (e.g., by intravitreal injection), by eye drop, orally, topically, transdermally, parenterally, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated).

A “therapeutically effective amount” refers to an amount of a therapeutic agent to treat or prevent a disease or disorder (e.g., a cancer, e.g., a kidney cancer (e.g., RCC)) in a mammal (e.g., a human). In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival (e.g., overall survival or progression-free survival), time to disease progression (TTP), response rates (e.g., overall response (ORR), complete response (CR) and partial response (PR)), duration of response, deterioration-free rate (DFR), and/or quality of life.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s). For example, in some embodiments, a VEGF antagonist and a PD-L1 axis binding antagonist may be administered concurrently.

By “reduce or inhibit” is meant the ability to cause an overall decrease of 20%, 30%, 40%, 50%, 60%, 70%, 75%, 80%, 85%, 90%, 95%, or greater. Reduce or inhibit can refer, for example, to the symptoms of the disorder being treated, the presence or size of metastases, or the size of the primary tumor.

A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

A “maintenance” dose or “extended” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks.

“Response to a treatment,” “responsiveness to treatment,” or “benefit from a treatment” can be assessed using any endpoint indicating a benefit to the individual, including, without limitation, (1) inhibition, to some extent, of disease progression (e.g., cancer progression), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the disease or disorder (e.g., cancer); (6) increase or extension in the length of survival, including overall survival (OS HR<1), progression free survival (PFS HR<1), and/or deterioration-free survival; (7) increase in the overall response rate (ORR), complete response (CR) rate, and/or deterioration-free rate (DFR); and/or (8) decreased mortality at a given point of time following treatment (e.g., treatment with an anti-cancer therapy that includes a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A)) or a PD-1 binding antagonist (e.g., an anti-PD-1 antibody), or treatment with an anti-cancer therapy that includes an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib))))).

An “objective response” refers to a measurable response, including complete response (CR) or partial response (PR). In some embodiments, the “objective response rate (ORR)” refers to the sum of complete response (CR) rate and partial response (PR) rate.

By “complete response” or “CR” is intended the disappearance of all signs of cancer (e.g., disappearance of all target lesions) in response to treatment. This does not always mean the cancer has been cured.

As used herein, “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration, or longer.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

The term “survival” refers to the patient remaining alive, and includes overall survival as well as progression-free survival.

As used herein, “progression-free survival” or “PFS” refers to the length of time during and after treatment during which the disease being treated (e.g., cancer, e.g., a kidney cancer (e.g., RCC)) does not progress or get worse. Progression-free survival may include the amount of time individuals have experienced a complete response or a partial response, as well as the amount of time individuals have experienced stable disease.

As used herein, “overall survival” or “OS” refers to the percentage of subjects in a group who are likely to be alive after a particular duration of time (e.g., 6 months, 1 year, 2 years, 3 years, 4 years, 5 years, 10 years, 15 years, 20 years, or more than 20 years from the time of diagnosis or treatment).

By “extending survival” is meant increasing overall or progression-free survival in a treated patient relative to an untreated patient (i.e. relative to a patient not treated with the medicament), or relative to a patient who does not express a biomarker at the designated level, and/or relative to a patient treated with an approved anti-tumor agent (e.g., an anti-VEGF antibody (e.g., bevacizumab), a PD-L1 axis binding antagonist (e.g., atezolizumab), and/or a multi-targeted tyrosine kinase inhibitor (e.g., sunitinib)).

As used herein, “hazard ratio” or “HR” is a statistical definition for rates of events. For the purpose of the invention, hazard ratio is defined as representing the probability of an event (e.g., PFS or OS) in the experimental (e.g., treatment) group/arm divided by the probability of an event in the control group/arm at any specific point in time. An HR with a value of 1 indicates that the relative risk of an endpoint (e.g., death) is equal in both the “treatment” and “control” groups; a value greater than 1 indicates that the risk is greater in the treatment group relative to the control group; and a value less than 1 indicates that the risk is greater in the control group relative to the treatment group. “Hazard ratio” in progression-free survival analysis (i.e., PFS HR) is a summary of the difference between two progression-free survival curves, representing the reduction in the risk of death on treatment compared to control, over a period of follow-up. “Hazard ratio” in overall survival analysis (i.e., OS HR) is a summary of the difference between two overall survival curves, representing the reduction in the risk of death on treatment compared to control, over a period of follow-up.

As used herein, “deterioration-free rate” or “DFR” refers to the probability that a patient will experience a clinically meaningful deterioration in a length of time, e.g., the time from onset of a therapy to a patient's first ≥2-point increase above baseline on the MD Anderson Symptom Inventory (MDASI) interference scale.

The “MD Anderson Symptom Inventory (MDASI) interference scale” refers to a patient-reported outcome measurement scoring system that assesses the severity and impact of multiple symptoms related to cancer and its treatment (see, e.g., Mendoza et al. Clin. Breast Cancer 13:325-334, 2013; Jones et al. Clin. Genitourin. Cancer 12:41-49, 2014; and Shi et al. Pain 158:1108-1112, 2017). In the MDASI interference scale, a patient rates the degree to which symptoms interfered with various aspects of life during the past 24 hours. Each interference item (work, general activity, walking, relations with others, enjoyment of life, and mood) is rated on a 0-10 scale, with 0 representing “did not interfere” and 10 representing “interfered completely.”

The term “anti-cancer therapy” refers to a therapy useful in treating cancer. Examples of anti-cancer therapeutic agents include, but are limited to, cytotoxic agents, chemotherapeutic agents, growth inhibitory agents, agents used in radiation therapy, anti-angiogenesis agents, apoptotic agents, anti-tubulin agents, and other agents to treat cancer, for example, anti-CD20 antibodies, platelet derived growth factor inhibitors (e.g., GLEEVEC™ (imatinib mesylate)), a COX-2 inhibitor (e.g., celecoxib), interferons, cytokines, antagonists (e.g., neutralizing antibodies) that bind to one or more of the following targets: PDGFR-β, BlyS, APRIL, BCMA receptor(s), TRAIL/Apo2, other bioactive and organic chemical agents, and the like. Combinations thereof are also included in the invention.

A “VEGF antagonist” or “VEGF-specific antagonist” refers to a molecule capable of binding to VEGF, reducing VEGF expression levels, or neutralizing, blocking, inhibiting, abrogating, reducing, or interfering with VEGF biological activities, including, but not limited to, VEGF binding to one or more VEGF receptors, VEGF signaling, and VEGF mediated angiogenesis and endothelial cell survival or proliferation. For example, a molecule capable of neutralizing, blocking, inhibiting, abrogating, reducing, or interfering with VEGF biological activities can exert its effects by binding to one or more VEGF receptor (VEGFR) (e.g., VEGFR1, VEGFR2, VEGFR3, membrane-bound VEGF receptor (mbVEGFR), or soluble VEGF receptor (sVEGFR)). Such antagonists are also referred to herein as “VEGFR inhibitors.” Included as VEGF-specific antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF₁₂₁-gelonin (Peregrine). VEGF-specific antagonists also include antagonist variants of VEGF polypeptides, antisense nucleobase oligomers complementary to at least a fragment of a nucleic acid molecule encoding a VEGF polypeptide; small RNAs complementary to at least a fragment of a nucleic acid molecule encoding a VEGF polypeptide; ribozymes that target VEGF; peptibodies to VEGF; and VEGF aptamers. VEGF antagonists also include polypeptides that bind to VEGFR, anti-VEGFR antibodies, and antigen-binding fragments thereof, and derivatives which bind to VEGFR thereby blocking, inhibiting, abrogating, reducing, or interfering with VEGF biological activities (e.g., VEGF signaling), or fusions proteins. VEGF-specific antagonists also include nonpeptide small molecules that bind to VEGF or VEGFR and are capable of blocking, inhibiting, abrogating, reducing, or interfering with VEGF biological activities. Thus, the term “VEGF activities” specifically includes VEGF mediated biological activities of VEGF. In certain embodiments, the VEGF antagonist reduces or inhibits, by at least 10%, 20%, 30%, 40%, 50%, 60%, 70%, 80%, 90% or more, the expression level or biological activity of VEGF. In some embodiments, the VEGF inhibited by the VEGF-specific antagonist is VEGF (8-109), VEGF (1-109), or VEGF₁₆₅.

As used herein VEGF antagonists can include, but are not limited to, anti-VEGFR2 antibodies and related molecules (e.g., ramucirumab, tanibirumab, aflibercept), anti-VEGFR1 antibodies and related molecules (e.g., icrucumab, aflibercept (VEGF Trap-Eye; EYLEA®), and ziv-aflibercept (VEGF Trap; ZALTRAP®)), bispecific VEGF antibodies (e.g., MP-0250, vanucizumab (VEGF-ANG2), and bispecific antibodies disclosed in US 2001/0236388), bispecific antibodies including combinations of two of anti-VEGF, anti-VEGFR1, and anti-VEGFR2 arms, anti-VEGFA antibodies (e.g., bevacizumab, sevacizumab), anti-VEGFB antibodies, anti-VEGFC antibodies (e.g., VGX-100), anti-VEGFD antibodies, and nonpeptide small molecule VEGF antagonists (e.g., pazopanib, axitinib, vandetanib, stivarga, cabozantinib, lenvatinib, nintedanib, orantinib, telatinib, dovitinig, cediranib, motesanib, sulfatinib, apatinib, foretinib, famitinib, and tivozanib).

An “anti-VEGF antibody” is an antibody that binds to VEGF with sufficient affinity and specificity. In certain embodiments, the antibody will have a sufficiently high binding affinity for VEGF, for example, the antibody may bind hVEGF with a Kd value of between 100 nM-1 μM. Antibody affinities may be determined, e.g., by a surface plasmon resonance based assay (such as the BIAcore® assay as described in PCT Application Publication No. WO2005/012359); enzyme-linked immunoabsorbent assay (ELISA); and competition assays (e.g. radioimmunoassays (RIAs)).

In certain embodiments, the anti-VEGF antibody can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as PIGF, PDGF, or bFGF. In one embodiment, anti-VEGF antibody is a monoclonal antibody that binds to the same epitope as the monoclonal anti-VEGF antibody A4.6.1 produced by hybridoma ATCC HB 10709. In another embodiment, the anti-VEGF antibody is a recombinant humanized anti-VEGF monoclonal antibody generated according to Presta et al. (Cancer Res. 57:4593-4599, 1997), including but not limited to the antibody known as bevacizumab (BV; AVASTIN®).

The anti-VEGF antibody “Bevacizumab (BV),” also known as “rhuMAb VEGF” or “AVASTIN®,” is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879 issued Feb. 26, 2005, the entire disclosure of which is expressly incorporated herein by reference. Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., (Journal of Immunological Methods 288:149-164, 2004). Other preferred antibodies include those that bind to a functional epitope on human VEGF comprising of residues F17, M18, D19, Y21, Y25, Q89, 191, K101, E103, and 0104 or, alternatively, comprising residues F17, Y21, Q22, Y25, D63, 183, and Q89.

The term “PD-L1 axis binding antagonist” refers to a molecule that inhibits the interaction of a PD-L1 axis binding partner with one or more of its binding partners, so as to remove T cell dysfunction resulting from signaling on the PD-1 signaling axis, with a result being restored or enhanced T cell function. As used herein, a PD-L1 axis binding antagonist includes a PD-L1 binding antagonist and a PD-1 binding antagonist as well as molecules that interfere with the interaction between PD-L1 and PD-1 (e.g., a PD-L2-Fc fusion).

The terms “anti-PD-L1 antibody” and “an antibody that binds to PD-L1” refer to an antibody that is capable of binding PD-L1 with sufficient affinity such that the antibody is useful as a diagnostic and/or therapeutic agent in targeting PD-L1. In one embodiment, the extent of binding of an anti-PD-L1 antibody to an unrelated, non-PD-L1 protein is less than about 10% of the binding of the antibody to PD-L1 as measured, for example, by a RIA. In certain embodiments, an anti-PD-L1 antibody binds to an epitope of PD-L1 that is conserved among PD-L1 from different species.

The terms “anti-PD-1 antibody” and “an antibody that binds to PD-1” refer to an antibody that is capable of binding PD-1 with sufficient affinity such that the antibody is useful as a diagnostic and/or therapeutic agent in targeting PD-1. In one embodiment, the extent of binding of an anti-PD-1 antibody to an unrelated, non-PD-1 protein is less than about 10% of the binding of the antibody to PD-1 as measured, for example, by a RIA. In certain embodiments, an anti-PD-1 antibody binds to an epitope of PD-1 that is conserved among PD-1 from different species.

The term “PD-L1 binding antagonist” refers to a molecule that decreases, blocks, inhibits, abrogates, or interferes with signal transduction resulting from the interaction of PD-L1 with either one or more of its binding partners, such as PD-1 or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen-binding fragments thereof, immunoadhesins, fusion proteins, oligopeptides, and other molecules that decrease, block, inhibit, abrogate, or interfere with signal transduction resulting from the interaction of PD-L1 with one or more of its binding partners, such as PD-1 or B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific embodiment, the anti-PD-L1 antibody is atezolizumab (CAS Registry Number: 1422185-06-5), also known as MPDL3280A, and described herein. In another specific embodiment, the anti-PD-L1 antibody is YW243.55.S70, described herein. In another specific embodiment, the anti-PD-L1 antibody is MDX-1105, described herein. In still another specific aspect, the anti-PD-L1 antibody is MED14736 (durvalumab), described herein. In still another specific aspect, the anti-PD-L1 antibody is MSB0010718C (avelumab), described herein.

As used herein, a “PD-1 binding antagonist” is a molecule that decreases, blocks, inhibits, abrogates or interferes with signal transduction resulting from the interaction of PD-1 with one or more of its binding partners, such as PD-L1 and/or PD-L2. In some embodiments, the PD-1 binding antagonist is a molecule that inhibits the binding of PD-1 to its binding partners. In a specific aspect, the PD-1 binding antagonist inhibits the binding of PD-1 to PD-L1 and/or PD-L2. For example, PD-1 binding antagonists include anti PD-1 antibodies and antigen-binding fragments thereof, immunoadhesins, fusion proteins, oligopeptides, small molecule antagonists, polynucleotide antagonists, and other molecules that decrease, block, inhibit, abrogate or interfere with signal transduction resulting from the interaction of PD-1 with PD-L1 and/or PD-L2. In one embodiment, a PD-1 binding antagonist reduces the negative signal mediated by or through cell surface proteins expressed on T lymphocytes, and other cells, mediated signaling through PD-1 or PD-L1 so as render a dysfunctional T cell less dysfunctional. In some embodiments, the PD-1 binding antagonist is an anti-PD-1 antibody. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is MEDI-0680 (AMP-514). In another specific aspect, a PD-1 binding antagonist is PDR001. In another specific aspect, a PD-1 binding antagonist is REGN2810. In another specific aspect, a PD-1 binding antagonist is BGB-108. In another specific aspect, a PD-1 binding antagonist is AMP-224.

An “angiogenesis inhibitor” or “anti-angiogenesis agent” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor.

For example, an anti-angiogenesis agent is an antibody or other antagonist to an angiogenic agent as defined above, e.g., antibodies to VEGF-A or the VEGF-A receptor (e.g., KDR receptor or Flt-1 receptor), anti-PDGFR inhibitors such as GLEEVEC™ (Imatinib Mesylate). Anti-angiogenesis agents also include native angiogenesis inhibitors, e.g., angiostatin, endostatin, etc. See, for example, Klagsbrun and D′Amore, Annu. Rev. PhysioL, 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12):1359-1364 (1999); Tonini et al., Oncogene, 22:6549-6556 (2003) and, Sato Int. J. Clin. Oncol., 8:200-206 (2003).

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, sm¹⁵³, Bi²¹², P³², and radioactive isotopes of Lu), chemotherapeutic agents, e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. A tumoricidal agent causes destruction of tumor cells.

A “chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Pfizer), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride; vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew. Chem. Intl. Ed. Engl. 33:183-186, 1994); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agents also include anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); protein kinase inhibitors; lipid kinase inhibitors; antisense oligonucleotides, particularly those which inhibit expression of genes in signaling pathways implicated in aberrant cell proliferation, such as, for example, PKC-alpha, Ralf and H-Ras; ribozymes such as VEGF expression inhibitors (e.g., ANGIOZYME®) and HER2 expression inhibitors; vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and pharmaceutically acceptable salts, acids and derivatives of any of the above.

Chemotherapeutic agents also include antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories), which is a recombinant, exclusively human-sequence, full-length IgG1 λ antibody genetically modified to recognize interleukin-12 p40 protein.

Chemotherapeutic agents also include “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyOmethoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396, WO 1999/09016, WO 1998/43960, WO 1997/38983, WO 1999/06378, WO 1999/06396, WO 1996/30347, WO 1996/33978, WO 1996/3397, and WO 1996/33980.

The term “multi-targeted tyrosine kinase inhibitor,” as used herein, refers to a tyrosine kinase inhibitor that inhibits multiple (i.e., more than one) tyrosine kinase proteins. The tyrosine kinase proteins may be receptor tyrosine kinases and/or cellular tyrosine kinases. For example, the multi-targeted tyrosine kinase inhibitor may inhibit platelet-derived growth factor receptors (e.g., PDGFR-αα, PDGFR-ββ, and/or PDGFR-αβ), VEGF receptors (e.g., VEGFR1 and/or VEGFR2), CD117 (c-Kit), RET, CD114, and/or CD135. Exemplary multi-targeted tyrosine kinase inhibitors include sunitinib (also known as N-[2-(Diethylamino)ethyl]-5-[(Z)-(5-fluoro-2-oxo-1,2-dihydro-3H-indol-3-ylidene)methyl]-2,4-dimethyl-1H-pyrrole-3-carboxamide, SUTENT® or SU11248), SU6656, motesanib, sorafenib (e.g., NEXEVAR® or BAY439006), axitinib, afatinib, bosutinib, crizotinib, cabozantinib, dasatinib, entrectinib, pazopanib, lapatinib, and vandetanib (also known as ZACTIMA® or ZD6474). It is to be understood that a multi-targeted tyrosine kinase inhibitor that inhibits a VEGF receptor may also be considered a VEGFR inhibitor.

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, all-trans retinoic acid (ATRA), valrubicin, zoledronate, and zoledronic acid, and pharmaceutically acceptable salts thereof.

The term “prodrug” as used herein refers to a precursor or derivative form of a pharmaceutically active substance that is less cytotoxic to tumor cells compared to the parent drug and is capable of being enzymatically activated or converted into the more active parent form. See, for example, Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

A “growth inhibitory agent” when used herein refers to a compound or composition which inhibits growth and/or proliferation of a cell (e.g., a cell whose growth is dependent on PD-L1 expression) either in vitro or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as the anthracycline antibiotic doxorubicin ((8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione), epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in “The Molecular Basis of Cancer,” Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

The term “pharmaceutical formulation” refers to a preparation which is in such form as to permit the biological activity of an active ingredient contained therein to be effective, and which contains no additional components which are unacceptably toxic to a patient to which the formulation would be administered.

A “pharmaceutically acceptable carrier” refers to an ingredient in a pharmaceutical formulation, other than an active ingredient, which is nontoxic to a patient. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications, and/or warnings concerning the use of such therapeutic products.

A “sterile” formulation is aseptic or free from all living microorganisms and their spores.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain embodiments, the manufacture or kit is promoted, distributed, or sold as a unit for performing the methods described herein.

The term “small molecule” refers to any molecule with a molecular weight of about 2000 daltons or less, preferably of about 500 daltons or less.

The word “label” when used herein refers to a compound or composition that is conjugated or fused directly or indirectly to a reagent such as a polynucleotide probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable. The term is intended to encompass direct labeling of a probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

An “isolated” antibody is one which has been identified and separated and/or recovered from a component of its natural environment. Contaminant components of its natural environment are materials which would interfere with research, diagnostic, and/or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

A “blocking” antibody or an antibody “antagonist” is one which inhibits or reduces biological activity of the antigen it binds. For example, a VEGF-specific antagonist antibody binds VEGF and inhibits the ability of VEGF to induce vascular endothelial cell proliferation. Preferred blocking antibodies or antagonist antibodies completely inhibit the biological activity of the antigen.

Unless indicated otherwise, the expression “multivalent antibody” is used throughout this specification to denote an antibody comprising three or more antigen binding sites. The multivalent antibody is preferably engineered to have the three or more antigen binding sites and is generally not a native sequence IgM or IgA antibody.

The “light chains” of antibodies (immunoglobulins) from any mammalian species can be assigned to one of two clearly distinct types, called kappa (“κ”) and lambda (“Δ”), based on the amino acid sequences of their constant domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2, and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain segments of the variable domains differ extensively in sequence among antibodies. The variable or “V” domain mediates antigen binding and defines specificity of a particular antibody for its particular antigen. However, the variability is not evenly distributed across the span of the variable domains. Instead, the V regions consist of relatively invariant stretches called framework regions (FRs) of 15-30 amino acids separated by shorter regions of extreme variability called “hypervariable regions” that are each 9-12 amino acids long. The term “hypervariable region” or “HVR” when used herein refers to the amino acid residues of an antibody which are responsible for antigen-binding. The hypervariable region generally comprises amino acid residues from, for example, around about residues 24-34 (L1), 50-56 (L2) and 89-97 (L3) in the VL, and around about residues 26-35 (H1), 49-65 (H2) and 95-102 (H3) in the VH (in one embodiment, H1 is around about residues 31-35); Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2), and 91-96 (L3) in the VL, and 26-32 (H1), 53-55 (H2), and 96-101 (H3) in the VH; Chothia and Lesk, J. Mol. Biol. 196:901-917 (1987). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). Accordingly, the HVR and FR sequences generally appear in the following sequence in VH (or VL): FR1-H1(L1)-FR2-H2(L2)-FR3-H3(L3)-FR4. The constant domains are not involved directly in binding an antibody to an antigen, but exhibit various effector functions, such as participation of the antibody in antibody dependent cellular cytotoxicity (ADCC).

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “hypervariable region,” “HVR,” or “HV,” as used herein, refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, for example, Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, for example, Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35b | H26-H35b | H26-H32 | H30-H35b |
| | | | | (Kabat Numbering) |
| H1 | H31-H35 | H26-H35 | H26-H32 | H30-H35 |
| | | | | (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3.

In one embodiment, for the VL, the subgroup is subgroup kappa I as in Kabat et al., supra. In one embodiment, for the VH, the subgroup is subgroup III as in Kabat et al., supra.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

Unless otherwise indicated, HVR residues and other residues in the variable domain (e.g., FR residues) are numbered herein according to Kabat et al., supra.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody, preferably comprising the antigen-binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md. (1991).

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down-regulation of cell surface receptors (e.g. B cell receptor); and B cell activation.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv, see, e.g., PluckthOn, in The Pharmacology of Monoclonal Antibodies, vol. 113, Rosenburg and Moore eds., (Springer-Verlag, New York, 1994), pp. 269-315.

The term “multispecific antibody” is used in the broadest sense and specifically covers an antibody comprising a heavy chain variable domain (VH) and a light chain variable domain (VL), where the VH-VL unit has polyepitopic specificity (i.e., is capable of binding to two different epitopes on one biological molecule or each epitope on a different biological molecule). Such multispecific antibodies include, but are not limited to, full-length antibodies, antibodies having two or more VL and VH domains, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). “Dual specificity” or “bispecificity” refers to the ability to specifically bind to two different epitopes on the same or different target(s). However, in contrast to bispecific antibodies, dual-specific antibodies have two antigen-binding arms that are identical in amino acid sequence and each Fab arm is capable of recognizing two antigens. Dual-specificity allows the antibodies to interact with high affinity with two different antigens as a single Fab or IgG molecule. According to one embodiment, the multispecific antibody in an IgG1 form binds to each epitope with an affinity of 5 μM to 0.001 μM, 3 μM to 0.001 μM, 1 μM to 0.001 μM, 0.5 μM to 0.001 μM or 0.1 μM to 0.001 μM. “Monospecific” refers to the ability to bind only one epitope.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of antibodies are called α, δ, ε, γ, and μ, respectively.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual (Cold Spring Harbor Laboratory Press, 2nd ed. 1988); Hammerling et al., in: Monoclonal Antibodies and T cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628, 1991; Marks et al., J. Mol. Biol. 222: 581-597, 1992; Sidhu et al., J. Mol. Biol. 338(2): 299-310, 2004; Lee et al., J. Mol. Biol. 340(5): 1073-1093, 2004; Fellouse, Proc. Natl. Acad. Sci. USA 101(34): 12467-12472, 2004; and Lee et al., J. Immunol. Methods 284(1-2): 119-132, 2004; and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551, 1993; Jakobovits et al., Nature 362: 255-258, 1993; Bruggemann et al., Year in Immunol. 7:33, 1993; U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859, 1994; Morrison, Nature 368: 812-813, 1994; Fishwild et al., Nature Biotechnol. 14: 845-851, 1996; Neuberger, Nature Biotechnol. 14: 826, 1996; and Lonberg et al., Intern. Rev. Immunol. 13: 65-93, 1995.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, FR residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525, 1986; Riechmann et al., Nature 332:323-329, 1988; and Presta, Curr. Op. Struct. Biol. 2:593-596, 1992.

A “wild-type (WT)” or “reference” sequence or the sequence of a “wild-type” or “reference” protein/polypeptide, such as an HVR or a variable domain of a reference antibody, may be the reference sequence from which variant polypeptides are derived through the introduction of mutations. In general, the “wild-type” sequence for a given protein is the sequence that is most common in nature. Similarly, a “wild-type” gene sequence is the sequence for that gene which is most commonly found in nature. Mutations may be introduced into a “wild-type” gene (and thus the protein it encodes) either through natural processes or through man-induced means. The products of such processes are “variant” or “mutant” forms of the original “wild-type” protein or gene.

A “variant” or “mutant” of a starting or reference polypeptide (e.g., a reference antibody or its variable domain(s)/HVR(s)), is a polypeptide that (1) has an amino acid sequence different from that of the starting or reference polypeptide and (2) was derived from the starting or reference polypeptide through either natural or artificial (man-made) mutagenesis. Such variants include, for example, deletions from, and/or insertions into and/or substitutions of, residues within the amino acid sequence of the polypeptide of interest, referred to herein as “amino acid residue alterations.” Thus, a variant HVR refers to a HVR comprising a variant sequence with respect to a starting or reference polypeptide sequence (such as that of a source antibody or antigen binding fragment). An amino acid residue alteration, in this context, refers to an amino acid different from the amino acid at the corresponding position in a starting or reference polypeptide sequence (such as that of a reference antibody or fragment thereof). Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described herein.

With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of 10⁻⁴M or lower, alternatively 10⁻⁵M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a Kd in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and Kd values are inversely related. A high affinity for an antigen is measured by a low Kd value. In one embodiment, the term “specific binding” refers to binding where a molecule binds to a particular polypeptide or epitope on a particular polypeptide without substantially binding to any other polypeptide or polypeptide epitope.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

An “immunoconjugate” is an antibody conjugated to one or more heterologous molecule(s), including but not limited to a cytotoxic agent.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG1, IgG2 (including IgG2A and IgG2B), IgG3, or IgG4 subtypes, IgA (including IgA1 and IgA2), IgE, IgD or IgM. The Ig fusions preferably include the substitution of a domain of a polypeptide or antibody described herein in the place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130. For example, useful immunoadhesins as medicaments useful for therapy herein include polypeptides that comprise the extracellular domain (ECD) or PD-1-binding portions of PD-L1 or PD-L2, or the extracellular or PD-L1- or PD-L2-binding portions of PD-1, fused to a constant domain of an immunoglobulin sequence, such as a PD-L1 ECD-Fc, a PD-L2 ECD-Fc, and a PD-1 ECD-Fc, respectively. Immunoadhesin combinations of Ig Fc and ECD of cell surface receptors are sometimes termed soluble receptors.

A “fusion protein” and a “fusion polypeptide” refer to a polypeptide having two portions covalently linked together, where each of the portions is a polypeptide having a different property. The property may be a biological property, such as activity in vitro or in vivo. The property may also be simple chemical or physical property, such as binding to a target molecule, catalysis of a reaction, and the like. The two portions may be linked directly by a single peptide bond or through a peptide linker but are in reading frame with each other.

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single-stranded polynucleotide that is capable of hybridizing to a nucleic acid and allowing polymerization of a complementary nucleic acid, generally by providing a free 3′—OH group.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “isolated” nucleic acid molecule is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

### II. DIAGNOSTIC METHODS

Provided herein are methods for identifying an individual having a cancer (e.g., a kidney cancer (e.g., a renal cell carcinoma (RCC))) who may benefit from a treatment with an anti-cancer therapy including a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A) or a PD-1 binding antagonist (e.g., an anti-PD-1 antibody)).

The methods described herein are based, at least in part, on the discovery that the presence of sarcomatoid cancer and/or an individual's Memorial Sloan Kettering Cancer Center (MSKCC) risk score may be used to identify whether the individual is likely to benefit from an anti-cancer therapy that includes a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A) or a PD-1 binding antagonist (e.g., an anti-PD-1 antibody)). The benefit may be, for example, in terms of improved progression-free survival (PFS), overall survival (OS), overall response rate (ORR), complete response (CR) rate, and/or deterioration-free rate (DFR). For example, in some instances, the benefit may be in terms of PFS. In other instances, the benefit may be in terms of OS. In yet other instances, the benefit may be in terms of ORR. In still other instances, the benefit may be in terms of CR rate. In still other instances, the benefit may be in terms of DFR.

The methods described herein are also based, at least in part, on the finding that the expression level of one or more genes (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and/or S100A9) in a sample from the individual may be used to predict the therapeutic efficacy of an anti-cancer therapy that includes a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A) or a PD-1 binding antagonist (e.g., an anti-PD-1 antibody)). In another aspect, methods and assays described herein are based, at least in part, on the finding that the expression level of one or more genes (e.g., VEGFA, KDR, ESM1, PECAM1, FLT1, ANGPTL4, and/or CD34) in a sample from the individual may be used to predict the therapeutic efficacy of a treatment including an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))). In some embodiments, sarcomatoid cancer and/or an individual's MSKCC risk score can be used in combination with the expression level of one or more genes (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and/or S100A9) in a sample from the individual, e.g., to identify individuals likely to benefit (e.g., in terms of PFS) from an anti-cancer therapy as described herein, to select individuals for an anti-cancer therapy as described herein, and/or to optimize therapeutic efficacy of an anti-cancer therapy as described herein.

Further provided herein are methods for selecting a therapy for an individual having a cancer (e.g., kidney cancer (e.g., RCC)); methods for determining whether an individual having a cancer is likely to respond to treatment including a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A) or a PD-1 binding antagonist (e.g., an anti-PD-1 antibody)); methods for determining whether an individual having a cancer is likely to respond to treatment including an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))); methods for predicting the responsiveness of an individual having a cancer to treatment comprising a VEGF antagonist and a PD-L1 axis binding antagonist; methods for predicting the responsiveness of an individual having a cancer to treatment comprising an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))); methods for monitoring the response of an individual having a cancer to treatment including a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A) or a PD-1 binding antagonist (e.g., an anti-PD-1 antibody)); and methods for monitoring the response of an individual having a cancer to treatment including an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))). Any of the methods provided herein may further include administering to the individual a VEGF antagonist and a PD-L1 axis binding antagonist (e.g., as described below in Section III) to the individual.

For example, provided herein is a method of identifying an individual having a cancer (e.g., a kidney cancer (e.g., RCC)) who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), the method comprising determining whether the individual has a sarcomatoid cancer, wherein the presence of a sarcomatoid cancer identifies the individual as one who may benefit from treatment with an anti-cancer therapy comprising a VEGF antagonist and a PD-L1 axis binding antagonist. In some embodiments, the method further comprises administering an effective amount of an anti-cancer therapy comprising a VEGF antagonist and a PD-L1 axis binding antagonist to the individual.

In another example, provided herein is a method for selecting a therapy for an individual having cancer (e.g., a kidney cancer (e.g., RCC)), the method comprising (a) determining whether the individual has a sarcomatoid cancer, wherein the presence of a sarcomatoid cancer identifies the individual as one who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)); and (b) selecting an anti-cancer therapy comprising a VEGF antagonist and a PD-L1 axis binding antagonist based on the presence of a sarcomatoid cancer. In some embodiments, the method further comprises administering an effective amount of an anti-cancer therapy comprising a VEGF antagonist and a PD-L1 axis binding antagonist to the individual.

The benefit may be, for example, in terms of improved progression-free survival (PFS), overall survival (OS), overall response rate (ORR), complete response (CR) rate, or deterioration-free rate (DFR). In some embodiments, the benefit is in terms of improved PFS. In some instances, the benefit is in terms of improved OS. In some instances, the benefit is in terms of improved ORR. In some instances, the benefit is in terms of improved CR rate. In some instances, the benefit is in terms of improved DFR. In some instances, DFR is determined in terms of the time from onset of treatment to the individual's first increase of greater than or equal to 2 points above baseline on the MD Anderson Symptom Inventory (MDASI) interference scale.

For example, provided herein is a method of identifying an individual having a cancer (e.g., a kidney cancer (e.g., RCC)) who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), the method comprising determining whether the individual has a sarcomatoid cancer, wherein the presence of a sarcomatoid cancer identifies the individual as one who may benefit from treatment with an anti-cancer therapy comprising a VEGF antagonist and a PD-L1 axis binding antagonist, wherein the benefit is in terms of improved PFS. In some embodiments, the method further comprises administering an effective amount of an anti-cancer therapy comprising a VEGF antagonist and a PD-L1 axis binding antagonist to the individual.

In another example, provided herein is a method for selecting a therapy for an individual having cancer (e.g., a kidney cancer (e.g., RCC)), the method comprising (a) determining whether the individual has a sarcomatoid cancer, wherein the presence of a sarcomatoid cancer identifies the individual as one who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved PFS; and (b) selecting an anti-cancer therapy comprising a VEGF antagonist and a PD-L1 axis binding antagonist based on the presence of a sarcomatoid cancer. In some embodiments, the method further comprises administering an effective amount of an anti-cancer therapy comprising a VEGF antagonist and a PD-L1 axis binding antagonist to the individual.

In another example, provided herein is a method of identifying an individual having a cancer (e.g., a kidney cancer (e.g., RCC)) who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), the method comprising determining whether the individual has a sarcomatoid cancer, wherein the presence of a sarcomatoid cancer identifies the individual as one who may benefit from treatment with an anti-cancer therapy comprising a VEGF antagonist and a PD-L1 axis binding antagonist, wherein the benefit is in terms of improved OS. In some embodiments, the method further comprises administering an effective amount of an anti-cancer therapy comprising a VEGF antagonist and a PD-L1 axis binding antagonist to the individual.

In yet another example, provided herein is a method for selecting a therapy for an individual having cancer (e.g., a kidney cancer (e.g., RCC)), the method comprising (a) determining whether the individual has a sarcomatoid cancer, wherein the presence of a sarcomatoid cancer identifies the individual as one who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved OS; and (b) selecting an anti-cancer therapy comprising a VEGF antagonist and a PD-L1 axis binding antagonist based on the presence of a sarcomatoid cancer. In some embodiments, the method further comprises administering an effective amount of an anti-cancer therapy comprising a VEGF antagonist and a PD-L1 axis binding antagonist to the individual.

In a further example, provided herein is a method of identifying an individual having a cancer (e.g., a kidney cancer (e.g., RCC)) who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), the method comprising determining whether the individual has a sarcomatoid cancer, wherein the presence of a sarcomatoid cancer identifies the individual as one who may benefit from treatment with an anti-cancer therapy comprising a VEGF antagonist and a PD-L1 axis binding antagonist, wherein the benefit is in terms of improved ORR. In some embodiments, the method further comprises administering an effective amount of an anti-cancer therapy comprising a VEGF antagonist and a PD-L1 axis binding antagonist to the individual.

In a still further example, provided herein is a method for selecting a therapy for an individual having cancer (e.g., a kidney cancer (e.g., RCC)), the method comprising (a) determining whether the individual has a sarcomatoid cancer, wherein the presence of a sarcomatoid cancer identifies the individual as one who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved ORR; and (b) selecting an anti-cancer therapy comprising a VEGF antagonist and a PD-L1 axis binding antagonist based on the presence of a sarcomatoid cancer. In some embodiments, the method further comprises administering an effective amount of an anti-cancer therapy comprising a VEGF antagonist and a PD-L1 axis binding antagonist to the individual.

In yet another example, provided herein is a method of identifying an individual having a cancer (e.g., a kidney cancer (e.g., RCC)) who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), the method comprising determining whether the individual has a sarcomatoid cancer, wherein the presence of a sarcomatoid cancer identifies the individual as one who may benefit from treatment with an anti-cancer therapy comprising a VEGF antagonist and a PD-L1 axis binding antagonist, wherein the benefit is in terms of improved CR rate. In some embodiments, the method further comprises administering an effective amount of an anti-cancer therapy comprising a VEGF antagonist and a PD-L1 axis binding antagonist to the individual.

In another example, provided herein is a method for selecting a therapy for an individual having cancer (e.g., a kidney cancer (e.g., RCC)), the method comprising (a) determining whether the individual has a sarcomatoid cancer, wherein the presence of a sarcomatoid cancer identifies the individual as one who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved CR rate; and (b) selecting an anti-cancer therapy comprising a VEGF antagonist and a PD-L1 axis binding antagonist based on the presence of a sarcomatoid cancer. In some embodiments, the method further comprises administering an effective amount of an anti-cancer therapy comprising a VEGF antagonist and a PD-L1 axis binding antagonist to the individual.

In yet another example, provided herein is a method of identifying an individual having a cancer (e.g., a kidney cancer (e.g., RCC)) who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), the method comprising determining whether the individual has a sarcomatoid cancer, wherein the presence of a sarcomatoid cancer identifies the individual as one who may benefit from treatment with an anti-cancer therapy comprising a VEGF antagonist and a PD-L1 axis binding antagonist, wherein the benefit is in terms of improved DFR. In some embodiments, the method further comprises administering an effective amount of an anti-cancer therapy comprising a VEGF antagonist and a PD-L1 axis binding antagonist to the individual.

In another example, provided herein is a method for selecting a therapy for an individual having cancer (e.g., a kidney cancer (e.g., RCC)), the method comprising (a) determining whether the individual has a sarcomatoid cancer, wherein the presence of a sarcomatoid cancer identifies the individual as one who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved DFR; and (b) selecting an anti-cancer therapy comprising a VEGF antagonist and a PD-L1 axis binding antagonist based on the presence of a sarcomatoid cancer. In some embodiments, the method further comprises administering an effective amount of an anti-cancer therapy comprising a VEGF antagonist and a PD-L1 axis binding antagonist to the individual.

The presence of a sarcomatoid cancer (e.g., a sarcomatoid kidney cancer (e.g., a sarcomatoid RCC)) can be determined using any suitable approach. See, e.g., El Mouallem et al. Urol. Oncol. 36:265-271, 2018. For example, in some embodiments, the presence of a sarcomatoid cancer (e.g., a sarcomatoid kidney cancer (e.g., a sarcomatoid RCC)) is assessed by histological analysis of a sample obtained from the individual. In some embodiments, the kidney cancer is sarcomatoid if a tumor sample from the individual contains a focus or foci of high-grade malignant spindle cells of any component relative to the entire tumor area. In some embodiments, the spindle cells show moderate to marked atypia and/or resemble any form of sarcoma. In some embodiments, the spindle cells show evidence of epithelial differentiation as assessed by immunohistological positivity for keratin or epithelial membrane antigen (EMA). In some embodiments, the kidney cancer is renal cell carcinoma, and the tumor sample has epithelial differentiation with concurrent areas of renal cell carcinoma.

In any of the preceding methods, the method may further include determining the individual's MSKCC risk score. In other embodiments, the individual's MSKCC risk score has previously been determined. In any of the preceding methods, the individual may have a poor or intermediate MSKCC risk score.

In another example, provided herein is a method of identifying an individual having a cancer (e.g., a kidney cancer (e.g., RCC)) who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), the method comprising determining the individual's MSKCC risk score, wherein a poor or intermediate MSKCC risk score identifies the individual as likely to benefit from an anti-cancer therapy comprising a VEGF antagonist and a PD-L1 axis binding antagonist.

In yet another example, provided herein is a method for selecting a therapy for an individual having a cancer (e.g., a kidney cancer (e.g., RCC)), the method comprising (a) determining the individual's MSKCC risk score, wherein a poor or intermediate MSKCC risk score identifies the individual as likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody; and (b) selecting an anti-cancer therapy comprising a VEGF antagonist and a PD-L1 axis binding antagonist based on the individual having a poor or intermediate MSKCC risk score.

The benefit may be, for example, in terms of improved progression-free survival (PFS), overall survival (OS), overall response rate (ORR), complete response (CR) rate, or deterioration-free rate (DFR). In some embodiments, the benefit is in terms of improved PFS. In some instances, the benefit is in terms of improved OS. In some instances, the benefit is in terms of improved ORR. In some instances, the benefit is in terms of improved CR rate. In some instances, the benefit is in terms of improved DFR. In some instances, DFR is determined in terms of the time from onset of treatment to the individual's first increase of greater than or equal to 2 points above baseline on the MD Anderson Symptom Inventory (MDASI) interference scale.

For example, provided herein is a method of identifying an individual having a cancer (e.g., a kidney cancer (e.g., RCC)) who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), the method comprising determining the individual's MSKCC risk score, wherein a poor or intermediate MSKCC risk score identifies the individual as likely to benefit from an anti-cancer therapy comprising a VEGF antagonist and a PD-L1 axis binding antagonist, wherein the benefit is in terms of improved PFS.

In another example, provided herein is a method for selecting a therapy for an individual having a cancer (e.g., a kidney cancer (e.g., RCC)), the method comprising (a) determining the individual's MSKCC risk score, wherein a poor or intermediate MSKCC risk score identifies the individual as likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved PFS; and (b) selecting an anti-cancer therapy comprising a VEGF antagonist and a PD-L1 axis binding antagonist based on the individual having a poor or intermediate MSKCC risk score.

In another example, provided herein is a method of identifying an individual having a cancer (e.g., a kidney cancer (e.g., RCC)) who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), the method comprising determining the individual's MSKCC risk score, wherein a poor or intermediate MSKCC risk score identifies the individual as likely to benefit from an anti-cancer therapy comprising a VEGF antagonist and a PD-L1 axis binding antagonist, wherein the benefit is in terms of improved OS.

In yet another example, provided herein is a method for selecting a therapy for an individual having a cancer (e.g., a kidney cancer (e.g., RCC)), the method comprising (a) determining the individual's MSKCC risk score, wherein a poor or intermediate MSKCC risk score identifies the individual as likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved OS; and (b) selecting an anti-cancer therapy comprising a VEGF antagonist and a PD-L1 axis binding antagonist based on the individual having a poor or intermediate MSKCC risk score.

In a further example, provided herein is a method of identifying an individual having a cancer (e.g., a kidney cancer (e.g., RCC)) who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), the method comprising determining the individual's MSKCC risk score, wherein a poor or intermediate MSKCC risk score identifies the individual as likely to benefit from an anti-cancer therapy comprising a VEGF antagonist and a PD-L1 axis binding antagonist, wherein the benefit is in terms of improved ORR.

In a still further example, provided herein is a method for selecting a therapy for an individual having a cancer (e.g., a kidney cancer (e.g., RCC)), the method comprising (a) determining the individual's MSKCC risk score, wherein a poor or intermediate MSKCC risk score identifies the individual as likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved ORR; and (b) selecting an anti-cancer therapy comprising a VEGF antagonist and a PD-L1 axis binding antagonist based on the individual having a poor or intermediate MSKCC risk score.

In yet a further example, provided herein is a method of identifying an individual having a cancer (e.g., a kidney cancer (e.g., RCC)) who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), the method comprising determining the individual's MSKCC risk score, wherein a poor or intermediate MSKCC risk score identifies the individual as likely to benefit from an anti-cancer therapy comprising a VEGF antagonist and a PD-L1 axis binding antagonist, wherein the benefit is in terms of improved CR rate.

In a still further example, provided herein is a method for selecting a therapy for an individual having a cancer (e.g., a kidney cancer (e.g., RCC)), the method comprising (a) determining the individual's MSKCC risk score, wherein a poor or intermediate MSKCC risk score identifies the individual as likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved CR rate; and (b) selecting an anti-cancer therapy comprising a VEGF antagonist and a PD-L1 axis binding antagonist based on the individual having a poor or intermediate MSKCC risk score.

In another example, provided herein is a method of identifying an individual having a cancer (e.g., a kidney cancer (e.g., RCC)) who may benefit from treatment with an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), the method comprising determining the individual's MSKCC risk score, wherein a poor or intermediate MSKCC risk score identifies the individual as likely to benefit from an anti-cancer therapy comprising a VEGF antagonist and a PD-L1 axis binding antagonist, wherein the benefit is in terms of improved DFR.

In yet another example, provided herein is a method for selecting a therapy for an individual having a cancer (e.g., a kidney cancer (e.g., RCC)), the method comprising (a) determining the individual's MSKCC risk score, wherein a poor or intermediate MSKCC risk score identifies the individual as likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved DFR; and (b) selecting an anti-cancer therapy comprising a VEGF antagonist and a PD-L1 axis binding antagonist based on the individual having a poor or intermediate MSKCC risk score.

In any of the preceding methods, the individual may have a poor MSKCC risk score if the individual has three or more (e.g., three, four, or all five) of the following characteristics: (i) a time from nephrectomy to systemic treatment of less than one year, a lack of a nephrectomy, or an initial diagnosis with metastatic disease; (ii) a hemoglobin level less than the lower limit of normal (LLN), optionally wherein the normal range for hemoglobin is between 13.5 and 17.5 g/dL for men and between 12 and 15.5 g/dL for women; (iii) a serum corrected calcium level greater than 10 mg/dL, optionally wherein the serum corrected calcium level is the serum calcium level (mg/dL)+0.8(4−serum albumin (g/dL)); (iv) a serum lactate dehydrogenase (LDH) level greater than 1.5 times the upper limit of normal (ULN), optionally wherein the ULN is 140 U/L; and/or (v) a Karnofsky Performance Status (KPS) score of <80. In some embodiments, the individual has three of the preceding characteristics. In other embodiments, the individual has four of the preceding characteristics. In yet other embodiments, the individual has all five of the preceding characteristics.

In any of the preceding methods, the individual may have an intermediate MSKCC risk score if the individual has one or two of the following characteristics: (i) a time from nephrectomy to systemic treatment of less than one year, a lack of a nephrectomy, or an initial diagnosis with metastatic disease; (ii) a hemoglobin level less than the LLN, optionally wherein the normal range for hemoglobin is between 13.5 and 17.5 g/dL for men and between 12 and 15.5 g/dL for women; (iii) a serum corrected calcium level greater than 10 mg/dL, optionally wherein the serum corrected calcium level is the serum calcium level (mg/dL)+0.8(4−serum albumin (g/dL)); (iv) a serum LDH level greater than 1.5 times the ULN, optionally wherein the ULN is 140 U/L; and/or (v) a KPS score of <80. In some embodiments, the individual has one of the preceding characteristics. In other embodiments, the individual has two of the preceding characteristics.

In any of the preceding methods, the individual may have a sarcomatoid cancer (e.g., a sarcomatoid kidney cancer (e.g., a sarcomatoid RCC)).

In some embodiments of any of the preceding methods, the method further comprises determining the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37) of the genes set forth in Table 1. In other embodiments, the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37) of the genes set forth in Table 1 has previously been determined.

TABLE 1
Exemplary Biomarkers
Biomarkers

CD8A
EOMES
GZMA
GZMB
PRF1
IFNG
PD-L1
CXCL9
CXCL10
CXCL11
CD27
FOXP3
PD-1
CTLA4
TIGIT
IDO1
PSMB8
PSMB9
TAP1
TAP2
VEGFA
KDR
ESM1
PECAM1
FLT1
ANGPTL4
CD34
IL6
CXCL1
CXCL2
CXCL3
CXCL8
PTGS2
CXCR1
CXCR2
S100A8
S100A9

For example, in some embodiments, the method further comprises determining the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33) of the following genes in a sample from the individual: CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2; VEGFA, KDR, ESM1, PECAM1, FLT1, ANGPTL4, or CD34; or IL6, CXCL1, CXCL2, CXCL3, CXCL8, or PTGS2. In other embodiments, the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33) of the following genes in a sample from the individual: CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2; VEGFA, KDR, ESM1, PECAM1, FLT1, ANGPTL4, or CD34; or IL6, CXCL1, CXCL2, CXCL3, CXCL8, or PTGS2 has previously been determined.

In some embodiments of any of the preceding methods, (i) an expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample that is at or above a reference expression level of the one or more genes; or (ii) an expression level of one or more (e.g., 1, 2, 3, 4, 5, 6, 7, 8, 9, 10, 11, 12, or 13) of VEGFA, KDR, ESM1, PECAM1, FLT1, ANGPTL4, or CD34; or IL6, CXCL1, CXCL2, CXCL3, CXCL8, or PTGS2 in the sample that is below a reference expression level of the one or more genes identifies the individual as one who may benefit from treatment with an anti-cancer therapy comprising a VEGF antagonist and a PD-L1 axis binding antagonist.

In any of the preceding methods, the method may include determining the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2. In some embodiments, the method includes determining the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2.

For example, any of the preceding methods may include determining the expression level of one or more (e.g., 1, 2, 3, 4, or 5) of CD8A, EOMES, PRF1, IFNG, or PD-L1. In some embodiments, the method includes determining the expression level of at least two, at least three, at least four, or all five of CD8A, EOMES, PRF1, IFNG, and PD-L1. In some embodiments, the method includes determining the expression level of two of CD8A, EOMES, PRF1, IFNG, and PD-L1, for example, any of the exemplary combinations shown in Table 2. In some embodiments, the method includes determining the expression level of three of CD8A, EOMES, PRF1, IFNG, and PD-L1, for example, any of the exemplary combinations shown in Table 3. In some embodiments, the method includes determining the expression level of four of CD8A, EOMES, PRF1, IFNG, and PD-L1, for example, any of the exemplary combinations shown in Table 4. In some embodiments, the method involves determining the expression level of CD8A, EOMES, PRF1, IFNG, and PD-L1.

TABLE 2
Two-Gene Combinations of CD8A, EOMES, PRF1, IFNG, and PD-L1

CD8A and EOMES
CD8A and PRF1
CD8A and IFNG
CD8A and PD-L1
EOMES and PRF1
EOMES and IFNG
EOMES and PD-L1
PRF1 and IFNG
PRF1 and PD-L1
IFNG and PD-L1

TABLE 3
Three-Gene Combinations of CD8A, EOMES, PRF1, IFNG, and PD-L1

CD8A, EOMES, and PRF1
CD8A, EOMES, and IFNG
CD8A, EOMES, and PD-L1
CD8A, PRF1, and IFNG
CD8A, PRF1, and PD-L1
CD8A, IFNG, and PD-L1
EOMES, PRF1, and IFNG
EOMES, PRF1, and PD-L1
EOMES, IFNG, and PD-L1
PRF1, IFNG, and PD-L1

TABLE 4
Four-Gene Combinations of CD8A, EOMES, PRF1, IFNG, and PD-L1

CD8A, EOMES, PRF1, and IFNG
CD8A, EOMES, PRF1, and PD-L1
CD8A, EOMES, IFNG, and PD-L1
CD8A, PRF1, IFNG, and PD-L1
EOMES, PRF1, IFNG, and PD-L1

In some embodiments, any of the preceding methods may include determining the expression level of PD-L1 and one or more additional genes, wherein the one or more additional genes is other than PD-L1. For example, in some embodiments, the method may include determining the expression level of PD-L1 and one or more additional genes (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36) selected from the group consisting of: CD8A, EOMES, GZMA, GZMB, PRF1, IFNG, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and S100A9. In some embodiments, the method includes determining the expression level of PD-L1 and one or more (e.g., 1, 2, 3, 4, 5, 6, 7, 8, 9, 10, 11, 12, 13, 14, 15, 16, 17, 18, or 19) additional genes selected from the group consisting of CD8A, EOMES, GZMA, GZMB, PRF1, IFNG, CXCL9, CXCL10, CXCL11, CD27, FOXP3, PD-1, CTLA4, TIGIT, IDO1, PSMB8, PSMB9, TAP1, and TAP2. In other embodiments, the method includes determining the expression level of PD-L1 and one or more (e.g., 1, 2, 3, 4, 5, 6, or 7) of VEGFA, KDR, ESM1, PECAM1, FLT1, ANGPTL4, or CD34. In other embodiments, the method includes determining the expression level of PD-L1 and one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9.

Any of the preceding methods may include determining the expression level of one or more (e.g., 1, 2, 3, 4, 5, 6, or 7) of VEGFA, KDR, ESM1, PECAM1, FLT1, ANGPTL4, or CD34. In some embodiments, the method includes determining the expression level of at least two, at least three, at least four, at least five, at least six, or all seven of VEGFA, KDR, ESM1, PECAM1, FLT1, ANGPTL4, or CD34. For example, in some embodiments, the method includes determining the expression level of one or more (e.g., 1, 2, 3, 4, 5, or 6) of VEGFA, KDR, ESM1, PECAM1, ANGPTL4, or CD34. In some embodiments, the method includes determining the expression level of at least two, at least three, at least four, at least five, or all six of VEGFA, KDR, ESM1, PECAM1, ANGPTL4, and CD34. In some embodiments, the method includes determining the expression level of two of VEGFA, KDR, ESM1, PECAM1, ANGPTL4, and CD34, for example, any of the exemplary combinations shown in Table 5. In some embodiments, the method includes determining the expression level of three of VEGFA, KDR, ESM1, PECAM1, ANGPTL4, and CD34, for example, any of the exemplary combinations shown in Table 6. In some embodiments, the method includes determining the expression level of four of VEGFA, KDR, ESM1, PECAM1, ANGPTL4, and CD34, for example, any of the exemplary combinations shown in Table 7. In some embodiments, the method includes determining the expression level of five of VEGFA, KDR, ESM1, PECAM1, ANGPTL4, and CD34, for example, any of the exemplary combinations shown in Table 8. In some embodiments, the method includes determining the expression level of VEGFA, KDR, ESM1, PECAM1, ANGPTL4, and CD34.

TABLE 5
Two-Gene Combinations of VEGFA, KDR, ESM1,
PECAM1, ANGPTL4, and CD34

VEGFA and KDR
VEGFA and ESM1
VEGFA and PECAM1
VEGFA and ANGPTL4
VEGFA and CD34
KDR and ESM1
KDR and PECAM1
KDR and ANGPTL4
KDR and CD34
ESM1 and PECAM1
ESM1 and ANGPTL4
ESM1 and CD34
PECAM1 and ANGPTL4
PECAM1 and CD34
ANGPTL4 and CD34

TABLE 6
Three-Gene Combinations of VEGFA, KDR, ESM1,
PECAM1, ANGPTL4, and CD34

VEGFA, KDR, and ESM1
VEGFA, KDR, and PECAM1
VEGFA, KDR, and ANGPTL4
VEGFA, KDR, and CD34
VEGFA, ESM1, and PECAM1
VEGFA, ESM1, and ANGPTL4
VEGFA, ESM1, and CD34
VEGFA, PECAM1, and ANGPTL4
VEGFA, PECAM1, and CD34
VEGFA, ANGPTL4, and CD34
KDR, ESM1, and PECAM1
KDR, ESM1, and ANGPTL4
KDR, ESM1, and CD34
KDR, PECAM1, and ANGPTL4
KDR, PECAM1, and CD34
KDR, ANGPTL4, and CD34
ESM1, PECAM1, and ANGPTL4
ESM1, PECAM1, and CD34
ESM1, ANGPTL4, and CD34
PECAM1, ANGPTL4, and CD34

TABLE 7
Four-Gene Combinations of VEGFA, KDR, ESM1,
PECAM1, ANGPTL4, and CD34

VEGFA, KDR, ESM1, and PECAM1
VEGFA, KDR, ESM1, and ANGPTL4
VEGFA, KDR, ESM1, and CD34
VEGFA, KDR, PECAM1, and ANGPTL4
VEGFA, KDR, PECAM1, and CD34
VEGFA, KDR, ANGPTL4, and CD34
VEGFA, ESM1, PECAM1, and ANGPTL4
VEGFA, ESM1, PECAM1, and CD34
VEGFA, ESM1, ANGPTL4, and CD34
VEGFA, PECAM1, ANGPTL4, and CD34
KDR, ESM1, PECAM1, and ANGPTL4
KDR, ESM1, PECAM1, and CD34
KDR, ESM1, ANGPTL4, and CD34
KDR, PECAM1, ANGPTL4, and CD34
ESM1, PECAM1, ANGPTL4, and CD34

TABLE 8
Five-Gene Combinations of VEGFA, KDR, ESM1,
PECAM1, ANGPTL4, and CD34

VEGFA, KDR, ESM1, PECAM1, and ANGPTL4
VEGFA, KDR, ESM1, PECAM1, and CD34
VEGFA, KDR, ESM1, ANGPTL4, and CD34
VEGFA, KDR, PECAM1, ANGPTL4, and CD34
VEGFA, ESM1, PECAM1, ANGPTL4, and CD34
KDR, ESM1, PECAM1, ANGPTL4, and CD34

Any of the preceding methods may include determining the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9. In some embodiments, the method includes determining the expression level of at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9. In some embodiments, the method includes determining the expression level of two of IL6, CXCL1, CXCL2, CXCL3, CXCL8, PTGS2, CXCR1, CXCR2, S100A8, and S100A9, for example, any of the exemplary combinations shown in Table 9. In some embodiments, the method includes determining the expression level of three of IL6, CXCL1, CXCL2, CXCL3, CXCL8, PTGS2, CXCR1, CXCR2, S100A8, and S100A9, for example, any of the exemplary combinations shown in Table 10. In some embodiments, the method includes determining the expression level of four of IL6, CXCL1, CXCL2, CXCL3, CXCL8, PTGS2, CXCR1, CXCR2, S100A8, and S100A9, for example, any of the exemplary combinations shown in Table 11. In some embodiments, the method includes determining the expression level of five of IL6, CXCL1, CXCL2, CXCL3, CXCL8, PTGS2, CXCR1, CXCR2, S100A8, and S100A9, for example, any of the exemplary combinations shown in Table 12. In some embodiments, the method includes determining the expression level of six of IL6, CXCL1, CXCL2, CXCL3, CXCL8, PTGS2, CXCR1, CXCR2, S100A8, and S100A9, for example, any of the exemplary combinations shown in Table 13. In some embodiments, the method includes determining the expression level of seven of IL6, CXCL1, CXCL2, CXCL3, CXCL8, PTGS2, CXCR1, CXCR2, S100A8, and S100A9, for example, any of the exemplary combinations shown in Table 14. In some embodiments, the method includes determining the expression level of eight of IL6, CXCL1, CXCL2, CXCL3, CXCL8, PTGS2, CXCR1, CXCR2, S100A8, and S100A9, for example, any of the exemplary combinations shown in Table 15. In some embodiments, the method includes determining the expression level of nine of IL6, CXCL1, CXCL2, CXCL3, CXCL8, PTGS2, CXCR1, CXCR2, S100A8, and S100A9, for example, any of the exemplary combinations shown in Table 16. In some embodiments, the method includes determining the expression level of IL6, CXCL1, CXCL2, CXCL3, CXCL8, PTGS2, CXCR1, CXCR2, S100A8, and S100A9.

TABLE 9
Two-Gene Combinations of IL6, CXCL1, CXCL2, CXCL3, CXCL8,
PTGS2, CXCR1, CXCR2, S100A8, and S100A9

IL6 and CXCL1
IL6 and CXCL2
IL6 and CXCL3
IL6 and CXCL8
IL6 and PTGS2
IL6 and CXCR1
IL6 and CXCR2
IL6 and S100A8
IL6 and S100A9
CXCL1 and CXCL2
CXCL1 and CXCL3
CXCL1 and CXCL8
CXCL1 and PTGS2
CXCL1 and CXCR1
CXCL1 and CXCR2
CXCL1 and S100A8
CXCL1 and S100A9
CXCL2 and CXCL3
CXCL2 and CXCL8
CXCL2 and PTGS2
CXCL2 and CXCR1
CXCL2 and CXCR2
CXCL2 and S100A8
CXCL2 and S100A9
CXCL3 and CXCL8
CXCL3 and PTGS2
CXCL3 and CXCR1
CXCL3 and CXCR2
CXCL3 and S100A8
CXCL3 and S100A9
CXCL8 and PTGS2
CXCL8 and CXCR1
CXCL8 and CXCR2
CXCL8 and S100A8
CXCL8 and S100A9
PTGS2 and CXCR1
PTGS2 and CXCR2
PTGS2 and S100A8
PTGS2 and S100A9
CXCR1 and CXCR2
CXCR1 and S100A8
CXCR1 and S100A9
CXCR2 and S100A8
CXCR2 and S100A9
S100A8 and S100A9

TABLE 10
Three-Gene Combinations of IL6, CXCL1, CXCL2, CXCL3,
CXCL8, PTGS2, CXCR1, CXCR2, S100A8, and S100A9

IL6, CXCL1, and CXCL2
IL6, CXCL1, and CXCL3
IL6, CXCL1, and CXCL8
IL6, CXCL1, and PTGS2
IL6, CXCL1, and CXCR1
IL6, CXCL1, and CXCR2
IL6, CXCL1, and S100A8
IL6, CXCL1, and S100A9
IL6, CXCL2, and CXCL3
IL6, CXCL2, and CXCL8
IL6, CXCL2, and PTGS2
IL6, CXCL2, and CXCR1
IL6, CXCL2, and CXCR2
IL6, CXCL2, and S100A8
IL6, CXCL2, and S100A9
IL6, CXCL3, and CXCL8
IL6, CXCL3, and PTGS2
IL6, CXCL3, and CXCR1
IL6, CXCL3, and CXCR2
IL6, CXCL3, and S100A8
IL6, CXCL3, and S100A9
IL6, CXCL8, and PTGS2
IL6, CXCL8, and CXCR1
IL6, CXCL8, and CXCR2
IL6, CXCL8, and S100A8
IL6, CXCL8, and S100A9
IL6, PTGS2, and CXCR1
IL6, PTGS2, and CXCR2
IL6, PTGS2, and S100A8
IL6, PTGS2, and S100A9
IL6, CXCR1, and CXCR2
IL6, CXCR1, and S100A8
IL6, CXCR1, and S100A9
IL6, CXCR2, and S100A8
IL6, CXCR2, and S100A9
IL6, S100A8, and S100A9
CXCL1, CXCL2, and CXCL3
CXCL1, CXCL2, and CXCL8
CXCL1, CXCL2, and PTGS2
CXCL1, CXCL2, and CXCR1
CXCL1, CXCL2, and CXCR2
CXCL1, CXCL2, and S100A8
CXCL1, CXCL2, and S100A9
CXCL1, CXCL3, and CXCL8
CXCL1, CXCL3, and PTGS2
CXCL1, CXCL3, and CXCR1
CXCL1, CXCL3, and CXCR2
CXCL1, CXCL3, and S100A8
CXCL1, CXCL3, and S100A9
CXCL1, CXCL8, and PTGS2
CXCL1, CXCL8, and CXCR1
CXCL1, CXCL8, and CXCR2
CXCL1, CXCL8, and S100A8
CXCL1, CXCL8, and S100A9
CXCL1, PTGS2, and CXCR1
CXCL1, PTGS2, and CXCR2
CXCL1, PTGS2, and S100A8
CXCL1, PTGS2, and S100A9
CXCL1, CXCR1, and CXCR2
CXCL1, CXCR1, and S100A8
CXCL1, CXCR1, and S100A9
CXCL1, CXCR2, and S100A8
CXCL1, CXCR2, and S100A9
CXCL1, S100A8, and S100A9
CXCL2, CXCL3, and CXCL8
CXCL2, CXCL3, and PTGS2
CXCL2, CXCL3, and CXCR1
CXCL2, CXCL3, and CXCR2
CXCL2, CXCL3, and S100A8
CXCL2, CXCL3, and S100A9
CXCL2, CXCL8, and PTGS2
CXCL2, CXCL8, and CXCR1
CXCL2, CXCL8, and CXCR2
CXCL2, CXCL8, and S100A8
CXCL2, CXCL8, and S100A9
CXCL2, PTGS2, and CXCR1
CXCL2, PTGS2, and CXCR2
CXCL2, PTGS2, and S100A8
CXCL2, PTGS2, and S100A9
CXCL2, CXCR1, and CXCR2
CXCL2, CXCR1, and S100A8
CXCL2, CXCR1, and S100A9
CXCL2, CXCR2, and S100A8
CXCL2, CXCR2, and S100A9
CXCL2, S100A8, and S100A9
CXCL3, CXCL8, and PTGS2
CXCL3, CXCL8, and CXCR1
CXCL3, CXCL8, and CXCR2
CXCL3, CXCL8, and S100A8
CXCL3, CXCL8, and S100A9
CXCL3, PTGS2, and CXCR1
CXCL3, PTGS2, and CXCR2
CXCL3, PTGS2, and S100A8
CXCL3, PTGS2, and S100A9
CXCL3, CXCR1, and CXCR2
CXCL3, CXCR1, and S100A8
CXCL3, CXCR1, and S100A9
CXCL3, CXCR2, and S100A8
CXCL3, CXCR2, and S100A9
CXCL3, S100A8, and S100A9
CXCL8, PTGS2, and CXCR1
CXCL8, PTGS2, and CXCR2
CXCL8, PTGS2, and S100A8
CXCL8, PTGS2, and S100A9
CXCL8, CXCR1, and CXCR2
CXCL8, CXCR1, and S100A8
CXCL8, CXCR1, and S100A9
CXCL8, CXCR2, and S100A8
CXCL8, CXCR2, and S100A9
CXCL8, S100A8, and S100A9
PTGS2, CXCR1, and CXCR2
PTGS2, CXCR1, and S100A8
PTGS2, CXCR1, and S100A9
PTGS2, CXCR2, and S100A8
PTGS2, CXCR2, and S100A9
PTGS2, S100A8, and S100A9
CXCR1, CXCR2, and S100A8
CXCR1, CXCR2, and S100A9
CXCR1, S100A8, and S100A9
CXCR2, S100A8, and S100A9

TABLE 11
Four-Gene Combinations of IL6, CXCL1,
CXCL2, CXCL3, CXCL8, PTGS2,
CXCR1, CXCR2, S100A8, and S100A9
IL6, CXCL1, CXCL2, and CXCL3
IL6, CXCL1, CXCL2, and CXCL8
IL6, CXCL1, CXCL2, and PTGS2
IL6, CXCL1, CXCL2, and CXCR1
IL6, CXCL1, CXCL2, and CXCR2
IL6, CXCL1, CXCL2, and S100A8
IL6, CXCL1, CXCL2, and S100A9
IL6, CXCL1, CXCL3, and CXCL8
IL6, CXCL1, CXCL3, and PTGS2
IL6, CXCL1, CXCL3, and CXCR1
IL6, CXCL1, CXCL3, and CXCR2
IL6, CXCL1, CXCL3, and S100A8
IL6, CXCL1, CXCL3, and S100A9
IL6, CXCL1, CXCL8, and PTGS2
IL6, CXCL1, CXCL8, and CXCR1
IL6, CXCL1, CXCL8, and CXCR2
IL6, CXCL1, CXCL8, and S100A8
IL6, CXCL1, CXCL8, and S100A9
IL6, CXCL1, PTGS2, and CXCR1
IL6, CXCL1, PTGS2, and CXCR2
IL6, CXCL1, PTGS2, and S100A8
IL6, CXCL1, PTGS2, and S100A9
IL6, CXCL1, CXCR1, and CXCR2
IL6, CXCL1, CXCR1, and S100A8
IL6, CXCL1, CXCR1, and S100A9
IL6, CXCL1, CXCR2, and S100A8
IL6, CXCL1, CXCR2, and S100A9
IL6, CXCL1, S100A8, and S100A9
IL6, CXCL2, CXCL3, and CXCL8
IL6, CXCL2, CXCL3, and PTGS2
IL6, CXCL2, CXCL3, and CXCR1
IL6, CXCL2, CXCL3, and CXCR2
IL6, CXCL2, CXCL3, and S100A8
IL6, CXCL2, CXCL3, and S100A9
IL6, CXCL2, CXCL8, and PTGS2
IL6, CXCL2, CXCL8, and CXCR1
IL6, CXCL2, CXCL8, and CXCR2
IL6, CXCL2, CXCL8, and S100A8
IL6, CXCL2, CXCL8, and S100A9
IL6, CXCL2, PTGS2, and CXCR1
IL6, CXCL2, PTGS2, and CXCR2
IL6, CXCL2, PTGS2, and S100A8
IL6, CXCL2, PTGS2, and S100A9
IL6, CXCL2, CXCR1, and CXCR2
IL6, CXCL2, CXCR1, and S100A8
IL6, CXCL2, CXCR1, and S100A9
IL6, CXCL2, CXCR2, and S100A8
IL6, CXCL2, CXCR2, and S100A9
IL6, CXCL2, S100A8, and S100A9
IL6, CXCL3, CXCL8, and PTGS2
IL6, CXCL3, CXCL8, and CXCR1
IL6, CXCL3, CXCL8, and CXCR2
IL6, CXCL3, CXCL8, and S100A8
IL6, CXCL3, CXCL8, and S100A9
IL6, CXCL3, PTGS2, and CXCR1
IL6, CXCL3, PTGS2, and CXCR2
IL6, CXCL3, PTGS2, and S100A8
IL6, CXCL3, PTGS2, and S100A9
IL6, CXCL3, CXCR1, and CXCR2
IL6, CXCL3, CXCR1, and S100A8
IL6, CXCL3, CXCR1, and S100A9
IL6, CXCL3, CXCR2, and S100A8
IL6, CXCL3, CXCR2, and S100A9
IL6, CXCL3, S100A8, and S100A9
IL6, CXCL8, PTGS2, and CXCR1
IL6, CXCL8, PTGS2, and CXCR2
IL6, CXCL8, PTGS2, and S100A8
IL6, CXCL8, PTGS2, and S100A9
IL6, CXCL8, CXCR1, and CXCR2
IL6, CXCL8, CXCR1, and S100A8
IL6, CXCL8, CXCR1, and S100A9
IL6, CXCL8, CXCR2, and S100A8
IL6, CXCL8, CXCR2, and S100A9
IL6, CXCL8, S100A8, and S100A9
IL6, PTGS2, CXCR1, and CXCR2
IL6, PTGS2, CXCR1, and S100A8
IL6, PTGS2, CXCR1, and S100A9
IL6, PTGS2, CXCR2, and S100A8
IL6, PTGS2, CXCR2, and S100A9
IL6, PTGS2, S100A8, and S100A9
IL6, CXCR1, CXCR2, and S100A8
IL6, CXCR1, CXCR2, and S100A9
IL6, CXCR1, S100A8, and S100A9
IL6, CXCR2, S100A8, and S100A9
CXCL1, CXCL2, CXCL3, and CXCL8
CXCL1, CXCL2, CXCL3, and PTGS2
CXCL1, CXCL2, CXCL3, and CXCR1
CXCL1, CXCL2, CXCL3, and CXCR2
CXCL1, CXCL2, CXCL3, and S100A8
CXCL1, CXCL2, CXCL3, and S100A9
CXCL1, CXCL2, CXCL8, and PTGS2
CXCL1, CXCL2, CXCL8, and CXCR1
CXCL1, CXCL2, CXCL8, and CXCR2
CXCL1, CXCL2, CXCL8, and S100A8
CXCL1, CXCL2, CXCL8, and S100A9
CXCL1, CXCL2, PTGS2, and CXCR1
CXCL1, CXCL2, PTGS2, and CXCR2
CXCL1, CXCL2, PTGS2, and S100A8
CXCL1, CXCL2, PTGS2, and S100A9
CXCL1, CXCL2, CXCR1, and CXCR2
CXCL1, CXCL2, CXCR1, and S100A8
CXCL1, CXCL2, CXCR1, and S100A9
CXCL1, CXCL2, CXCR2, and S100A8
CXCL1, CXCL2, CXCR2, and S100A9
CXCL1, CXCL2, S100A8, and S100A9
CXCL1, CXCL3, CXCL8, and PTGS2
CXCL1, CXCL3, CXCL8, and CXCR1
CXCL1, CXCL3, CXCL8, and CXCR2
CXCL1, CXCL3, CXCL8, and S100A8
CXCL1, CXCL3, CXCL8, and S100A9
CXCL1, CXCL3, PTGS2, and CXCR1
CXCL1, CXCL3, PTGS2, and CXCR2
CXCL1, CXCL3, PTGS2, and S100A8
CXCL1, CXCL3, PTGS2, and S100A9
CXCL1, CXCL3, CXCR1, and CXCR2
CXCL1, CXCL3, CXCR1, and S100A8
CXCL1, CXCL3, CXCR1, and S100A9
CXCL1, CXCL3, CXCR2, and S100A8
CXCL1, CXCL3, CXCR2, and S100A9
CXCL1, CXCL3, S100A8, and S100A9
CXCL1, CXCL8, PTGS2, and CXCR1
CXCL1, CXCL8, PTGS2, and CXCR2
CXCL1, CXCL8, PTGS2, and S100A8
CXCL1, CXCL8, PTGS2, and S100A9
CXCL1, CXCL8, CXCR1, and CXCR2
CXCL1, CXCL8, CXCR1, and S100A8
CXCL1, CXCL8, CXCR1, and S100A9
CXCL1, CXCL8, CXCR2, and S100A8
CXCL1, CXCL8, CXCR2, and S100A9
CXCL1, CXCL8, S100A8, and S100A9
CXCL1, PTGS2, CXCR1, and CXCR2
CXCL1, PTGS2, CXCR1, and S100A8
CXCL1, PTGS2, CXCR1, and S100A9
CXCL1, PTGS2, CXCR2, and S100A8
CXCL1, PTGS2, CXCR2, and S100A9
CXCL1, PTGS2, S100A8, and S100A9
CXCL1, CXCR1, CXCR2, and S100A8
CXCL1, CXCR1, CXCR2, and S100A9
CXCL1, CXCR1, S100A8, and S100A9
CXCL1, CXCR2, S100A8, and S100A9
CXCL2, CXCL3, CXCL8, and PTGS2
CXCL2, CXCL3, CXCL8, and CXCR1
CXCL2, CXCL3, CXCL8, and CXCR2
CXCL2, CXCL3, CXCL8, and S100A8
CXCL2, CXCL3, CXCL8, and S100A9
CXCL2, CXCL3, PTGS2, and CXCR1
CXCL2, CXCL3, PTGS2, and CXCR2
CXCL2, CXCL3, PTGS2, and S100A8
CXCL2, CXCL3, PTGS2, and S100A9
CXCL2, CXCL3, CXCR1, and CXCR2
CXCL2, CXCL3, CXCR1, and S100A8
CXCL2, CXCL3, CXCR1, and S100A9
CXCL2, CXCL3, CXCR2, and S100A8
CXCL2, CXCL3, CXCR2, and S100A9
CXCL2, CXCL3, S100A8, and S100A9
CXCL2, CXCL8, PTGS2, and CXCR1
CXCL2, CXCL8, PTGS2, and CXCR2
CXCL2, CXCL8, PTGS2, and S100A8
CXCL2, CXCL8, PTGS2, and S100A9
CXCL2, CXCL8, CXCR1, and CXCR2
CXCL2, CXCL8, CXCR1, and S100A8
CXCL2, CXCL8, CXCR1, and S100A9
CXCL2, CXCL8, CXCR2, and S100A8
CXCL2, CXCL8, CXCR2, and S100A9
CXCL2, CXCL8, S100A8, and S100A9
CXCL2, PTGS2, CXCR1, and CXCR2
CXCL2, PTGS2, CXCR1, and S100A8
CXCL2, PTGS2, CXCR1, and S100A9
CXCL2, PTGS2, CXCR2, and S100A8
CXCL2, PTGS2, CXCR2, and S100A9
CXCL2, PTGS2, S100A8, and S100A9
CXCL2, CXCR1, CXCR2, and S100A8
CXCL2, CXCR1, CXCR2, and S100A9
CXCL2, CXCR1, S100A8, and S100A9
CXCL2, CXCR2, S100A8, and S100A9
CXCL3, CXCL8, PTGS2, and CXCR1
CXCL3, CXCL8, PTGS2, and CXCR2
CXCL3, CXCL8, PTGS2, and S100A8
CXCL3, CXCL8, PTGS2, and S100A9
CXCL3, CXCL8, CXCR1, and CXCR2
CXCL3, CXCL8, CXCR1, and S100A8
CXCL3, CXCL8, CXCR1, and S100A9
CXCL3, CXCL8, CXCR2, and S100A8
CXCL3, CXCL8, CXCR2, and S100A9
CXCL3, CXCL8, S100A8, and S100A9
CXCL3, PTGS2, CXCR1, and CXCR2
CXCL3, PTGS2, CXCR1, and S100A8
CXCL3, PTGS2, CXCR1, and S100A9
CXCL3, PTGS2, CXCR2, and S100A8
CXCL3, PTGS2, CXCR2, and S100A9
CXCL3, PTGS2, S100A8, and S100A9
CXCL3, CXCR1, CXCR2, and S100A8
CXCL3, CXCR1, CXCR2, and S100A9
CXCL3, CXCR1, S100A8, and S100A9
CXCL3, CXCR2, S100A8, and S100A9
CXCL8, PTGS2, CXCR1, and CXCR2
CXCL8, PTGS2, CXCR1, and S100A8
CXCL8, PTGS2, CXCR1, and S100A9
CXCL8, PTGS2, CXCR2, and S100A8
CXCL8, PTGS2, CXCR2, and S100A9
CXCL8, PTGS2, S100A8, and S100A9
CXCL8, CXCR1, CXCR2, and S100A8
CXCL8, CXCR1, CXCR2, and S100A9
CXCL8, CXCR1, S100A8, and S100A9
CXCL8, CXCR2, S100A8, and S100A9
PTGS2, CXCR1, CXCR2, and S100A8
PTGS2, CXCR1, CXCR2, and S100A9
PTGS2, CXCR1, S100A8, and S100A9
PTGS2, CXCR2, S100A8, and S100A9
CXCR1, CXCR2, S100A8, and S100A9

TABLE 12
Five-Gene Combinations of IL6, CXCL1,
CXCL2, CXCL3, CXCL8, PTGS2, CXCR1,
CXCR2, S100A8, and S100A9
IL6, CXCL1, CXCL2, CXCL3, and CXCL8
IL6, CXCL1, CXCL2, CXCL3, and PTGS2
IL6, CXCL1, CXCL2, CXCL3, and CXCR1
IL6, CXCL1, CXCL2, CXCL3, and CXCR2
IL6, CXCL1, CXCL2, CXCL3, and S100A8
IL6, CXCL1, CXCL2, CXCL3, and S100A9
IL6, CXCL1, CXCL2, CXCL8, and PTGS2
IL6, CXCL1, CXCL2, CXCL8, and CXCR1
IL6, CXCL1, CXCL2, CXCL8, and CXCR2
IL6, CXCL1, CXCL2, CXCL8, and S100A8
IL6, CXCL1, CXCL2, CXCL8, and S100A9
IL6, CXCL1, CXCL2, PTGS2, and CXCR1
IL6, CXCL1, CXCL2, PTGS2, and CXCR2
IL6, CXCL1, CXCL2, PTGS2, and S100A8
IL6, CXCL1, CXCL2, PTGS2, and S100A9
IL6, CXCL1, CXCL2, CXCR1, and CXCR2
IL6, CXCL1, CXCL2, CXCR1, and S100A8
IL6, CXCL1, CXCL2, CXCR1, and S100A9
IL6, CXCL1, CXCL2, CXCR2, and S100A8
IL6, CXCL1, CXCL2, CXCR2, and S100A9
IL6, CXCL1, CXCL2, S100A8, and S100A9
IL6, CXCL1, CXCL3, CXCL8, and PTGS2
IL6, CXCL1, CXCL3, CXCL8, and CXCR1
IL6, CXCL1, CXCL3, CXCL8, and CXCR2
IL6, CXCL1, CXCL3, CXCL8, and S100A8
IL6, CXCL1, CXCL3, CXCL8, and S100A9
IL6, CXCL1, CXCL3, PTGS2, and CXCR1
IL6, CXCL1, CXCL3, PTGS2, and CXCR2
IL6, CXCL1, CXCL3, PTGS2, and S100A8
IL6, CXCL1, CXCL3, PTGS2, and S100A9
IL6, CXCL1, CXCL3, CXCR1, and CXCR2
IL6, CXCL1, CXCL3, CXCR1, and S100A8
IL6, CXCL1, CXCL3, CXCR1, and S100A9
IL6, CXCL1, CXCL3, CXCR2, and S100A8
IL6, CXCL1, CXCL3, CXCR2, and S100A9
IL6, CXCL1, CXCL3, S100A8, and S100A9
IL6, CXCL1, CXCL8, PTGS2, and CXCR1
IL6, CXCL1, CXCL8, PTGS2, and CXCR2
IL6, CXCL1, CXCL8, PTGS2, and S100A8
IL6, CXCL1, CXCL8, PTGS2, and S100A9
IL6, CXCL1, CXCL8, CXCR1, and CXCR2
IL6, CXCL1, CXCL8, CXCR1, and S100A8
IL6, CXCL1, CXCL8, CXCR1, and S100A9
IL6, CXCL1, CXCL8, CXCR2, and S100A8
IL6, CXCL1, CXCL8, CXCR2, and S100A9
IL6, CXCL1, CXCL8, S100A8, and S100A9
IL6, CXCL1, PTGS2, CXCR1, and CXCR2
IL6, CXCL1, PTGS2, CXCR1, and S100A8
IL6, CXCL1, PTGS2, CXCR1, and S100A9
IL6, CXCL1, PTGS2, CXCR2, and S100A8
IL6, CXCL1, PTGS2, CXCR2, and S100A9
IL6, CXCL1, PTGS2, S100A8, and S100A9
IL6, CXCL1, CXCR1, CXCR2, and S100A8
IL6, CXCL1, CXCR1, CXCR2, and S100A9
IL6, CXCL1, CXCR1, S100A8, and S100A9
IL6, CXCL1, CXCR2, S100A8, and S100A9
IL6, CXCL2, CXCL3, CXCL8, and PTGS2
IL6, CXCL2, CXCL3, CXCL8, and CXCR1
IL6, CXCL2, CXCL3, CXCL8, and CXCR2
IL6, CXCL2, CXCL3, CXCL8, and S100A8
IL6, CXCL2, CXCL3, CXCL8, and S100A9
IL6, CXCL2, CXCL3, PTGS2, and CXCR1
IL6, CXCL2, CXCL3, PTGS2, and CXCR2
IL6, CXCL2, CXCL3, PTGS2, and S100A8
IL6, CXCL2, CXCL3, PTGS2, and S100A9
IL6, CXCL2, CXCL3, CXCR1, and CXCR2
IL6, CXCL2, CXCL3, CXCR1, and S100A8
IL6, CXCL2, CXCL3, CXCR1, and S100A9
IL6, CXCL2, CXCL3, CXCR2, and S100A8
IL6, CXCL2, CXCL3, CXCR2, and S100A9
IL6, CXCL2, CXCL3, S100A8, and S100A9
IL6, CXCL2, CXCL8, PTGS2, and CXCR1
IL6, CXCL2, CXCL8, PTGS2, and CXCR2
IL6, CXCL2, CXCL8, PTGS2, and S100A8
IL6, CXCL2, CXCL8, PTGS2, and S100A9
IL6, CXCL2, CXCL8, CXCR1, and CXCR2
IL6, CXCL2, CXCL8, CXCR1, and S100A8
IL6, CXCL2, CXCL8, CXCR1, and S100A9
IL6, CXCL2, CXCL8, CXCR2, and S100A8
IL6, CXCL2, CXCL8, CXCR2, and S100A9
IL6, CXCL2, CXCL8, S100A8, and S100A9
IL6, CXCL2, PTGS2, CXCR1, and CXCR2
IL6, CXCL2, PTGS2, CXCR1, and S100A8
IL6, CXCL2, PTGS2, CXCR1, and S100A9
IL6, CXCL2, PTGS2, CXCR2, and S100A8
IL6, CXCL2, PTGS2, CXCR2, and S100A9
IL6, CXCL2, PTGS2, S100A8, and S100A9
IL6, CXCL2, CXCR1, CXCR2, and S100A8
IL6, CXCL2, CXCR1, CXCR2, and S100A9
IL6, CXCL2, CXCR1, S100A8, and S100A9
IL6, CXCL2, CXCR2, S100A8, and S100A9
IL6, CXCL3, CXCL8, PTGS2, and CXCR1
IL6, CXCL3, CXCL8, PTGS2, and CXCR2
IL6, CXCL3, CXCL8, PTGS2, and S100A8
IL6, CXCL3, CXCL8, PTGS2, and S100A9
IL6, CXCL3, CXCL8, CXCR1, and CXCR2
IL6, CXCL3, CXCL8, CXCR1, and S100A8
IL6, CXCL3, CXCL8, CXCR1, and S100A9
IL6, CXCL3, CXCL8, CXCR2, and S100A8
IL6, CXCL3, CXCL8, CXCR2, and S100A9
IL6, CXCL3, CXCL8, S100A8, and S100A9
IL6, CXCL3, PTGS2, CXCR1, and CXCR2
IL6, CXCL3, PTGS2, CXCR1, and S100A8
IL6, CXCL3, PTGS2, CXCR1, and S100A9
IL6, CXCL3, PTGS2, CXCR2, and S100A8
IL6, CXCL3, PTGS2, CXCR2, and S100A9
IL6, CXCL3, PTGS2, S100A8, and S100A9
IL6, CXCL3, CXCR1, CXCR2, and S100A8
IL6, CXCL3, CXCR1, CXCR2, and S100A9
IL6, CXCL3, CXCR1, S100A8, and S100A9
IL6, CXCL3, CXCR2, S100A8, and S100A9
IL6, CXCL8, PTGS2, CXCR1, and CXCR2
IL6, CXCL8, PTGS2, CXCR1, and S100A8
IL6, CXCL8, PTGS2, CXCR1, and S100A9
IL6, CXCL8, PTGS2, CXCR2, and S100A8
IL6, CXCL8, PTGS2, CXCR2, and S100A9
IL6, CXCL8, PTGS2, S100A8, and S100A9
IL6, CXCL8, CXCR1, CXCR2, and S100A8
IL6, CXCL8, CXCR1, CXCR2, and S100A9
IL6, CXCL8, CXCR1, S100A8, and S100A9
IL6, CXCL8, CXCR2, S100A8, and S100A9
IL6, PTGS2, CXCR1, CXCR2, and S100A8
IL6, PTGS2, CXCR1, CXCR2, and S100A9
IL6, PTGS2, CXCR1, S100A8, and S100A9
IL6, PTGS2, CXCR2, S100A8, and S100A9
IL6, CXCR1, CXCR2, S100A8, and S100A9
CXCL1, CXCL2, CXCL3, CXCL8, and PTGS2
CXCL1, CXCL2, CXCL3, CXCL8, and CXCR1
CXCL1, CXCL2, CXCL3, CXCL8, and CXCR2
CXCL1, CXCL2, CXCL3, CXCL8, and S100A8
CXCL1, CXCL2, CXCL3, CXCL8, and S100A9
CXCL1, CXCL2, CXCL3, PTGS2, and CXCR1
CXCL1, CXCL2, CXCL3, PTGS2, and CXCR2
CXCL1, CXCL2, CXCL3, PTGS2, and S100A8
CXCL1, CXCL2, CXCL3, PTGS2, and S100A9
CXCL1, CXCL2, CXCL3, CXCR1, and CXCR2
CXCL1, CXCL2, CXCL3, CXCR1, and S100A8
CXCL1, CXCL2, CXCL3, CXCR1, and S100A9
CXCL1, CXCL2, CXCL3, CXCR2, and S100A8
CXCL1, CXCL2, CXCL3, CXCR2, and S100A9
CXCL1, CXCL2, CXCL3, S100A8, and S100A9
CXCL1, CXCL2, CXCL8, PTGS2, and CXCR1
CXCL1, CXCL2, CXCL8, PTGS2, and CXCR2
CXCL1, CXCL2, CXCL8, PTGS2, and S100A8
CXCL1, CXCL2, CXCL8, PTGS2, and S100A9
CXCL1, CXCL2, CXCL8, CXCR1, and CXCR2
CXCL1, CXCL2, CXCL8, CXCR1, and S100A8
CXCL1, CXCL2, CXCL8, CXCR1, and S100A9
CXCL1, CXCL2, CXCL8, CXCR2, and S100A8
CXCL1, CXCL2, CXCL8, CXCR2, and S100A9
CXCL1, CXCL2, CXCL8, S100A8, and S100A9
CXCL1, CXCL2, PTGS2, CXCR1, and CXCR2
CXCL1, CXCL2, PTGS2, CXCR1, and S100A8
CXCL1, CXCL2, PTGS2, CXCR1, and S100A9
CXCL1, CXCL2, PTGS2, CXCR2, and S100A8
CXCL1, CXCL2, PTGS2, CXCR2, and S100A9
CXCL1, CXCL2, PTGS2, S100A8, and S100A9
CXCL1, CXCL2, CXCR1, CXCR2, and S100A8
CXCL1, CXCL2, CXCR1, CXCR2, and S100A9
CXCL1, CXCL2, CXCR1, S100A8, and S100A9
CXCL1, CXCL2, CXCR2, S100A8, and S100A9
CXCL1, CXCL3, CXCL8, PTGS2, and CXCR1
CXCL1, CXCL3, CXCL8, PTGS2, and CXCR2
CXCL1, CXCL3, CXCL8, PTGS2, and S100A8
CXCL1, CXCL3, CXCL8, PTGS2, and S100A9
CXCL1, CXCL3, CXCL8, CXCR1, and CXCR2
CXCL1, CXCL3, CXCL8, CXCR1, and S100A8
CXCL1, CXCL3, CXCL8, CXCR1, and S100A9
CXCL1, CXCL3, CXCL8, CXCR2, and S100A8
CXCL1, CXCL3, CXCL8, CXCR2, and S100A9
CXCL1, CXCL3, CXCL8, S100A8, and S100A9
CXCL1, CXCL3, PTGS2, CXCR1, and CXCR2
CXCL1, CXCL3, PTGS2, CXCR1, and S100A8
CXCL1, CXCL3, PTGS2, CXCR1, and S100A9
CXCL1, CXCL3, PTGS2, CXCR2, and S100A8
CXCL1, CXCL3, PTGS2, CXCR2, and S100A9
CXCL1, CXCL3, PTGS2, S100A8, and S100A9
CXCL1, CXCL3, CXCR1, CXCR2, and S100A8
CXCL1, CXCL3, CXCR1, CXCR2, and S100A9
CXCL1, CXCL3, CXCR1, S100A8, and S100A9
CXCL1, CXCL3, CXCR2, S100A8, and S100A9
CXCL1, CXCL8, PTGS2, CXCR1, and CXCR2
CXCL1, CXCL8, PTGS2, CXCR1, and S100A8
CXCL1, CXCL8, PTGS2, CXCR1, and S100A9
CXCL1, CXCL8, PTGS2, CXCR2, and S100A8
CXCL1, CXCL8, PTGS2, CXCR2, and S100A9
CXCL1, CXCL8, PTGS2, S100A8, and S100A9
CXCL1, CXCL8, CXCR1, CXCR2, and S100A8
CXCL1, CXCL8, CXCR1, CXCR2, and S100A9
CXCL1, CXCL8, CXCR1, S100A8, and S100A9
CXCL1, CXCL8, CXCR2, S100A8, and S100A9
CXCL1, PTGS2, CXCR1, CXCR2, and S100A8
CXCL1, PTGS2, CXCR1, CXCR2, and S100A9
CXCL1, PTGS2, CXCR1, S100A8, and S100A9
CXCL1, PTGS2, CXCR2, S100A8, and S100A9
CXCL1, CXCR1, CXCR2, S100A8, and S100A9
CXCL2, CXCL3, CXCL8, PTGS2, and CXCR1
CXCL2, CXCL3, CXCL8, PTGS2, and CXCR2
CXCL2, CXCL3, CXCL8, PTGS2, and S100A8
CXCL2, CXCL3, CXCL8, PTGS2, and S100A9
CXCL2, CXCL3, CXCL8, CXCR1, and CXCR2
CXCL2, CXCL3, CXCL8, CXCR1, and S100A8
CXCL2, CXCL3, CXCL8, CXCR1, and S100A9
CXCL2, CXCL3, CXCL8, CXCR2, and S100A8
CXCL2, CXCL3, CXCL8, CXCR2, and S100A9
CXCL2, CXCL3, CXCL8, S100A8, and S100A9
CXCL2, CXCL3, PTGS2, CXCR1, and CXCR2
CXCL2, CXCL3, PTGS2, CXCR1, and S100A8
CXCL2, CXCL3, PTGS2, CXCR1, and S100A9
CXCL2, CXCL3, PTGS2, CXCR2, and S100A8
CXCL2, CXCL3, PTGS2, CXCR2, and S100A9
CXCL2, CXCL3, PTGS2, S100A8, and S100A9
CXCL2, CXCL3, CXCR1, CXCR2, and S100A8
CXCL2, CXCL3, CXCR1, CXCR2, and S100A9
CXCL2, CXCL3, CXCR1, S100A8, and S100A9
CXCL2, CXCL3, CXCR2, S100A8, and S100A9
CXCL2, CXCL8, PTGS2, CXCR1, and CXCR2
CXCL2, CXCL8, PTGS2, CXCR1, and S100A8
CXCL2, CXCL8, PTGS2, CXCR1, and S100A9
CXCL2, CXCL8, PTGS2, CXCR2, and S100A8
CXCL2, CXCL8, PTGS2, CXCR2, and S100A9
CXCL2, CXCL8, PTGS2, S100A8, and S100A9
CXCL2, CXCL8, CXCR1, CXCR2, and S100A8
CXCL2, CXCL8, CXCR1, CXCR2, and S100A9
CXCL2, CXCL8, CXCR1, S100A8, and S100A9
CXCL2, CXCL8, CXCR2, S100A8, and S100A9
CXCL2, PTGS2, CXCR1, CXCR2, and S100A8
CXCL2, PTGS2, CXCR1, CXCR2, and S100A9
CXCL2, PTGS2, CXCR1, S100A8, and S100A9
CXCL2, PTGS2, CXCR2, S100A8, and S100A9
CXCL2, CXCR1, CXCR2, S100A8, and S100A9
CXCL3, CXCL8, PTGS2, CXCR1, and CXCR2
CXCL3, CXCL8, PTGS2, CXCR1, and S100A8
CXCL3, CXCL8, PTGS2, CXCR1, and S100A9
CXCL3, CXCL8, PTGS2, CXCR2, and S100A8
CXCL3, CXCL8, PTGS2, CXCR2, and S100A9
CXCL3, CXCL8, PTGS2, S100A8, and S100A9
CXCL3, CXCL8, CXCR1, CXCR2, and S100A8
CXCL3, CXCL8, CXCR1, CXCR2, and S100A9
CXCL3, CXCL8, CXCR1, S100A8, and S100A9
CXCL3, CXCL8, CXCR2, S100A8, and S100A9
CXCL3, PTGS2, CXCR1, CXCR2, and S100A8
CXCL3, PTGS2, CXCR1, CXCR2, and S100A9
CXCL3, PTGS2, CXCR1, S100A8, and S100A9
CXCL3, PTGS2, CXCR2, S100A8, and S100A9
CXCL3, CXCR1, CXCR2, S100A8, and S100A9
CXCL8, PTGS2, CXCR1, CXCR2, and S100A8
CXCL8, PTGS2, CXCR1, CXCR2, and S100A9
CXCL8, PTGS2, CXCR1, S100A8, and S100A9
CXCL8, PTGS2, CXCR2, S100A8, and S100A9
CXCL8, CXCR1, CXCR2, S100A8, and S100A9
PTGS2, CXCR1, CXCR2, S100A8, and S100A9

TABLE 13
Six-Gene Combinations of IL6, CXCL1, CXCL2, CXCL3,
CXCL8, PTGS2, CXCR1, CXCR2, S100A8, and S100A9
IL6, CXCL1, CXCL2, CXCL3, CXCL8, and PTGS2
IL6, CXCL1, CXCL2, CXCL3, CXCL8, and CXCR1
IL6, CXCL1, CXCL2, CXCL3, CXCL8, and CXCR2
IL6, CXCL1, CXCL2, CXCL3, CXCL8, and S100A8
IL6, CXCL1, CXCL2, CXCL3, CXCL8, and S100A9
IL6, CXCL1, CXCL2, CXCL3, PTGS2, and CXCR1
IL6, CXCL1, CXCL2, CXCL3, PTGS2, and CXCR2
IL6, CXCL1, CXCL2, CXCL3, PTGS2, and S100A8
IL6, CXCL1, CXCL2, CXCL3, PTGS2, and S100A9
IL6, CXCL1, CXCL2, CXCL3, CXCR1, and CXCR2
IL6, CXCL1, CXCL2, CXCL3, CXCR1, and S100A8
IL6, CXCL1, CXCL2, CXCL3, CXCR1, and S100A9
IL6, CXCL1, CXCL2, CXCL3, CXCR2, and S100A8
IL6, CXCL1, CXCL2, CXCL3, CXCR2, and S100A9
IL6, CXCL1, CXCL2, CXCL3, S100A8, and S100A9
IL6, CXCL1, CXCL2, CXCL8, PTGS2, and CXCR1
IL6, CXCL1, CXCL2, CXCL8, PTGS2, and CXCR2
IL6, CXCL1, CXCL2, CXCL8, PTGS2, and S100A8
IL6, CXCL1, CXCL2, CXCL8, PTGS2, and S100A9
IL6, CXCL1, CXCL2, CXCL8, CXCR1, and CXCR2
IL6, CXCL1, CXCL2, CXCL8, CXCR1, and S100A8
IL6, CXCL1, CXCL2, CXCL8, CXCR1, and S100A9
IL6, CXCL1, CXCL2, CXCL8, CXCR2, and S100A8
IL6, CXCL1, CXCL2, CXCL8, CXCR2, and S100A9
IL6, CXCL1, CXCL2, CXCL8, S100A8, and S100A9
IL6, CXCL1, CXCL2, PTGS2, CXCR1, and CXCR2
IL6, CXCL1, CXCL2, PTGS2, CXCR1, and S100A8
IL6, CXCL1, CXCL2, PTGS2, CXCR1, and S100A9
IL6, CXCL1, CXCL2, PTGS2, CXCR2, and S100A8
IL6, CXCL1, CXCL2, PTGS2, CXCR2, and S100A9
IL6, CXCL1, CXCL2, PTGS2, S100A8, and S100A9
IL6, CXCL1, CXCL2, CXCR1, CXCR2, and S100A8
IL6, CXCL1, CXCL2, CXCR1, CXCR2, and S100A9
IL6, CXCL1, CXCL2, CXCR1, S100A8, and S100A9
IL6, CXCL1, CXCL2, CXCR2, S100A8, and S100A9
IL6, CXCL1, CXCL3, CXCL8, PTGS2, and CXCR1
IL6, CXCL1, CXCL3, CXCL8, PTGS2, and CXCR2
IL6, CXCL1, CXCL3, CXCL8, PTGS2, and S100A8
IL6, CXCL1, CXCL3, CXCL8, PTGS2, and S100A9
IL6, CXCL1, CXCL3, CXCL8, CXCR1, and CXCR2
IL6, CXCL1, CXCL3, CXCL8, CXCR1, and S100A8
IL6, CXCL1, CXCL3, CXCL8, CXCR1, and S100A9
IL6, CXCL1, CXCL3, CXCL8, CXCR2, and S100A8
IL6, CXCL1, CXCL3, CXCL8, CXCR2, and S100A9
IL6, CXCL1, CXCL3, CXCL8, S100A8, and S100A9
IL6, CXCL1, CXCL3, PTGS2, CXCR1, and CXCR2
IL6, CXCL1, CXCL3, PTGS2, CXCR1, and S100A8
IL6, CXCL1, CXCL3, PTGS2, CXCR1, and S100A9
IL6, CXCL1, CXCL3, PTGS2, CXCR2, and S100A8
IL6, CXCL1, CXCL3, PTGS2, CXCR2, and S100A9
IL6, CXCL1, CXCL3, PTGS2, S100A8, and S100A9
IL6, CXCL1, CXCL3, CXCR1, CXCR2, and S100A8
IL6, CXCL1, CXCL3, CXCR1, CXCR2, and S100A9
IL6, CXCL1, CXCL3, CXCR1, S100A8, and S100A9
IL6, CXCL1, CXCL3, CXCR2, S100A8, and S100A9
IL6, CXCL1, CXCL8, PTGS2, CXCR1, and CXCR2
IL6, CXCL1, CXCL8, PTGS2, CXCR1, and S100A8
IL6, CXCL1, CXCL8, PTGS2, CXCR1, and S100A9
IL6, CXCL1, CXCL8, PTGS2, CXCR2, and S100A8
IL6, CXCL1, CXCL8, PTGS2, CXCR2, and S100A9
IL6, CXCL1, CXCL8, PTGS2, S100A8, and S100A9
IL6, CXCL1, CXCL8, CXCR1, CXCR2, and S100A8
IL6, CXCL1, CXCL8, CXCR1, CXCR2, and S100A9
IL6, CXCL1, CXCL8, CXCR1, S100A8, and S100A9
IL6, CXCL1, CXCL8, CXCR2, S100A8, and S100A9
IL6, CXCL1, PTGS2, CXCR1, CXCR2, and S100A8
IL6, CXCL1, PTGS2, CXCR1, CXCR2, and S100A9
IL6, CXCL1, PTGS2, CXCR1, S100A8, and S100A9
IL6, CXCL1, PTGS2, CXCR2, S100A8, and S100A9
IL6, CXCL1, CXCR1, CXCR2, S100A8, and S100A9
IL6, CXCL2, CXCL3, CXCL8, PTGS2, and CXCR1
IL6, CXCL2, CXCL3, CXCL8, PTGS2, and CXCR2
IL6, CXCL2, CXCL3, CXCL8, PTGS2, and S100A8
IL6, CXCL2, CXCL3, CXCL8, PTGS2, and S100A9
IL6, CXCL2, CXCL3, CXCL8, CXCR1, and CXCR2
IL6, CXCL2, CXCL3, CXCL8, CXCR1, and S100A8
IL6, CXCL2, CXCL3, CXCL8, CXCR1, and S100A9
IL6, CXCL2, CXCL3, CXCL8, CXCR2, and S100A8
IL6, CXCL2, CXCL3, CXCL8, CXCR2, and S100A9
IL6, CXCL2, CXCL3, CXCL8, S100A8, and S100A9
IL6, CXCL2, CXCL3, PTGS2, CXCR1, and CXCR2
IL6, CXCL2, CXCL3, PTGS2, CXCR1, and S100A8
IL6, CXCL2, CXCL3, PTGS2, CXCR1, and S100A9
IL6, CXCL2, CXCL3, PTGS2, CXCR2, and S100A8
IL6, CXCL2, CXCL3, PTGS2, CXCR2, and S100A9
IL6, CXCL2, CXCL3, PTGS2, S100A8, and S100A9
IL6, CXCL2, CXCL3, CXCR1, CXCR2, and S100A8
IL6, CXCL2, CXCL3, CXCR1, CXCR2, and S100A9
IL6, CXCL2, CXCL3, CXCR1, S100A8, and S100A9
IL6, CXCL2, CXCL3, CXCR2, S100A8, and S100A9
IL6, CXCL2, CXCL8, PTGS2, CXCR1, and CXCR2
IL6, CXCL2, CXCL8, PTGS2, CXCR1, and S100A8
IL6, CXCL2, CXCL8, PTGS2, CXCR1, and S100A9
IL6, CXCL2, CXCL8, PTGS2, CXCR2, and S100A8
IL6, CXCL2, CXCL8, PTGS2, CXCR2, and S100A9
IL6, CXCL2, CXCL8, PTGS2, S100A8, and S100A9
IL6, CXCL2, CXCL8, CXCR1, CXCR2, and S100A8
IL6, CXCL2, CXCL8, CXCR1, CXCR2, and S100A9
IL6, CXCL2, CXCL8, CXCR1, S100A8, and S100A9
IL6, CXCL2, CXCL8, CXCR2, S100A8, and S100A9
IL6, CXCL2, PTGS2, CXCR1, CXCR2, and S100A8
IL6, CXCL2, PTGS2, CXCR1, CXCR2, and S100A9
IL6, CXCL2, PTGS2, CXCR1, S100A8, and S100A9
IL6, CXCL2, PTGS2, CXCR2, S100A8, and S100A9
IL6, CXCL2, CXCR1, CXCR2, S100A8, and S100A9
IL6, CXCL3, CXCL8, PTGS2, CXCR1, and CXCR2
IL6, CXCL3, CXCL8, PTGS2, CXCR1, and 100A8
IL6, CXCL3, CXCL8, PTGS2, CXCR1, and S100A9
IL6, CXCL3, CXCL8, PTGS2, CXCR2, and S100A8
IL6, CXCL3, CXCL8, PTGS2, CXCR2, and S100A9
IL6, CXCL3, CXCL8, PTGS2, S100A8, and S100A9
IL6, CXCL3, CXCL8, CXCR1, CXCR2, and S100A8
IL6, CXCL3, CXCL8, CXCR1, CXCR2, and S100A9
IL6, CXCL3, CXCL8, CXCR1, S100A8, and S100A9
IL6, CXCL3, CXCL8, CXCR2, S100A8, and S100A9
IL6, CXCL3, PTGS2, CXCR1, CXCR2, and S100A8
IL6, CXCL3, PTGS2, CXCR1, CXCR2, and S100A9
IL6, CXCL3, PTGS2, CXCR1, S100A8, and S100A9
IL6, CXCL3, PTGS2, CXCR2, S100A8, and S100A9
IL6, CXCL3, CXCR1, CXCR2, S100A8, and S100A9
IL6, CXCL8, PTGS2, CXCR1, CXCR2, and S100A8
IL6, CXCL8, PTGS2, CXCR1, CXCR2, and S100A9
IL6, CXCL8, PTGS2, CXCR1, S100A8, and S100A9
IL6, CXCL8, PTGS2, CXCR2, S100A8, and S100A9
IL6, CXCL8, CXCR1, CXCR2, S100A8, and S100A9
IL6, PTGS2, CXCR1, CXCR2, S100A8, and S100A9
CXCL1, CXCL2, CXCL3, CXCL8, PTGS2, and CXCR1
CXCL1, CXCL2, CXCL3, CXCL8, PTGS2, and CXCR2
CXCL1, CXCL2, CXCL3, CXCL8, PTGS2, and S100A8
CXCL1, CXCL2, CXCL3, CXCL8, PTGS2, and S100A9
CXCL1, CXCL2, CXCL3, CXCL8, CXCR1, and CXCR2
CXCL1, CXCL2, CXCL3, CXCL8, CXCR1, and S100A8
CXCL1, CXCL2, CXCL3, CXCL8, CXCR1, and S100A9
CXCL1, CXCL2, CXCL3, CXCL8, CXCR2, and S100A8
CXCL1, CXCL2, CXCL3, CXCL8, CXCR2, and S100A9
CXCL1, CXCL2, CXCL3, CXCL8, S100A8, and S100A9
CXCL1, CXCL2, CXCL3, PTGS2, CXCR1, and CXCR2
CXCL1, CXCL2, CXCL3, PTGS2, CXCR1, and S100A8
CXCL1, CXCL2, CXCL3, PTGS2, CXCR1, and S100A9
CXCL1, CXCL2, CXCL3, PTGS2, CXCR2, and S100A8
CXCL1, CXCL2, CXCL3, PTGS2, CXCR2, and S100A9
CXCL1, CXCL2, CXCL3, PTGS2, S100A8, and S100A9
CXCL1, CXCL2, CXCL3, CXCR1, CXCR2, and S100A8
CXCL1, CXCL2, CXCL3, CXCR1, CXCR2, and S100A9
CXCL1, CXCL2, CXCL3, CXCR1, S100A8, and S100A9
CXCL1, CXCL2, CXCL3, CXCR2, S100A8, and S100A9
CXCL1, CXCL2, CXCL8, PTGS2, CXCR1, and CXCR2
CXCL1, CXCL2, CXCL8, PTGS2, CXCR1, and S100A8
CXCL1, CXCL2, CXCL8, PTGS2, CXCR1, and S100A9
CXCL1, CXCL2, CXCL8, PTGS2, CXCR2, and S100A8
CXCL1, CXCL2, CXCL8, PTGS2, CXCR2, and S100A9
CXCL1, CXCL2, CXCL8, PTGS2, S100A8, and S100A9
CXCL1, CXCL2, CXCL8, CXCR1, CXCR2, and S100A8
CXCL1, CXCL2, CXCL8, CXCR1, CXCR2, and S100A9
CXCL1, CXCL2, CXCL8, CXCR1, S100A8, and S100A9
CXCL1, CXCL2, CXCL8, CXCR2, S100A8, and S100A9
CXCL1, CXCL2, PTGS2, CXCR1, CXCR2, and S100A8
CXCL1, CXCL2, PTGS2, CXCR1, CXCR2, and S100A9
CXCL1, CXCL2, PTGS2, CXCR1, S100A8, and S100A9
CXCL1, CXCL2, PTGS2, CXCR2, S100A8, and S100A9
CXCL1, CXCL2, CXCR1, CXCR2, S100A8, and S100A9
CXCL1, CXCL3, CXCL8, PTGS2, CXCR1, and CXCR2
CXCL1, CXCL3, CXCL8, PTGS2, CXCR1, and S100A8
CXCL1, CXCL3, CXCL8, PTGS2, CXCR1, and S100A9
CXCL1, CXCL3, CXCL8, PTGS2, CXCR2, and S100A8
CXCL1, CXCL3, CXCL8, PTGS2, CXCR2, and S100A9
CXCL1, CXCL3, CXCL8, PTGS2, S100A8, and S100A9
CXCL1, CXCL3, CXCL8, CXCR1, CXCR2, and S100A8
CXCL1, CXCL3, CXCL8, CXCR1, CXCR2, and S100A9
CXCL1, CXCL3, CXCL8, CXCR1, S100A8, and S100A9
CXCL1, CXCL3, CXCL8, CXCR2, S100A8, and S100A9
CXCL1, CXCL3, PTGS2, CXCR1, CXCR2, and S100A8
CXCL1, CXCL3, PTGS2, CXCR1, CXCR2, and S100A9
CXCL1, CXCL3, PTGS2, CXCR1, S100A8, and S100A9
CXCL1, CXCL3, PTGS2, CXCR2, S100A8, and S100A9
CXCL1, CXCL3, CXCR1, CXCR2, S100A8, and S100A9
CXCL1, CXCL8, PTGS2, CXCR1, CXCR2, and S100A8
CXCL1, CXCL8, PTGS2, CXCR1, CXCR2, and S100A9
CXCL1, CXCL8, PTGS2, CXCR1, S100A8, and S100A9
CXCL1, CXCL8, PTGS2, CXCR2, S100A8, and S100A9
CXCL1, CXCL8, CXCR1, CXCR2, S100A8, and S100A9
CXCL1, PTGS2, CXCR1, CXCR2, S100A8, and S100A9
CXCL2, CXCL3, CXCL8, PTGS2, CXCR1, and CXCR2
CXCL2, CXCL3, CXCL8, PTGS2, CXCR1, and S100A8
CXCL2, CXCL3, CXCL8, PTGS2, CXCR1, and S100A9
CXCL2, CXCL3, CXCL8, PTGS2, CXCR2, and S100A8
CXCL2, CXCL3, CXCL8, PTGS2, CXCR2, and S100A9
CXCL2, CXCL3, CXCL8, PTGS2, S100A8, and S100A9
CXCL2, CXCL3, CXCL8, CXCR1, CXCR2, and S100A8
CXCL2, CXCL3, CXCL8, CXCR1, CXCR2, and S100A9
CXCL2, CXCL3, CXCL8, CXCR1, S100A8, and S100A9
CXCL2, CXCL3, CXCL8, CXCR2, S100A8, and S100A9
CXCL2, CXCL3, PTGS2, CXCR1, CXCR2, and S100A8
CXCL2, CXCL3, PTGS2, CXCR1, CXCR2, and S100A9
CXCL2, CXCL3, PTGS2, CXCR1, S100A8, and S100A9
CXCL2, CXCL3, PTGS2, CXCR2, S100A8, and S100A9
CXCL2, CXCL3, CXCR1, CXCR2, S100A8, and S100A9
CXCL2, CXCL8, PTGS2, CXCR1, CXCR2, and S100A8
CXCL2, CXCL8, PTGS2, CXCR1, CXCR2, and S100A9
CXCL2, CXCL8, PTGS2, CXCR1, S100A8, and S100A9
CXCL2, CXCL8, PTGS2, CXCR2, S100A8, and S100A9
CXCL2, CXCL8, CXCR1, CXCR2, S100A8, and S100A9
CXCL2, PTGS2, CXCR1, CXCR2, S100A8, and S100A9
CXCL3, CXCL8, PTGS2, CXCR1, CXCR2, and S100A8
CXCL3, CXCL8, PTGS2, CXCR1, CXCR2, and S100A9
CXCL3, CXCL8, PTGS2, CXCR1, S100A8, and S100A9
CXCL3, CXCL8, PTGS2, CXCR2, S100A8, and S100A9
CXCL3, CXCL8, CXCR1, CXCR2, S100A8, and S100A9
CXCL3, PTGS2, CXCR1, CXCR2, S100A8, and S100A9
CXCL8, PTGS2, CXCR1, CXCR2, S100A8, and S100A9

TABLE 14
Seven-Gene Combinations of IL6, CXCL1, CXCL2, CXCL3,
CXCL8, PTGS2, CXCR1, CXCR2, S100A8, and S100A9
IL6, CXCL1, CXCL2, CXCL3, CXCL8, PTGS2, and CXCR1
IL6, CXCL1, CXCL2, CXCL3, CXCL8, PTGS2, and CXCR2
IL6, CXCL1, CXCL2, CXCL3, CXCL8, PTGS2, and S100A8
IL6, CXCL1, CXCL2, CXCL3, CXCL8, PTGS2, and S100A9
IL6, CXCL1, CXCL2, CXCL3, CXCL8, CXCR1, and CXCR2
IL6, CXCL1, CXCL2, CXCL3, CXCL8, CXCR1, and S100A8
IL6, CXCL1, CXCL2, CXCL3, CXCL8, CXCR1, and S100A9
IL6, CXCL1, CXCL2, CXCL3, CXCL8, CXCR2, and S100A8
IL6, CXCL1, CXCL2, CXCL3, CXCL8, CXCR2, and S100A9
IL6, CXCL1, CXCL2, CXCL3, CXCL8, S100A8, and S100A9
IL6, CXCL1, CXCL2, CXCL3, PTGS2, CXCR1, and CXCR2
IL6, CXCL1, CXCL2, CXCL3, PTGS2, CXCR1, and S100A8
IL6, CXCL1, CXCL2, CXCL3, PTGS2, CXCR1, and S100A9
IL6, CXCL1, CXCL2, CXCL3, PTGS2, CXCR2, and S100A8
IL6, CXCL1, CXCL2, CXCL3, PTGS2, CXCR2, and S100A9
IL6, CXCL1, CXCL2, CXCL3, PTGS2, S100A8, and S100A9
IL6, CXCL1, CXCL2, CXCL3, CXCR1, CXCR2, and S100A8
IL6, CXCL1, CXCL2, CXCL3, CXCR1, CXCR2, and S100A9
IL6, CXCL1, CXCL2, CXCL3, CXCR1, S100A8, and S100A9
IL6, CXCL1, CXCL2, CXCL3, CXCR2, S100A8, and S100A9
IL6, CXCL1, CXCL2, CXCL8, PTGS2, CXCR1, and CXCR2
IL6, CXCL1, CXCL2, CXCL8, PTGS2, CXCR1, and S100A8
IL6, CXCL1, CXCL2, CXCL8, PTGS2, CXCR1, and S100A9
IL6, CXCL1, CXCL2, CXCL8, PTGS2, CXCR2, and S100A8
IL6, CXCL1, CXCL2, CXCL8, PTGS2, CXCR2, and S100A9
IL6, CXCL1, CXCL2, CXCL8, PTGS2, S100A8, and S100A9
IL6, CXCL1, CXCL2, CXCL8, CXCR1, CXCR2, and S100A8
IL6, CXCL1, CXCL2, CXCL8, CXCR1, CXCR2, and S100A9
IL6, CXCL1, CXCL2, CXCL8, CXCR1, S100A8, and S100A9
IL6, CXCL1, CXCL2, CXCL8, CXCR2, S100A8, and S100A9
IL6, CXCL1, CXCL2, PTGS2, CXCR1, CXCR2, and S100A8
IL6, CXCL1, CXCL2, PTGS2, CXCR1, CXCR2, and S100A9
IL6, CXCL1, CXCL2, PTGS2, CXCR1, S100A8, and S100A9
IL6, CXCL1, CXCL2, PTGS2, CXCR2, S100A8, and S100A9
IL6, CXCL1, CXCL2, CXCR1, CXCR2, S100A8, and S100A9
IL6, CXCL1, CXCL3, CXCL8, PTGS2, CXCR1, and CXCR2
IL6, CXCL1, CXCL3, CXCL8, PTGS2, CXCR1, and S100A8
IL6, CXCL1, CXCL3, CXCL8, PTGS2, CXCR1, and S100A9
IL6, CXCL1, CXCL3, CXCL8, PTGS2, CXCR2, and S100A8
IL6, CXCL1, CXCL3, CXCL8, PTGS2, CXCR2, and S100A9
IL6, CXCL1, CXCL3, CXCL8, PTGS2, S100A8, and S100A9
IL6, CXCL1, CXCL3, CXCL8, CXCR1, CXCR2, and S100A8
IL6, CXCL1, CXCL3, CXCL8, CXCR1, CXCR2, and S100A9
IL6, CXCL1, CXCL3, CXCL8, CXCR1, S100A8, and S100A9
IL6, CXCL1, CXCL3, CXCL8, CXCR2, S100A8, and S100A9
IL6, CXCL1, CXCL3, PTGS2, CXCR1, CXCR2, and S100A8
IL6, CXCL1, CXCL3, PTGS2, CXCR1, CXCR2, and S100A9
IL6, CXCL1, CXCL3, PTGS2, CXCR1, S100A8, and S100A9
IL6, CXCL1, CXCL3, PTGS2, CXCR2, S100A8, and S100A9
IL6, CXCL1, CXCL3, CXCR1, CXCR2, S100A8, and S100A9
IL6, CXCL1, CXCL8, PTGS2, CXCR1, CXCR2, and S100A8
IL6, CXCL1, CXCL8, PTGS2, CXCR1, CXCR2, and S100A9
IL6, CXCL1, CXCL8, PTGS2, CXCR1, S100A8, and S100A9
IL6, CXCL1, CXCL8, PTGS2, CXCR2, S100A8, and S100A9
IL6, CXCL1, CXCL8, CXCR1, CXCR2, S100A8, and S100A9
IL6, CXCL1, PTGS2, CXCR1, CXCR2, S100A8, and S100A9
IL6, CXCL2, CXCL3, CXCL8, PTGS2, CXCR1, and CXCR2
IL6, CXCL2, CXCL3, CXCL8, PTGS2, CXCR1, and S100A8
IL6, CXCL2, CXCL3, CXCL8, PTGS2, CXCR1, and S100A9
IL6, CXCL2, CXCL3, CXCL8, PTGS2, CXCR2, and S100A8
IL6, CXCL2, CXCL3, CXCL8, PTGS2, CXCR2, and S100A9
IL6, CXCL2, CXCL3, CXCL8, PTGS2, S100A8, and S100A9
IL6, CXCL2, CXCL3, CXCL8, CXCR1, CXCR2, and S100A8
IL6, CXCL2, CXCL3, CXCL8, CXCR1, CXCR2, and S100A9
IL6, CXCL2, CXCL3, CXCL8, CXCR1, S100A8, and S100A9
IL6, CXCL2, CXCL3, CXCL8, CXCR2, S100A8, and S100A9
IL6, CXCL2, CXCL3, PTGS2, CXCR1, CXCR2, and S100A8
IL6, CXCL2, CXCL3, PTGS2, CXCR1, CXCR2, and S100A9
IL6, CXCL2, CXCL3, PTGS2, CXCR1, S100A8, and S100A9
IL6, CXCL2, CXCL3, PTGS2, CXCR2, S100A8, and S100A9
IL6, CXCL2, CXCL3, CXCR1, CXCR2, S100A8, and S100A9
IL6, CXCL2, CXCL8, PTGS2, CXCR1, CXCR2, and S100A8
IL6, CXCL2, CXCL8, PTGS2, CXCR1, CXCR2, and S100A9
IL6, CXCL2, CXCL8, PTGS2, CXCR1, S100A8, and S100A9
IL6, CXCL2, CXCL8, PTGS2, CXCR2, S100A8, and S100A9
IL6, CXCL2, CXCL8, CXCR1, CXCR2, S100A8, and S100A9
IL6, CXCL2, PTGS2, CXCR1, CXCR2, S100A8, and S100A9
IL6, CXCL3, CXCL8, PTGS2, CXCR1, CXCR2, and S100A8
IL6, CXCL3, CXCL8, PTGS2, CXCR1, CXCR2, and S100A9
IL6, CXCL3, CXCL8, PTGS2, CXCR1, S100A8, and S100A9
IL6, CXCL3, CXCL8, PTGS2, CXCR2, S100A8, and S100A9
IL6, CXCL3, CXCL8, CXCR1, CXCR2, S100A8, and S100A9
IL6, CXCL3, PTGS2, CXCR1, CXCR2, S100A8, and S100A9
IL6, CXCL8, PTGS2, CXCR1, CXCR2, S100A8, and S100A9
CXCL1, CXCL2, CXCL3, CXCL8, PTGS2, CXCR1, and CXCR2
CXCL1, CXCL2, CXCL3, CXCL8, PTGS2, CXCR1, and S100A8
CXCL1, CXCL2, CXCL3, CXCL8, PTGS2, CXCR1, and S100A9
CXCL1, CXCL2, CXCL3, CXCL8, PTGS2, CXCR2, and S100A8
CXCL1, CXCL2, CXCL3, CXCL8, PTGS2, CXCR2, and S100A9
CXCL1, CXCL2, CXCL3, CXCL8, PTGS2, S100A8, and S100A9
CXCL1, CXCL2, CXCL3, CXCL8, CXCR1, CXCR2, and S100A8
CXCL1, CXCL2, CXCL3, CXCL8, CXCR1, CXCR2, and S100A9
CXCL1, CXCL2, CXCL3, CXCL8, CXCR1, S100A8, and S100A9
CXCL1, CXCL2, CXCL3, CXCL8, CXCR2, S100A8, and S100A9
CXCL1, CXCL2, CXCL3, PTGS2, CXCR1, CXCR2, and S100A8
CXCL1, CXCL2, CXCL3, PTGS2, CXCR1, CXCR2, and S100A9
CXCL1, CXCL2, CXCL3, PTGS2, CXCR1, S100A8, and S100A9
CXCL1, CXCL2, CXCL3, PTGS2, CXCR2, S100A8, and S100A9
CXCL1, CXCL2, CXCL3, CXCR1, CXCR2, S100A8, and S100A9
CXCL1, CXCL2, CXCL8, PTGS2, CXCR1, CXCR2, and S100A8
CXCL1, CXCL2, CXCL8, PTGS2, CXCR1, CXCR2, and S100A9
CXCL1, CXCL2, CXCL8, PTGS2, CXCR1, S100A8, and S100A9
CXCL1, CXCL2, CXCL8, PTGS2, CXCR2, S100A8, and S100A9
CXCL1, CXCL2, CXCL8, CXCR1, CXCR2, S100A8, and S100A9
CXCL1, CXCL2, PTGS2, CXCR1, CXCR2, S100A8, and S100A9
CXCL1, CXCL3, CXCL8, PTGS2, CXCR1, CXCR2, and S100A8
CXCL1, CXCL3, CXCL8, PTGS2, CXCR1, CXCR2, and S100A9
CXCL1, CXCL3, CXCL8, PTGS2, CXCR1, S100A8, and S100A9
CXCL1, CXCL3, CXCL8, PTGS2, CXCR2, S100A8, and S100A9
CXCL1, CXCL3, CXCL8, CXCR1, CXCR2, S100A8, and S100A9
CXCL1, CXCL3, PTGS2, CXCR1, CXCR2, S100A8, and S100A9
CXCL1, CXCL8, PTGS2, CXCR1, CXCR2, S100A8, and S100A9
CXCL2, CXCL3, CXCL8, PTGS2, CXCR1, CXCR2, and S100A8
CXCL2, CXCL3, CXCL8, PTGS2, CXCR1, CXCR2, and S100A9
CXCL2, CXCL3, CXCL8, PTGS2, CXCR1, S100A8, and S100A9
CXCL2, CXCL3, CXCL8, PTGS2, CXCR2, S100A8, and S100A9
CXCL2, CXCL3, CXCL8, CXCR1, CXCR2, S100A8, and S100A9
CXCL2, CXCL3, PTGS2, CXCR1, CXCR2, S100A8, and S100A9
CXCL2, CXCL8, PTGS2, CXCR1, CXCR2, S100A8, and S100A9
CXCL3, CXCL8, PTGS2, CXCR1, CXCR2, S100A8, and S100A9

TABLE 15
Eight-Gene Combinations of IL6, CXCL1, CXCL2, CXCL3, CXCL8,
PTGS2, CXCR1, CXCR2, S100A8, and S100A9
IL6, CXCL1, CXCL2, CXCL3, CXCL8, PTGS2, CXCR1, and CXCR2
IL6, CXCL1, CXCL2, CXCL3, CXCL8, PTGS2, CXCR1, and S100A8
IL6, CXCL1, CXCL2, CXCL3, CXCL8, PTGS2, CXCR1, and S100A9
IL6, CXCL1, CXCL2, CXCL3, CXCL8, PTGS2, CXCR2, and S100A8
IL6, CXCL1, CXCL2, CXCL3, CXCL8, PTGS2, CXCR2, and S100A9
IL6, CXCL1, CXCL2, CXCL3, CXCL8, PTGS2, S100A8, and S100A9
IL6, CXCL1, CXCL2, CXCL3, CXCL8, CXCR1, CXCR2, and S100A8
IL6, CXCL1, CXCL2, CXCL3, CXCL8, CXCR1, CXCR2, and S100A9
IL6, CXCL1, CXCL2, CXCL3, CXCL8, CXCR1, S100A8, and S100A9
IL6, CXCL1, CXCL2, CXCL3, CXCL8, CXCR2, S100A8, and S100A9
IL6, CXCL1, CXCL2, CXCL3, PTGS2, CXCR1, CXCR2, and S100A8
IL6, CXCL1, CXCL2, CXCL3, PTGS2, CXCR1, CXCR2, and S100A9
IL6, CXCL1, CXCL2, CXCL3, PTGS2, CXCR1, S100A8, and S100A9
IL6, CXCL1, CXCL2, CXCL3, PTGS2, CXCR2, S100A8, and S100A9
IL6, CXCL1, CXCL2, CXCL3, CXCR1, CXCR2, S100A8, and S100A9
IL6, CXCL1, CXCL2, CXCL8, PTGS2, CXCR1, CXCR2, and S100A8
IL6, CXCL1, CXCL2, CXCL8, PTGS2, CXCR1, CXCR2, and S100A9
IL6, CXCL1, CXCL2, CXCL8, PTGS2, CXCR1, S100A8, and S100A9
IL6, CXCL1, CXCL2, CXCL8, PTGS2, CXCR2, S100A8, and S100A9
IL6, CXCL1, CXCL2, CXCL8, CXCR1, CXCR2, S100A8, and S100A9
IL6, CXCL1, CXCL2, PTGS2, CXCR1, CXCR2, S100A8, and S100A9
IL6, CXCL1, CXCL3, CXCL8, PTGS2, CXCR1, CXCR2, and S100A8
IL6, CXCL1, CXCL3, CXCL8, PTGS2, CXCR1, CXCR2, and S100A9
IL6, CXCL1, CXCL3, CXCL8, PTGS2, CXCR1, S100A8, and S100A9
IL6, CXCL1, CXCL3, CXCL8, PTGS2, CXCR2, S100A8, and S100A9
IL6, CXCL1, CXCL3, CXCL8, CXCR1, CXCR2, S100A8, and S100A9
IL6, CXCL1, CXCL3, PTGS2, CXCR1, CXCR2, S100A8, and S100A9
IL6, CXCL1, CXCL8, PTGS2, CXCR1, CXCR2, S100A8, and S100A9
IL6, CXCL2, CXCL3, CXCL8, PTGS2, CXCR1, CXCR2, and S100A8
IL6, CXCL2, CXCL3, CXCL8, PTGS2, CXCR1, CXCR2, and S100A9
IL6, CXCL2, CXCL3, CXCL8, PTGS2, CXCR1, S100A8, and S100A9
IL6, CXCL2, CXCL3, CXCL8, PTGS2, CXCR2, S100A8, and S100A9
IL6, CXCL2, CXCL3, CXCL8, CXCR1, CXCR2, S100A8, and S100A9
IL6, CXCL2, CXCL3, PTGS2, CXCR1, CXCR2, S100A8, and S100A9
IL6, CXCL2, CXCL8, PTGS2, CXCR1, CXCR2, S100A8, and S100A9
IL6, CXCL3, CXCL8, PTGS2, CXCR1, CXCR2, S100A8, and S100A9
CXCL1, CXCL2, CXCL3, CXCL8, PTGS2, CXCR1, CXCR2, and S100A8
CXCL1, CXCL2, CXCL3, CXCL8, PTGS2, CXCR1, CXCR2, and S100A9
CXCL1, CXCL2, CXCL3, CXCL8, PTGS2, CXCR1, S100A8, and S100A9
CXCL1, CXCL2, CXCL3, CXCL8, PTGS2, CXCR2, S100A8, and S100A9
CXCL1, CXCL2, CXCL3, CXCL8, CXCR1, CXCR2, S100A8, and S100A9
CXCL1, CXCL2, CXCL3, PTGS2, CXCR1, CXCR2, S100A8, and S100A9
CXCL1, CXCL2, CXCL8, PTGS2, CXCR1, CXCR2, S100A8, and S100A9
CXCL1, CXCL3, CXCL8, PTGS2, CXCR1, CXCR2, S100A8, and S100A9
CXCL2, CXCL3, CXCL8, PTGS2, CXCR1, CXCR2, S100A8, and S100A9

TABLE 16
Nine-Gene Combinations of IL6, CXCL1, CXCL2, CXCL3,
CXCL8, PTGS2, CXCR1, CXCR2, S100A8, and S100A9
IL6, CXCL1, CXCL2, CXCL3, CXCL8, PTGS2, CXCR1,
CXCR2, and S100A8
IL6, CXCL1, CXCL2, CXCL3, CXCL8, PTGS2, CXCR1,
CXCR2, and S100A9
IL6, CXCL1, CXCL2, CXCL3, CXCL8, PTGS2, CXCR1,
S100A8, and S100A9
IL6, CXCL1, CXCL2, CXCL3, CXCL8, PTGS2, CXCR2,
S100A8, and S100A9
IL6, CXCL1, CXCL2, CXCL3, CXCL8, CXCR1, CXCR2,
S100A8, and S100A9
IL6, CXCL1, CXCL2, CXCL3, PTGS2, CXCR1, CXCR2,
S100A8, and S100A9
IL6, CXCL1, CXCL2, CXCL8, PTGS2, CXCR1, CXCR2,
S100A8, and S100A9
IL6, CXCL1, CXCL3, CXCL8, PTGS2, CXCR1, CXCR2,
S100A8, and S100A9
IL6, CXCL2, CXCL3, CXCL8, PTGS2, CXCR1, CXCR2,
S100A8, and S100A9
CXCL1, CXCL2, CXCL3, CXCL8, PTGS2, CXCR1, CXCR2,
S100A8, and S100A9

In any of the preceding methods, the method may include determining the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2, and one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9. For example, in some embodiments, the method includes determining the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2, and at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9.

For example, any of the preceding methods may include determining the expression level of one or more (e.g., 1, 2, 3, 4, or 5) of CD8A, EOMES, PRF1, IFNG, or PD-L1, and one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9. In some embodiments, the method includes determining the expression level of at least two, at least three, at least four, or all five of CD8A, EOMES, PRF1, IFNG, and PD-L1, and at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9. In some embodiments, the method comprises determining the expression level of any one of the combinations set forth in Tables 2-4 and any one of the combinations set forth in Tables 9-16. For example, in some embodiments, the method includes determining the expression level of two of CD8A, EOMES, PRF1, IFNG, and PD-L1, for example, any of the exemplary combinations shown in Table 2, and two of IL6, CXCL1, CXCL2, CXCL3, CXCL8, PTGS2, CXCR1, CXCR2, S100A8, and S100A9, for example, any of the exemplary combinations shown in Table 9. In some embodiments, the method includes determining the expression level of three of CD8A, EOMES, PRF1, IFNG, and PD-L1, for example, any of the exemplary combinations shown in Table 3, and three of IL6, CXCL1, CXCL2, CXCL3, CXCL8, PTGS2, CXCR1, CXCR2, S100A8, and S100A9, for example, any of the exemplary combinations shown in Table 10. In some embodiments, the method includes determining the expression level of four of CD8A, EOMES, PRF1, IFNG, and PD-L1, for example, any of the exemplary combinations shown in Table 4, and four of IL6, CXCL1, CXCL2, CXCL3, CXCL8, PTGS2, CXCR1, CXCR2, S100A8, and S100A9, for example, any of the exemplary combinations shown in Table 11. In some embodiments, the method involves determining the expression level of CD8A, EOMES, PRF1, IFNG, and PD-L1, and five of IL6, CXCL1, CXCL2, CXCL3, CXCL8, PTGS2, CXCR1, CXCR2, S100A8, and S100A9, for example, any of the exemplary combinations shown in Table 12. In some embodiments, the method involves determining the expression level of CD8A, EOMES, PRF1, IFNG, and PD-L1, and six of IL6, CXCL1, CXCL2, CXCL3, CXCL8, PTGS2, CXCR1, CXCR2, S100A8, and S100A9, for example, any of the exemplary combinations shown in Table 13. In some embodiments, the method involves determining the expression level of CD8A, EOMES, PRF1, IFNG, and PD-L1, and seven of IL6, CXCL1, CXCL2, CXCL3, CXCL8, PTGS2, CXCR1, CXCR2, S100A8, and S100A9, for example, any of the exemplary combinations shown in Table 14. In some embodiments, the method involves determining the expression level of CD8A, EOMES, PRF1, IFNG, and PD-L1, and eight of IL6, CXCL1, CXCL2, CXCL3, CXCL8, PTGS2, CXCR1, CXCR2, S100A8, and S100A9, for example, any of the exemplary combinations shown in Table 15. In some embodiments, the method involves determining the expression level of CD8A, EOMES, PRF1, IFNG, and PD-L1, and nine of IL6, CXCL1, CXCL2, CXCL3, CXCL8, PTGS2, CXCR1, CXCR2, S100A8, and S100A9, for example, any of the exemplary combinations shown in Table 16. In some embodiments, the method involves determining the expression level of CD8A, EOMES, PRF1, IFNG, PD-L1, IL6, CXCL1, CXCL2, CXCL3, CXCL8, PTGS2, CXCR1, CXCR2, S100A8, and S100A9.

In other embodiments, in any of the preceding methods, the method may include determining the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2, and one or more (e.g., 1, 2, 3, 4, 5, 6, or 7) of VEGFA, KDR, ESM1, PECAM1, FLT1, ANGPTL4, or CD34. For example, in some embodiments, the method includes determining the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2, and at least two, at least three, at least four, at least five, at least six, or all seven of VEGFA, KDR, ESM1, PECAM1, FLT1, ANGPTL4, or CD34.

For example, any of the preceding methods may include determining the expression level of one or more (e.g., 1, 2, 3, 4, or 5) of CD8A, EOMES, PRF1, IFNG, or PD-L1, and one or more (e.g., 1, 2, 3, 4, 5, or 6) of VEGFA, KDR, ESM1, PECAM1, ANGPTL4, or CD34. In some embodiments, the method includes determining the expression level of at least two, at least three, at least four, or all five of CD8A, EOMES, PRF1, IFNG, and PD-L1, and at least one, at least two, at least three, at least four, at least five, or all six of VEGFA, KDR, ESM1, PECAM1, ANGPTL4, and CD34. In some embodiments, the method comprises determining the expression level of any one of the combinations set forth in Tables 2-4 and any one of the combinations set forth in Tables 5-8. For example, in some embodiments, the method includes determining the expression level of two of CD8A, EOMES, PRF1, IFNG, and PD-L1, for example, any of the exemplary combinations shown in Table 2, and two of VEGFA, KDR, ESM1, PECAM1, ANGPTL4, and CD34, for example, any of the exemplary combinations shown in Table 5. In some embodiments, the method includes determining the expression level of three of CD8A, EOMES, PRF1, IFNG, and PD-L1, for example, any of the exemplary combinations shown in Table 3, and three of VEGFA, KDR, ESM1, PECAM1, ANGPTL4, and CD34, for example, any of the exemplary combinations shown in Table 6. In some embodiments, the method includes determining the expression level of four of CD8A, EOMES, PRF1, IFNG, and PD-L1, for example, any of the exemplary combinations shown in Table 4, and four of VEGFA, KDR, ESM1, PECAM1, ANGPTL4, and CD34, for example, any of the exemplary combinations shown in Table 7. In some embodiments, the method involves determining the expression level of CD8A, EOMES, PRF1, IFNG, and PD-L1, and five of VEGFA, KDR, ESM1, PECAM1, ANGPTL4, and CD34, for example, any of the exemplary combinations shown in Table 8. In some embodiments, the method involves determining the expression level of CD8A, EOMES, PRF1, IFNG, PD-L1, VEGFA, KDR, ESM1, PECAM1, ANGPTL4, and CD34.

In a further embodiment, in any of the preceding methods, the method may include determining the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9, and one or more (e.g., 1, 2, 3, 4, 5, 6, or 7) of VEGFA, KDR, ESM1, PECAM1, FLT1, ANGPTL4, or CD34. For example, in some embodiments, the method includes determining the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9, and at least two, at least three, at least four, at least five, at least six, or all seven of VEGFA, KDR, ESM1, PECAM1, FLT1, ANGPTL4, or CD34.

For example, any of the preceding methods may include determining the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9, and one or more (e.g., 1, 2, 3, 4, 5, or 6) of VEGFA, KDR, ESM1, PECAM1, ANGPTL4, or CD34. In some embodiments, the method includes determining the expression level of at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9, and at least two, at least three, at least four, at least five, or all six of VEGFA, KDR, ESM1, PECAM1, ANGPTL4, and CD34. In some embodiments, the method comprises determining the expression level of any one of the combinations set forth in Tables 9-16 and any one of the combinations set forth in Tables 5-8. For example, in some embodiments, the method includes determining the expression level of two of IL6, CXCL1, CXCL2, CXCL3, CXCL8, PTGS2, CXCR1, CXCR2, S100A8, and S100A9, for example, any of the exemplary combinations shown in Table 9, and two of VEGFA, KDR, ESM1, PECAM1, ANGPTL4, and CD34, for example, any of the exemplary combinations shown in Table 5. In some embodiments, the method includes determining the expression level of three of IL6, CXCL1, CXCL2, CXCL3, CXCL8, PTGS2, CXCR1, CXCR2, S100A8, and S100A9, for example, any of the exemplary combinations shown in Table 10, and three of VEGFA, KDR, ESM1, PECAM1, ANGPTL4, and CD34, for example, any of the exemplary combinations shown in Table 6. In some embodiments, the method includes determining the expression level of four of IL6, CXCL1, CXCL2, CXCL3, CXCL8, PTGS2, CXCR1, CXCR2, S100A8, and S100A9, for example, any of the exemplary combinations shown in Table 11, and four of VEGFA, KDR, ESM1, PECAM1, ANGPTL4, and CD34, for example, any of the exemplary combinations shown in Table 7. In some embodiments, the method involves determining the expression level of five of IL6, CXCL1, CXCL2, CXCL3, CXCL8, PTGS2, CXCR1, CXCR2, S100A8, and S100A9, for example, any of the exemplary combinations shown in Table 12, and five of VEGFA, KDR, ESM1, PECAM1, ANGPTL4, and CD34, for example, any of the exemplary combinations shown in Table 8. In some embodiments, the method involves determining the expression level of six of IL6, CXCL1, CXCL2, CXCL3, CXCL8, PTGS2, CXCR1, CXCR2, S100A8, and S100A9, for example, any of the exemplary combinations shown in Table 13, and VEGFA, KDR, ESM1, PECAM1, ANGPTL4, and CD34. In some embodiments, the method involves determining the expression level of seven of IL6, CXCL1, CXCL2, CXCL3, CXCL8, PTGS2, CXCR1, CXCR2, S100A8, and S100A9, for example, any of the exemplary combinations shown in Table 14, and VEGFA, KDR, ESM1, PECAM1, ANGPTL4, and CD34. In some embodiments, the method involves determining the expression level of eight of IL6, CXCL1, CXCL2, CXCL3, CXCL8, PTGS2, CXCR1, CXCR2, S100A8, and S100A9, for example, any of the exemplary combinations shown in Table 15, and VEGFA, KDR, ESM1, PECAM1, ANGPTL4, and CD34. In some embodiments, the method involves determining the expression level of nine of IL6, CXCL1, CXCL2, CXCL3, CXCL8, PTGS2, CXCR1, CXCR2, S100A8, and S100A9, for example, any of the exemplary combinations shown in Table 16, and VEGFA, KDR, ESM1, PECAM1, ANGPTL4, and CD34. In some embodiments, the method involves determining the expression level of IL6, CXCL1, CXCL2, CXCL3, CXCL8, PTGS2, CXCR1, CXCR2, S100A8, S100A9, VEGFA, KDR, ESM1, PECAM1, ANGPTL4, and CD34.

In some embodiments of any of the preceding methods, the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample is at or above a reference expression level of the one or more genes, and the method further includes administering to the individual an effective amount of the anti-cancer therapy. For example, in some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 is at or above a reference expression level of the one or more genes. In some instances, the expression level of one or more (e.g., 1, 2, 3, 4, or 5) of CD8A, EOMES, PRF1, IFNG, or PD-L1 in the sample is at or above a reference expression level of the one or more genes. In some embodiments, the expression level of one or more of the exemplary combinations set forth in Tables 2-4 in the sample is at or above a reference expression level of the one or more genes. In some embodiments, the expression level of CD8A, EOMES, PRF1, IFNG, and PD-L1 in the sample is at or above a reference expression level of CD8A, EOMES, PRF1, IFNG, and PD-L1.

In some embodiments of any of the preceding methods, the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 in the sample is at or above a reference expression level of the one or more genes. In some embodiments, the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, or S100A9 in the sample is at or above a reference expression level of the one or more genes. In some embodiments, the expression level of one or more of the exemplary combinations set forth in Tables 9-16 in the sample is at or above a reference expression level of the one or more genes. In some embodiments, the expression level of IL6, CXCL1, CXCL2, CXCL3, CXCL8, PTGS2, CXCR1, CXCR2, S100A8, and S100A9 in the sample is at or above a reference expression level of IL6, CXCL1, CXCL2, CXCL3, CXCL8, PTGS2, CXCR1, CXCR2, S100A8, and S100A9.

In some embodiments of any of the preceding methods, the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample is at or above a reference expression level of the one or more genes, and the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 is at or above a reference expression level of the one or more genes, and the method further includes administering to the individual an effective amount of the anti-cancer therapy. For example, in some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2 is at or above a reference expression level of the one or more genes, and the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9 is at or above a reference expression level of the one or more genes. In some embodiments, an expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 at or above a reference expression level of the one or more genes identifies the presence of myeloid inflammation in a tumor.

For example, in some embodiments, the expression level of one or more (e.g., 1, 2, 3, 4, or 5) of CD8A, EOMES, PRF1, IFNG, or PD-L1 is at or above a reference expression level of the one or more genes, and the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 is at or above a reference expression level of the one or more genes. In some embodiments, the expression level of at least two, at least three, at least four, or all five of CD8A, EOMES, PRF1, IFNG, and PD-L1 is at or above a reference expression level of the one or more genes, and the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9 is at or above a reference expression level of the one or more genes. In some embodiments, the expression level of any one of the combinations set forth in Tables 2-4 is at or above a reference expression level of the one or more genes and the expression level of any one of the combinations set forth in Tables 9-16 is at or above a reference expression level of the one or more genes. For example, in some embodiments, the expression level of two of CD8A, EOMES, PRF1, IFNG, and PD-L1, for example, any of the exemplary combinations shown in Table 2, is at or above a reference expression level of the one or more genes, and the expression level of two of IL6, CXCL1, CXCL2, CXCL3, CXCL8, PTGS2, CXCR1, CXCR2, S100A8, and S100A9, for example, any of the exemplary combinations shown in Table 9, is at or above a reference expression level of the one or more genes. In some embodiments, the expression level of three of CD8A, EOMES, PRF1, IFNG, and PD-L1, for example, any of the exemplary combinations shown in Table 3, is at or above a reference expression level of the one or more genes, and the expression level of three of IL6, CXCL1, CXCL2, CXCL3, CXCL8, PTGS2, CXCR1, CXCR2, S100A8, and S100A9, for example, any of the exemplary combinations shown in Table 10, is at or above a reference expression level of the one or more genes. In some embodiments, the expression level of four of CD8A, EOMES, PRF1, IFNG, and PD-L1, for example, any of the exemplary combinations shown in Table 4, is at or above a reference expression level of the one or more genes, and the expression level of four of IL6, CXCL1, CXCL2, CXCL3, CXCL8, PTGS2, CXCR1, CXCR2, S100A8, and S100A9, for example, any of the exemplary combinations shown in Table 11, is at or above a reference expression level of the one or more genes. In some embodiments, the expression level of CD8A, EOMES, PRF1, IFNG, and PD-L1 is at or above a reference expression level of CD8A, EOMES, PRF1, IFNG, and PD-L1, and the expression level of five of IL6, CXCL1, CXCL2, CXCL3, CXCL8, PTGS2, CXCR1, CXCR2, S100A8, and S100A9, for example, any of the exemplary combinations shown in Table 12, is at or above a reference level of the one or more genes. In some embodiments, the expression level of CD8A, EOMES, PRF1, IFNG, and PD-L1 is at or above a reference expression level of CD8A, EOMES, PRF1, IFNG, and PD-L1, and the expression level of six of IL6, CXCL1, CXCL2, CXCL3, CXCL8, PTGS2, CXCR1, CXCR2, S100A8, and S100A9, for example, any of the exemplary combinations shown in Table 13, is at or above a reference level of the one or more genes. In some embodiments, the expression level of CD8A, EOMES, PRF1, IFNG, and PD-L1 is at or above a reference expression level of CD8A, EOMES, PRF1, IFNG, and PD-L1, and the expression level of seven of IL6, CXCL1, CXCL2, CXCL3, CXCL8, PTGS2, CXCR1, CXCR2, S100A8, and S100A9, for example, any of the exemplary combinations shown in Table 14, is at or above a reference level of the one or more genes. In some embodiments, the expression level of CD8A, EOMES, PRF1, IFNG, and PD-L1 is at or above a reference expression level of CD8A, EOMES, PRF1, IFNG, and PD-L1, and the expression level of eight of IL6, CXCL1, CXCL2, CXCL3, CXCL8, PTGS2, CXCR1, CXCR2, S100A8, and S100A9, for example, any of the exemplary combinations shown in Table 15, is at or above a reference level of the one or more genes. In some embodiments, the expression level of CD8A, EOMES, PRF1, IFNG, and PD-L1 is at or above a reference expression level of CD8A, EOMES, PRF1, IFNG, and PD-L1, and the expression level of nine of IL6, CXCL1, CXCL2, CXCL3, CXCL8, PTGS2, CXCR1, CXCR2, S100A8, and S100A9, for example, any of the exemplary combinations shown in Table 16, is at or above a reference level of the one or more genes. In some embodiments, the expression level of CD8A, EOMES, PRF1, IFNG, PD-L1, IL6, CXCL1, CXCL2, CXCL3, CXCL8, PTGS2, CXCR1, CXCR2, S100A8, and S100A9 is at or above a reference expression level of CD8A, EOMES, PRF1, IFNG, PD-L1, IL6, CXCL1, CXCL2, CXCL3, CXCL8, PTGS2, CXCR1, CXCR2, S100A8, and S100A9. In some embodiments, an expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 at or above a reference expression level of the one or more genes identifies the presence of myeloid inflammation in a tumor. In some embodiments, an expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample at or above a reference expression level of the one or more genes, and an expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 at or above a reference expression level of the one or more genes indicates that the individual is less likely to benefit (e.g., is resistant to) a PD-L1 axis binding antagonist (e.g., an anti-PD-L1 antibody, e.g., atezolizumab) monotherapy.

In other embodiments of any of the preceding methods, the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample is at or above a reference expression level of the one or more genes, and the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 is below a reference expression level of the one or more genes, and the method further includes administering to the individual an effective amount of a PD-L1 axis binding antagonist (e.g., an anti-PD-L1 antibody (e.g., atezolizumab) or an anti-PD-1 antibody) monotherapy. For example, in some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2 is at or above a reference expression level of the one or more genes, and the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9 is below a reference expression level of the one or more genes.

For example, in some embodiments, the expression level of one or more (e.g., 1, 2, 3, 4, or 5) of CD8A, EOMES, PRF1, IFNG, or PD-L1 is at or above a reference expression level of the one or more genes, and the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 is below a reference expression level of the one or more genes. In some embodiments, the expression level of at least two, at least three, at least four, or all five of CD8A, EOMES, PRF1, IFNG, and PD-L1 is at or above a reference expression level of the one or more genes, and the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9 is below a reference expression level of the one or more genes. In some embodiments, the expression level of any one of the combinations set forth in Tables 2-4 is at or above a reference expression level of the one or more genes and the expression level of any one of the combinations set forth in Tables 9-16 is below a reference expression level of the one or more genes. For example, in some embodiments, the expression level of two of CD8A, EOMES, PRF1, IFNG, and PD-L1, for example, any of the exemplary combinations shown in Table 2, is at or above a reference expression level of the one or more genes, and the expression level of two of IL6, CXCL1, CXCL2, CXCL3, CXCL8, PTGS2, CXCR1, CXCR2, S100A8, and S100A9, for example, any of the exemplary combinations shown in Table 9, is below a reference expression level of the one or more genes. In some embodiments, the expression level of three of CD8A, EOMES, PRF1, IFNG, and PD-L1, for example, any of the exemplary combinations shown in Table 3, is at or above a reference expression level of the one or more genes, and the expression level of three of IL6, CXCL1, CXCL2, CXCL3, CXCL8, PTGS2, CXCR1, CXCR2, S100A8, and S100A9, for example, any of the exemplary combinations shown in Table 10, is below a reference expression level of the one or more genes. In some embodiments, the expression level of four of CD8A, EOMES, PRF1, IFNG, and PD-L1, for example, any of the exemplary combinations shown in Table 4, is at or above a reference expression level of the one or more genes, and the expression level of four of IL6, CXCL1, CXCL2, CXCL3, CXCL8, PTGS2, CXCR1, CXCR2, S100A8, and S100A9, for example, any of the exemplary combinations shown in Table 11, is below a reference expression level of the one or more genes. In some embodiments, the expression level of CD8A, EOMES, PRF1, IFNG, and PD-L1 is at or above a reference expression level of CD8A, EOMES, PRF1, IFNG, and PD-L1, and the expression level of five of IL6, CXCL1, CXCL2, CXCL3, CXCL8, PTGS2, CXCR1, CXCR2, S100A8, and S100A9, for example, any of the exemplary combinations shown in Table 12, is below a reference level of the one or more genes. In some embodiments, the expression level of CD8A, EOMES, PRF1, IFNG, and PD-L1 is at or above a reference expression level of CD8A, EOMES, PRF1, IFNG, and PD-L1, and the expression level of six of IL6, CXCL1, CXCL2, CXCL3, CXCL8, PTGS2, CXCR1, CXCR2, S100A8, and S100A9, for example, any of the exemplary combinations shown in Table 13, is below a reference level of the one or more genes. In some embodiments, the expression level of CD8A, EOMES, PRF1, IFNG, and PD-L1 is at or above a reference expression level of CD8A, EOMES, PRF1, IFNG, and PD-L1, and the expression level of seven of IL6, CXCL1, CXCL2, CXCL3, CXCL8, PTGS2, CXCR1, CXCR2, S100A8, and S100A9, for example, any of the exemplary combinations shown in Table 14, is below a reference level of the one or more genes. In some embodiments, the expression level of CD8A, EOMES, PRF1, IFNG, and PD-L1 is at or above a reference expression level of CD8A, EOMES, PRF1, IFNG, and PD-L1, and the expression level of eight of IL6, CXCL1, CXCL2, CXCL3, CXCL8, PTGS2, CXCR1, CXCR2, S100A8, and S100A9, for example, any of the exemplary combinations shown in Table 15, is below a reference level of the one or more genes. In some embodiments, the expression level of CD8A, EOMES, PRF1, IFNG, and PD-L1 is at or above a reference expression level of CD8A, EOMES, PRF1, IFNG, and PD-L1, and the expression level of nine of IL6, CXCL1, CXCL2, CXCL3, CXCL8, PTGS2, CXCR1, CXCR2, S100A8, and S100A9, for example, any of the exemplary combinations shown in Table 16, is below a reference level of the one or more genes. In some embodiments, the expression level of CD8A, EOMES, PRF1, IFNG, and PD-L1 is at or above a reference level of CD8A, EOMES, PRF1, IFNG, and PD-L1, and the expression level of IL6, CXCL1, CXCL2, CXCL3, CXCL8, PTGS2, CXCR1, CXCR2, S100A8, and S100A9 is below a reference expression level of IL6, CXCL1, CXCL2, CXCL3, CXCL8, PTGS2, CXCR1, CXCR2, S100A8, and S100A9.

In some embodiments of any of the preceding methods, the expression level of PD-L1 in the sample is at or above a reference expression level of PD-L1, and the expression level of one or more (e.g., 1, 2, 3, 4, 5, 6, 7, 8, 9, 10, 11, 12, 13, 14, 15, 16, 17, 18, or 19) additional genes selected from the group consisting of CD8A, EOMES, GZMA, GZMB, PRF1, IFNG, CXCL9, CXCL10, CXCL11, CD27, FOXP3, PD-1, CTLA4, TIGIT, IDO1, PSMB8, PSMB9, TAP1, or TAP2 in the sample is at or above a reference expression level of the one or more additional genes.

In some embodiments of any of the preceding methods, the expression level of one or more (e.g., 1, 2, 3, 4, 5, 6, or 7) of VEGFA, KDR, ESM1, PECAM1, FLT1, ANGPTL4, or CD34 in the sample is below a reference level of the one or more genes, and the method further comprises administering to the individual an effective amount of the anti-cancer therapy. For example, in some embodiments, the expression level of at least one, at least two, at least three, at least four, at least five, at least six, or all seven of VEGFA, KDR, ESM1, PECAM1, FLT1, ANGPTL4, or CD34 in the sample is below a reference level of the one or more genes. In some embodiments, the expression level of one or more of the exemplary combinations set forth in Tables 5-8 in the sample is below a reference expression level of the one or more genes. In some embodiments, the expression level of one or more of VEGFA, KDR, ESM1, PECAM1, ANGPTL4, or CD34 in the sample is below a reference level of the one or more genes. For example, in some embodiments, the expression level of VEGFA, KDR, ESM1, PECAM1, ANGPTL4, and CD34 in the sample is below a reference level of VEGFA, KDR, ESM1, PECAM1, ANGPTL4, and CD34.

In other embodiments, in any of the preceding methods, the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 is at or above a reference level of the one or more genes, and the expression level of one or more (e.g., 1, 2, 3, 4, 5, 6, or 7) of VEGFA, KDR, ESM1, PECAM1, FLT1, ANGPTL4, or CD34 is below a reference level of the one or more genes, and the method further comprises administering to the individual an effective amount of the anti-cancer therapy. For example, in some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2 is at or above a reference level of the one or more genes, and the expression level of at least two, at least three, at least four, at least five, at least six, or all seven of VEGFA, KDR, ESM1, PECAM1, FLT1, ANGPTL4, or CD34 is below a reference level of the one or more genes.

For example, in some embodiments, the expression level of one or more (e.g., 1, 2, 3, 4, or 5) of CD8A, EOMES, PRF1, IFNG, or PD-L1 is at or above a reference level of the one or more genes, and the expression level of one or more (e.g., 1, 2, 3, 4, 5, or 6) of VEGFA, KDR, ESM1, PECAM1, ANGPTL4, or CD34 is below a reference level of the one or more genes. In some embodiments, the expression level of at least two, at least three, at least four, or all five of CD8A, EOMES, PRF1, IFNG, and PD-L1 is at or above a reference level of the one or more genes, and the expression level of at least one, at least two, at least three, at least four, at least five, or all six of VEGFA, KDR, ESM1, PECAM1, ANGPTL4, and CD34 is below a reference level fo the one or more genes. In some embodiments, the expression level of any one of the combinations set forth in Tables 2-4 is at or above a reference level of the one or more genes, and the expression level of any one of the combinations set forth in Tables 5-8 is below a reference level of the one or more genes. For example, in some embodiments, the expression level of two of CD8A, EOMES, PRF1, IFNG, and PD-L1, for example, any of the exemplary combinations shown in Table 2, is at or above a reference level of the one or more genes, and the expression level of two of VEGFA, KDR, ESM1, PECAM1, ANGPTL4, and CD34, for example, any of the exemplary combinations shown in Table 5, is below a reference level of the one or more genes. In some embodiments, the expression level of three of CD8A, EOMES, PRF1, IFNG, and PD-L1, for example, any of the exemplary combinations shown in Table 3, is at or above a reference level of the one or more genes, and the expression level of three of VEGFA, KDR, ESM1, PECAM1, ANGPTL4, and CD34, for example, any of the exemplary combinations shown in Table 6, is below a reference level of the one or more genes. In some embodiments, the expression level of four of CD8A, EOMES, PRF1, IFNG, and PD-L1, for example, any of the exemplary combinations shown in Table 4, is at or above a reference level of the one or more genes, and the expression level of four of VEGFA, KDR, ESM1, PECAM1, ANGPTL4, and CD34, for example, any of the exemplary combinations shown in Table 7, is below a reference level of the one or more genes. In some embodiments, the expression level of CD8A, EOMES, PRF1, IFNG, and PD-L1 is at or above a reference level of CD8A, EOMES, PRF1, IFNG, and PD-L1, and the expression level of five of VEGFA, KDR, ESM1, PECAM1, ANGPTL4, and CD34, for example, any of the exemplary combinations shown in Table 8, is below a reference level of the one or more genes. In some embodiments, the expression level of CD8A, EOMES, PRF1, IFNG, and PD-L1 is at or above a reference level of CD8A, EOMES, PRF1, IFNG, and PD-L1, and the expression level of VEGFA, KDR, ESM1, PECAM1, ANGPTL4, and CD34 is below a reference level of VEGFA, KDR, ESM1, PECAM1, ANGPTL4, and CD34.

In some embodiments of any of the preceding methods, the expression level of one or more (e.g., 1, 2, 3, 4, 5, or 6) of IL6, CXCL1, CXCL2, CXCL3, CXCL8, PTGS2, CXCR1, CXCR2, S100A8, or S100A9 in the sample is below a reference level of the one or more genes, and the method further comprises administering to the individual an effective amount of the anti-cancer therapy. In some embodiments, the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, or S100A9 in the sample is below a reference level of the one or more genes. For example, in some embodiments, the expression level of one or more of the exemplary combinations set forth in Tables 9-16 in the sample is below a reference expression level of the one or more genes. In some embodiments, the expression level of IL6, CXCL1, CXCL2, CXCL3, CXCL8, PTGS2, CXCR1, CXCR2, S100A8, and S100A9 in the sample is below a reference level of IL6, CXCL1, CXCL2, CXCL3, CXCL8, PTGS2, CXCR1, CXCR2, S100A8, and S100A9.

In other embodiments of any of the preceding methods, the expression level of one or more (e.g., 1, 2, 3, 4, 5, 6, or 7) of VEGFA, KDR, ESM1, PECAM1, FLT1, ANGPTL4, or CD34 in the sample is at or above a reference level of the one or more genes, and the method further includes administering to the individual an effective amount of an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))). In some embodiments, the expression level of at least one, at least two, at least three, at least four, at least five, at least six, or all seven of VEGFA, KDR, ESM1, PECAM1, FLT1, ANGPTL4, or CD34 is at or above a reference level of the one or more genes. In some embodiments, the expression level of one or more (e.g., 1, 2, 3, 4, 5, or 6) of VEGFA, KDR, ESM1, PECAM1, ANGPTL4, or CD34 in the sample is at or above a reference level of the one or more genes. In some embodiments, the expression level of one or more of the exemplary combinations set forth in Tables 5-8 in the sample is at or above a reference expression level of the one or more genes. In some embodiments, the expression level of VEGFA, KDR, ESM1, PECAM1, ANGPTL4, and CD34 in the sample is at or above a reference level of VEGFA, KDR, ESM1, PECAM1, ANGPTL4, and CD34.

In certain embodiments of any of the preceding methods, a reference level is the expression level of the one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37) genes (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2; VEGFA, KDR, ESM1, PECAM1, FLT1, ANGPTL4, or CD34; or IL6, CXCL1, CXCL2, CXCL3, CXCL8, PTGS2, CXCR1, CXCR2, S100A8, or S100A9) in a reference population, for example, a population of individuals having a cancer (e.g., a kidney cancer (e.g., RCC)). In particular embodiments, the cancer is a kidney cancer (e.g., RCC, e.g., mRCC). In certain embodiments, a reference level is the median expression level of the one or more genes in a reference population, for example, a population of individuals having a cancer. In other embodiments, the reference level may be the top 40%, the top 30%, the top 20%, the top 10%, the top 5%, or the top 1% of the expression level in the reference population. In certain embodiments, the reference level is a pre-assigned expression level for the one or more genes. In some embodiments, the reference level is a median of a Z-score of the normalized expression level of the one or more genes. In some embodiments, the reference level is the expression level of the one or more genes in a biological sample obtained from the patient at a previous time point, wherein the previous time point is following administration of the anti-cancer therapy. In some embodiments of any of the preceding methods, a reference level is the expression level of the one or more genes (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2; VEGFA, KDR, ESM1, PECAM1, FLT1, ANGPTL4, or CD34; or IL6, CXCL1, CXCL2, CXCL3, CXCL8, PTGS2, CXCR1, CXCR2, S100A8, or S100A9) in a biological sample from the patient obtained prior to (e.g., minutes, hours, days, weeks (e.g., 1, 2, 3, 4, 5, 6, or 7 weeks), months, or years prior to) administration of the anti-cancer therapy. In other embodiments, the reference level is the expression level of the one or more genes in a biological sample obtained from the patient at a subsequent time point (e.g., minutes, hours, days, weeks, months, or years after administration of an anti-cancer therapy).

The presence and/or expression level of any of the biomarkers described above may be assessed qualitatively and/or quantitatively based on any suitable criterion known in the art, including but not limited to DNA, mRNA, cDNA, proteins, protein fragments, and/or gene copy number. Methodologies for measuring such biomarkers are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (e.g., Serum ELISA), biochemical enzymatic activity assays, in situ hybridization (ISH), fluorescence in situ hybridization (FISH), Southern analysis, Northern analysis, whole genome sequencing, polymerase chain reaction (PCR) including quantitative real time PCR (qRT-PCR) and other amplification type detection methods, such as, for example, branched DNA, SISBA, TMA and the like, RNA-Seq, microarray analysis, gene expression profiling, whole-genome sequencing (WGS), and/or serial analysis of gene expression (“SAGE”), as well as any one of the wide variety of assays that can be performed by protein, gene, and/or tissue array analysis. Typical protocols for evaluating the status of genes and gene products are found, for example, in Ausubel et al. eds. (Current Protocols In Molecular Biology, 1995), Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In some embodiments of any of the preceding methods, the expression level of a biomarker may be a nucleic acid expression level (e.g., a DNA expression level or an RNA expression level (e.g., an mRNA expression level)). Any suitable method of determining a nucleic acid expression level may be used. In some embodiments, the nucleic acid expression level is determined using RNA-seq, RT-qPCR, qPCR, multiplex qPCR or RT-qPCR, microarray analysis, SAGE, MassARRAY technique, ISH, or a combination thereof.

Methods for the evaluation of mRNAs in cells are well known and include, for example, serial analysis of gene expression (SAGE), whole genome sequencing (WGS), hybridization assays using complementary DNA probes (such as in situ hybridization using labeled riboprobes specific for the one or more genes, Northern blot and related techniques) and various nucleic acid amplification assays (such as RT-PCR (e.g., qRT-PCR) using complementary primers specific for one or more of the genes, and other amplification type detection methods, such as, for example, branched DNA, SISBA, TMA and the like). In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined. Optional methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of treatment comprising a VEGF antagonist and a PD-L1 axis binding antagonist may be arrayed on a solid support. Hybridization of a labeled probe with a particular array member indicates that the sample from which the probe was derived expresses that gene.

In other embodiments of any of the preceding methods, the expression level of a biomarker may be a protein expression level. In certain embodiments, the method comprises contacting the sample with antibodies that specifically bind to a biomarker described herein under conditions permissive for binding of the biomarker, and detecting whether a complex is formed between the antibodies and biomarker.

Such method may be an in vitro or in vivo method. In some instances, an antibody is used to select patients eligible for therapy with a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A) or a PD-1 binding antagonist (e.g., an anti-PD-1 antibody)), e.g., a biomarker for selection of individuals. In other instances, an antibody is used to select patients eligible for therapy with an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))), e.g., a biomarker for selection of individuals. Any method of measuring protein expression levels known in the art or provided herein may be used. For example, in some embodiments, a protein expression level of a biomarker is determined using a method selected from the group consisting of flow cytometry (e.g., fluorescence-activated cell sorting (FACS™)), Western blot, enzyme-linked immunosorbent assay (ELISA), immunoprecipitation, immunohistochemistry (IHC), immunofluorescence, radioimmunoassay, dot blotting, immunodetection methods, HPLC, surface plasmon resonance, optical spectroscopy, mass spectrometry, and HPLC. In some embodiments, the protein expression level of the biomarker is determined in tumor-infiltrating immune cells. In some embodiments, the protein expression level of the biomarker is determined in tumor cells. In some embodiments, the protein expression level of the biomarker is determined in tumor-infiltrating immune cells and/or in tumor cells. In some embodiments, the protein expression level of the biomarker is determined in peripheral blood mononuclear cells (PBMCs).

In certain embodiments, the presence and/or expression level/amount of a biomarker protein in a sample is examined using IHC and staining protocols. IHC staining of tissue sections has been shown to be a reliable method of determining or detecting the presence of proteins in a sample. In some embodiments of any of the methods, assays and/or kits, the biomarker is one or more of the protein expression products of the following genes: CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and/or S100A9. In one embodiment, an expression level of biomarker is determined using a method comprising: (a) performing IHC analysis of a sample (such as a tumor sample obtained from a patient) with an antibody; and (b) determining expression level of a biomarker in the sample. In some embodiments, IHC staining intensity is determined relative to a reference. In some embodiments, the reference is a reference value. In some embodiments, the reference is a reference sample (e.g., a control cell line staining sample, a tissue sample from non-cancerous patient, or a tumor sample that is determined to be negative for the biomarker of interest).

IHC may be performed in combination with additional techniques such as morphological staining and/or in situ hybridization (e.g., ISH). Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for IHC typically will be labeled with a detectable moiety. Numerous labels are available which can be generally grouped into the following categories: (a) radioisotopes, such as ³⁵S, ¹⁴c, ¹²⁵I, ³H, and ¹³¹I; (b) colloidal gold particles; (c) fluorescent labels including, but are not limited to, rare earth chelates (europium chelates), Texas Red, rhodamine, fluorescein, dansyl, lissamine, umbelliferone, phycocrytherin, phycocyanin, or commercially-available fluorophores such as SPECTRUM ORANGE7 and SPECTRUM GREEN7 and/or derivatives of any one or more of the above; (d) various enzyme-substrate labels are available and U.S. Pat. No. 4,275,149 provides a review of some of these. Examples of enzymatic labels include luciferases (e.g., firefly luciferase and bacterial luciferase; see, e.g., U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of enzyme-substrate combinations include, for example, horseradish peroxidase (HRPO) with hydrogen peroxidase as a substrate; alkaline phosphatase (AP) with para-Nitrophenyl phosphate as chromogenic substrate; and β-D-galactosidase (β-D-Gal) with a chromogenic substrate (e.g., p-nitrophenyl-β-D-galactosidase) or fluorogenic substrate (e.g., 4-methylumbelliferyl-ρ-D-galactosidase). For a general review of these, see, for example, U.S. Pat. Nos. 4,275,149 and 4,318,980.

Specimens may be prepared, for example, manually, or using an automated staining instrument (e.g., a Ventana BenchMark XT or Benchmark ULTRA instrument). Specimens thus prepared may be mounted and coverslipped. Slide evaluation is then determined, for example, using a microscope, and staining intensity criteria, routinely used in the art, may be employed. In one embodiment, it is to be understood that when cells and/or tissue from a tumor is examined using IHC, staining is generally determined or assessed in tumor cell(s) and/or tissue (as opposed to stromal or surrounding tissue that may be present in the sample). In some embodiments, it is understood that when cells and/or tissue from a tumor is examined using IHC, staining includes determining or assessing in tumor-infiltrating immune cells, including intratumoral or peritumoral immune cells. In some embodiments, the presence of a biomarker is detected by IHC in >0% of the sample, in at least 1% of the sample, in at least 5% of the sample, in at least 10% of the sample, in at least 15% of the sample, in at least 15% of the sample, in at least 20% of the sample, in at least 25% of the sample, in at least 30% of the sample, in at least 35% of the sample, in at least 40% of the sample, in at least 45% of the sample, in at least 50% of the sample, in at least 55% of the sample, in at least 60% of the sample, in at least 65% of the sample, in at least 70% of the sample, in at least 75% of the sample, in at least 80% of the sample, in at least 85% of the sample, in at least 90% of the sample, in at least 95% of the sample, or more. Samples may be scored using any method known in the art, for example, by a pathologist or automated image analysis.

In some embodiments of any of the methods, the biomarker is detected by immunohistochemistry using a diagnostic antibody (i.e., primary antibody). In some embodiments, the diagnostic antibody specifically binds human antigen. In some embodiments, the diagnostic antibody is a non-human antibody. In some embodiments, the diagnostic antibody is a rat, mouse, or rabbit antibody. In some embodiments, the diagnostic antibody is a rabbit antibody. In some embodiments, the diagnostic antibody is a monoclonal antibody. In some embodiments, the diagnostic antibody is directly labeled. In other embodiments, the diagnostic antibody is indirectly labeled.

In some embodiments of any of the preceding embodiments, the sample is obtained from the individual prior to (e.g., minutes, hours, days, weeks (e.g., 1, 2, 3, 4, 5, 6, or 7 weeks), months, or years prior to) administration of the anti-cancer therapy. In some embodiments of any of the preceding methods, the sample from the individual is obtained about 2 to about 10 weeks (e.g., 2, 3, 4, 5, 6, 7, 8, 9, or 10 weeks) following administration of the anti-cancer therapy. In some embodiments, the sample from the individual is obtained about 4 to about 6 weeks following administration of the anti-cancer therapy.

In some embodiments of any of the preceding methods, the expression level or number of a biomarker is detected in a tissue sample, a primary or cultured cells or cell line, a cell supernatant, a cell lysate, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, or any combination thereof. In some embodiments, the sample is a tissue sample (e.g., a tumor tissue sample), a cell sample, a whole blood sample, a plasma sample, a serum sample, or a combination thereof. In some embodiments, the tumor tissue sample wherein the tumor tissue sample includes tumor cells, tumor-infiltrating immune cells, stromal cells, or a combination thereof. In some embodiments, the tumor tissue sample is a formalin-fixed and paraffin-embedded (FFPE) sample, an archival sample, a fresh sample, or a frozen sample.

For example, in some embodiments of any of the preceding methods, the expression level of a biomarker is detected in tumor-infiltrating immune cells, tumor cells, PBMCs, or combinations thereof using known techniques (e.g., flow cytometry or IHC). Tumor-infiltrating immune cells include, but are not limited to, intratumoral immune cells, peritumoral immune cells or any combinations thereof, and other tumor stroma cells (e.g., fibroblasts). Such tumor infiltrating immune cells may be T lymphocytes (such as CD8⁺ T lymphocytes (e.g., CD8⁺ T effector (Ten) cells) and/or CD4⁺ T lymphocytes (e.g., CD4⁺ T_{eff} cells), B lymphocytes, or other bone marrow-lineage cells including granulocytes (neutrophils, eosinophils, basophils), monocytes, macrophages, dendritic cells (e.g., interdigitating dendritic cells), histiocytes, and natural killer (NK) cells. In some embodiments, the staining for a biomarker is detected as membrane staining, cytoplasmic staining, or combinations thereof. In other embodiments, the absence of a biomarker is detected as absent or no staining in the sample, relative to a reference sample.

In particular embodiments of any of the preceding methods, the expression level of a biomarker is assessed in a sample that contains or is suspected to contain cancer cells. The sample may be, for example, a tissue biopsy or a metastatic lesion obtained from a patient suffering from, suspected to suffer from, or diagnosed with cancer (e.g., a kidney cancer, in particular renal cell carcinoma (RCC), such as advanced RCC or metastatic RCC (mRCC)). In some embodiments, the sample is a sample of kidney tissue, a biopsy of an kidney tumor, a known or suspected metastatic kidney cancer lesion or section, or a blood sample, e.g., a peripheral blood sample, known or suspected to comprise circulating cancer cells, e.g., kidney cancer cells. The sample may comprise both cancer cells, i.e., tumor cells, and non-cancerous cells (e.g., lymphocytes, such as T cells or NK cells), and, in certain embodiments, comprises both cancerous and non-cancerous cells. Methods of obtaining biological samples including tissue resections, biopsies, and body fluids, e.g., blood samples comprising cancer/tumor cells, are well known in the art.

In some embodiments of any of the preceding methods, the patient has carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. In some embodiments, the cancer is kidney cancer (e.g., renal cell carcinoma (RCC), e.g., advanced RCC or metastatic RCC (mRCC)), squamous cell cancer (e.g., epithelial squamous cell cancer), lung cancer (including small-cell lung cancer (SCLC), non-small cell lung cancer (NSCLC), adenocarcinoma of the lung, and squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer (e.g., HCC), hepatoma, breast cancer (including metastatic breast cancer), bladder cancer, colon cancer, rectal cancer, colorectal cancer, endometrial or uterine carcinoma, salivary gland carcinoma, prostate cancer, vulval cancer, thyroid cancer, hepatic carcinoma, anal carcinoma, penile carcinoma, Merkel cell cancer, mycoses fungoids, testicular cancer, esophageal cancer, tumors of the biliary tract, head and neck cancer, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), abnormal vascular proliferation associated with phakomatoses, edema (such as that associated with brain tumors), or Meigs' syndrome. In some embodiments, the cancer is a kidney cancer (e.g., RCC), a lung cancer (e.g., NSCLC), a bladder cancer (e.g., UBC), a liver cancer (e.g., HCC), an ovarian cancer, or a breast cancer (e.g., TNBC). In preferred embodiments, the patient has a kidney cancer (e.g., RCC, e.g., advanced RCC or mRCC, e.g., previously untreated advanced RCC or mRCC). The patient may optionally have an advanced, refractory, recurrent, chemotherapy-resistant, and/or platinum-resistant form of the cancer.

In certain embodiments, the presence and/or expression levels/amount of a biomarker in a first sample is increased or elevated as compared to presence/absence and/or expression levels/amount in a second sample. In certain embodiments, the presence/absence and/or expression levels/amount of a biomarker in a first sample is decreased or reduced as compared to presence and/or expression levels/amount in a second sample. In certain embodiments, the second sample is a reference sample, reference cell, reference tissue, control sample, control cell, or control tissue.

In certain embodiments, a reference sample, reference cell, reference tissue, control sample, control cell, or control tissue is a single sample or combined multiple samples from the same patient or individual that are obtained at one or more different time points than when the test sample is obtained. For example, a reference sample, reference cell, reference tissue, control sample, control cell, or control tissue is obtained at an earlier time point from the same patient or individual than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference sample, reference cell, reference tissue, control sample, control cell, or control tissue is a combined multiple samples from one or more healthy individuals who are not the patient. In certain embodiments, a reference sample, reference cell, reference tissue, control sample, control cell, or control tissue is a combined multiple samples from one or more individuals with a disease or disorder (e.g., cancer) who are not the patient or individual. In certain embodiments, a reference sample, reference cell, reference tissue, control sample, control cell, or control tissue is pooled RNA samples from normal tissues or pooled plasma or serum samples from one or more individuals who are not the patient. In certain embodiments, a reference sample, reference cell, reference tissue, control sample, control cell, or control tissue is pooled RNA samples from tumor tissues or pooled plasma or serum samples from one or more individuals with a disease or disorder (e.g., cancer) who are not the patient.

In some embodiments of any of the preceding methods, an expression level above a reference level, or an elevated or increased expression or number, refers to an overall increase of about any of 10%, 20%, 30%, 40%, 50%, 60%, 70%, 80%, 90%, 95%, 96%, 97%, 98%, 99% or greater, in the level or number of a biomarker (e.g., protein, nucleic acid (e.g., gene or mRNA), or cell), detected by methods such as those described herein and/or known in the art, as compared to a reference level, reference sample, reference cell, reference tissue, control sample, control cell, or control tissue. In certain embodiments, the elevated expression or number refers to the increase in expression level/amount of a biomarker (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2 CXCR1, CXCR2, S100A8, and/or S100A9) in the sample wherein the increase is at least about any of 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, 3.5×, 4×, 4.5×, 5×, 6×, 7×, 8×, 9×, 10×, 15×, 20×, 30×, 40×, 50×, 100×, 500×, or 1000× the expression level/amount of the respective biomarker in a reference level, reference sample, reference cell, reference tissue, control sample, control cell, or control tissue. In some embodiments, elevated expression or number refers to an overall increase in expression level/amount of a biomarker (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2 CXCR1, CXCR2, S100A8, and/or S100A9) of greater than about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, about 3-fold, about 3.5-fold, about 4-fold, about 4.5-fold, about 5-fold, about 6-fold, about 7-fold, about 8-fold, about 9-fold, about 10-fold, about 15-fold, about 20-fold, about 30-fold, about 40-fold, about 50-fold, about 100-fold, about 500-fold, about 1,000-fold or greater as compared to a reference level, reference sample, reference cell, reference tissue, control sample, control cell, control tissue, or internal control (e.g., housekeeping gene).

In some embodiments of any of the preceding methods, an expression level below a reference level, or a reduced (decreased) expression or number, refers to an overall reduction of about any of 10%, 20%, 30%, 40%, 50%, 60%, 70%, 80%, 90%, 95%, 96%, 97%, 98%, 99% or greater, in the level of biomarker (e.g., protein, nucleic acid (e.g., gene or mRNA), or cell), detected by standard art known methods such as those described herein, as compared to a reference level, reference sample, reference cell, reference tissue, control sample, control cell, or control tissue. In certain embodiments, reduced expression or number refers to the decrease in expression level/amount of a biomarker (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and/or S100A9) in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference level, reference sample, reference cell, reference tissue, control sample, control cell, or control tissue. In some embodiments, reduced (decreased) expression or number refers to an overall decrease in expression level/amount of a biomarker (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and/or S100A9) of greater than about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, about 3-fold, about 3.5-fold, about 4-fold, about 4.5-fold, about 5-fold, about 6-fold, about 7-fold, about 8-fold, about 9-fold, about 10-fold, about 15-fold, about 20-fold, about 30-fold, about 40-fold, about 50-fold, about 100-fold, about 500-fold, about 1,000-fold or greater as compared to a reference level, reference sample, reference cell, reference tissue, control sample, control cell, control tissue, or internal control (e.g., housekeeping gene).

### III. THERAPEUTIC METHODS AND USES

Provided herein are methods for treating an individual having a cancer (e.g., a kidney cancer (e.g., RCC)). In particular embodiments, the cancer is a kidney cancer, such as RCC, e.g., advanced RCC or mRCC, e.g., previously untreated advanced RCC or mRCC. In other particular embodiments, the cancer is a sarcomatoid cancer, such as sarcomatoid kidney cancer, e.g., sarcomatoid RCC, e.g., advanced sarcomatoid RCC or sarcomatoid mRCC, e.g., previously untreated advanced sarcomatoid RCC or sarcomatoid mRCC. In some instances, the methods of the invention include administering to the individual an anti-cancer therapy that includes a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) based on the expression level of a biomarker of the invention (e.g., the presence of a sarcomatoid cancer (e.g., a sarcomatoid kidney cancer (e.g., a sarcomatoid RCC)), an individual's MSKCC risk score, or one or more genes set forth in Table 1). In other embodiments, the methods of the invention include administering to the individual an anti-cancer therapy that includes an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))). Any of the VEGF antagonists, PD-L1 axis binding antagonists, angiogenesis inhibitors (e.g., multi-targeted tyrosine kinase inhibitors), or other anti-cancer agents described herein (e.g., as described below in Section V and/or the Examples) or known in the art may be used in the methods. Such a treatment may benefit the individual, for example, in terms of improved progression-free survival (PFS), overall survival (OS), overall response rate (ORR), complete response (CR) rate, and/or deterioration-free rate (DFR). For example, in some instances, the benefit may be in terms of PFS. In other instances, the benefit may be in terms of OS. In yet other instances, the benefit may be in terms of ORR. In still other instances, the benefit may be in terms of CR rate. In still other instances, the benefit may be in terms of DFR.

The invention further relates to methods for improving PFS, OS, ORR, CR rate, and/or DFR of a patient suffering from a cancer (e.g., a kidney cancer (e.g., RCC)) by administration of an anti-cancer therapy that includes a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab) or a PD-1 binding antagonist (e.g., an anti-PD-1 antibody)). The invention further relates to methods for improving PFS, OS, ORR, CR rate, and/or DFR of a patient suffering from a cancer (e.g., a kidney cancer (e.g., RCC)) by administration of an anti-cancer therapy that includes an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))).

The presence, expression level, or number of any of the biomarkers described herein may be determined using any method known in the art and/or described herein, for example, in Section II above and/or in the working Examples.

In one example, provided herein is a method of treating an individual having a sarcomatoid cancer (e.g., a sarcomatoid kidney cancer (e.g., a sarcomatoid RCC, including locally advanced or metastatic sarcomatoid RCC)), the method comprising administering to the individual an effective amount of an anti-cancer therapy comprising a VEGF antagonist and a PD-L1 axis binding antagonist. In some embodiments, the individual is previously untreated for the sarcomatoid cancer.

In another example, provided herein is a method of treating an individual having a cancer (e.g., a kidney cancer (e.g., RCC)), the method comprising administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the individual has been identified as likely to benefit from the anti-cancer therapy based on having a sarcomatoid cancer (e.g., a sarcomatoid kidney cancer (e.g., a sarcomatoid RCC)).

In another example, provided herein is a method of treating an individual having a cancer (e.g., a kidney cancer (e.g., RCC)), the method comprising: (a) determining whether the individual has a sarcomatoid cancer (e.g., a sarcomatoid kidney cancer (e.g., a sarcomatoid RCC)), wherein the presence of a sarcomatoid kidney cancer indicates that the individual is likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)); and (b) administering an effective amount of an anti-cancer therapy comprising a VEGF antagonist and a PD-L1 axis binding antagonist to the individual based on the presence of a sarcomatoid kidney cancer.

The benefit may be, for example, in terms of improved progression-free survival (PFS), overall survival (OS), overall response rate (ORR), complete response (CR) rate, or deterioration-free rate (DFR). In some embodiments, the benefit is in terms of improved PFS. In some instances, the benefit is in terms of improved OS. In some instances, the benefit is in terms of improved ORR. In some instances, the benefit is in terms of improved CR rate. In some instances, the benefit is in terms of improved DFR. In some instances, DFR is determined in terms of the time from onset of treatment to the individual's first increase of greater than or equal to 2 points above baseline on the MD Anderson Symptom Inventory (MDASI) interference scale.

For example, provided herein is a method of treating an individual having a cancer (e.g., a kidney cancer (e.g., RCC)), the method comprising administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the individual has been identified as likely to benefit from the anti-cancer therapy based on having a sarcomatoid cancer (e.g., a sarcomatoid kidney cancer (e.g., a sarcomatoid RCC)), wherein the benefit is in terms of improved PFS.

In another example, provided herein is a method of treating an individual having a cancer (e.g., a kidney cancer (e.g., RCC)), the method comprising: (a) determining whether the individual has a sarcomatoid cancer (e.g., a sarcomatoid kidney cancer (e.g., a sarcomatoid RCC)), wherein the presence of a sarcomatoid kidney cancer indicates that the individual is likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved PFS; and (b) administering an effective amount of an anti-cancer therapy comprising a VEGF antagonist and a PD-L1 axis binding antagonist to the individual based on the presence of a sarcomatoid kidney cancer.

In yet another example, provided herein is a method of treating an individual having a cancer (e.g., a kidney cancer (e.g., RCC)), the method comprising administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the individual has been identified as likely to benefit from the anti-cancer therapy based on having a sarcomatoid cancer (e.g., a sarcomatoid kidney cancer (e.g., a sarcomatoid RCC)), wherein the benefit is in terms of improved OS.

In another example, provided herein is a method of treating an individual having a cancer (e.g., a kidney cancer (e.g., RCC)), the method comprising: (a) determining whether the individual has a sarcomatoid cancer (e.g., a sarcomatoid kidney cancer (e.g., a sarcomatoid RCC)), wherein the presence of a sarcomatoid kidney cancer indicates that the individual is likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved OS; and (b) administering an effective amount of an anti-cancer therapy comprising a VEGF antagonist and a PD-L1 axis binding antagonist to the individual based on the presence of a sarcomatoid kidney cancer.

In a further example, provided herein is a method of treating an individual having a cancer (e.g., a kidney cancer (e.g., RCC)), the method comprising administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the individual has been identified as likely to benefit from the anti-cancer therapy based on having a sarcomatoid cancer (e.g., a sarcomatoid kidney cancer (e.g., a sarcomatoid RCC)), wherein the benefit is in terms of improved ORR.

In a still further example, provided herein is a method of treating an individual having a cancer (e.g., a kidney cancer (e.g., RCC)), the method comprising: (a) determining whether the individual has a sarcomatoid cancer (e.g., a sarcomatoid kidney cancer (e.g., a sarcomatoid RCC)), wherein the presence of a sarcomatoid kidney cancer indicates that the individual is likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved ORR; and (b) administering an effective amount of an anti-cancer therapy comprising a VEGF antagonist and a PD-L1 axis binding antagonist to the individual based on the presence of a sarcomatoid kidney cancer.

In yet another example, provided herein is a method of treating an individual having a cancer (e.g., a kidney cancer (e.g., RCC)), the method comprising administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the individual has been identified as likely to benefit from the anti-cancer therapy based on having a sarcomatoid cancer (e.g., a sarcomatoid kidney cancer (e.g., a sarcomatoid RCC)), wherein the benefit is in terms of improved CR rate.

In a still further example, provided herein is a method of treating an individual having a cancer (e.g., a kidney cancer (e.g., RCC)), the method comprising: (a) determining whether the individual has a sarcomatoid cancer (e.g., a sarcomatoid kidney cancer (e.g., a sarcomatoid RCC)), wherein the presence of a sarcomatoid kidney cancer indicates that the individual is likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved CR rate; and (b) administering an effective amount of an anti-cancer therapy comprising a VEGF antagonist and a PD-L1 axis binding antagonist to the individual based on the presence of a sarcomatoid kidney cancer.

In another example, provided herein is a method of treating an individual having a cancer (e.g., a kidney cancer (e.g., RCC)), the method comprising administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the individual has been identified as likely to benefit from the anti-cancer therapy based on having a sarcomatoid cancer (e.g., a sarcomatoid kidney cancer (e.g., a sarcomatoid RCC)), wherein the benefit is in terms of improved DFR.

In yet another example, provided herein is a method of treating an individual having a cancer (e.g., a kidney cancer (e.g., RCC)), the method comprising: (a) determining whether the individual has a sarcomatoid cancer (e.g., a sarcomatoid kidney cancer (e.g., a sarcomatoid RCC)), wherein the presence of a sarcomatoid kidney cancer indicates that the individual is likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved DFR; and (b) administering an effective amount of an anti-cancer therapy comprising a VEGF antagonist and a PD-L1 axis binding antagonist to the individual based on the presence of a sarcomatoid kidney cancer.

The presence of a sarcomatoid cancer (e.g., kidney cancer (e.g., RCC)) can be determined using any suitable approach. See, e.g., El Mouallem et al. Urol. Oncol. 36:265-271, 2018, which is incorporated herein by reference in its entirety. For example, in some embodiments, the presence of a sarcomatoid cancer (e.g., a sarcomatoid kidney cancer (e.g., a sarcomatoid RCC)) is assessed by histological analysis of a sample obtained from the individual. In some embodiments, the kidney cancer is sarcomatoid if a tumor sample from the individual contains a focus or foci of high-grade malignant spindle cells of any component relative to the entire tumor area. In some embodiments, the spindle cells show moderate to marked atypia and/or resemble any form of sarcoma. In some embodiments, the spindle cells show evidence of epithelial differentiation as assessed by immunohistological positivity for keratin or epithelial membrane antigen (EMA). In some embodiments, the kidney cancer is renal cell carcinoma, and the tumor sample has epithelial differentiation with concurrent areas of renal cell carcinoma.

In any of the preceding methods, the method may further include determining the individual's MSKCC risk score. In other embodiments, the individual's MSKCC risk score has previously been determined. In any of the preceding methods, the individual may have a poor or intermediate MSKCC risk score.

In another example, provided herein is a method of treating an individual having a cancer (e.g., a kidney cancer (e.g., an RCC, including locally advanced or metastatic RCC)) with a poor or intermediate Memorial Sloan Kettering Cancer Center (MSKCC) risk score, the method comprising administering to the individual an effective amount of an anti-cancer therapy comprising a VEGF antagonist and a PD-L1 axis binding antagonist. In some embodiments, the individual is previously untreated for the cancer.

In yet another example, provided herein is a method of treating an individual having a cancer (e.g., a kidney cancer (e.g., RCC)), the method comprising administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the individual has been identified as likely to benefit from the anti-cancer therapy based on the individual having a poor or intermediate MSKCC risk score.

In yet a further example, provided herein is a method of treating an individual having a cancer (e.g., a kidney cancer (e.g., RCC)), the method comprising: (a) determining the individual's MSKCC risk score, wherein a poor or intermediate MSKCC risk score indicates that the individual is likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) and (b) administering an effective amount of an anti-cancer therapy comprising a VEGF antagonist and a PD-L1 axis binding antagonist to the individual based on the individual having a poor or intermediate MSKCC risk score.

The benefit may be, for example, in terms of improved progression-free survival (PFS), overall survival (OS), overall response rate (ORR), complete response (CR) rate, or deterioration-free rate (DFR). In some embodiments, the benefit is in terms of improved PFS. In some instances, the benefit is in terms of improved OS. In some instances, the benefit is in terms of improved ORR. In some instances, the benefit is in terms of improved CR rate. In some instances, the benefit is in terms of improved DFR. In some instances, DFR is determined in terms of the time from onset of treatment to the individual's first increase of greater than or equal to 2 points above baseline on the MD Anderson Symptom Inventory (MDASI) interference scale.

For example, provided herein is a method of treating an individual having a cancer (e.g., a kidney cancer (e.g., RCC)), the method comprising administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the individual has been identified as likely to benefit from the anti-cancer therapy based on the individual having a poor or intermediate MSKCC risk score, wherein the benefit is in terms of improved PFS.

In another example, provided herein is a method of treating an individual having a cancer (e.g., a kidney cancer (e.g., RCC)), the method comprising: (a) determining the individual's MSKCC risk score, wherein a poor or intermediate MSKCC risk score indicates that the individual is likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved PFS; and (b) administering an effective amount of an anti-cancer therapy comprising a VEGF antagonist and a PD-L1 axis binding antagonist to the individual based on the individual having a poor or intermediate MSKCC risk score.

In yet another example, provided herein is a method of treating an individual having a cancer (e.g., a kidney cancer (e.g., RCC)), the method comprising administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the individual has been identified as likely to benefit from the anti-cancer therapy based on the individual having a poor or intermediate MSKCC risk score, wherein the benefit is in terms of improved OS.

In another example, provided herein is a method of treating an individual having a cancer (e.g., a kidney cancer (e.g., RCC)), the method comprising: (a) determining the individual's MSKCC risk score, wherein a poor or intermediate MSKCC risk score indicates that the individual is likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved OS; and (b) administering an effective amount of an anti-cancer therapy comprising a VEGF antagonist and a PD-L1 axis binding antagonist to the individual based on the individual having a poor or intermediate MSKCC risk score.

In a further example, provided herein is a method of treating an individual having a cancer (e.g., a kidney cancer (e.g., RCC)), the method comprising administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the individual has been identified as likely to benefit from the anti-cancer therapy based on the individual having a poor or intermediate MSKCC risk score, wherein the benefit is in terms of improved ORR.

In yet a further example, provided herein is a method of treating an individual having a cancer (e.g., a kidney cancer (e.g., RCC)), the method comprising: (a) determining the individual's MSKCC risk score, wherein a poor or intermediate MSKCC risk score indicates that the individual is likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved ORR; and (b) administering an effective amount of an anti-cancer therapy comprising a VEGF antagonist and a PD-L1 axis binding antagonist to the individual based on the individual having a poor or intermediate MSKCC risk score.

In a further example still, provided herein is a method of treating an individual having a cancer (e.g., a kidney cancer (e.g., RCC)), the method comprising administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the individual has been identified as likely to benefit from the anti-cancer therapy based on the individual having a poor or intermediate MSKCC risk score, wherein the benefit is in terms of improved CR rate.

In another example, provided herein is a method of treating an individual having a cancer (e.g., a kidney cancer (e.g., RCC)), the method comprising: (a) determining the individual's MSKCC risk score, wherein a poor or intermediate MSKCC risk score indicates that the individual is likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved CR rate; and (b) administering an effective amount of an anti-cancer therapy comprising a VEGF antagonist and a PD-L1 axis binding antagonist to the individual based on the individual having a poor or intermediate MSKCC risk score.

In another example, provided herein is a method of treating an individual having a cancer (e.g., a kidney cancer (e.g., RCC)), the method comprising administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the individual has been identified as likely to benefit from the anti-cancer therapy based on the individual having a poor or intermediate MSKCC risk score, wherein the benefit is in terms of improved DFR.

In yet another example, provided herein is a method of treating an individual having a cancer (e.g., a kidney cancer (e.g., RCC)), the method comprising: (a) determining the individual's MSKCC risk score, wherein a poor or intermediate MSKCC risk score indicates that the individual is likely to benefit from an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), wherein the benefit is in terms of improved DFR; and (b) administering an effective amount of an anti-cancer therapy comprising a VEGF antagonist and a PD-L1 axis binding antagonist to the individual based on the individual having a poor or intermediate MSKCC risk score.

In any of the preceding methods, the individual may have a poor MSKCC risk score if the individual has three or more (e.g., three, four, or all five) of the following characteristics: (i) a time from nephrectomy to systemic treatment of less than one year, a lack of a nephrectomy, or an initial diagnosis with metastatic disease; (ii) a hemoglobin level less than the lower limit of normal (LLN), optionally wherein the normal range for hemoglobin is between 13.5 and 17.5 g/dL for men and between 12 and 15.5 g/dL for women; (iii) a serum corrected calcium level greater than 10 mg/dL, optionally wherein the serum corrected calcium level is the serum calcium level (mg/dL)+0.8(4−serum albumin (g/dL)); (iv) a serum lactate dehydrogenase (LDH) level greater than 1.5 times the upper limit of normal (ULN), optionally wherein the ULN is 140 U/L; and/or (v) a Karnofsky Performance Status (KPS) score of <80. In some embodiments, the individual has three of the preceding characteristics. In other embodiments, the individual has four of the preceding characteristics. In yet other embodiments, the individual has all five of the preceding characteristics.

In any of the preceding methods, the individual may have an intermediate MSKCC risk score if the individual has one or two of the following characteristics: (i) a time from nephrectomy to systemic treatment of less than one year, a lack of a nephrectomy, or an initial diagnosis with metastatic disease; (ii) a hemoglobin level less than the LLN, optionally wherein the normal range for hemoglobin is between 13.5 and 17.5 g/dL for men and between 12 and 15.5 g/dL for women; (iii) a serum corrected calcium level greater than 10 mg/dL, optionally wherein the serum corrected calcium level is the serum calcium level (mg/dL)+0.8(4−serum albumin (g/dL)); (iv) a serum LDH level greater than 1.5 times the ULN, optionally wherein the ULN is 140 U/L; and/or (v) a KPS score of <80. In some embodiments, the individual has one of the preceding characteristics. In other embodiments, the individual has two of the preceding characteristics,

In any of the preceding methods, the individual may have a sarcomatoid cancer (e.g., a sarcomatoid kidney cancer (e.g., a sarcomatoid RCC)).

In some embodiments of any of the preceding methods, the method further comprises determining the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37) of the genes set forth in Table 1. In other embodiments, the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37) of the genes set forth in Table 1 has previously been determined.

For example, in some embodiments, the method further comprises determining the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33) of the following genes in a sample from the individual: CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2; VEGFA, KDR, ESM1, PECAM1, FLT1, ANGPTL4, or CD34; or IL6, CXCL1, CXCL2, CXCL3, CXCL8, or PTGS2. In other embodiments, the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33) of the following genes in a sample from the individual: CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2; VEGFA, KDR, ESM1, PECAM1, FLT1, ANGPTL4, or CD34; or IL6, CXCL1, CXCL2, CXCL3, CXCL8, or PTGS2 has previously been determined.

In some embodiments of any of the preceding methods, (i) an expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample that is at or above a reference expression level of the one or more genes; or (ii) an expression level of one or more (e.g., 1, 2, 3, 4, 5, 6, 7, 8, 9, 10, 11, 12, or 13) of VEGFA, KDR, ESM1, PECAM1, FLT1, ANGPTL4, or CD34; or IL6, CXCL1, CXCL2, CXCL3, CXCL8, or PTGS2 in the sample that is below a reference expression level of the one or more genes identifies the individual as one who may benefit from treatment with an anti-cancer therapy comprising a VEGF antagonist and a PD-L1 axis binding antagonist.

Any of the preceding methods may include determining the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2. In some embodiments, the method includes determining the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2.

For example, any of the preceding methods may include determining the expression level of one or more (e.g., 1, 2, 3, 4, or 5) of CD8A, EOMES, PRF1, IFNG, or PD-L1. In some embodiments, the method includes determining the expression level of at least two, at least three, at least four, or all five of CD8A, EOMES, PRF1, IFNG, and PD-L1. In some embodiments, the method includes determining the expression level of two of CD8A, EOMES, PRF1, IFNG, and PD-L1, for example, any of the exemplary combinations shown in Table 2. In some embodiments, the method includes determining the expression level of three of CD8A, EOMES, PRF1, IFNG, and PD-L1, for example, any of the exemplary combinations shown in Table 3. In some embodiments, the method includes determining the expression level of four of CD8A, EOMES, PRF1, IFNG, and PD-L1, for example, any of the exemplary combinations shown in Table 4. In some embodiments, the method involves determining the expression level of CD8A, EOMES, PRF1, IFNG, and PD-L1.

In some embodiments, any of the preceding methods may include determining the expression level of PD-L1 and one or more additional genes, wherein the one or more additional genes is other than PD-L1. For example, in some embodiments, the method may include determining the expression level of PD-L1 and one or more additional genes (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36) selected from the group consisting of: CD8A, EOMES, GZMA, GZMB, PRF1, IFNG, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and S100A9. In some embodiments, the method includes determining the expression level of PD-L1 and one or more (e.g., 1, 2, 3, 4, 5, 6, 7, 8, 9, 10, 11, 12, 13, 14, 15, 16, 17, 18, or 19) additional genes selected from the group consisting of CD8A, EOMES, GZMA, GZMB, PRF1, IFNG, CXCL9, CXCL10, CXCL11, CD27, FOXP3, PD-1, CTLA4, TIGIT, IDO1, PSMB8, PSMB9, TAP1, and TAP2. In other embodiments, the method includes determining the expression level of PD-L1 and one or more (e.g., 1, 2, 3, 4, 5, 6, or 7) of VEGFA, KDR, ESM1, PECAM1, FLT1, ANGPTL4, or CD34. In other embodiments, the method includes determining the expression level of PD-L1 and one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9.

Any of the preceding methods may include determining the expression level of one or more (e.g., 1, 2, 3, 4, 5, 6, or 7) of VEGFA, KDR, ESM1, PECAM1, FLT1, ANGPTL4, or CD34. In some embodiments, the method includes determining the expression level of at least two, at least three, at least four, at least five, at least six, or all seven of VEGFA, KDR, ESM1, PECAM1, FLT1, ANGPTL4, or CD34. For example, in some embodiments, the method includes determining the expression level of one or more of VEGFA, KDR, ESM1, PECAM1, ANGPTL4, or CD34. In some embodiments, the method includes determining the expression level of at least two, at least three, at least four, at least five, or all six of VEGFA, KDR, ESM1, PECAM1, ANGPTL4, and CD34. In some embodiments, the method includes determining the expression level of two of VEGFA, KDR, ESM1, PECAM1, ANGPTL4, and CD34, for example, any of the exemplary combinations shown in Table 5. In some embodiments, the method includes determining the expression level of three of VEGFA, KDR, ESM1, PECAM1, ANGPTL4, and CD34, for example, any of the exemplary combinations shown in Table 6. In some embodiments, the method includes determining the expression level of four of VEGFA, KDR, ESM1, PECAM1, ANGPTL4, and CD34, for example, any of the exemplary combinations shown in Table 7. In some embodiments, the method includes determining the expression level of five of VEGFA, KDR, ESM1, PECAM1, ANGPTL4, and CD34, for example, any of the exemplary combinations shown in Table 8. In some embodiments, the method includes determining the expression level of VEGFA, KDR, ESM1, PECAM1, ANGPTL4, and CD34.

Any of the preceding methods may include determining the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9. In some embodiments, the method includes determining the expression level of at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9. In some embodiments, the method includes determining the expression level of two of IL6, CXCL1, CXCL2, CXCL3, CXCL8, PTGS2, CXCR1, CXCR2, S100A8, and S100A9, for example, any of the exemplary combinations shown in Table 9. In some embodiments, the method includes determining the expression level of three of IL6, CXCL1, CXCL2, CXCL3, CXCL8, PTGS2, CXCR1, CXCR2, S100A8, and S100A9, for example, any of the exemplary combinations shown in Table 10. In some embodiments, the method includes determining the expression level of four of IL6, CXCL1, CXCL2, CXCL3, CXCL8, PTGS2, CXCR1, CXCR2, S100A8, and S100A9, for example, any of the exemplary combinations shown in Table 11. In some embodiments, the method includes determining the expression level of five of IL6, CXCL1, CXCL2, CXCL3, CXCL8, PTGS2, CXCR1, CXCR2, S100A8, and S100A9, for example, any of the exemplary combinations shown in Table 12. In some embodiments, the method includes determining the expression level of six of IL6, CXCL1, CXCL2, CXCL3, CXCL8, PTGS2, CXCR1, CXCR2, S100A8, and S100A9, for example, any of the exemplary combinations shown in Table 13. In some embodiments, the method includes determining the expression level of seven of IL6, CXCL1, CXCL2, CXCL3, CXCL8, PTGS2, CXCR1, CXCR2, S100A8, and S100A9, for example, any of the exemplary combinations shown in Table 14. In some embodiments, the method includes determining the expression level of eight of IL6, CXCL1, CXCL2, CXCL3, CXCL8, PTGS2, CXCR1, CXCR2, S100A8, and S100A9, for example, any of the exemplary combinations shown in Table 15. In some embodiments, the method includes determining the expression level of nine of IL6, CXCL1, CXCL2, CXCL3, CXCL8, PTGS2, CXCR1, CXCR2, S100A8, and S100A9, for example, any of the exemplary combinations shown in Table 16. In some embodiments, the method includes determining the expression level of IL6, CXCL1, CXCL2, CXCL3, CXCL8, PTGS2, CXCR1, CXCR2, S100A8, and S100A9.

In any of the preceding methods, the method may include determining the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2, and one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9. For example, in some embodiments, the method includes determining the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2, and at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9.

For example, any of the preceding methods may include determining the expression level of one or more (e.g., 1, 2, 3, 4, or 5) of CD8A, EOMES, PRF1, IFNG, or PD-L1, and one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9. In some embodiments, the method includes determining the expression level of at least two, at least three, at least four, or all five of CD8A, EOMES, PRF1, IFNG, and PD-L1, and at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9. In some embodiments, the method comprises determining the expression level of any one of the combinations set forth in Tables 2-4 and any one of the combinations set forth in Tables 9-16. For example, in some embodiments, the method includes determining the expression level of two of CD8A, EOMES, PRF1, IFNG, and PD-L1, for example, any of the exemplary combinations shown in Table 2, and two of IL6, CXCL1, CXCL2, CXCL3, CXCL8, PTGS2, CXCR1, CXCR2, S100A8, and S100A9, for example, any of the exemplary combinations shown in Table 9. In some embodiments, the method includes determining the expression level of three of CD8A, EOMES, PRF1, IFNG, and PD-L1, for example, any of the exemplary combinations shown in Table 3, and three of IL6, CXCL1, CXCL2, CXCL3, CXCL8, PTGS2, CXCR1, CXCR2, S100A8, and S100A9, for example, any of the exemplary combinations shown in Table 10. In some embodiments, the method includes determining the expression level of four of CD8A, EOMES, PRF1, IFNG, and PD-L1, for example, any of the exemplary combinations shown in Table 4, and four of IL6, CXCL1, CXCL2, CXCL3, CXCL8, PTGS2, CXCR1, CXCR2, S100A8, and S100A9, for example, any of the exemplary combinations shown in Table 11. In some embodiments, the method involves determining the expression level of CD8A, EOMES, PRF1, IFNG, and PD-L1, and five of IL6, CXCL1, CXCL2, CXCL3, CXCL8, PTGS2, CXCR1, CXCR2, S100A8, and S100A9, for example, any of the exemplary combinations shown in Table 12. In some embodiments, the method involves determining the expression level of CD8A, EOMES, PRF1, IFNG, and PD-L1, and six of IL6, CXCL1, CXCL2, CXCL3, CXCL8, PTGS2, CXCR1, CXCR2, S100A8, and S100A9, for example, any of the exemplary combinations shown in Table 13. In some embodiments, the method involves determining the expression level of CD8A, EOMES, PRF1, IFNG, and PD-L1, and seven of IL6, CXCL1, CXCL2, CXCL3, CXCL8, PTGS2, CXCR1, CXCR2, S100A8, and S100A9, for example, any of the exemplary combinations shown in Table 14. In some embodiments, the method involves determining the expression level of CD8A, EOMES, PRF1, IFNG, and PD-L1, and eight of IL6, CXCL1, CXCL2, CXCL3, CXCL8, PTGS2, CXCR1, CXCR2, S100A8, and S100A9, for example, any of the exemplary combinations shown in Table 15. In some embodiments, the method involves determining the expression level of CD8A, EOMES, PRF1, IFNG, and PD-L1, and nine of IL6, CXCL1, CXCL2, CXCL3, CXCL8, PTGS2, CXCR1, CXCR2, S100A8, and S100A9, for example, any of the exemplary combinations shown in Table 16. In some embodiments, the method involves determining the expression level of CD8A, EOMES, PRF1, IFNG, PD-L1, IL6, CXCL1, CXCL2, CXCL3, CXCL8, PTGS2, CXCR1, CXCR2, S100A8, and S100A9.

In other embodiments, in any of the preceding methods, the method may include determining the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2, and one or more (e.g., 1, 2, 3, 4, 5, 6, or 7) of VEGFA, KDR, ESM1, PECAM1, FLT1, ANGPTL4, or CD34. For example, in some embodiments, the method includes determining the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2, and at least two, at least three, at least four, at least five, at least six, or all seven of VEGFA, KDR, ESM1, PECAM1, FLT1, ANGPTL4, or CD34.

For example, any of the preceding methods may include determining the expression level of one or more (e.g., 1, 2, 3, 4, or 5) of CD8A, EOMES, PRF1, IFNG, or PD-L1, and one or more (e.g., 1, 2, 3, 4, 5, or 6) of VEGFA, KDR, ESM1, PECAM1, ANGPTL4, or CD34. In some embodiments, the method includes determining the expression level of at least two, at least three, at least four, or all five of CD8A, EOMES, PRF1, IFNG, and PD-L1, and at least one, at least two, at least three, at least four, at least five, or all six of VEGFA, KDR, ESM1, PECAM1, ANGPTL4, and CD34. In some embodiments, the method comprises determining the expression level of any one of the combinations set forth in Tables 2-4 and any one of the combinations set forth in Tables 5-8. For example, in some embodiments, the method includes determining the expression level of two of CD8A, EOMES, PRF1, IFNG, and PD-L1, for example, any of the exemplary combinations shown in Table 2, and two of VEGFA, KDR, ESM1, PECAM1, ANGPTL4, and CD34, for example, any of the exemplary combinations shown in Table 5. In some embodiments, the method includes determining the expression level of three of CD8A, EOMES, PRF1, IFNG, and PD-L1, for example, any of the exemplary combinations shown in Table 3, and three of VEGFA, KDR, ESM1, PECAM1, ANGPTL4, and CD34, for example, any of the exemplary combinations shown in Table 6. In some embodiments, the method includes determining the expression level of four of CD8A, EOMES, PRF1, IFNG, and PD-L1, for example, any of the exemplary combinations shown in Table 4, and four of VEGFA, KDR, ESM1, PECAM1, ANGPTL4, and CD34, for example, any of the exemplary combinations shown in Table 7. In some embodiments, the method involves determining the expression level of CD8A, EOMES, PRF1, IFNG, and PD-L1, and five of VEGFA, KDR, ESM1, PECAM1, ANGPTL4, and CD34, for example, any of the exemplary combinations shown in Table 8. In some embodiments, the method involves determining the expression level of CD8A, EOMES, PRF1, IFNG, PD-L1, VEGFA, KDR, ESM1, PECAM1, ANGPTL4, and CD34.

In a further embodiment, in any of the preceding methods, the method may include determining the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9, and one or more (e.g., 1, 2, 3, 4, 5, 6, or 7) of VEGFA, KDR, ESM1, PECAM1, FLT1, ANGPTL4, or CD34. For example, in some embodiments, the method includes determining the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9, and at least two, at least three, at least four, at least five, at least six, or all seven of VEGFA, KDR, ESM1, PECAM1, FLT1, ANGPTL4, or CD34.

For example, any of the preceding methods may include determining the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9, and one or more (e.g., 1, 2, 3, 4, 5, or 6) of VEGFA, KDR, ESM1, PECAM1, ANGPTL4, or CD34. In some embodiments, the method includes determining the expression level of at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9, and at least two, at least three, at least four, at least five, or all six of VEGFA, KDR, ESM1, PECAM1, ANGPTL4, and CD34. In some embodiments, the method comprises determining the expression level of any one of the combinations set forth in Tables 9-16 and any one of the combinations set forth in Tables 5-8. For example, in some embodiments, the method includes determining the expression level of two of IL6, CXCL1, CXCL2, CXCL3, CXCL8, PTGS2, CXCR1, CXCR2, S100A8, and S100A9, for example, any of the exemplary combinations shown in Table 9, and two of VEGFA, KDR, ESM1, PECAM1, ANGPTL4, and CD34, for example, any of the exemplary combinations shown in Table 5. In some embodiments, the method includes determining the expression level of three of IL6, CXCL1, CXCL2, CXCL3, CXCL8, PTGS2, CXCR1, CXCR2, S100A8, and S100A9, for example, any of the exemplary combinations shown in Table 10, and three of VEGFA, KDR, ESM1, PECAM1, ANGPTL4, and CD34, for example, any of the exemplary combinations shown in Table 6. In some embodiments, the method includes determining the expression level of four of IL6, CXCL1, CXCL2, CXCL3, CXCL8, PTGS2, CXCR1, CXCR2, S100A8, and S100A9 for example, any of the exemplary combinations shown in Table 11, and four of VEGFA, KDR, ESM1, PECAM1, ANGPTL4, and CD34, for example, any of the exemplary combinations shown in Table 7. In some embodiments, the method involves determining the expression level of five of IL6, CXCL1, CXCL2, CXCL3, CXCL8, PTGS2, CXCR1, CXCR2, S100A8, and S100A9, for example, any of the exemplary combinations shown in Table 12, and five of VEGFA, KDR, ESM1, PECAM1, ANGPTL4, and CD34, for example, any of the exemplary combinations shown in Table 8. In some embodiments, the method involves determining the expression level of six of IL6, CXCL1, CXCL2, CXCL3, CXCL8, PTGS2, CXCR1, CXCR2, S100A8, and S100A9, for example, any of the exemplary combinations shown in Table 13, and and at least two, at least three, at least four, at least five, or all six of VEGFA, KDR, ESM1, PECAM1, ANGPTL4, and CD34. In some embodiments, the method involves determining the expression level of seven of IL6, CXCL1, CXCL2, CXCL3, CXCL8, PTGS2, CXCR1, CXCR2, S100A8, and S100A9, for example, any of the exemplary combinations shown in Table 14, and and at least two, at least three, at least four, at least five, or all six of VEGFA, KDR, ESM1, PECAM1, ANGPTL4, and CD34. In some embodiments, the method involves determining the expression level of eight of IL6, CXCL1, CXCL2, CXCL3, CXCL8, PTGS2, CXCR1, CXCR2, S100A8, and S100A9, for example, any of the exemplary combinations shown in Table 15, and and at least two, at least three, at least four, at least five, or all six of VEGFA, KDR, ESM1, PECAM1, ANGPTL4, and CD34. In some embodiments, the method involves determining the expression level of nine of IL6, CXCL1, CXCL2, CXCL3, CXCL8, PTGS2, CXCR1, CXCR2, S100A8, and S100A9, for example, any of the exemplary combinations shown in Table 16, and and at least two, at least three, at least four, at least five, or all six of VEGFA, KDR, ESM1, PECAM1, ANGPTL4, and CD34. In some embodiments, the method involves determining the expression level of IL6, CXCL1, CXCL2, CXCL3, CXCL8, PTGS2, CXCR1, CXCR2, S100A8, S100A9, VEGFA, KDR, ESM1, PECAM1, ANGPTL4, and CD34.

In some embodiments of any of the preceding methods, the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample is determined to be at or above a reference expression level of the one or more genes. For example, in some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 is determined to be at or above a reference expression level of the one or more genes. In some instances, the expression level of one or more (e.g., 1, 2, 3, 4, or 5) of CD8A, EOMES, PRF1, IFNG, or PD-L1 in the sample is determined to be at or above a reference expression level of the one or more genes. In some embodiments, the expression level of one or more of the exemplary combinations set forth in Tables 2-4 in the sample is determined to be at or above a reference expression level of the one or more genes. In some embodiments, the expression level of CD8A, EOMES, PRF1, IFNG, and PD-L1 in the sample is determined to be at or above a reference expression level of CD8A, EOMES, PRF1, IFNG, and PD-L1.

In some embodiments of any of the preceding methods, the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 in the sample is determined to be at or above a reference expression level of the one or more genes. In some embodiments, the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, or S100A9 in the sample is determined to be at or above a reference expression level of the one or more genes. In some embodiments, the expression level of one or more of the exemplary combinations set forth in Tables 9-16 in the sample is determined to be at or above a reference expression level of the one or more genes. In some embodiments, the expression level of IL6, CXCL1, CXCL2, CXCL3, CXCL8, PTGS2, CXCR1, CXCR2, S100A8, and S100A9 in the sample is determined to be at or above a reference expression level of IL6, CXCL1, CXCL2, CXCL3, CXCL8, PTGS2, CXCR1, CXCR2, S100A8, and S100A9.

In some embodiments of any of the preceding methods, the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample is determined to be at or above a reference expression level of the one or more genes, and the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 is determined to be at or above a reference expression level of the one or more genes. For example, in some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2 is determined to be at or above a reference expression level of the one or more genes, and the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9 is determined to be at or above a reference expression level of the one or more genes.

For example, in some embodiments, the expression level of one or more (e.g., 1, 2, 3, 4, or 5) of CD8A, EOMES, PRF1, IFNG, or PD-L1 is determined to be at or above a reference expression level of the one or more genes, and the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 is determined to be at or above a reference expression level of the one or more genes. In some embodiments, the expression level of at least two, at least three, at least four, or all five of CD8A, EOMES, PRF1, IFNG, and PD-L1 is determined to be at or above a reference expression level of the one or more genes, and the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9 is determined to be at or above a reference expression level of the one or more genes. In some embodiments, the expression level of any one of the combinations set forth in Tables 2-4 is determined to be at or above a reference expression level of the one or more genes and the expression level of any one of the combinations set forth in Tables 9-16 is determined to be at or above a reference expression level of the one or more genes. For example, in some embodiments, the expression level of two of CD8A, EOMES, PRF1, IFNG, and PD-L1, for example, any of the exemplary combinations shown in Table 2, is determined to be at or above a reference expression level of the one or more genes, and the expression level of two of IL6, CXCL1, CXCL2, CXCL3, CXCL8, PTGS2, CXCR1, CXCR2, S100A8, and S100A9, for example, any of the exemplary combinations shown in Table 9, is determined to be at or above a reference expression level of the one or more genes. In some embodiments, the expression level of three of CD8A, EOMES, PRF1, IFNG, and PD-L1, for example, any of the exemplary combinations shown in Table 3, is determined to be at or above a reference expression level of the one or more genes, and the expression level of three of IL6, CXCL1, CXCL2, CXCL3, CXCL8, PTGS2, CXCR1, CXCR2, S100A8, and S100A9, for example, any of the exemplary combinations shown in Table 10, is determined to be at or above a reference expression level of the one or more genes. In some embodiments, the expression level of four of CD8A, EOMES, PRF1, IFNG, and PD-L1, for example, any of the exemplary combinations shown in Table 4, is determined to be at or above a reference expression level of the one or more genes, and the expression level of four of IL6, CXCL1, CXCL2, CXCL3, CXCL8, PTGS2, CXCR1, CXCR2, S100A8, and S100A9, for example, any of the exemplary combinations shown in Table 11, is determined to be at or above a reference expression level of the one or more genes. In some embodiments, the expression level of CD8A, EOMES, PRF1, IFNG, and PD-L1 is determined to be at or above a reference expression level of CD8A, EOMES, PRF1, IFNG, and PD-L1, and the expression level of five of IL6, CXCL1, CXCL2, CXCL3, CXCL8, PTGS2, CXCR1, CXCR2, S100A8, and S100A9, for example, any of the exemplary combinations shown in Table 12, is determined to be at or above a reference level of the one or more genes. In some embodiments, the expression level of CD8A, EOMES, PRF1, IFNG, and PD-L1 is determined to be at or above a reference expression level of CD8A, EOMES, PRF1, IFNG, and PD-L1, and the expression level of six of IL6, CXCL1, CXCL2, CXCL3, CXCL8, PTGS2, CXCR1, CXCR2, S100A8, and S100A9, for example, any of the exemplary combinations shown in Table 13, is determined to be at or above a reference level of the one or more genes. In some embodiments, the expression level of CD8A, EOMES, PRF1, IFNG, and PD-L1 is determined to be at or above a reference expression level of CD8A, EOMES, PRF1, IFNG, and PD-L1, and the expression level of seven of IL6, CXCL1, CXCL2, CXCL3, CXCL8, PTGS2, CXCR1, CXCR2, S100A8, and S100A9, for example, any of the exemplary combinations shown in Table 14, is determined to be at or above a reference level of the one or more genes. In some embodiments, the expression level of CD8A, EOMES, PRF1, IFNG, and PD-L1 is determined to be at or above a reference expression level of CD8A, EOMES, PRF1, IFNG, and PD-L1, and the expression level of eight of IL6, CXCL1, CXCL2, CXCL3, CXCL8, PTGS2, CXCR1, CXCR2, S100A8, and S100A9, for example, any of the exemplary combinations shown in Table 15, is determined to be at or above a reference level of the one or more genes. In some embodiments, the expression level of CD8A, EOMES, PRF1, IFNG, and PD-L1 is determined to be at or above a reference expression level of CD8A, EOMES, PRF1, IFNG, and PD-L1, and the expression level of nine of IL6, CXCL1, CXCL2, CXCL3, CXCL8, PTGS2, CXCR1, CXCR2, S100A8, and S100A9, for example, any of the exemplary combinations shown in Table 16, is determined to be at or above a reference level of the one or more genes. In some embodiments, the expression level of CD8A, EOMES, PRF1, IFNG, PD-L1, IL6, CXCL1, CXCL2, CXCL3, CXCL8, PTGS2, CXCR1, CXCR2, S100A8, and S100A9 is determined to be at or above a reference expression level of CD8A, EOMES, PRF1, IFNG, PD-L1, IL6, CXCL1, CXCL2, CXCL3, CXCL8, PTGS2, CXCR1, CXCR2, S100A8, and S100A9.

In some embodiments of any of the preceding methods, the expression level of PD-L1 in the sample is determined to be at or above a reference expression level of PD-L1, and the expression level of one or more (e.g., 1, 2, 3, 4, 5, 6, 7, 8, 9, 10, 11, 12, 13, 14, 15, 16, 17, 18, or 19) additional genes selected from the group consisting of CD8A, EOMES, GZMA, GZMB, PRF1, IFNG, CXCL9, CXCL10, CXCL11, CD27, FOXP3, PD-1, CTLA4, TIGIT, IDO1, PSMB8, PSMB9, TAP1, or TAP2 in the sample is determined to be at or above a reference expression level of the one or more additional genes.

In some embodiments of any of the preceding methods, the expression level of one or more (e.g., 1, 2, 3, 4, 5, 6, or 7) of VEGFA, KDR, ESM1, PECAM1, FLT1, ANGPTL4, or CD34 in the sample is determined to be below a reference level of the one or more genes. For example, in some embodiments, the expression level of at least one, at least two, at least three, at least four, at least five, at least six, or all seven of VEGFA, KDR, ESM1, PECAM1, FLT1, ANGPTL4, or CD34 in the sample is determined to be below a reference level of the one or more genes. In some embodiments, the expression level of one or more of the exemplary combinations set forth in Tables 5-8 in the sample is determined to be below a reference expression level of the one or more genes. In some embodiments, the expression level of one or more of VEGFA, KDR, ESM1, PECAM1, ANGPTL4, or CD34 in the sample is determined to be below a reference level of the one or more genes. For example, in some embodiments, the expression level of VEGFA, KDR, ESM1, PECAM1, ANGPTL4, and CD34 in the sample is determined to be below a reference level of VEGFA, KDR, ESM1, PECAM1, ANGPTL4, and CD34.

In other embodiments, in any of the preceding methods, the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 is determined to be at or above a reference level of the one or more genes, and the expression level of one or more (e.g., 1, 2, 3, 4, 5, 6, or 7) of VEGFA, KDR, ESM1, PECAM1, FLT1, ANGPTL4, or CD34 is determined to be below a reference level of the one or more genes. For example, in some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2 is determined to be at or above a reference level of the one or more genes, and the expression level of at least two, at least three, at least four, at least five, at least six, or all seven of VEGFA, KDR, ESM1, PECAM1, FLT1, ANGPTL4, or CD34 is determined to be below a reference level of the one or more genes.

For example, in some embodiments, the expression level of one or more (e.g., 1, 2, 3, 4, or 5) of CD8A, EOMES, PRF1, IFNG, or PD-L1 is determined to be at or above a reference level of the one or more genes, and the expression level of one or more (e.g., 1, 2, 3, 4, 5, or 6) of VEGFA, KDR, ESM1, PECAM1, ANGPTL4, or CD34 is determined to be below a reference level of the one or more genes. In some embodiments, the expression level of at least two, at least three, at least four, or all five of CD8A, EOMES, PRF1, IFNG, and PD-L1 is determined to be at or above a reference level of the one or more genes, and the expression level of at least one, at least two, at least three, at least four, at least five, or all six of VEGFA, KDR, ESM1, PECAM1, ANGPTL4, and CD34 is determined to be below a reference level fo the one or more genes. In some embodiments, the expression level of any one of the combinations set forth in Tables 2-4 is determined to be at or above a reference level of the one or more genes, and the expression level of any one of the combinations set forth in Tables 5-8 is determined to be below a reference level of the one or more genes. For example, in some embodiments, the expression level of two of CD8A, EOMES, PRF1, IFNG, and PD-L1, for example, any of the exemplary combinations shown in Table 2, is determined to be at or above a reference level of the one or more genes, and the expression level of two of VEGFA, KDR, ESM1, PECAM1, ANGPTL4, and CD34, for example, any of the exemplary combinations shown in Table 5, is determined to be below a reference level of the one or more genes. In some embodiments, the expression level of three of CD8A, EOMES, PRF1, IFNG, and PD-L1, for example, any of the exemplary combinations shown in Table 3, is determined to be at or above a reference level of the one or more genes, and the expression level of three of VEGFA, KDR, ESM1, PECAM1, ANGPTL4, and CD34, for example, any of the exemplary combinations shown in Table 6, is determined to be below a reference level of the one or more genes. In some embodiments, the expression level of four of CD8A, EOMES, PRF1, IFNG, and PD-L1, for example, any of the exemplary combinations shown in Table 4, is determined to be at or above a reference level of the one or more genes, and the expression level of four of VEGFA, KDR, ESM1, PECAM1, ANGPTL4, and CD34, for example, any of the exemplary combinations shown in Table 7, is determined to be below a reference level of the one or more genes. In some embodiments, the expression level of CD8A, EOMES, PRF1, IFNG, and PD-L1 is determined to be at or above a reference level of CD8A, EOMES, PRF1, IFNG, and PD-L1, and the expression level of five of VEGFA, KDR, ESM1, PECAM1, ANGPTL4, and CD34, for example, any of the exemplary combinations shown in Table 8, is determined to be below a reference level of the one or more genes. In some embodiments, the expression level of CD8A, EOMES, PRF1, IFNG, and PD-L1 is determined to be at or above a reference level of CD8A, EOMES, PRF1, IFNG, and PD-L1, and the expression level of VEGFA, KDR, ESM1, PECAM1, ANGPTL4, and CD34 is determined to be below a reference level of VEGFA, KDR, ESM1, PECAM1, ANGPTL4, and CD34.

In some embodiments of any of the preceding methods, the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 in the sample is determined to be below a reference level of the one or more genes. In some embodiments, the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, or S100A9 in the sample is determined to be below a reference level of the one or more genes. For example, in some embodiments, the expression level of one or more of the exemplary combinations set forth in Tables 9-16 in the sample is determined to be below a reference expression level of the one or more genes. In some embodiments, the expression level of IL6, CXCL1, CXCL2, CXCL3, CXCL8, PTGS2, CXCR1, CXCR2, S100A8, and S100A9 in the sample is determined to be below a reference level of IL6, CXCL1, CXCL2, CXCL3, CXCL8, PTGS2, CXCR1, CXCR2, S100A8, and S100A9.

In another aspect, provided herein is a method of treating an individual having a cancer (e.g., a kidney cancer (e.g., RCC), a lung cancer (e.g., NSCLC), a bladder cancer (e.g., UBC), a liver cancer (e.g., HCC), an ovarian cancer, or a breast cancer (e.g., TNBC)) that includes (a) determining the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37) of the following genes in a sample from the individual: CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2; or IL6, CXCL1, CXCL2, CXCL3, CXCL8, PTGS2, CXCR1, CXCR2, S100A8, or S100A9, wherein (i) the expression level of one or more of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample is determined to be at or above a reference expression level of the one or more genes; and (ii) the expression level of one or more of IL6, CXCL1, CXCL2, CXCL3, CXCL8, PTGS2, CXCR1, CXCR2, S100A8, or S100A9 in the sample is determined to be below a reference expression level of the one or more genes; and (b) administering an effective amount of a PD-L1 axis binding antagonist (e.g., a PD-L1 binding antagonist (e.g., an anti-PD-L1 antibody, e.g., atezolizumab (MPDL3280A)) or a PD-1 binding antagonist (e.g., an anti-PD-1 antibody)) monotherapy to the individual based on the expression level of the one or more genes determined in step (a).

In any of the preceding methods, the method may include determining the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2, and one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9. For example, in some embodiments, the method includes determining the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2, and at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9.

For example, any of the preceding methods may include determining the expression level of one or more (e.g., 1, 2, 3, 4, or 5) of CD8A, EOMES, PRF1, IFNG, or PD-L1, and one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9. In some embodiments, the method includes determining the expression level of at least two, at least three, at least four, or all five of CD8A, EOMES, PRF1, IFNG, and PD-L1, and at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9. In some embodiments, the method comprises determining the expression level of any one of the combinations set forth in Tables 2-4 and any one of the combinations set forth in Tables 9-16. For example, in some embodiments, the method includes determining the expression level of two of CD8A, EOMES, PRF1, IFNG, and PD-L1, for example, any of the exemplary combinations shown in Table 2, and two of IL6, CXCL1, CXCL2, CXCL3, CXCL8, PTGS2, CXCR1, CXCR2, S100A8, and S100A9, for example, any of the exemplary combinations shown in Table 9. In some embodiments, the method includes determining the expression level of three of CD8A, EOMES, PRF1, IFNG, and PD-L1, for example, any of the exemplary combinations shown in Table 3, and three of IL6, CXCL1, CXCL2, CXCL3, CXCL8, PTGS2, CXCR1, CXCR2, S100A8, and S100A9, for example, any of the exemplary combinations shown in Table 10. In some embodiments, the method includes determining the expression level of four of CD8A, EOMES, PRF1, IFNG, and PD-L1, for example, any of the exemplary combinations shown in Table 4, and four of IL6, CXCL1, CXCL2, CXCL3, CXCL8, PTGS2, CXCR1, CXCR2, S100A8, and S100A9, for example, any of the exemplary combinations shown in Table 11. In some embodiments, the method involves determining the expression level of CD8A, EOMES, PRF1, IFNG, and PD-L1, and five of IL6, CXCL1, CXCL2, CXCL3, CXCL8, and PTGS2, for example, any of the exemplary combinations shown in Table 12. In some embodiments, the method involves determining the expression level of CD8A, EOMES, PRF1, IFNG, and PD-L1, and six of IL6, CXCL1, CXCL2, CXCL3, CXCL8, and PTGS2, for example, any of the exemplary combinations shown in Table 13. In some embodiments, the method involves determining the expression level of CD8A, EOMES, PRF1, IFNG, and PD-L1, and seven of IL6, CXCL1, CXCL2, CXCL3, CXCL8, and PTGS2, for example, any of the exemplary combinations shown in Table 14. In some embodiments, the method involves determining the expression level of CD8A, EOMES, PRF1, IFNG, and PD-L1, and eight of IL6, CXCL1, CXCL2, CXCL3, CXCL8, and PTGS2, for example, any of the exemplary combinations shown in Table 15. In some embodiments, the method involves determining the expression level of CD8A, EOMES, PRF1, IFNG, and PD-L1, and nine of IL6, CXCL1, CXCL2, CXCL3, CXCL8, and PTGS2, for example, any of the exemplary combinations shown in Table 16. In some embodiments, the method involves determining the expression level of CD8A, EOMES, PRF1, IFNG, PD-L1, IL6, CXCL1, CXCL2, CXCL3, CXCL8, PTGS2, CXCR1, CXCR2, S100A8, and S100A9.

In some of any of the preceding methods, the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample is determined to be at or above a reference expression level of the one or more genes, and the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 is determined to be below a reference expression level of the one or more genes. For example, in some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2 is determined to be at or above a reference expression level of the one or more genes, and the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9 is determined to be below a reference expression level of the one or more genes.

For example, in some embodiments, the expression level of one or more (e.g., 1, 2, 3, 4, or 5) of CD8A, EOMES, PRF1, IFNG, or PD-L1 is determined to be at or above a reference expression level of the one or more genes, and the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 is determined to be below a reference expression level of the one or more genes. In some embodiments, the expression level of at least two, at least three, at least four, or all five of CD8A, EOMES, PRF1, IFNG, and PD-L1 is determined to be at or above a reference expression level of the one or more genes, and the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9 is determined to be below a reference expression level of the one or more genes. In some embodiments, the expression level of any one of the combinations set forth in Tables 2-4 is determined to be at or above a reference expression level of the one or more genes and the expression level of any one of the combinations set forth in Tables 9-16 is determined to be below a reference expression level of the one or more genes. For example, in some embodiments, the expression level of two of CD8A, EOMES, PRF1, IFNG, and PD-L1, for example, any of the exemplary combinations shown in Table 2, is determined to be at or above a reference expression level of the one or more genes, and the expression level of two of IL6, CXCL1, CXCL2, CXCL3, CXCL8, PTGS2, CXCR1, CXCR2, S100A8, and S100A9, for example, any of the exemplary combinations shown in Table 9, is determined to be below a reference expression level of the one or more genes. In some embodiments, the expression level of three of CD8A, EOMES, PRF1, IFNG, and PD-L1, for example, any of the exemplary combinations shown in Table 3, is determined to be at or above a reference expression level of the one or more genes, and the expression level of three of IL6, CXCL1, CXCL2, CXCL3, CXCL8, PTGS2, CXCR1, CXCR2, S100A8, and S100A9, for example, any of the exemplary combinations shown in Table 10, is determined to be below a reference expression level of the one or more genes. In some embodiments, the expression level of four of CD8A, EOMES, PRF1, IFNG, and PD-L1, for example, any of the exemplary combinations shown in Table 4, is determined to be at or above a reference expression level of the one or more genes, and the expression level of four of IL6, CXCL1, CXCL2, CXCL3, CXCL8, PTGS2, CXCR1, CXCR2, S100A8, and S100A9, for example, any of the exemplary combinations shown in Table 11, is determined to be below a reference expression level of the one or more genes. In some embodiments, the expression level of CD8A, EOMES, PRF1, IFNG, and PD-L1 is determined to be at or above a reference expression level of CD8A, EOMES, PRF1, IFNG, and PD-L1, and the expression level of five of IL6, CXCL1, CXCL2, CXCL3, CXCL8, PTGS2, CXCR1, CXCR2, S100A8, and S100A9, for example, any of the exemplary combinations shown in Table 12, is determined to be below a reference level of the one or more genes. In some embodiments, the expression level of CD8A, EOMES, PRF1, IFNG, and PD-L1 is determined to be at or above a reference expression level of CD8A, EOMES, PRF1, IFNG, and PD-L1, and the expression level of six of IL6, CXCL1, CXCL2, CXCL3, CXCL8, PTGS2, CXCR1, CXCR2, S100A8, and S100A9, for example, any of the exemplary combinations shown in Table 13, is determined to be below a reference level of the one or more genes. In some embodiments, the expression level of CD8A, EOMES, PRF1, IFNG, and PD-L1 is determined to be at or above a reference expression level of CD8A, EOMES, PRF1, IFNG, and PD-L1, and the expression level of seven of IL6, CXCL1, CXCL2, CXCL3, CXCL8, PTGS2, CXCR1, CXCR2, S100A8, and S100A9, for example, any of the exemplary combinations shown in Table 14, is determined to be below a reference level of the one or more genes. In some embodiments, the expression level of CD8A, EOMES, PRF1, IFNG, and PD-L1 is determined to be at or above a reference expression level of CD8A, EOMES, PRF1, IFNG, and PD-L1, and the expression level of eight of IL6, CXCL1, CXCL2, CXCL3, CXCL8, PTGS2, CXCR1, CXCR2, S100A8, and S100A9, for example, any of the exemplary combinations shown in Table 15, is determined to be below a reference level of the one or more genes. In some embodiments, the expression level of CD8A, EOMES, PRF1, IFNG, and PD-L1 is determined to be at or above a reference expression level of CD8A, EOMES, PRF1, IFNG, and PD-L1, and the expression level of nine of IL6, CXCL1, CXCL2, CXCL3, CXCL8, PTGS2, CXCR1, CXCR2, S100A8, and S100A9, for example, any of the exemplary combinations shown in Table 16, is determined to be below a reference level of the one or more genes. In some embodiments, the expression level of CD8A, EOMES, PRF1, IFNG, and PD-L1 is determined to be at or above a reference level of CD8A, EOMES, PRF1, IFNG, and PD-L1, and the expression level of IL6, CXCL1, CXCL2, CXCL3, CXCL8, PTGS2, CXCR1, CXCR2, S100A8, and S100A9 is determined to be below a reference expression level of IL6, CXCL1, CXCL2, CXCL3, CXCL8, PTGS2, CXCR1, CXCR2, S100A8, and S100A9.

In another aspect, provided herein is a method of treating an individual having (e.g., a kidney cancer (e.g., RCC), a lung cancer (e.g., NSCLC), a bladder cancer (e.g., UBC), a liver cancer (e.g., HCC), an ovarian cancer, or a breast cancer (e.g., TNBC)), the method including administering to the individual an effective amount of an anti-cancer therapy comprising a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist, wherein (i) the expression level of one or more of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample has been determined to be at or above a reference expression level of the one or more genes; or (ii) the expression level of one or more of VEGFA, KDR, ESM1, PECAM1, FLT1, ANGPTL4, or CD34; or IL6, CXCL1, CXCL2, CXCL3, CXCL8, PTGS2, CXCR1, CXCR2, S100A8, or S100A9 in the sample has been determined to be below a reference expression level of the one or more genes. In some embodiments, the expression level of one or more of the genes has been determined prior to treatment with the anti-cancer therapy. In other embodiments, the expression level of one or more of the genes has been determined after treatment with the anti-cancer therapy.

In some embodiments of any of the preceding methods, the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample has been determined to be at or above a reference expression level of the one or more genes. For example, in some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 has been determined to be at or above a reference expression level of the one or more genes. In some instances, the expression level of one or more (e.g., 1, 2, 3, 4, or 5) of CD8A, EOMES, PRF1, IFNG, or PD-L1 in the sample has been determined to be at or above a reference expression level of the one or more genes. In some embodiments, the expression level of one or more of the exemplary combinations set forth in Tables 2-4 in the sample has been determined to be at or above a reference expression level of the one or more genes. In some embodiments, the expression level of CD8A, EOMES, PRF1, IFNG, and PD-L1 in the sample has been determined to be at or above a reference expression level of CD8A, EOMES, PRF1, IFNG, and PD-L1.

In some embodiments of any of the preceding methods, the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 in the sample has been determined to be at or above a reference expression level of the one or more genes. In some embodiments, the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, or S100A9 in the sample has been determined to be at or above a reference expression level of the one or more genes. In some embodiments, the expression level of one or more of the exemplary combinations set forth in Tables 9-16 in the sample has been determined to be at or above a reference expression level of the one or more genes. In some embodiments, the expression level of IL6, CXCL1, CXCL2, CXCL3, CXCL8, PTGS2, CXCR1, CXCR2, S100A8, and S100A9 in the sample has been determined to be at or above a reference expression level of IL6, CXCL1, CXCL2, CXCL3, CXCL8, PTGS2, CXCR1, CXCR2, S100A8, and S100A9.

In some embodiments of any of the preceding methods, the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample has been determined to be at or above a reference expression level of the one or more genes, and the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 has been determined to be at or above a reference expression level of the one or more genes. For example, in some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2 has been determined to be at or above a reference expression level of the one or more genes, and the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9 has been determined to be at or above a reference expression level of the one or more genes.

For example, in some embodiments, the expression level of one or more (e.g., 1, 2, 3, 4, or 5) of CD8A, EOMES, PRF1, IFNG, or PD-L1 has been determined to be at or above a reference expression level of the one or more genes, and the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 has been determined to be at or above a reference expression level of the one or more genes. In some embodiments, the expression level of at least two, at least three, at least four, or all five of CD8A, EOMES, PRF1, IFNG, and PD-L1 has been determined to be at or above a reference expression level of the one or more genes, and the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9 has been determined to be at or above a reference expression level of the one or more genes. In some embodiments, the expression level of any one of the combinations set forth in Tables 2-4 has been determined to be at or above a reference expression level of the one or more genes and the expression level of any one of the combinations set forth in Tables 9-16 has been determined to be at or above a reference expression level of the one or more genes. For example, in some embodiments, the expression level of two of CD8A, EOMES, PRF1, IFNG, and PD-L1, for example, any of the exemplary combinations shown in Table 2, has been determined to be at or above a reference expression level of the one or more genes, and the expression level of two of IL6, CXCL1, CXCL2, CXCL3, CXCL8, PTGS2, CXCR1, CXCR2, S100A8, and S100A9, for example, any of the exemplary combinations shown in Table 9, has been determined to be at or above a reference expression level of the one or more genes. In some embodiments, the expression level of three of CD8A, EOMES, PRF1, IFNG, and PD-L1, for example, any of the exemplary combinations shown in Table 3, has been determined to be at or above a reference expression level of the one or more genes, and the expression level of three of IL6, CXCL1, CXCL2, CXCL3, CXCL8, PTGS2, CXCR1, CXCR2, S100A8, and S100A9, for example, any of the exemplary combinations shown in Table 10, has been determined to be at or above a reference expression level of the one or more genes. In some embodiments, the expression level of four of CD8A, EOMES, PRF1, IFNG, and PD-L1, for example, any of the exemplary combinations shown in Table 4, has been determined to be at or above a reference expression level of the one or more genes, and the expression level of four of IL6, CXCL1, CXCL2, CXCL3, CXCL8, PTGS2, CXCR1, CXCR2, S100A8, and S100A9, for example, any of the exemplary combinations shown in Table 11, has been determined to be at or above a reference expression level of the one or more genes. In some embodiments, the expression level of CD8A, EOMES, PRF1, IFNG, and PD-L1 has been determined to be at or above a reference expression level of CD8A, EOMES, PRF1, IFNG, and PD-L1, and the expression level of five of IL6, CXCL1, CXCL2, CXCL3, CXCL8, PTGS2, CXCR1, CXCR2, S100A8, and S100A9, for example, any of the exemplary combinations shown in Table 12, has been determined to be at or above a reference level of the one or more genes. In some embodiments, the expression level of CD8A, EOMES, PRF1, IFNG, and PD-L1 has been determined to be at or above a reference expression level of CD8A, EOMES, PRF1, IFNG, and PD-L1, and the expression level of six of IL6, CXCL1, CXCL2, CXCL3, CXCL8, PTGS2, CXCR1, CXCR2, S100A8, and S100A9, for example, any of the exemplary combinations shown in Table 13, has been determined to be at or above a reference level of the one or more genes. In some embodiments, the expression level of CD8A, EOMES, PRF1, IFNG, and PD-L1 has been determined to be at or above a reference expression level of CD8A, EOMES, PRF1, IFNG, and PD-L1, and the expression level of seven of IL6, CXCL1, CXCL2, CXCL3, CXCL8, PTGS2, CXCR1, CXCR2, S100A8, and S100A9, for example, any of the exemplary combinations shown in Table 14, has been determined to be at or above a reference level of the one or more genes. In some embodiments, the expression level of CD8A, EOMES, PRF1, IFNG, and PD-L1 has been determined to be at or above a reference expression level of CD8A, EOMES, PRF1, IFNG, and PD-L1, and the expression level of eight of IL6, CXCL1, CXCL2, CXCL3, CXCL8, PTGS2, CXCR1, CXCR2, S100A8, and S100A9, for example, any of the exemplary combinations shown in Table 15, has been determined to be at or above a reference level of the one or more genes. In some embodiments, the expression level of CD8A, EOMES, PRF1, IFNG, and PD-L1 has been determined to be at or above a reference expression level of CD8A, EOMES, PRF1, IFNG, and PD-L1, and the expression level of nine of IL6, CXCL1, CXCL2, CXCL3, CXCL8, PTGS2, CXCR1, CXCR2, S100A8, and S100A9, for example, any of the exemplary combinations shown in Table 16, has been determined to be at or above a reference level of the one or more genes. In some embodiments, the expression level of CD8A, EOMES, PRF1, IFNG, PD-L1, IL6, CXCL1, CXCL2, CXCL3, CXCL8, PTGS2, CXCR1, CXCR2, S100A8, and S100A9 has been determined to be at or above a reference expression level of CD8A, EOMES, PRF1, IFNG, PD-L1, IL6, CXCL1, CXCL2, CXCL3, CXCL8, PTGS2, CXCR1, CXCR2, S100A8, and S100A9.

In some embodiments of any of the preceding methods, the expression level of PD-L1 in the sample has been determined to be at or above a reference expression level of PD-L1, and the expression level of one or more (e.g., 1, 2, 3, 4, 5, 6, 7, 8, 9, 10, 11, 12, 13, 14, 15, 16, 17, 18, or 19) additional genes selected from the group consisting of CD8A, EOMES, GZMA, GZMB, PRF1, IFNG, CXCL9, CXCL10, CXCL11, CD27, FOXP3, PD-1, CTLA4, TIGIT, IDO1, PSMB8, PSMB9, TAP1, or TAP2 in the sample has been determined to be at or above a reference expression level of the one or more additional genes.

In some embodiments of any of the preceding methods, the expression level of one or more (e.g., 1, 2, 3, 4, 5, 6, or 7) of VEGFA, KDR, ESM1, PECAM1, FLT1, ANGPTL4, or CD34 in the sample has been determined to be below a reference level of the one or more genes. For example, in some embodiments, the expression level of at least one, at least two, at least three, at least four, at least five, at least six, or all seven of VEGFA, KDR, ESM1, PECAM1, FLT1, ANGPTL4, or CD34 in the sample has been determined to be below a reference level of the one or more genes. In some embodiments, the expression level of one or more of the exemplary combinations set forth in Tables 5-8 in the sample has been determined to be below a reference expression level of the one or more genes. In some embodiments, the expression level of one or more of VEGFA, KDR, ESM1, PECAM1, ANGPTL4, or CD34 in the sample has been determined to be below a reference level of the one or more genes. For example, in some embodiments, the expression level of VEGFA, KDR, ESM1, PECAM1, ANGPTL4, and CD34 in the sample has been determined to be below a reference level of VEGFA, KDR, ESM1, PECAM1, ANGPTL4, and CD34.

In other embodiments, in any of the preceding methods, the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 has been determined to be at or above a reference level of the one or more genes, and the expression level of one or more (e.g., 1, 2, 3, 4, 5, 6, or 7) of VEGFA, KDR, ESM1, PECAM1, FLT1, ANGPTL4, or CD34 has been determined to be below a reference level of the one or more genes. For example, in some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2 has been determined to be at or above a reference level of the one or more genes, and the expression level of at least two, at least three, at least four, at least five, at least six, or all seven of VEGFA, KDR, ESM1, PECAM1, FLT1, ANGPTL4, or CD34 has been determined to be below a reference level of the one or more genes.

For example, in some embodiments, the expression level of one or more (e.g., 1, 2, 3, 4, or 5) of CD8A, EOMES, PRF1, IFNG, or PD-L1 has been determined to be at or above a reference level of the one or more genes, and the expression level of one or more (e.g., 1, 2, 3, 4, 5, or 6) of VEGFA, KDR, ESM1, PECAM1, ANGPTL4, or CD34 has been determined to be below a reference level of the one or more genes. In some embodiments, the expression level of at least two, at least three, at least four, or all five of CD8A, EOMES, PRF1, IFNG, and PD-L1 has been determined to be at or above a reference level of the one or more genes, and the expression level of at least one, at least two, at least three, at least four, at least five, or all six of VEGFA, KDR, ESM1, PECAM1, ANGPTL4, and CD34 has been determined to be below a reference level fo the one or more genes. In some embodiments, the expression level of any one of the combinations set forth in Tables 2-4 has been determined to be at or above a reference level of the one or more genes, and the expression level of any one of the combinations set forth in Tables 5-8 has been determined to be below a reference level of the one or more genes. For example, in some embodiments, the expression level of two of CD8A, EOMES, PRF1, IFNG, and PD-L1, for example, any of the exemplary combinations shown in Table 2, has been determined to be at or above a reference level of the one or more genes, and the expression level of two of VEGFA, KDR, ESM1, PECAM1, ANGPTL4, and CD34, for example, any of the exemplary combinations shown in Table 5, has been determined to be below a reference level of the one or more genes. In some embodiments, the expression level of three of CD8A, EOMES, PRF1, IFNG, and PD-L1, for example, any of the exemplary combinations shown in Table 3, has been determined to be at or above a reference level of the one or more genes, and the expression level of three of VEGFA, KDR, ESM1, PECAM1, ANGPTL4, and CD34, for example, any of the exemplary combinations shown in Table 6, has been determined to be below a reference level of the one or more genes. In some embodiments, the expression level of four of CD8A, EOMES, PRF1, IFNG, and PD-L1, for example, any of the exemplary combinations shown in Table 4, has been determined to be at or above a reference level of the one or more genes, and the expression level of four of VEGFA, KDR, ESM1, PECAM1, ANGPTL4, and CD34, for example, any of the exemplary combinations shown in Table 7, has been determined to be below a reference level of the one or more genes. In some embodiments, the expression level of CD8A, EOMES, PRF1, IFNG, and PD-L1 has been determined to be at or above a reference level of CD8A, EOMES, PRF1, IFNG, and PD-L1, and the expression level of five of VEGFA, KDR, ESM1, PECAM1, ANGPTL4, and CD34, for example, any of the exemplary combinations shown in Table 8, has been determined to be below a reference level of the one or more genes. In some embodiments, the expression level of CD8A, EOMES, PRF1, IFNG, and PD-L1 has been determined to be at or above a reference level of CD8A, EOMES, PRF1, IFNG, and PD-L1, and the expression level of VEGFA, KDR, ESM1, PECAM1, ANGPTL4, and CD34 has been determined to be below a reference level of VEGFA, KDR, ESM1, PECAM1, ANGPTL4, and CD34.

In some embodiments of any of the preceding methods, the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 in the sample has been determined to be below a reference level of the one or more genes. In some embodiments, the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, or S100A9 in the sample has been determined to be below a reference level of the one or more genes. For example, in some embodiments, the expression level of one or more of the exemplary combinations set forth in Tables 9-16 in the sample has been determined to be below a reference expression level of the one or more genes. In some embodiments, the expression level of IL6, CXCL1, CXCL2, CXCL3, CXCL8, PTGS2, CXCR1, CXCR2, S100A8, and S100A9 in the sample has been determined to be below a reference level of IL6, CXCL1, CXCL2, CXCL3, CXCL8, PTGS2, CXCR1, CXCR2, S100A8, and S100A9.

In another aspect, provided herein is a method of treating an individual having a cancer (e.g., a kidney cancer (e.g., RCC), a lung cancer (e.g., NSCLC), a bladder cancer (e.g., UBC), a liver cancer (e.g., HCC), an ovarian cancer, or a breast cancer (e.g., TNBC)) that includes (a) determining the expression level of one or more (e.g., 1, 2, 3, 4, 5, 6, or 7) of the following genes in a sample from the individual: VEGFA, KDR, ESM1, PECAM1, FLT1, ANGPTL4, or CD34, wherein the expression level of one or more of VEGFA, KDR, ESM1, PECAM1, FLT1, ANGPTL4, or CD34 in the sample is determined to be at or above a reference expression level of the one or more genes; and (b) administering an effective amount of an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))) to the individual based on the expression level of the one or more genes determined in step (a).

In some embodiments, the method includes determining the expression level of at least two, at least three, at least four, at least five, at least six, or all seven of VEGFA, KDR, ESM1, PECAM1, FLT1, ANGPTL4, or CD34. For example, in some embodiments, the method includes determining the expression level of one or more of VEGFA, KDR, ESM1, PECAM1, ANGPTL4, or CD34. In some embodiments, the method includes determining the expression level of at least two, at least three, at least four, at least five, or all six of VEGFA, KDR, ESM1, PECAM1, ANGPTL4, and CD34. In some embodiments, the method includes determining the expression level of two of VEGFA, KDR, ESM1, PECAM1, ANGPTL4, and CD34, for example, any of the exemplary combinations shown in Table 5. In some embodiments, the method includes determining the expression level of three of VEGFA, KDR, ESM1, PECAM1, ANGPTL4, and CD34, for example, any of the exemplary combinations shown in Table 6. In some embodiments, the method includes determining the expression level of four of VEGFA, KDR, ESM1, PECAM1, ANGPTL4, and CD34, for example, any of the exemplary combinations shown in Table 7. In some embodiments, the method includes determining the expression level of five of VEGFA, KDR, ESM1, PECAM1, ANGPTL4, and CD34, for example, any of the exemplary combinations shown in Table 8. In some embodiments, the method includes determining the expression level of VEGFA, KDR, ESM1, PECAM1, ANGPTL4, and CD34.

In some embodiments, the expression level of one or more (e.g., 1, 2, 3, 4, 5, 6, or 7) of VEGFA, KDR, ESM1, PECAM1, FLT1, ANGPTL4, or CD34 in the sample is determined to be at or above a reference level of the one or more genes. For example, in some embodiments, the expression level of at least one, at least two, at least three, at least four, at least five, at least six, or all seven of VEGFA, KDR, ESM1, PECAM1, FLT1, ANGPTL4, or CD34 in the sample is determined to be at or above a reference level of the one or more genes. In some embodiments, the expression level of one or more of the exemplary combinations set forth in Tables 5-8 in the sample is determined to be at or above a reference expression level of the one or more genes. In some embodiments, the expression level of one or more of VEGFA, KDR, ESM1, PECAM1, ANGPTL4, or CD34 in the sample is determined to be at or above a reference level of the one or more genes. For example, in some embodiments, the expression level of VEGFA, KDR, ESM1, PECAM1, ANGPTL4, and CD34 in the sample is determined to be at or above a reference level of VEGFA, KDR, ESM1, PECAM1, ANGPTL4, and CD34.

In some of any of the preceding methods, the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample has been determined to be at or above a reference expression level of the one or more genes, and the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 has been determined to be below a reference expression level of the one or more genes. For example, in some embodiments, the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2 has been determined to be at or above a reference expression level of the one or more genes, and the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9 has been determined to be below a reference expression level of the one or more genes.

For example, in some embodiments, the expression level of one or more (e.g., 1, 2, 3, 4, or 5) of CD8A, EOMES, PRF1, IFNG, or PD-L1 has been determined to be at or above a reference expression level of the one or more genes, and the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9 has been determined to be below a reference expression level of the one or more genes. In some embodiments, the expression level of at least two, at least three, at least four, or all five of CD8A, EOMES, PRF1, IFNG, and PD-L1 has been determined to be at or above a reference expression level of the one or more genes, and the expression level of at least one, at least two, at least three, at least four, at least five, at least six, at least seven, at least eight, at least nine, or all ten of IL6, CXCL1, CXCL2, CXCL3, CXCL8, PTGS2, CXCR1, CXCR2, S100A8, and S100A9 has been determined to be below a reference expression level of the one or more genes. In some embodiments, the expression level of any one of the combinations set forth in Tables 2-4 has been determined to be at or above a reference expression level of the one or more genes and the expression level of any one of the combinations set forth in Tables 9-12 has been determined to be below a reference expression level of the one or more genes. For example, in some embodiments, the expression level of two of CD8A, EOMES, PRF1, IFNG, and PD-L1, for example, any of the exemplary combinations shown in Table 2, has been determined to be at or above a reference expression level of the one or more genes, and the expression level of two of IL6, CXCL1, CXCL2, CXCL3, CXCL8, PTGS2, CXCR1, CXCR2, S100A8, and S100A9, for example, any of the exemplary combinations shown in Table 9, has been determined to be below a reference expression level of the one or more genes. In some embodiments, the expression level of three of CD8A, EOMES, PRF1, IFNG, and PD-L1, for example, any of the exemplary combinations shown in Table 3, has been determined to be at or above a reference expression level of the one or more genes, and the expression level of three of IL6, CXCL1, CXCL2, CXCL3, CXCL8, PTGS2, CXCR1, CXCR2, S100A8, and S100A9, for example, any of the exemplary combinations shown in Table 10, has been determined to be below a reference expression level of the one or more genes. In some embodiments, the expression level of four of CD8A, EOMES, PRF1, IFNG, and PD-L1, for example, any of the exemplary combinations shown in Table 4, has been determined to be at or above a reference expression level of the one or more genes, and the expression level of four of IL6, CXCL1, CXCL2, CXCL3, CXCL8, PTGS2, CXCR1, CXCR2, S100A8, and S100A9, for example, any of the exemplary combinations shown in Table 11, has been determined to be below a reference expression level of the one or more genes. In some embodiments, the expression level of CD8A, EOMES, PRF1, IFNG, and PD-L1 has been determined to be at or above a reference expression level of CD8A, EOMES, PRF1, IFNG, and PD-L1, and the expression level of five of IL6, CXCL1, CXCL2, CXCL3, CXCL8, PTGS2, CXCR1, CXCR2, S100A8, and S100A9, for example, any of the exemplary combinations shown in Table 12, has been determined to be below a reference level of the one or more genes. In some embodiments, the expression level of CD8A, EOMES, PRF1, IFNG, and PD-L1 has been determined to be at or above a reference expression level of CD8A, EOMES, PRF1, IFNG, and PD-L1, and the expression level of six of IL6, CXCL1, CXCL2, CXCL3, CXCL8, PTGS2, CXCR1, CXCR2, S100A8, and S100A9, for example, any of the exemplary combinations shown in Table 13, has been determined to be below a reference level of the one or more genes. In some embodiments, the expression level of CD8A, EOMES, PRF1, IFNG, and PD-L1 has been determined to be at or above a reference expression level of CD8A, EOMES, PRF1, IFNG, and PD-L1, and the expression level of seven of IL6, CXCL1, CXCL2, CXCL3, CXCL8, PTGS2, CXCR1, CXCR2, S100A8, and S100A9, for example, any of the exemplary combinations shown in Table 14, has been determined to be below a reference level of the one or more genes. In some embodiments, the expression level of CD8A, EOMES, PRF1, IFNG, and PD-L1 has been determined to be at or above a reference expression level of CD8A, EOMES, PRF1, IFNG, and PD-L1, and the expression level of eight of IL6, CXCL1, CXCL2, CXCL3, CXCL8, PTGS2, CXCR1, CXCR2, S100A8, and S100A9, for example, any of the exemplary combinations shown in Table 15, has been determined to be below a reference level of the one or more genes. In some embodiments, the expression level of CD8A, EOMES, PRF1, IFNG, and PD-L1 has been determined to be at or above a reference expression level of CD8A, EOMES, PRF1, IFNG, and PD-L1, and the expression level of nine of IL6, CXCL1, CXCL2, CXCL3, CXCL8, PTGS2, CXCR1, CXCR2, S100A8, and S100A9, for example, any of the exemplary combinations shown in Table 16, has been determined to be below a reference level of the one or more genes. In some embodiments, the expression level of CD8A, EOMES, PRF1, IFNG, and PD-L1 has been determined to be at or above a reference level of CD8A, EOMES, PRF1, IFNG, and PD-L1, and the expression level of IL6, CXCL1, CXCL2, CXCL3, CXCL8, PTGS2, CXCR1, CXCR2, S100A8, and S100A9 has been determined to be below a reference expression level of IL6, CXCL1, CXCL2, CXCL3, CXCL8, PTGS2, CXCR1, CXCR2, S100A8, and S100A9.

In some embodiments of any of the preceding methods, therapy with a VEGF antagonist (e.g., an anti-VEGF antibody, such as bevacizumab) in combination with a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) preferably extends and/or improves survival, including progression free survival (PFS), overall survival (OS), and/or deterioration-free survival. In one embodiment, therapy with the VEGF antagonist (e.g., an anti-VEGF antibody, such as bevacizumab) in combination with a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) extends survival by at least about 10%, 20%, 30%, 40%, 50%, 60%, 70%, 80%, 90%, 100%, 200%, or more, relative to the survival achieved by administering an approved anti-tumor agent, or standard of care, for the cancer being treated.

In other embodiments of any of the preceding methods, therapy with the angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))) preferably extends and/or improves survival, including progression free survival (PFS), overall survival (OS), and/or deterioration-free survival. In one embodiment, therapy with the angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))) extends survival (e.g., PFS) by at least about 10%, 20%, 30%, 40%, 50%, 60%, 70%, 80%, 90%, 100%, 200%, or more, relative to the survival achieved by administering an approved anti-tumor agent, or standard of care, for the cancer being treated.

In certain embodiments of any of the preceding methods, a reference level is the expression level of the one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37) genes (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2; VEGFA, KDR, ESM1, PECAM1, FLT1, ANGPTL4, or CD34; or IL6, CXCL1, CXCL2, CXCL3, CXCL8, PTGS2, CXCR1, CXCR2, S100A8, or S100A9) in a reference population, for example, a population of individuals having a cancer (e.g., a kidney cancer (e.g., RCC)). In particular embodiments, the cancer is a kidney cancer (e.g., RCC, e.g., mRCC). In certain embodiments, a reference level is the median expression level of the one or more genes in a reference population, for example, a population of individuals having a cancer. In other embodiments, the reference level may be the top 40%, the top 30%, the top 20%, the top 10%, the top 5%, or the top 1% of the expression level in the reference population. In certain embodiments, the reference level is a pre-assigned expression level for the one or more genes. In some embodiments, the reference level is a median of a Z-score of the normalized expression level of the one or more genes. In some embodiments, the reference level is the expression level of the one or more genes in a biological sample obtained from the patient at a previous time point, wherein the previous time point is following administration of the anti-cancer therapy. In some embodiments of any of the preceding methods, a reference level is the expression level of the one or more genes (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2; VEGFA, KDR, ESM1, PECAM1, FLT1, ANGPTL4, or CD34; or IL6, CXCL1, CXCL2, CXCL3, CXCL8, PTGS2, CXCR1, CXCR2, S100A8, or S100A9) in a biological sample from the patient obtained prior to (e.g., minutes, hours, days, weeks (e.g., 1, 2, 3, 4, 5, 6, or 7 weeks), months, or years prior to) administration of the anti-cancer therapy. In other embodiments, the reference level is the expression level of the one or more genes in a biological sample obtained from the patient at a subsequent time point (e.g., minutes, hours, days, weeks, months, or years after administration of an anti-cancer therapy).

In some embodiments of any of the preceding embodiments, the sample is obtained from the individual prior to (e.g., minutes, hours, days, weeks (e.g., 1, 2, 3, 4, 5, 6, or 7 weeks), months, or years prior to) administration of the anti-cancer therapy. In some embodiments of any of the preceding methods, the sample from the individual is obtained about 2 to about 10 weeks (e.g., 2, 3, 4, 5, 6, 7, 8, 9, or 10 weeks) following administration of the anti-cancer therapy. In some embodiments, the sample from the individual is obtained about 4 to about 6 weeks following administration of the anti-cancer therapy.

In some embodiments of any of the preceding methods, the expression level or number of a biomarker is detected in a tissue sample, a primary or cultured cells or cell line, a cell supernatant, a cell lysate, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, or any combination thereof. In some embodiments, the sample is a tissue sample (e.g., a tumor tissue sample), a cell sample, a whole blood sample, a plasma sample, a serum sample, or a combination thereof. In some embodiments, the tumor tissue sample wherein the tumor tissue sample includes tumor cells, tumor-infiltrating immune cells, stromal cells, or a combination thereof. In some embodiments, the tumor tissue sample is a formalin-fixed and paraffin-embedded (FFPE) sample, an archival sample, a fresh sample, or a frozen sample.

For example, in some embodiments of any of the preceding methods, the expression level of a biomarker is detected in tumor-infiltrating immune cells, tumor cells, PBMCs, or combinations thereof using known techniques (e.g., flow cytometry or IHC). Tumor-infiltrating immune cells include, but are not limited to, intratumoral immune cells, peritumoral immune cells or any combinations thereof, and other tumor stroma cells (e.g., fibroblasts). Such tumor infiltrating immune cells may be T lymphocytes (such as CD8⁺ T lymphocytes (e.g., CD8⁺ T effector (Ten) cells) and/or CD4⁺ T lymphocytes (e.g., CD4⁺ Teff cells), B lymphocytes, or other bone marrow-lineage cells including granulocytes (neutrophils, eosinophils, basophils), monocytes, macrophages, dendritic cells (e.g., interdigitating dendritic cells), histiocytes, and natural killer (NK) cells. In some embodiments, the staining for a biomarker is detected as membrane staining, cytoplasmic staining, or combinations thereof. In other embodiments, the absence of a biomarker is detected as absent or no staining in the sample, relative to a reference sample.

In particular embodiments of any of the preceding methods, the expression level of a biomarker is assessed in a sample that contains or is suspected to contain cancer cells. The sample may be, for example, a tissue biopsy or a metastatic lesion obtained from a patient suffering from, suspected to suffer from, or diagnosed with cancer (e.g., a kidney cancer, in particular renal cell carcinoma (RCC), such as advanced RCC or metastatic RCC (mRCC)). In some embodiments, the sample is a sample of kidney tissue, a biopsy of an kidney tumor, a known or suspected metastatic kidney cancer lesion or section, or a blood sample, e.g., a peripheral blood sample, known or suspected to comprise circulating cancer cells, e.g., kidney cancer cells. The sample may comprise both cancer cells, i.e., tumor cells, and non-cancerous cells (e.g., lymphocytes, such as T cells or NK cells), and, in certain embodiments, comprises both cancerous and non-cancerous cells. Methods of obtaining biological samples including tissue resections, biopsies, and body fluids, e.g., blood samples comprising cancer/tumor cells, are well known in the art.

In some embodiments of any of the preceding methods, the individual has carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. In some embodiments, the cancer is kidney cancer (e.g., renal cell carcinoma (RCC), e.g., advanced RCC or metastatic RCC (mRCC)), squamous cell cancer (e.g., epithelial squamous cell cancer), lung cancer (including small-cell lung cancer (SCLC), non-small cell lung cancer (NSCLC), adenocarcinoma of the lung, and squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer (e.g., HCC), hepatoma, breast cancer (including TNBC and metastatic breast cancer), bladder cancer, colon cancer, rectal cancer, colorectal cancer, endometrial or uterine carcinoma, salivary gland carcinoma, prostate cancer, vulval cancer, thyroid cancer, hepatic carcinoma, anal carcinoma, penile carcinoma, Merkel cell cancer, mycoses fungoids, testicular cancer, esophageal cancer, tumors of the biliary tract, head and neck cancer, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), abnormal vascular proliferation associated with phakomatoses, edema (such as that associated with brain tumors), or Meigs' syndrome. In some embodiments, the cancer is a kidney cancer (e.g., RCC), a lung cancer (e.g., NSCLC), a bladder cancer (e.g., UBC), a liver cancer (e.g., HCC), an ovarian cancer, or a breast cancer (e.g., TNBC). In preferred embodiments, the patient has a kidney cancer (e.g., RCC, e.g., advanced RCC or mRCC, e.g., previously untreated advanced RCC or mRCC). The patient may optionally have an advanced, refractory, recurrent, chemotherapy-resistant, and/or platinum-resistant form of the cancer.

In certain embodiments, the presence and/or expression levels/amount of a biomarker in a first sample is increased or elevated as compared to presence/absence and/or expression levels/amount in a second sample. In certain embodiments, the presence/absence and/or expression levels/amount of a biomarker in a first sample is decreased or reduced as compared to presence and/or expression levels/amount in a second sample. In certain embodiments, the second sample is a reference sample, reference cell, reference tissue, control sample, control cell, or control tissue.

In certain embodiments, a reference sample, reference cell, reference tissue, control sample, control cell, or control tissue is a single sample or combined multiple samples from the same patient or individual that are obtained at one or more different time points than when the test sample is obtained. For example, a reference sample, reference cell, reference tissue, control sample, control cell, or control tissue is obtained at an earlier time point from the same patient or individual than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference sample, reference cell, reference tissue, control sample, control cell, or control tissue is a combined multiple samples from one or more healthy individuals who are not the patient. In certain embodiments, a reference sample, reference cell, reference tissue, control sample, control cell, or control tissue is a combined multiple samples from one or more individuals with a disease or disorder (e.g., cancer) who are not the patient or individual. In certain embodiments, a reference sample, reference cell, reference tissue, control sample, control cell, or control tissue is pooled RNA samples from normal tissues or pooled plasma or serum samples from one or more individuals who are not the patient. In certain embodiments, a reference sample, reference cell, reference tissue, control sample, control cell, or control tissue is pooled RNA samples from tumor tissues or pooled plasma or serum samples from one or more individuals with a disease or disorder (e.g., cancer) who are not the patient.

In some embodiments of any of the preceding methods, an expression level above a reference level, or an elevated or increased expression or number, refers to an overall increase of about any of 10%, 20%, 30%, 40%, 50%, 60%, 70%, 80%, 90%, 95%, 96%, 97%, 98%, 99% or greater, in the level or number of a biomarker (e.g., protein, nucleic acid (e.g., gene or mRNA), or cell), detected by methods such as those described herein and/or known in the art, as compared to a reference level, reference sample, reference cell, reference tissue, control sample, control cell, or control tissue. In certain embodiments, the elevated expression or number refers to the increase in expression level/amount of a biomarker (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and/or S100A9) in the sample wherein the increase is at least about any of 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, 3.5×, 4×, 4.5×, 5×, 6×, 7×, 8×, 9×, 10×, 15×, 20×, 30×, 40×, 50×, 100×, 500×, or 1000× the expression level/amount of the respective biomarker in a reference level, reference sample, reference cell, reference tissue, control sample, control cell, or control tissue. In some embodiments, elevated expression or number refers to an overall increase in expression level/amount of a biomarker (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and/or S100A9) of greater than about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, about 3-fold, about 3.5-fold, about 4-fold, about 4.5-fold, about 5-fold, about 6-fold, about 7-fold, about 8-fold, about 9-fold, about 10-fold, about 15-fold, about 20-fold, about 30-fold, about 40-fold, about 50-fold, about 100-fold, about 500-fold, about 1,000-fold or greater as compared to a reference level, reference sample, reference cell, reference tissue, control sample, control cell, control tissue, or internal control (e.g., housekeeping gene).

In some embodiments of any of the preceding methods, an expression level below a reference level, or reduced (decreased) expression or number, refers to an overall reduction of about any of 10%, 20%, 30%, 40%, 50%, 60%, 70%, 80%, 90%, 95%, 96%, 97%, 98%, 99% or greater, in the level of biomarker (e.g., protein, nucleic acid (e.g., gene or mRNA), or cell), detected by standard art known methods such as those described herein, as compared to a reference level, reference sample, reference cell, reference tissue, control sample, control cell, or control tissue. In certain embodiments, reduced expression or number refers to the decrease in expression level/amount of a biomarker (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and/or S100A9) in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference level, reference sample, reference cell, reference tissue, control sample, control cell, or control tissue. In some embodiments, reduced (decreased) expression or number refers to an overall decrease in expression level/amount of a biomarker (e.g., CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and/or S100A9) of greater than about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, about 3-fold, about 3.5-fold, about 4-fold, about 4.5-fold, about 5-fold, about 6-fold, about 7-fold, about 8-fold, about 9-fold, about 10-fold, about 15-fold, about 20-fold, about 30-fold, about 40-fold, about 50-fold, about 100-fold, about 500-fold, about 1,000-fold or greater as compared to a reference level, reference sample, reference cell, reference tissue, control sample, control cell, control tissue, or internal control (e.g., housekeeping gene).

For the prevention or treatment of cancer, the dose of an anti-cancer therapy (e.g., a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), or an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib))))) will depend on the type of cancer to be treated, as defined above, the severity and course of the cancer, whether the anti-cancer therapy is administered for preventive or therapeutic purposes, previous therapy, the patient's clinical history and response to the drug, and the discretion of the attending physician.

In some embodiments, the anti-cancer therapy (e.g., a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)), or an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib))))) may be suitably administered to the patient at one time or over a series of treatments. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g., every week or every three weeks (e.g., such that the patient receives, for example, from about two to about twenty, or e.g., about six doses of the anti-cancer therapy). An initial higher loading dose, followed by one or more lower doses may be administered. However, other dosage regimens may be useful. The progress of this therapy is easily monitored by conventional techniques and assays.

For example, as a general proposition, the therapeutically effective amount of a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and/or PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) administered to human will be in the range of about 0.01 to about 50 mg/kg of patient body weight, whether by one or more administrations. In some embodiments, the therapeutic agent (e.g., antibody) used is about 0.01 mg/kg to about 45 mg/kg, about 0.01 mg/kg to about 40 mg/kg, about 0.01 mg/kg to about 35 mg/kg, about 0.01 mg/kg to about 30 mg/kg, about 0.01 mg/kg to about 25 mg/kg, about 0.01 mg/kg to about 20 mg/kg, about 0.01 mg/kg to about 15 mg/kg, about 0.01 mg/kg to about 10 mg/kg, about 0.01 mg/kg to about 5 mg/kg, or about 0.01 mg/kg to about 1 mg/kg administered daily, weekly, every two weeks, every three weeks, or monthly, for example. In some embodiments, the antibody is administered at 15 mg/kg. However, other dosage regimens may be useful. In one embodiment, an VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and/or PD-L1 axis binding antagonist (e.g., a PD-L1 binding antagonist, such as atezolizumab) is administered to a human at a dose of about 50 mg, about 100 mg, about 200 mg, about 300 mg, about 400 mg, about 420 mg, about 500 mg, about 525 mg, about 600 mg, about 700 mg, about 800 mg, about 840 mg, about 900 mg, about 1000 mg, about 1050 mg, about 1100 mg, about 1200 mg, about 1300 mg, about 1400 mg, about 1500 mg, about 1600 mg, about 1700 mg, or about 1800 mg on day 1 of 21-day cycles (every three weeks, q3w).

In some embodiments, atezolizumab is administered at 1200 mg intravenously every three weeks (q3w). In some embodiments, bevacizumab is administered at a fixed dose at one time or over a series of treatments. Where a fixed dose is administered, preferably it is in the range from about 5 mg to about 2000 mg. For example, the fixed dose may be approximately 420 mg, approximately 525 mg, approximately 840 mg, or approximately 1050 mg. In some embodiments, bevacizumab is administered at 10 mg/kg intravenously every two weeks. In some embodiments, bevacizumab is administered at 15 mg/kg intravenously every three weeks. The dose of VEGF antagonist and/or PD-L1 axis binding antagonist may be administered as a single dose or as multiple doses (e.g., 2, 3, 4, 5, 6, 7, 8, 9, or 10 or more doses). Where a series of doses are administered, these may, for example, be administered approximately every week, approximately every 2 weeks, approximately every 3 weeks, or approximately every 4 weeks. The dose of the antibody administered in a combination treatment may be reduced as compared to a single treatment. The progress of this therapy is easily monitored by conventional techniques.

Any suitable dose of a VEGFR inhibitor (e.g., a multi-targeted tyrosine kinase inhibitor (e.g., sunitinib, axitinib, pazopanib, or cabozantinib))) may be used in the methods described herein. Suitable dosages are well known in the art. For example, with respect to sunitib, capsules of 12.5 mg, 25 mg, and 50 mg of sunitinib are commercially available. For example, for treatment of metastatic renal cell carcinoma or gastrointestinal stromal tumor, sunitinib may be administered at 50 mg by mouth (PO) once a day (qDay) for 4 weeks, followed by 2 weeks drug-free, with further repeats of the cycle. For treatment of pancreatic neuroendocrine tumors, a standard dose is 37.5 mg PO qDay continuously without a scheduled off-treatment period.

VEGF antagonists (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and PD-L1 axis binding antagonists (e.g., an antibody (e.g., an anti-PD-L1 antibody, e.g., atezolizumab), binding polypeptide, and/or small molecule) described herein (any additional therapeutic agent) may be formulated, dosed, and administered in a fashion consistent with good medical practice. Likewise, angiogenesis inhibitors (e.g., a VEGF antagonist (e.g., a VEGFR inhibitor, (e.g., a multi-targeted tyrosine kinase inhibitor (e.g., sunitinib, axitinib, pazopanib, or cabozantinib)))) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The VEGF antagonist and PD-L1 antagonist, or the angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))), need not be, but is optionally formulated with and/or administered concurrently with one or more agents currently used to prevent or treat the disorder in question. The effective amount of such other agents depends on the amount of the VEGF antagonist, PD-L1 antagonist, and/or angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))) present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) is administered concurrently with a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)). In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) are administered as part of the same formulation. In other embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) is administered separately from a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)).

In some embodiments, any of the preceding methods may further include administering an additional therapeutic agent. In some embodiments, the additional therapeutic agent is selected from the group consisting of an immunotherapy agent, a cytotoxic agent, a growth inhibitory agent, a radiation therapy agent, an anti-angiogenic agent, and combinations thereof.

In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) is administered concurrently with an agonist directed against an activating co-stimulatory molecule. In some embodiments, an activating co-stimulatory molecule may include CD40, CD226, CD28, OX40, GITR, CD137, CD27, HVEM, or CD127. In some embodiments, the agonist directed against an activating co-stimulatory molecule is an agonist antibody that binds to CD40, CD226, CD28, OX40, GITR, CD137, CD27, HVEM, or CD127. In some embodiments, VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an antagonist directed against an inhibitory co-stimulatory molecule. In some embodiments, an inhibitory co-stimulatory molecule may include CTLA-4 (also known as CD152), TIM-3, BTLA, VISTA, LAG-3, B7-H3, B7-H4, IDO, TIGIT, MICA/B, or arginase. In some embodiments, the antagonist directed against an inhibitory co-stimulatory molecule is an antagonist antibody that binds to CTLA-4, TIM-3, BTLA, VISTA, LAG-3, B7-H3, B7-H4, IDO, TIGIT, MICA/B, or arginase.

In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an antagonist directed against CTLA-4 (also known as CD152), e.g., a blocking antibody. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with ipilimumab (also known as MDX-010, MDX-101, or YERVOY®).

In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with tremelimumab (also known as ticilimumab or CP-675,206). In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an antagonist directed against B7-H3 (also known as CD276), e.g., a blocking antibody. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with MGA271. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an antagonist directed against a TGF-beta, e.g., metelimumab (also known as CAT-192), fresolimumab (also known as GC1008), or LY2157299.

In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an agonist directed against CD137 (also known as TNFRSF9, 4-1 BB, or ILA), e.g., an activating antibody. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with urelumab (also known as BMS-663513). In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an agonist directed against CD40, e.g., an activating antibody. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with CP-870893. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an agonist directed against OX40 (also known as CD134), e.g., an activating antibody. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an anti-OX40 antibody (e.g., AgonOX). In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an agonist directed against CD27, e.g., an activating antibody. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A) or a PD-1 binding antagonist (e.g., an anti-PD-1 antibody)) may be administered in conjunction with CDX-1127. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an antagonist directed against TIGIT, for example, an anti-TIGIT antibody. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an antagonist directed against indoleamine-2,3-dioxygenase (IDO). In some embodiments, the IDO antagonist is 1-methyl-D-tryptophan (also known as 1-D-MT).

In some embodiments, VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with a cancer vaccine. In some embodiments, the cancer vaccine is a peptide cancer vaccine, which in some embodiments is a personalized peptide vaccine. In some embodiments the peptide cancer vaccine is a multivalent long peptide, a multi-peptide, a peptide cocktail, a hybrid peptide, or a peptide-pulsed dendritic cell vaccine (see, e.g., Yamada et al., Cancer Sci. 104:14-21, 2013). In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an adjuvant. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with a treatment comprising a TLR agonist, e.g., Poly-ICLC (also known as HILTONOL®), LPS, MPL, or CpG ODN. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with tumor necrosis factor (TNF) alpha. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with IL-1. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with HMGB1. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an IL-10 antagonist. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an IL-4 antagonist. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an IL-13 antagonist. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an HVEM antagonist. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an ICOS agonist, e.g., by administration of ICOS-L, or an agonistic antibody directed against ICOS. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with a treatment targeting CX3CL1. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with a treatment targeting CXCL9. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with a treatment targeting CXCL10. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with a treatment targeting CCLS. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with an LFA-1 or ICAM1 agonist. In some embodiments, a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab or a PD-1 binding antagonist (e.g., an anti-PD-1 antibody)) may be administered in conjunction with a Selectin agonist.

A chemotherapeutic agent, if administered, is usually administered at dosages known therefore, or optionally lowered due to combined action of the drugs or negative side effects attributable to administration of the chemotherapeutic agent. Preparation and dosing schedules for such chemotherapeutic agents may be used according to manufacturers' instructions or as determined empirically by the skilled practitioner. Where the chemotherapeutic agent is paclitaxel, preferably, it is administered at a dose between about 130 mg/m² to 200 mg/m² (e.g., approximately 175 mg/m²), for instance, over 3 hours, once every 3 weeks. Where the chemotherapeutic agent is carboplatin, preferably it is administered by calculating the dose of carboplatin using the Calvert formula which is based on a patients preexisting renal function or renal function and desired platelet nadir. Renal excretion is the major route of elimination for carboplatin. The use of this dosing formula, as compared to empirical dose calculation based on body surface area, allows compensation for patient variations in pretreatment renal function that might otherwise result in either underdosing (in patients with above average renal function) or overdosing (in patients with impaired renal function). The target AUC of 4-6 mg/mL/min using single agent carboplatin appears to provide the most appropriate dose range in previously treated patients.

In addition to the above therapeutic regimes, the patient may be subjected to surgical removal of tumors and/or cancer cells.

Such combination therapies noted above encompass combined administration (where two or more therapeutic agents are included in the same or separate formulations), and separate administration, in which case, administration of a VEGF antagonist and/or a PD-L1 axis binding antagonist, or an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))), can occur prior to, simultaneously, and/or following, administration of the additional therapeutic agent or agents. In one embodiment, administration of VEGF antagonist and/or a PD-L1 axis binding antagonist, or a an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))), and administration of an additional therapeutic agent occur within about one month, or within about one, two or three weeks, or within about one, two, three, four, five, or six days, of each other.

In embodiments where either the VEGF antagonist or the PD-L1 axis binding antagonist is an antibody (e.g., bevacizumab or atezolizumab), the administered antibody may be a naked antibody. The VEGF antagonist (e.g., an anti-VEGF antibody, such as bevacizumab) and/or the PD-L1 axis binding antagonist (e.g., a PD-L1 binding antagonist, such as atezolizumab) administered may be conjugated with a cytotoxic agent. Preferably, the conjugated and/or antigen to which it is bound is/are internalized by the cell, resulting in increased therapeutic efficacy of the conjugate in killing the cancer cell to which it binds. In a preferred embodiment, the cytotoxic agent targets or interferes with nucleic acid in the cancer cell. Examples of such cytotoxic agents include maytansinoids, calicheamicins, ribonucleases, and DNA endonucleases.

The compositions utilized in the methods described herein can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), parenterally, by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some embodiments, the PD-L1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some embodiments, the multi-targeted tyrosine kinase inhibitor is administered orally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

### IV. METHODS OF DETERMINING PD-L1 EXPRESSION

Any of the preceding methods may include determining an expression level of PD-L1 in a sample (e.g., a tumor sample) obtained from the individual. In other embodiments, an expression level of PD-L1 in a sample (e.g., a tumor sample) obtained from the individual may have been previously determined. Any suitable approach to determine an expression level of PD-L1 may be used, for example, immunohistochemistry (IHC). An exemplary PD-L1 IHC assay is described, for example, in WO 2016/183326 (see, e.g., Examples 1 and 2, particularly in Tables 2 and 3), which is incorporated herein by reference in its entirety, and others are known in the art.

In some instances of any of the preceding methods, a tumor sample obtained from the patient is or has been determined to have a detectable expression level of PD-L1 in tumor-infiltrating immune cells that comprise less than about 1% of the tumor sample. In other instances, the tumor sample obtained from the patient is or has been determined to have a detectable expression level of PD-L1 in tumor-infiltrating immune cells that comprise about 1% or more (e.g., about 1% or more, 2% or more, 3% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, about 50% or more, about 60% or more, about 70% or more, about 80% or more, about 90% or more, about 95% or more, about 96% or more, about 97% or more, about 98% or more, about 99% or more, or 100%) of the tumor sample. For example, in some instances, the tumor sample obtained from the patient is or has been determined to have a detectable expression level of PD-L1 in tumor-infiltrating immune cells that comprise from about 1% to less than about 5% (e.g., from 1% to 4.9%, from 1% to 4.5%, from 1% to 4%, from 1% to 3.5%, from 1% to 3%, from 1% to 2.5%, or from 1% to 2%) of the tumor sample.

In some instances of any of the preceding methods, a tumor sample obtained from the patient is or has been determined to have a detectable expression level of PD-L1 less than about 1% of the tumor-infiltrating immune cells in the tumor sample. In other instances, the tumor sample obtained from the patient is or has has been determined to have a detectable expression level of PD-L1 in about 1% or more (e.g., about 1% or more, 2% or more, 3% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, about 50% or more, about 60% or more, about 70% or more, about 80% or more, about 90% or more, about 95% or more, about 96% or more, about 97% or more, about 98% or more, about 99% or more, or 100%) of the tumor-infiltrating immune cells in the tumor sample. For example, in some instances, the tumor sample obtained from the patient is or has has been determined to have a detectable expression level of PD-L1 in from about 1% to less than about 5% (e.g., from 1% to 4.9%, from 1% to 4.5%, from 1% to 4%, from 1% to 3.5%, from 1% to 3%, from 1% to 2.5%, or from 1% to 2%) of the tumor-infiltrating immune cells in the tumor sample.

In other instances, the tumor sample obtained from the patient is or has been determined to have a detectable expression level of PD-L1 in tumor-infiltrating immune cells that comprise about 5% or more of the tumor sample. For example, in some instances, the tumor sample obtained from the patient is or has been determined to have a detectable expression level of PD-L1 in tumor-infiltrating immune cells that comprise from about 5% to less than about 10% (e.g., from 5% to 9.5%, from 5% to 9%, from 5% to 8.5%, from 5% to 8%, from 5% to 7.5%, from 5% to 7%, from 5% to 6.5%, from 5% to 6%, from 5% to 5.5%, from 6% to 9.5%, from 6% to 9%, from 6% to 8.5%, from 6% to 8%, from 6% to 7.5%, from 6% to 7%, from 6% to 6.5%, from 7% to 9.5%, from 7% to 9%, from 7% to 7.5%, from 8% to 9.5%, from 8% to 9%, or from 8% to 8.5%) of the tumor sample.

In yet other instances, the tumor sample obtained from the patient is or has been determined to have a detectable expression level of PD-L1 in about 5% or more of the tumor-infiltrating immune cells in the tumor sample. For example, in some instances, the tumor sample obtained from the patient is or has been determined to have a detectable expression level of PD-L1 in from about 5% to less than about 10% (e.g., from 5% to 9.5%, from 5% to 9%, from 5% to 8.5%, from 5% to 8%, from 5% to 7.5%, from 5% to 7%, from 5% to 6.5%, from 5% to 6%, from 5% to 5.5%, from 6% to 9.5%, from 6% to 9%, from 6% to 8.5%, from 6% to 8%, from 6% to 7.5%, from 6% to 7%, from 6% to 6.5%, from 7% to 9.5%, from 7% to 9%, from 7% to 7.5%, from 8% to 9.5%, from 8% to 9%, or from 8% to 8.5%) of the tumor-infiltrating immune cells in the tumor sample.

In still further instances, the tumor sample obtained from the patient is or has been determined to have a detectable expression level of PD-L1 in tumor-infiltrating immune cells that comprise about 10% or more (e.g., 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 60% or more, 70% or more, 80% or more, 90% or more, 95% or more, 96% or more, 97% or more, 98% or more, 99% or more, or 100%) of the tumor sample.

In still further instances, the tumor sample obtained from the patient is or has been determined to have a detectable expression level of PD-L1 in about 10% or more (e.g., 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 60% or more, 70% or more, 80% or more, 90% or more, 95% or more, 96% or more, 97% or more, 98% or more, 99% or more, or 100%) of the tumor-infiltrating immune cells in the tumor sample.

In yet other instances, the tumor sample obtained from the patient is or has been determined to have a detectable expression level of PD-L1 in about 50% or more (e.g., about 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more) of the tumor cells in the tumor sample and/or a detectable expression level of PD-L1 in tumor-infiltrating immune cells that comprise about 10% or more (e.g., 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 60% or more, 70% or more, 80% or more, 90% or more, 95% or more, 96% or more, 97% or more, 98% or more, 99% or more, or 100%) of the tumor sample.

In some embodiments, a tumor sample obtained from the patient is or has been determined to have a detectable expression level of PD-L1 in less than about 1% of the tumor cells in the tumor sample. In other instances, a tumor sample obtained from the patient is or has been determined to have a detectable expression level of PD-L1 in about 1% or more (e.g., about 1% or more, 2% or more, 3% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 21% or more, 22% or more, 23% or more, 24% or more, 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more) of the tumor cells in the tumor sample. For example, in some instances, the tumor sample obtained from the patient is or has been determined to have a detectable expression level of PD-L1 in from about 1% to less than about 5% (e.g., from 1% to 4.9%, from 1% to 4.5%, from 1% to 4%, from 1% to 3.5%, from 1% to 3%, from 1% to 2.5%, or from 1% to 2%) of the tumor cells in the tumor sample.

In other instances, the tumor sample obtained from the patient is or has been determined to have a detectable expression level of PD-L1 in about 5% or more of the tumor cells in the tumor sample. For example, in some instances, the tumor sample obtained from the patient is or has been determined to have a detectable expression level of PD-L1 in from about 5% to less than 50% (e.g., from 5% to 49.5%, from 5% to 45%, from 5% to 40%, from 5% to 35%, from 5% to 30%, from 5% to 25%, from 5% to 20%, from 5% to 15%, from 5% to 10%, from 5% to 9%, from 5% to 8%, from 5% to 7%, from 5% to 6%, from 10% to 49.5%, from 10% to 40%, from 10% to 35%, from 10% to 30%, from 10% to 25%, from 10% to 20%, from 10% to 15%, from 15% to 49.5%, from 15% to 45%, from 15% to 40%, from 15% to 35%, from 15% to 30%, from 15% to 30%, from 15% to 25%, from 15% to 20%, from 20% to 49.5%, from 20% to 45%, from 20% to 40%, from 20% to 35%, from 20% to 30%, from 20% to 25%, from 25% to 49.5%, from 25% to 45%, from 25% to 40%, from 25% to 35%, from 25% to 30%, from 30% to 49.5%, from 30% to 45%, from 30% to 40%, from 30% to 35%, from 35% to 49.5%, from 35% to 45%, from 35% to 40%, from 40% to 49.5%, from 40% to 45%, or from 45% to 49.5%) of the tumor cells in the tumor sample.

In yet other instances, the tumor sample obtained from the patient is or has been determined to have a detectable expression level of PD-L1 in about 50% or more (e.g., about 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more) of the tumor cells in the tumor sample. In some instances, the tumor sample obtained from the patient has been determined to have a detectable expression level of PD-L1 in from about 50% to about 99% (e.g., from 50% to 99%, from 50% to 95%, from 50% to 90%, from 50% to 85%, from 50% to 80%, from 50% to 75%, from 50% to 70%, from 50% to 65%, from 50% to 60%, from 50% to 55%, from 55% to 99%, from 55% to 95%, from 55% to 90%, from 55% to 85%, from 55% to 80%, from 55% to 75%, from 55% to 70%, from 55% to 65%, from 55% to 60%, from 60% to 99%, from 60% to 95%, from 60% to 90%, from 60% to 85%, from 60% to 80%, from 60% to 75%, from 60% to 70%, from 60% to 65%, from 65% to 99%, from 65% to 95%, from 65% to 90%, from 65% to 85%, from 65% to 80%, from 65% to 75%, from 65% to 70%, from 70% to 99%, from 70% to 95%, from 70% to 90%, from 70% to 85%, from 70% to 80%, from 70% to 75%, from 75% to 99%, from 75% to 95%, from 75% to 90%, from 75% to 85%, from 75% to 80%, from 80% to 99%, from 80% to 95%, from 80% to 90%, from 80% to 85%, from 85% to 99%, from 85% to 95%, from 85% to 90%, from 90% to 99%, or from 90% to 95%) of the tumor cells in the tumor sample.

It is to be understood that in any of the preceding methods, the percentage of the tumor sample comprised by tumor-infiltrating immune cells may be in terms of the percentage of tumor area covered by tumor-infiltrating immune cells in a section of the tumor sample obtained from the patient, for example, as assessed by IHC using an anti-PD-L1 antibody (e.g., the SP142 antibody).

### V. COMPOSITIONS AND PHARMACEUTICAL FORMULATIONS

In one aspect, the invention is based, in part, on the discovery that biomarkers of the invention (including sarcomatoid cancer and/or a patient's MSKCC risk score) can be used to identify individuals having a cancer (e.g., a kidney cancer (e.g., RCC)) who may benefit from anti-cancer therapies that include VEGF antagonists and PD-L1 axis binding antagonists. In another aspect, the invention is based, in part, on the discovery that individuals with sarcomatoid cancer (e.g., sarcomatoid kidney cancer) are likely to benefit from anti-cancer therapies that include VEGF antagonists and PD-L1 axis binding antagonists. In another aspect, the invention is based, in part, on the discovery that biomarkers of the invention can be used to identify individuals having a cancer (e.g., a kidney cancer (e.g., RCC)) who may benefit from anti-cancer therapies that include an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))). The benefit may be, for example, in terms of improved progression-free survival (PFS), overall survival (OS), overall response rate (ORR), complete response (CR) rate, or deterioration-free rate (DFR). In some embodiments, the benefit is in terms of improved PFS. In some instances, the benefit is in terms of improved OS. In some instances, the benefit is in terms of improved ORR. In some instances, the benefit is in terms of improved CR rate. In some instances, the benefit is in terms of improved DFR. In some instances, DFR is determined in terms of the time from onset of treatment to the individual's first increase of greater than or equal to 2 points above baseline on the MD Anderson Symptom Inventory (MDASI) interference scale. These agents, and combinations thereof, are useful for the treatment of cancer, e.g., as part of any of the methods described herein, for example, in Sections II and III above. Any suitable VEGF antagonist, PD-L1 axis binding antagonist, and/or angiogenesis inhibitor can be used in the methods and assays described herein. Non-limiting examples suitable for use in the methods and assays of the invention are described further below.

A. Exemplary VEGF Antagonists

VEGF antagonists include any molecule capable of binding VEGF, reducing VEGF expression levels, or neutralizing, blocking, inhibiting, abrogating, reducing, or interfering with VEGF biological activities. An exemplary human VEGF is shown under UniProtKB/Swiss-Prot Accession No. P15692, Gene ID (NCBI): 7422.

In some instances, the VEGF antagonist is an anti-VEGF antibody. In some embodiments, the anti-VEGF antibody is bevacizumab, also known as “rhuMab VEGF” or “AVASTIN®.” Bevacizumab is a recombinant humanized anti-VEGF monoclonal antibody generated according to Presta et al. (Cancer Res. 57:4593-4599, 1997). It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879 issued Feb. 26, 2005, the entire disclosure of which is expressly incorporated herein by reference. Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al. (Journal of Immunological Methods 288:149-164, 2004). Other preferred antibodies include those that bind to a functional epitope on human VEGF comprising of residues F17, M18, D19, Y21, Y25, Q89, 191, K101, E103, and 0104 or, alternatively, comprising residues F17, Y21, Q22, Y25, D63, 183, and Q89.

In other instances, the VEGF antagonist is an anti-VEGFR2 antibody or related molecule (e.g., ramucirumab, tanibirumab, aflibercept); an anti-VEGFR1 antibody or related molecules (e.g., icrucumab, aflibercept (VEGF Trap-Eye; EYLEA®), or ziv-aflibercept (VEGF Trap; ZALTRAP®)); a bispecific VEGF antibody (e.g., MP-0250, vanucizumab (VEGF-ANG2), or bispecific antibodies disclosed in US 2001/0236388); a bispecific antibody including a combination of two of anti-VEGF, anti-VEGFR1, and anti-VEGFR2 arms; an anti-VEGFA antibody (e.g., bevacizumab, sevacizumab); an anti-VEGFB antibody; an anti-VEGFC antibody (e.g., VGX-100), an anti-VEGFD antibody; or a nonpeptide small molecule VEGF antagonist (e.g., pazopanib, axitinib, vandetanib, stivarga, cabozantinib, lenvatinib, nintedanib, orantinib, telatinib, dovitinib, cediranib, motesanib, sulfatinib, apatinib, foretinib, famitinib, or tivozanib).

It is expressly contemplated that such VEGF antagonist antibodies or other antibodies described herein (e.g., anti-VEGF antibodies for detection of VEGF expression levels) for use in any of the embodiments enumerated above may have any of the features, singly or in combination, described in Sections i-vii of Subsection C below.

B. Exemplary PD-L1 Axis Binding Antagonists

PD-L1 axis binding antagonists include PD-1 binding antagonists, PD-L1 binding antagonists, and PD-L2 binding antagonists. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. 015116. PD-L1 (programmed death ligand 1) is also referred to in the art as “programmed cell death 1 ligand 1,” “PDCD1LG1,” “CD274,” “B7-H,” and “PDL1.” An exemplary human PD-L1 is shown in UniProtKB/Swiss-Prot Accession No.Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some embodiments, PD-1, PD-L1, and PD-L2 are human PD-1, PD-L1, and PD-L2. The PD-1 axis binding antagonist may, in some instances, be a PD-1 binding antagonist, a PD-L1 binding antagonist, or a PD-L2 binding antagonist.

(i) PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. In some instances, the PD-L1 binding antagonist is an antibody. In some instances, the antibody is selected from the group consisting of: atezolizumab, YW243.55.570, MDX-1105, MED14736 (durvalumab), and MSB0010718C (avelumab).

In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)₂ fragments. In some instances, the anti-PD-L1 antibody is a humanized antibody. In some instances, the anti-PD-L1 antibody is a human antibody. In some instances, the anti-PD-L1 antibody described herein binds to human PD-L1. In some particular instances, the anti-PD-L1 antibody is atezolizumab (CAS Registry Number: 1422185-06-5). Atezolizumab (Genentech) is also known as MPDL3280A.

In some instances, the anti-PD-L1 antibody comprises a heavy chain variable region (HVR-H) comprising an HVR-H1, HVR-H2, and HVR-H3 sequence, wherein:

(a) the HVR-H1 sequence is
(SEQ ID NO: 62)
GFTFSDSWIH;
(b) the HVR-H2 sequence is
(SEQ ID NO: 63)
AWISPYGGSTYYADSVKG;
and
(c) the HVR-H3 sequence is
(SEQ ID NO: 64)
RHWPGGFDY.

In some instances, the anti-PD-L1 antibody further comprises a light chain variable region (HVR-L) comprising an HVR-L1, HVR-L2, and HVR-L3 sequence, wherein:

(a) the HVR-L1 sequence is
(SEQ ID NO: 65)
RASQDVSTAVA;
(b) the HVR-L2 sequence is
(SEQ ID NO: 66)
SASFLYS;
and
(c) the HVR-L3 sequence is
(SEQ ID NO: 67)
QQYLYHPAT.

In some instances, the anti-PD-L1 antibody comprises a heavy chain and a light chain sequence, wherein:

(a) the heavy chain variable (VH) region
sequence comprises the amino acid sequence:
(SEQ ID NO: 69)
EVQLVESGGGLVQPGGSLRLSCAASGFTFSDSWIHWVRQAPG
KGLEWVAWISPYGGSTYYADSVKGRFTISADTSKNTAYLQMN
SLRAEDTAVYYCARRHWPGGFDYWGQGTLVTVSS;
and
(b) the light chain variable (VL) region
sequence comprises the amino acid sequence:
((SEQ ID NO: 70)
DIQMTQSPSSLSASVGDRVTITCRASQDVSTAVAWYQQKPGKA
PKLLIYSASFLYSGVPSRFSGSGSGTDFTLTISSLQPEDFATY
YCQQYLYHPATFGQGTKVEIKR.

In some instances, the anti-PD-L1 antibody comprises a heavy chain and a light chain sequence, wherein:

(a) the heavy chain comprises the
amino acid sequence:
(SEQ ID NO: 71)
EVQLVESGGGLVQPGGSLRLSCAASGFTFSDSWIHWVRQA
PGKGLEWVAWISPYGGSTYYADSVKGRFTISADTSKNTAY
LQMNSLRAEDTAVYYCARRHWPGGFDYWGQGTLVTVSSAS
TKGPSVFPLAPSSKSTSGGTAALGCLVKDYFPEPVTVSWN
SGALTSGVHTFPAVLQSSGLYSLSSVVTVPSSSLGTQTYI
CNVNHKPSNTKVDKKVEPKSCDKTHTCPPCPAPELLGGPS
VFLFPPKPKDTLMISRTPEVTCVVVDVSHEDPEVKFNWYV
DGVEVHNAKTKPREEQYASTYRVVSVLTVLHQDWLNGKEY
KCKVSNKALPAPIEKTISKAKGQPREPQVYTLPPSREEMT
KNQVSLTCLVKGFYPSDIAVEWESNGQPENNYKTTPPVLD
SDGSFFLYSKLTVDKSRWQQGNVFSCSVMHEALHNHYTQK
SLSLSPG;
and
(b) the light chain comprises the
amino acid sequence:
(SEQ ID NO: 72)
DIQMTQSPSSLSASVGDRVTITCRASQDVSTAVAWYQQKP
GKAPKLLIYSASFLYSGVPSRFSGSGSGTDFTLTISSLQP
EDFATYYCQQYLYHPATFGQGTKVEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC.

In some instances, the anti-PD-L1 antibody comprises (a) a VH domain comprising an amino acid sequence comprising having at least 95% sequence identity (e.g., at least 95%, 96%, 97%, 98%, or 99% sequence identity) to, or the sequence of (SEQ ID NO: 69); (b) a VL domain comprising an amino acid sequence comprising having at least 95% sequence identity (e.g., at least 95%, 96%, 97%, 98%, or 99% sequence identity) to, or the sequence of (SEQ ID NO: 70); or (c) a VH domain as in (a) and a VL domain as in (b). In other instances, the anti-PD-L1 antibody is selected from the group consisting of YW243.55.570, MDX-1105, MED14736 (durvalumab), and MSB0010718C (avelumab). Antibody YW243.55.570 is an anti-PD-L1 described in PCT Pub. No. WO 2010/077634. MDX-1105, also known as BMS-936559, is an anti-PD-L1 antibody described in PCT Pub. No. WO 2007/005874. MED14736 (durvalumab) is an anti-PD-L1 monoclonal antibody described in PCT Pub. No. WO 2011/066389 and U.S. Pub. No. 2013/034559. Examples of anti-PD-L1 antibodies useful for the methods of this invention, and methods for making thereof are described in PCT Pub. Nos. WO 2010/077634, WO 2007/005874, and WO 2011/066389, and also in U.S. Pat. No. 8,217,149, and U.S. Pub. No. 2013/034559, which are incorporated herein by reference.

PD-1 Binding Antagonists

In some instances, the PD-L1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. In some instances, the PD-1 binding antagonist is an antibody. In some instances, the antibody is selected from the group consisting of: MDX 1106 (nivolumab), MK-3475 (pembrolizumab), MEDI-0680 (AMP-514), PDR001, REGN2810, and BGB-108. In some instances, the PD-1 binding antagonist is an Fc-fusion protein. For example, in some instances, the Fc-fusion protein is AMP-224.

In a further aspect, the invention provides for the use of a PD-L1 axis binding antagonist in the manufacture or preparation of a medicament. In one embodiment, the medicament is for treatment of a cancer. In a further embodiment, the medicament is for use in a method of treating a cancer (e.g., a kidney cancer (e.g., RCC), a lung cancer (e.g., NSCLC), a bladder cancer (e.g., UBC), a liver cancer (e.g., HCC), an ovarian cancer, or a breast cancer (e.g., TNBC)) comprising administering to a patient suffering from cancer an effective amount of the medicament. In one such embodiment, the method further comprises administering to the individual an effective amount of at least one additional therapeutic agent, e.g., as described below.

In some embodiments, the PD-1 binding antagonist is a molecule that inhibits the binding of PD-1 to its ligand binding partners. In a specific aspect the PD-1 ligand binding partners are PD-L1 and/or PD-L2. In another embodiment, a PD-L1 binding antagonist is a molecule that inhibits the binding of PD-L1 to its binding ligands. In a specific aspect, PD-L1 binding partners are PD-1 and/or B7-1. In another embodiment, the PD-L2 binding antagonist is a molecule that inhibits the binding of PD-L2 to its ligand binding partners. In a specific aspect, the PD-L2 binding ligand partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody), for example, as described below. In some embodiments, the anti-PD-1 antibody is selected from the group consisting of MDX-1106 (nivolumab), MK-3475 (pembrolizumab), MEDI-0680 (AMP-514), PDR001, REGN2810, and BGB-108. MDX-1106, also known as MDX-1106-04, ONO-4538, BMS-936558, or nivolumab, is an anti-PD-1 antibody described in WO2006/121168. MK-3475, also known as pembrolizumab or lambrolizumab, is an anti-PD-1 antibody described in WO 2009/114335. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 binding antagonist is AMP-224. AMP-224, also known as B7-DCIg, is a PD-L2-Fc fusion soluble receptor described in WO 2010/027827 and WO 2011/066342.

In some embodiments, the anti-PD-1 antibody is MDX-1106. Alternative names for “MDX-1106” include MDX-1106-04, ONO-4538, BMS-936558, and nivolumab. In some embodiments, the anti-PD-1 antibody is nivolumab (CAS Registry Number: 946414-94-4). In a still further embodiment, provided is an isolated anti-PD-1 antibody comprising a heavy chain variable region comprising the heavy chain variable region amino acid sequence from SEQ ID NO: 73 and/or a light chain variable region comprising the light chain variable region amino acid sequence from SEQ ID NO: 74.

In a still further embodiment, provided is an isolated anti-PD-1 antibody comprising a heavy chain and/or a light chain sequence, wherein:

(a) the heavy chain sequence has at least 85%,
at least 90%, at least 91%, at least 92%, at
least 93%, at least 94%, at least 95%, at least
96%, at least 97%, at least 98%, at least 99%
or 100% sequence identity to the heavy
chain sequence:
(SEQ ID NO: 73)
QVQLVESGGGVVQPGRSLRLDCKASGITFSNSGMHWVRQAPGKGLE
WVAVIWYDGSKRYYADSVKGRFTISRDNSKNTLFLQMNSLRAEDTA
VYYCATNDDYWGQGTLVTVSSASTKGPSVFPLAPCSRSTSESTAAL
GCLVKDYFPEPVTVSWNSGALTSGVHTFPAVLQSSGLYSLSSVVTV
PSSSLGTKTYTCNVDHKPSNTKVDKRVESKYGPPCPPCPAPEFLGG
PSVFLFPPKPKDTLMISRTPEVTCVVVDVSQEDPEVQFNWYVDGVE
VHNAKTKPREEQFNSTYRVVSVLTVLHQDWLNGKEYKCKVSNKGLP
SSIEKTISKAKGQPREPQVYTLPPSQEEMTKNQVSLTCLVKGFYPS
DIAVEWESNGQPENNYKTTPPVLDSDGSFFLYSRLTVDKSRWQEGN
VFSCSVMHEALHNHYTQKSLSLSLGK,
and
(b) the light chain sequences has at least 85%,
at least 90%, at least 91%, at least 92%, at
least 93%, at least 94%, at least 95%, at least
96%, at least 97%, at least 98%, at least 99%
or 100% sequence identity to the light
chain sequence:
(SEQ ID NO: 74)
EIVLTQSPATLSLSPGERATLSCRASQSVSSYLAWYQQKPGQAPRL
LIYDASNRATGIPARFSGSGSGTDFTLTISSLEPEDFAVYYCQQSS
NWPRTFGQGTKVEIKRTVAAPSVFIFPPSDEQLKSGTASVVCLLNN
FYPREAKVQWKVDNALQSGNSQESVTEQDSKDSTYSLSSTLTLSKA
DYEKHKVYACEVTHQGLSSPVTKSFNRGEC.

It is expressly contemplated that such PD-L1 axis binding antagonist antibodies (e.g., anti-PD-L1 antibodies, anti-PD-1 antibodies, and anti-PD-L2 antibodies), or other antibodies described herein (e.g., anti-PD-L1 antibodies for detection of PD-L1 expression levels) for use in any of the embodiments enumerated above may have any of the features, singly or in combination, described in Sections i-vii of Subsection C below.

C. Antibodies

i. Antibody Affinity

In certain embodiments, an antibody provided herein (e.g., an anti-VEGF antibody, an anti-PD-L1 antibody or an anti-PD-1 antibody) has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹M to 10⁻¹³ M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881, 1999). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one embodiment, carboxymethylated dextran biosensor chips (CM5, BIACORE, Inc.) are activated with N-ethyl-N′(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, for example, Chen et al., (J. Mol. Biol. 293:865-881, 1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

ii. Antibody Fragments

In certain embodiments, an antibody (e.g., an anti-PD-L1 antibody or an anti-PD-1 antibody) provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. (Nat. Med. 9:129-134, 2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994). See also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097, WO 1993/01161, Hudson et al. Nat. Med. 9:129-134, 2003, and Hollinger et al. Proc. Natl. Acad. Sci. USA 90: 6444-6448, 1993. Triabodies and tetrabodies are also described in Hudson et al. (Nat. Med. 9:129-134, 2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), according to known methods.

iii. Chimeric and Humanized Antibodies

In certain embodiments, an antibody (e.g., an anti-VEGF antibody, an anti-PD-L1 antibody or an anti-PD-1 antibody) provided herein is a chimeric antibody. Certain chimeric antibodies are described, e.g., in U.S. Pat. No. 4,816,567; and Morrison et al. (Proc. Natl. Acad. Sci. USA, 81:6851-6855, 1984). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, (Front. Biosci. 13:1619-1633, 2008), and are further described, e.g., in Riechmann et al. (Nature 332:323-329, 1988); Queen et al. (Proc. Natl. Acad. Sci. USA 86:10029-10033, 1989); U.S. Pat. Nos. 5,821,337, 7,527,791, 6,982,321, and 7,087,409; Kashmiri et al. (Methods 36:25-34, 2005) (describing specificity determining region (SDR) grafting); Padlan, (Mol. Immunol. 28:489-498, 1991) (describing “resurfacing”); Dall'Acqua et al. (Methods 36:43-60, 2005) (describing “FR shuffling”); Osbourn et al. (Methods 36:61-68, 2005), and Klimka et al. (Br. J. Cancer, 83:252-260, 2000) (describing the “guided selection” approach to FR shuffling).

Human framework regions that may be used for humanization include but are not limited to: framework regions selected using the “best-fit” method (see, e.g., Sims et al. J. Immunol. 151:2296, 1993); framework regions derived from the consensus sequence of human antibodies of a particular subgroup of light or heavy chain variable regions (see, e.g., Carter et al. Proc. Natl. Acad. Sci. USA, 89:4285, 1992; and Presta et al. J. Immunol., 151:2623, 1993); human mature (somatically mutated) framework regions or human germline framework regions (see, e.g., Almagro and Fransson, Front. Biosci. 13:1619-1633, 2008); and framework regions derived from screening FR libraries (see, e.g., Baca et al., J. Biol. Chem. 272:10678-10684, 1997; and Rosok et al. J. Biol. Chem. 271:22611-22618, 1996).

iv. Human Antibodies

In certain embodiments, an antibody (e.g., an anti-VEGF antibody, an anti-PD-L1 antibody or an anti-PD-1 antibody) provided herein is a human antibody. Human antibodies can be produced using various techniques known in the art. Human antibodies are described generally in van Dijk and van de Winkel, (Curr. Opin. PharmacoL 5: 368-74, 2001) and Lonberg (Curr. Opin. ImmunoL 20:450-459, 2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, (Nat. Biotech. 23:1117-1125, 2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. See, e.g., Kozbor, (J. ImmunoL 133: 3001, 1984); Brodeur et al. (Monoclonal Antibody Production Techniques and Applications, pp. 51-63, Marcel Dekker, Inc., New York, 1987); and Boerner et al. (J. Immunol., 147: 86, 1991). Human antibodies generated via human B-cell hybridoma technology are also described in Li et al., Proc. Natl. Acad. Sci. USA, 103:3557-3562, 2006. Additional methods include those described, for example, in U.S. Pat. No. 7,189,826 (describing production of monoclonal human IgM antibodies from hybridoma cell lines) and Ni, Xiandai Mianyixue, 26(4):265-268, 2006 (describing human-human hybridomas). Human hybridoma technology (Trioma technology) is also described in Vollmers and Brandlein, Histology and Histopathology, 20(3):927-937, 2005 and Vollmers and Brandlein, Methods and Findings in Experimental and Clinical Pharmacology, 27(3):185-91, 2005.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

v. Library-Derived Antibodies

Antibodies of the invention (e.g., anti-VEGF antibodies, anti-PD-L1 antibodies, or anti-PD-1 antibodies) may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. Methods in Molecular Biology 178:1-37, O'Brien et al., ed., Human Press, Totowa, N.J., 2001 and further described, e.g., in McCafferty et al. Nature 348:552-554, 1990; Clackson et al. Nature 352: 624-628, 1991; Marks et al. J. Mol. Biol. 222: 581-597, 1992; Marks and Bradbury, Methods in Molecular Biology 248:161-175, Lo, ed., Human Press, Totowa, N.J., 2003; Sidhu et al. J. Mol. Biol. 338(2): 299-310, 2004; Lee et al. J. Mol. Biol. 340(5): 1073-1093, 2004; Fellouse, Proc. Natl. Acad. Sci. USA 101(34): 12467-12472, 2004; and Lee et al. J. Immunol. Methods 284(1-2): 119-132, 2004.

In certain phage display methods, repertoires of VH and VL genes are separately cloned by polymerase chain reaction (PCR) and recombined randomly in phage libraries, which can then be screened for antigen-binding phage as described in Winter et al. Ann. Rev. ImmunoL, 12: 433-455, 1994. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and self antigens without any immunization as described by Griffiths et al. EMBO J, 12: 725-734, 1993. Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388, 1992. Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

vi. Multispecific Antibodies

In any one of the above aspects, an antibody (e.g., an anti-VEGF antibody, an anti-PD-L1 antibody, or an anti-PD-1 antibody) provided herein may be a multispecific antibody, for example, a bispecific antibody. Multispecific antibodies are monoclonal antibodies that have binding specificities for at least two different sites. In certain embodiments, an antibody provided herein is a multispecific antibody, e.g., a bispecific antibody. In certain embodiments, one of the binding specificities is for PD-L1 and the other is for any other antigen. In certain embodiments, one of the binding specificities is for VEGF and the other is for any other antigen. In certain embodiments, bispecific antibodies may bind to two different epitopes of PD-L1. In certain embodiments, bispecific antibodies may bind to two different epitopes of VEGF. Bispecific antibodies may also be used to localize cytotoxic agents to cells which express PD-L1 or VEGF. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537, 1983), WO 93/08829 and Traunecker et al. EMBO J. 10: 3655, 1991) and “knob-in-hole” engineering (see, e.g., U.S. Pat. No. 5,731,168). Multi-specific antibodies may also be made by engineering electrostatic steering effects for making antibody Fc-heterodimeric molecules (see, e.g., WO 2009/089004A1); cross-linking two or more antibodies or fragments (see, e.g., U.S. Pat. No. 4,676,980, and Brennan et al. Science 229: 81, 1985); using leucine zippers to produce bi-specific antibodies (see, e.g., Kostelny et al. J. Immunol. 148(5): 1547-1553, 1992); using “diabody” technology for making bispecific antibody fragments (see, e.g., Hollinger et al. Proc. Natl. Acad. Sci. USA 90:6444-6448, 1993); and using single-chain Fv (sFv) dimers (see, e.g., Gruber et al. J. Immunol. 152:5368, 1994); and preparing trispecific antibodies as described, e.g., in Tutt et al. J. Immunol. 147: 60, 1991).

Engineered antibodies with three or more functional antigen binding sites, including “Octopus antibodies,” are also included herein (see, e.g., US 2006/0025576A1).

The antibody or fragment herein includes a “Dual Acting FAb” or “DAF” comprising an antigen binding site that binds to PD-L1 and another, different antigen. The antibody or fragment herein also includes a DAF comprising an antigen binding site that binds to VEGF and another, different antigen.

vii. Antibody Variants

In certain embodiments, amino acid sequence variants of the antibodies of the invention (e.g., anti-VEGF antibodies, anti-PD-L1 antibodies, and anti-PD-1 antibodies) are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

a. Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 17 under the heading of “preferred substitutions.” More substantial changes are provided in Table 17 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 17
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity and/or reduced immunogenicity) relative to the parent antibody and/or will have substantially retained certain biological properties of the parent antibody. An exemplary substitutional variant is an affinity matured antibody, which may be conveniently generated, for example, using phage display-based affinity maturation techniques such as those described herein. Briefly, one or more HVR residues are mutated and the variant antibodies displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196, 2008), and/or residues that contact antigen, with the resulting variant VH or VL being tested for binding affinity. Affinity maturation by constructing and reselecting from secondary libraries has been described, e.g., in Hoogenboom et al. Methods in Molecular Biology 178:1-37, O'Brien et al., ed., Human Press, Totowa, N.J., 2001. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen-contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells Science, 244:1081-1085, 1989. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

b. Glycosylation variants

In certain embodiments, antibodies useful in the invention can be altered to increase or decrease the extent to which the antibody is glycosylated. Addition or deletion of glycosylation sites to an antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, for example, U.S. Patent Publication Nos. US 2003/0157108; US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. (J. Mol. Biol. 336:1239-1249, 2004); and Yamane-Ohnuki et al. (Biotech. Bioeng. 87: 614, 2004). Examples of cell lines capable of producing defucosylated antibodies include Lec13 CHO cells deficient in protein fucosylation (Ripka et al. Arch. Biochem. Biophys. 249:533-545, 1986); U.S. Pat. Appl. No. US 2003/0157108 A1; and WO 2004/056312 A1, especially at Example 11), and knockout cell lines, such as alpha-1,6-fucosyltransferase gene, FUT8, knockout CHO cells (see, e.g., Yamane-Ohnuki et al. Biotech. Bioeng. 87: 614, 2004; Kanda, Y. et al. Biotechnol. Bioeng. 94(4):680-688, 2006; and WO 2003/085107).

Antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

c. Fc Region Variants

In certain embodiments, one or more amino acid modifications may be introduced into the Fc region of an antibody of the invention, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, (Annu. Rev. Immunol. 9:457-492, 1991). Non-limiting examples of in vitro assays to assess ADCC activity of a molecule of interest is described in U.S. Pat. No. 5,500,362 (see, e.g., Hellstrom, I. et al. Proc. Natl. Acad. Sci. USA 83:7059-7063, 1986) and Hellstrom, I et al. Proc. Natl. Acad. Sci. USA 82:1499-1502, 1985; U.S. Pat. No. 5,821,337; Bruggemann et al. J. Exp. Med. 166:1351-1361, 1987). Alternatively, non-radioactive assays methods may be employed (see, for example, ACTI™ non-radioactive cytotoxicity assay for flow cytometry (CellTechnology, Inc. Mountain View, Calif.; and CYTOTOX 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. (Proc. Natl. Acad. Sci. USA 95:652-656, 1998). C1q binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, e.g., Gazzano-Santoro et al. J. Immunol. Methods 202:163, 1996; Cragg et al. Blood. 101:1045-1052, 2003; and Cragg et al. Blood. 103:2738-2743, 2004). FcRn binding and in vivo clearance/half-life determinations can also be performed using methods known in the art (see, e.g., Petkova et al. Intl. Immunol. 18(12):1759-1769, 2006).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

Certain antibody variants with improved or diminished binding to FcRs are described (see, e.g., U.S. Pat. No. 6,737,056; WO 2004/056312, and Shields et al., J. Biol. Chem. 9(2): 6591-6604, 2001).

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184, 2000.

Antibodies with increased half-lives and improved binding to the neonatal Fc receptor (FcRn), which is responsible for the transfer of maternal IgGs to the fetus (Guyer et al., J. Immunol. 117:587, 1976; and Kim et al. J. Immunol. 24:249, 1994), are described in U.S. Pub. No. 2005/0014934A1. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan and Winter, Nature 322:738-40, 1988; U.S. Pat. Nos. 5,648,260; 5,624,821; and WO 94/29351, concerning other examples of Fc region variants.

d. Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

e. Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al. Proc. Natl. Acad. Sci. USA 102: 11600-11605, 2005). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

f. Immunoconjugates

The invention also provides immunoconjugates comprising an antibody herein (e.g., an anti-VEGF antibody, an anti-PD-L1 antibody, or an anti-PD-1 antibody) conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020 and 5,416,064 and European Patent EP 0 425 235 B1); an auristatin such as monomethylauristatin drug moieties DE and DF (MMAE and MMAF) (see U.S. Pat. Nos. 5,635,483, 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al. Cancer Res. 53:3336-3342, 1993; and Lode et al. Cancer Res. 58:2925-2928, 1998); an anthracycline such as daunomycin or doxorubicin (see Kratz et al. Current Med. Chem. 13:477-523, 2006; Jeffrey et al. Bioorganic & Med. Chem. Letters 16:358-362, 2006; Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532, 2002; King et al., J. Med. Chem. 45:4336-4343, 2002; and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Smi⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron. Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al. (Science 238:1098, 1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al. Cancer Res. 52:127-131, 1992; and U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

D. Multi-Targeted Tyrosine Kinase Inhibitors

Any suitable multi-targeted tyrosine kinase inhibitor can be used in the methods described herein. For example, the multi-targeted tyrosine kinase inhibitor may inhibit platelet-derived growth factor receptors (e.g., PDGFR-αα, PDGFR-ββ, and PDGFR-αβ), VEGF receptors (e.g., VEGFR1 and VEGFR2), CD117 (c-Kit), RET, CD114, and/or CD135. Exemplary multi-targeted tyrosine kinase inhibitors include sunitinib (also known as N-[2-(Diethylamino)ethyl]-5-[(Z)-(5-fluoro-2-oxo-1,2-dihydro-3H-indol-3-ylidene)methyl]-2,4-dimethyl-1H-pyrrole-3-carboxamide, SUTENT®, or SU11248), SU6656, motesanib, sorafenib (e.g., NEXEVAR® or BAY439006), axitinib, afatinib, bosutinib, crizotinib, cabozantinib, dasatinib, entrectinib, pazopanib, lapatinib, and vandetanib (also known as ZACTIMA® or ZD6474). In some embodiments, the multi-targeted tyrosine kinase inhibitor is a VEGFR inhibitor.

E. Pharmaceutical Formulations

Therapeutic formulations of the VEGF antagonists and the PD-L1 axis binding antagonists used in accordance with the present invention (e.g., an anti-VEGF antibody, such as bevacizumab, and an anti-PD-L1 antibody, such as atezolizumab) are prepared for storage by mixing the antagonist having the desired degree of purity with optional pharmaceutically acceptable carriers, excipients, or stabilizers in the form of lyophilized formulations or aqueous solutions. Therapeutic formulations of the multi-targeted tyrosine kinase inhibitors used in accordance with the present invention (e.g., sunitinib) are also prepared for storage by mixing the antagonist having the desired degree of purity with optional pharmaceutically acceptable carriers, excipients, or stabilizers in the form of lyophilized formulations or aqueous solutions. For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 119, Marcel Dekker, 2002.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

The formulation herein may also contain more than one active compound, preferably those with complementary activities that do not adversely affect each other. The type and effective amounts of such medicaments depend, for example, on the amount and type of antagonist present in the formulation, and clinical parameters of the patients.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed., 1980.

Sustained-release preparations may be prepared. Suitable examples of sustained-release preparations include semi-permeable matrices of solid hydrophobic polymers containing the antagonist, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### VI. ARTICLES OF MANUFACTURE AND KITS

In another aspect of the invention, a kit or an article of manufacture containing materials useful for the treatment, prevention, and/or diagnosis of individuals is provided.

In some instances, such kits or articles of manufacture can be used to identify an individual having a cancer (e.g., kidney cancer (e.g., RCC)) who may benefit from an anti-cancer therapy that includes a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A)) or a PD-1 binding antagonist (e.g., anti-PD-1 antibody)). In other instances, such articles of manufacture or kits can be used to identify an individual having a cancer (e.g., kidney cancer (e.g., RCC)) who may benefit from an anti-cancer therapy that includes an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))). Such articles of manufacture or kits may include (a) reagents for determining whether the patient has a sarcomatoid cancer and (b) instructions for using the reagents to identify an individual having a cancer (e.g., kidney cancer (e.g., RCC)) who may benefit from a treatment including a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A)) or a PD-1 binding antagonist (e.g., anti-PD-1 antibody)), or with an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))). In other embodiments, such articles of manufacture or kits may include (a) reagents for determining an individual's MSKCC risk score and (b) instructions for using the reagents to identify an individual having a cancer (e.g., kidney cancer (e.g., RCC)) who may benefit from a treatment including a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A)) or a PD-1 binding antagonist (e.g., anti-PD-1 antibody)), or with an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))). The benefit may be, for example, in terms of improved progression-free survival (PFS), overall survival (OS), overall response rate (ORR), complete response (CR) rate, or deterioration-free rate (DFR). In some embodiments, the benefit is in terms of improved PFS. In some instances, the benefit is in terms of improved OS. In some instances, the benefit is in terms of improved ORR. In some instances, the benefit is in terms of improved CR rate. In some instances, the benefit is in terms of improved DFR. In some instances, DFR is determined in terms of the time from onset of treatment to the individual's first increase of greater than or equal to 2 points above baseline on the MD Anderson Symptom Inventory (MDASI) interference scale.

Any of the articles of manufacture or kits may further include (a) reagents for determining the expression level of one or more genes set forth in Table 1, or any combination thereof (e.g., any combination set forth in any one of Tables 2-12) in a sample from the individual and (b) instructions for using the reagents to identify an individual having a cancer (e.g., kidney cancer (e.g., RCC)) who may benefit from a treatment including a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist (e.g., an anti-PD-L1 antibody, e.g., atezolizumab (MPDL3280A)) or a PD-1 binding antagonist (e.g., anti-PD-1 antibody)), or with an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))).

In some embodiments, the kit includes (a) reagents for determining the expression level of determining the expression level of one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37) of the following genes in a sample from the individual: CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2; VEGFA, KDR, ESM1, PECAM1, FLT1, ANGPTL4, or CD34; or IL6, CXCL1, CXCL2, CXCL3, CXCL8, PTGS2, CXCR1, CXCR2, S100A8, or S100A9; and, optionally, (b) instructions for using the reagents to identify an individual having a cancer who may benefit from a treatment with an anti-cancer therapy comprising a VEGF antagonist and a PD-L1 axis binding antagonist.

Any of the preceding kits may include reagents for determining the expression level of one or more (e.g., 1, 2, 3, 4, 5, 6, 7, 8, 9, 10, 11, 12, 13, 14, 15, 16, 17, 18, 19, or 20) of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2. In some embodiments, the kit includes reagents for determining the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, and TAP2.

For example, any of the preceding kits may include reagents for determining the expression level of one or more (e.g., 1, 2, 3, 4, or 5) of CD8A, EOMES, PRF1, IFNG, or PD-L1. In some embodiments, the kit includes determining the expression level of at least two, at least three, at least four, or all five of CD8A, EOMES, PRF1, IFNG, and PD-L1. In some embodiments, the kit includes reagents for determining the expression level of two of CD8A, EOMES, PRF1, IFNG, and PD-L1, for example, any of the exemplary combinations shown in Table 2. In some embodiments, the kit includes reagents for determining the expression level of three of CD8A, EOMES, PRF1, IFNG, and PD-L1, for example, any of the exemplary combinations shown in Table 3. In some embodiments, the kit includes reagents for determining the expression level of four of CD8A, EOMES, PRF1, IFNG, and PD-L1, for example, any of the exemplary combinations shown in Table 4. In some embodiments, the kit includes reagents for determining the expression level of CD8A, EOMES, PRF1, IFNG, and PD-L1.

In some embodiments, any of the preceding kits may include reagents for determining the expression level of PD-L1 and one or more additional genes, wherein the one or more additional genes is other than PD-L1. For example, in some embodiments, the kit may include reagents for determining the expression level of PD-L1 and one or more additional genes (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36) selected from the group consisting of: CD8A, EOMES, GZMA, GZMB, PRF1, IFNG, CXCL9, CXCL10, CXCL11, CD27, FOXP3, PD-1, CTLA4, TIGIT, IDO1, PSMB8, PSMB9, TAP1, TAP2, VEGFA, KDR, ESM1, PECAM1, FLT1, ANGPTL4, CD34, IL6, CXCL1, CXCL2, CXCL3, CXCL8, PTGS2, CXCR1, CXCR2, S100A8, and S100A9. In some embodiments, the kit includes reagents for determining the expression level of PD-L1 and one or more (e.g., 1, 2, 3, 4, 5, 6, 7, 8, 9, 10, 11, 12, 13, 14, 15, 16, 17, 18, or 19) additional genes selected from the group consisting of CD8A, EOMES, GZMA, GZMB, PRF1, IFNG, CXCL9, CXCL10, CXCL11, CD27, FOXP3, PD-1, CTLA4, TIGIT, IDO1, PSMB8, PSMB9, TAP1, and TAP2. In other embodiments, the kit includes reagents for determining the expression level of PD-L1 and one or more (e.g., 1, 2, 3, 4, 5, 6, or 7) of VEGFA, KDR, ESM1, PECAM1, FLT1, ANGPTL4, or CD34. In other embodiments, the kit includes determining the expression level of PD-L1 and one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9.

Any of the preceding kits may include reagents for determining the expression level of one or more (e.g., 1, 2, 3, 4, 5, 6, or 7) of VEGFA, KDR, ESM1, PECAM1, FLT1, ANGPTL4, or CD34. In some embodiments, the kit includes reagents for determining the expression level of at least two, at least three, at least four, at least five, at least six, or all seven of VEGFA, KDR, ESM1, PECAM1, FLT1, ANGPTL4, or CD34. For example, in some embodiments, the kit includes reagents for determining the expression level of one or more of VEGFA, KDR, ESM1, PECAM1, ANGPTL4, or CD34. In some embodiments, the kit includes reagents for determining the expression level of at least two, at least three, at least four, at least five, or all six of VEGFA, KDR, ESM1, PECAM1, ANGPTL4, and CD34. In some embodiments, the kit includes reagents for determining the expression level of two of VEGFA, KDR, ESM1, PECAM1, ANGPTL4, and CD34, for example, any of the exemplary combinations shown in Table 5. In some embodiments, the kit includes reagents for determining the expression level of three of VEGFA, KDR, ESM1, PECAM1, ANGPTL4, and CD34, for example, any of the exemplary combinations shown in Table 6. In some embodiments, the kit includes reagents for determining the expression level of four of VEGFA, KDR, ESM1, PECAM1, ANGPTL4, and CD34, for example, any of the exemplary combinations shown in Table 7. In some embodiments, the kit includes reagents for determining the expression level of five of VEGFA, KDR, ESM1, PECAM1, ANGPTL4, and CD34, for example, any of the exemplary combinations shown in Table 8. In some embodiments, the kit includes reagents for determining the expression level of VEGFA, KDR, ESM1, PECAM1, ANGPTL4, and CD34.

Any of the preceding kits may include reagents for determining the expression level of one or more (e.g., 1, 2, 3, 4, 5, 6, 7, 8, 9, or 10) of IL6, CXCL1, CXCL2, CXCL3, CXCL8, PTGS2, CXCR1, CXCR2, S100A8, or S100A9. In some embodiments, the kit includes reagents for determining the expression level of at least two, at least three, at least four, at least five, or all six of IL6, CXCL1, CXCL2, CXCL3, CXCL8, PTGS2, CXCR1, CXCR2, S100A8, and S100A9. In some embodiments, the kit includes reagents for determining the expression level of two of IL6, CXCL1, CXCL2, CXCL3, CXCL8, PTGS2, CXCR1, CXCR2, S100A8, and S100A9, for example, any of the exemplary combinations shown in Table 9. In some embodiments, the kit includes reagents for determining the expression level of three of IL6, CXCL1, CXCL2, CXCL3, CXCL8, PTGS2, CXCR1, CXCR2, S100A8, and S100A9, for example, any of the exemplary combinations shown in Table 10. In some embodiments, the kit includes reagents for determining the expression level of four of IL6, CXCL1, CXCL2, CXCL3, CXCL8, PTGS2, CXCR1, CXCR2, S100A8, and S100A9, for example, any of the exemplary combinations shown in Table 11. In some embodiments, the kit includes reagents for determining the expression level of five of IL6, CXCL1, CXCL2, CXCL3, CXCL8, PTGS2, CXCR1, CXCR2, S100A8, and S100A9, for example, any of the exemplary combinations shown in Table 12. In some embodiments, the kit includes reagents for determining the expression level of six of IL6, CXCL1, CXCL2, CXCL3, CXCL8, PTGS2, CXCR1, CXCR2, S100A8, and S100A9, for example, any of the exemplary combinations shown in Table 13. In some embodiments, the kit includes reagents for determining the expression level of seven of IL6, CXCL1, CXCL2, CXCL3, CXCL8, PTGS2, CXCR1, CXCR2, S100A8, and S100A9, for example, any of the exemplary combinations shown in Table 14. In some embodiments, the kit includes reagents for determining the expression level of eight of IL6, CXCL1, CXCL2, CXCL3, CXCL8, PTGS2, CXCR1, CXCR2, S100A8, and S100A9, for example, any of the exemplary combinations shown in Table 15. In some embodiments, the kit includes reagents for determining the expression level of nine of IL6, CXCL1, CXCL2, CXCL3, CXCL8, PTGS2, CXCR1, CXCR2, S100A8, and S100A9, for example, any of the exemplary combinations shown in Table 16. In some embodiments, the kit includes reagents for determining the expression level of IL6, CXCL1, CXCL2, CXCL3, CXCL8, PTGS2, CXCR1, CXCR2, S100A8, and S100A9.

In some instances, such kits or articles of manufacture include a VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and a PD-L1 axis binding antagonist (e.g., a PD-L1 binding antagonist, e.g., an anti-PD-L1 antibody, e.g., atezolizumab (MPDL3280A)) for treating an individual with a cancer (e.g., kidney cancer (e.g., RCC)). In some instances, such articles of manufacture or kits further include a package insert including instructions for administration of an anti-cancer therapy comprising the VEGF antagonist (e.g., an anti-VEGF antibody, (e.g., bevacizumab) or a VEGFR inhibitor (e.g., a multi-targeted tyrosine kinase inhibitor (e.g., sunitinib, axitinib, pazopanib, or cabozantinib))) and the PD-L1 axis binding antagonist (e.g., a PD-L1 binding antagonist, e.g., an anti-PD-L1 antibody, e.g., atezolizumab (MPDL3280A)) to an individual having a cancer (e.g., kidney cancer (e.g., RCC)), wherein the patient is identified as one who may benefit from the anti-cancer therapy by any of the methods and/or kits described herein.

In other instances, such kits or articles of manufacture include an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))) for treating an individual with a cancer (e.g., kidney cancer (e.g., RCC)). In some instances, such articles of manufacture or kits further include a package insert including instructions for administration of an anti-cancer therapy comprising the an angiogenesis inhibitor (e.g., a VEGF antagonist (e.g., a VEGFR inhibitor, (e.g., a multi-targeted tyrosine kinase inhibitor (e.g., sunitinib, axitinib, pazopanib, or cabozantinib)))), wherein the patient is identified as one who may benefit from the anti-cancer therapy by any of the methods and/or kits described herein.

In other instances, such kits or articles of manufacture include a PD-L1 axis binding antagonist (e.g., a PD-L1 binding antagonist, e.g., an anti-PD-L1 antibody, e.g., atezolizumab (MPDL3280A), or a PD-1 binding antagonist, e.g., an anti-PD-1 antibody) monotherapy for treating an individual with a cancer (e.g., kidney cancer (e.g., RCC)). In some instances, such articles of manufacture or kits further include a package insert including instructions for administration of the PD-L1 axis binding antagonist monotherapy, wherein the patient is identified as one who may benefit from the anti-cancer therapy by any of the methods and/or kits described herein.

Any of the kits or articles of manufacture described may include a carrier means being compartmentalized to receive in close confinement one or more container means such as vials, tubes, and the like, each of the container means comprising one of the separate elements to be used in the method. Where the article of manufacture or kit utilizes nucleic acid hybridization to detect the target nucleic acid, the kit may also have containers containing nucleotide(s) for amplification of the target nucleic acid sequence and/or a container comprising a reporter-means, such as an enzymatic, florescent, or radioisotope label.

In some instances, the article of manufacture or kit includes the container described above and one or more other containers including materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above. For example, the article of manufacture or kit may further include a container including a pharmaceutically-acceptable buffer, such as bacteriostatic water for injection (SWFI), phosphate-buffered saline, Ringer's solution, and dextrose solution.

The kits or articles of manufacture described herein may have a number of embodiments. In one instance, the kits or articles of manufacture includes a container, a label on said container, and a composition contained within said container, wherein the composition includes one or more polynucleotides that hybridize to a complement of a gene listed herein (e.g., a gene set forth in Table 1, or any combination of genes set forth in Tables 2-12) under stringent conditions, and the label on said container indicates that the composition can be used to evaluate the presence of a gene listed herein (e.g., a gene set forth in Table 1, or any combination of genes set forth in Tables 2-12) in a sample, and wherein the kit includes instructions for using the polynucleotide(s) for evaluating the presence of the gene RNA or DNA in a particular sample type.

For oligonucleotide-based articles of manufacture or kits, the article of manufacture or kit can include, for example: (1) an oligonucleotide, e.g., a detectably labeled oligonucleotide, which hybridizes to a nucleic acid sequence encoding a protein or (2) a pair of primers useful for amplifying a nucleic acid molecule. The article of manufacture or kit can also include, e.g., a buffering agent, a preservative, or a protein stabilizing agent. The article of manufacture or kit can further include components necessary for detecting the detectable label (e.g., an enzyme or a substrate). The article of manufacture or kit can also contain a control sample or a series of control samples that can be assayed and compared to the test sample. Each component of the article of manufacture or kit can be enclosed within an individual container and all of the various containers can be within a single package, along with instructions for interpreting the results of the assays performed using the kit.

### VII. EXAMPLES

The following is an example of the methods of the invention. It is understood that various other embodiments may be practiced, given the general description provided above.

### Example 1: Sarcomatoid Histology, MSKCC Risk Scores, and Molecular Correlates Differentiate Response to Atezolizumab+Bevacizumab Versus Sunitinib: Results from a Phase III Study (IMmotion151) in Untreated Metastatic Renal Cell Carcinoma

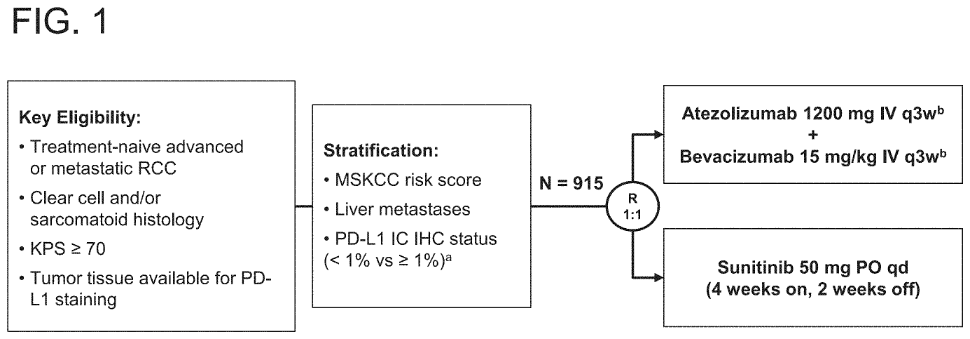

The IMmotion151 study (ClinicalTrials.gov Identifier NCT02420821) is a multi-center, randomized, open-label study to evaluate the efficacy and safety of atezolizumab plus bevacizumab versus sunitinib in patients with inoperable, locally advanced, or metastatic RCC who have not received prior systemic active or experimental therapy in either the adjuvant or metastatic setting. See FIG. 1. The co-primary endpoints of the study were PFS in the PD-L1⁺ subgroup, and OS in the ITT population. The exploratory endpoints included biomarker characterization in sarcomatoid tumors and MSKCC risk subgroups, as well as validation of gene signatures from the IMmotion150 study and their association with PFS.

Inclusion criteria for the IMmotion151 study included a definitive diagnosis of unresectable locally advanced or metastatic RCC with clear-cell histology and/or a component of sarcomatoid carcinoma, with no prior treatment in the metastatic setting; an evaluable MSKCC risk score; measurable disease, as defined by RECIST v1.1; a Karnofsky performance status 70%, and adequate hematologic and end-organ function prior to randomization. Disease-specific exclusions for the IMmotion151 study included radiotherapy for RCC within 14 days prior to treatment; active central nervous system disease, uncontrolled pleural effusion, pericardial effusion, or ascites; uncontrolled hypercalcemia; and any other malignancies within five years except for low-risk prostate cancer or those with negligible risk of metastasis or death. Exclusion criteria related to medications for the IMmotion151 study included prior treatment with cluster of differentiation 137 (CD137) agonists, anti-cytotoxic T-lymphocyte associated protein-4 (CTLA4), anti-programmed death (PD)-1, or anti-PD-L1 therapeutic antibody or pathway-targeting agents; treatment with immunostimulatory agents for non-malignant conditions within 6 weeks or immunosuppressive agents within 2 weeks prior to treatment; history of hypertensive crisis or hypertensive encephalopathy; and baseline electrocardiogram showing corrected QT interval greater than 460 milliseconds.

Sarcomatoid renal carcinoma was defined as any histologic type of renal cell carcinoma containing a focus/foci of high-grade malignant spindle cells of any component relative to the entire tumor area. Such a classification required evidence of epithelial differentiation with concurrent areas of renal cell carcinoma or evidence of epithelial differentiation in the spindle cells with immunohistochemical positivity for keratin or epithelial membrane antigen (EMA). Frequent patterns include fibrosarcoma, malignant fibrous histiocytoma, and rhabdomyosarcoma. Focal spindling due to noncohesion of tumor cells was not considered to represent sarcomatoid differentiation. Any spindle component relative to the entire tumor area was sufficient. The degree of sarcomatoid differentiation was recorded as 1) any component, 2) >20% component, or 3) predominant sarcomatoid component.

The MSKCC (Motzer) criteria used were as follows. The risk factors included 1) a Karnofsky Performance Status (KPS) score <80; 2) a corrected serum calcium >10 mg/dL; 3) an LDH level >1.5 times the upper limit of normal; 4) a hemoglobin level <the lower limit of normal; 5) a time from nephrectomy to systemic therapy of ≥12 months (an individual also was scored as having this risk factor if they were initially assessed with metastatic disease or if they have had no nephrectomy). The risk stratification was as follows: individuals having 3 risk factors belong to the poor risk subgroup; individuals having 1 or 2 risk factors belong to the intermediate risk subgroup; and individuals having 0 risk factors belong to the favorable group. For study purposes, systemic therapy was designated as the date of initial study screening. The formula for corrected calcium was Corrected calcium=serum calcium (mg/dL)+0.8 (4 0 serum albumin (g/dL)).

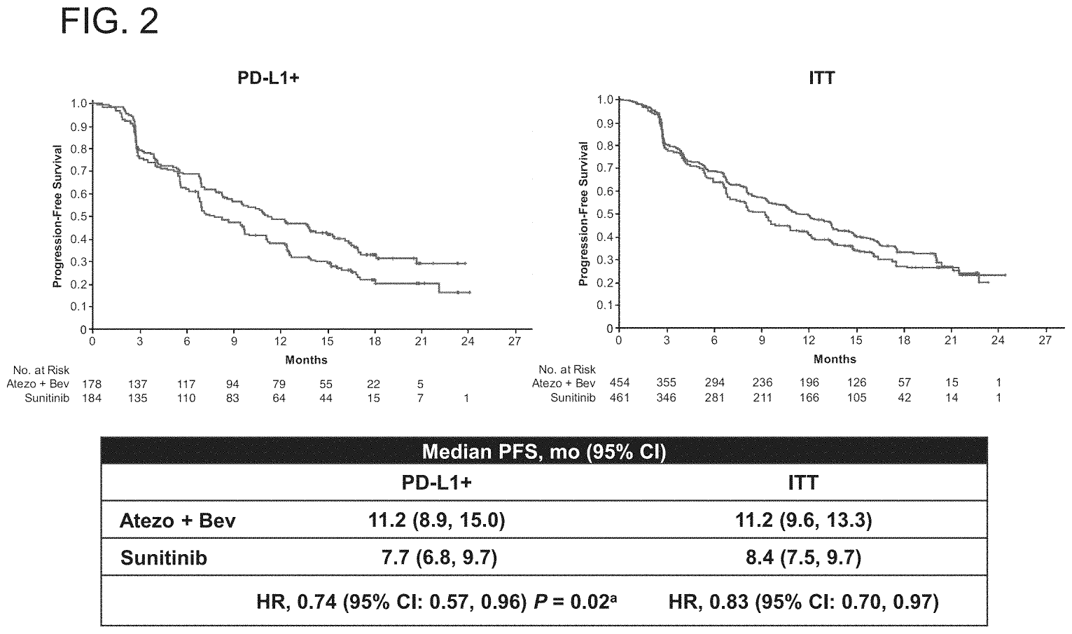

Patients in the Atezolizumab+Bevacizumab (“Atezo+Bev”) arm received both atezolizumab and bevacizumab until loss of clinical benefit, unacceptable toxicity or symptomatic deterioration attributed to disease progression, withdrawal of consent, or death, whichever occured first. Atezolizumab was administered at a fixed dose of 1200 milligrams (mg) via intravenous (IV) infusion on Days 1 and 22 of each 42-day cycle. Bevacizumab was be administered at a dose of 15 milligrams per kilogram (mg/kg) via IV infusion on Days 1 and 22 of each 42-day cycle. Patients in the sunitinib arm received sunitinib until loss of clinical benefit, unacceptable toxicity or symptomatic deterioration attributed to disease progression, withdrawal of consent, or death, whichever occured first. Sunitinib was administered at a dose of 50 mg once daily, orally via capsule, on Day 1 through Day 28 of each 42-day cycle. A summary of the PFS results in the PD-L1+ subgroup and the ITT population is shown in FIG. 2. For PD-L1+ patients, the median PFS in the Atezo+Bev arm was 11.2 months, compared to 7.7 months in the sunitinib arm, with a hazard ratio of 0.74 (P=0.02). The PFS analysis passed the pre-specified P value boundary of α=0.04. In the ITT population, the median PFS in the Atezo+Bev arm was 11.2 months, compared to 8.4 months in the sunitinib arm, with a hazard ratio of 0.83.

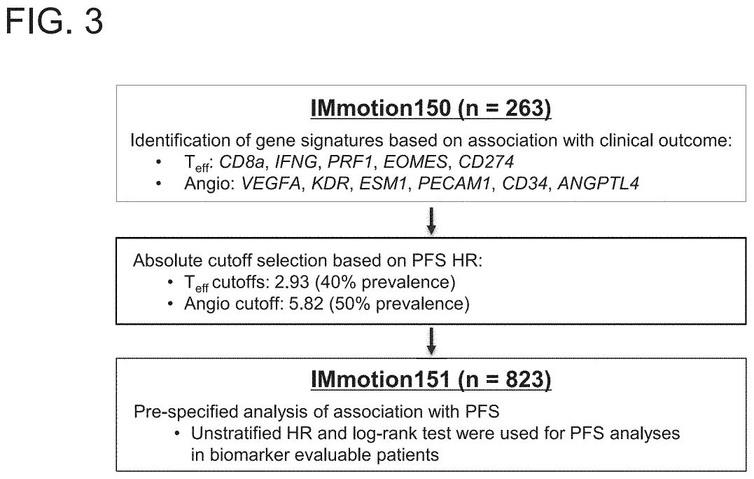

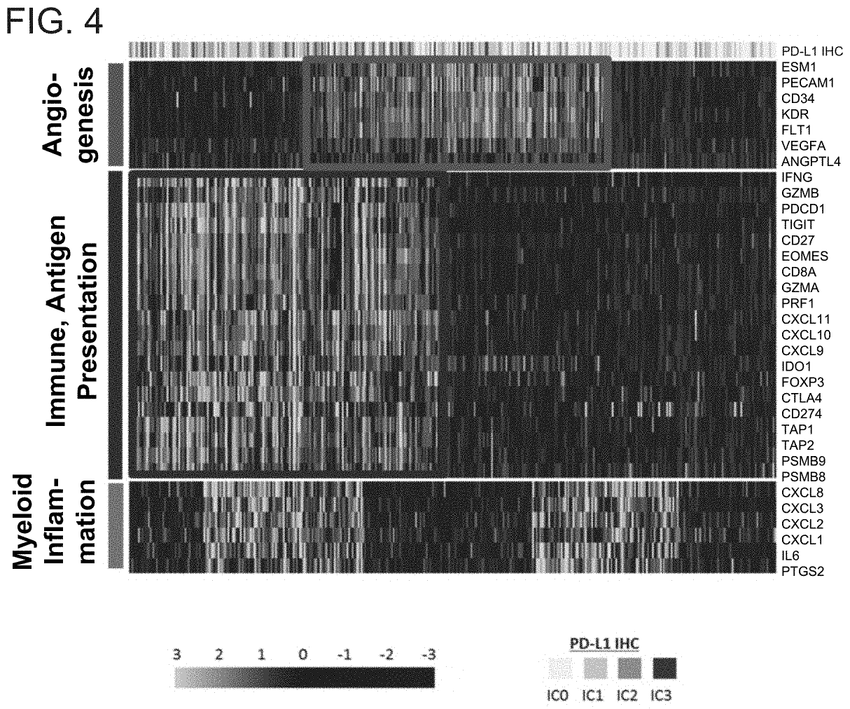

The gene signature analysis scheme for the IMmotion151 study is shown in FIG. 3. In the phase II IMmotion150 study, gene signatures were identified based on association with clinical outcome. The T-effector (“Teff”) signature included CD8a, IFNG, PRF1, EOMES, and CD274. The Angiogenesis (“Angio”) signature included VEGFA, KDR, ESM1, PECAM1, CD34, and ANGPTL4. Absolute cutoff selection based on PFS HR resulted in a Teff cutoff of 2.93 (40% prevalence) and an Angio cutoff of 5.82 (50% prevalence). In the IMmotion151 study, a pre-specified analysis of association with PFS was performed. Unstratified HR and log-rank test were used for PFS analyses in biomarker-evaluable patients. The IMmotion151 transcriptome map confirmed biological subgroups identified in IMmotion151, including the angiogenesis, immune and antigen presentation (including the Teff signature), and myeloid inflammation subgroups (FIG. 4).

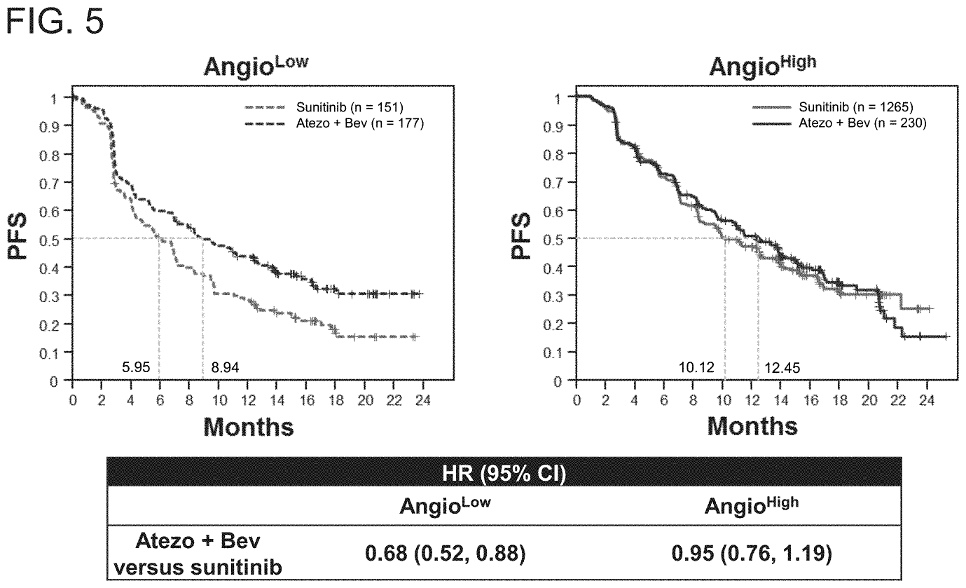

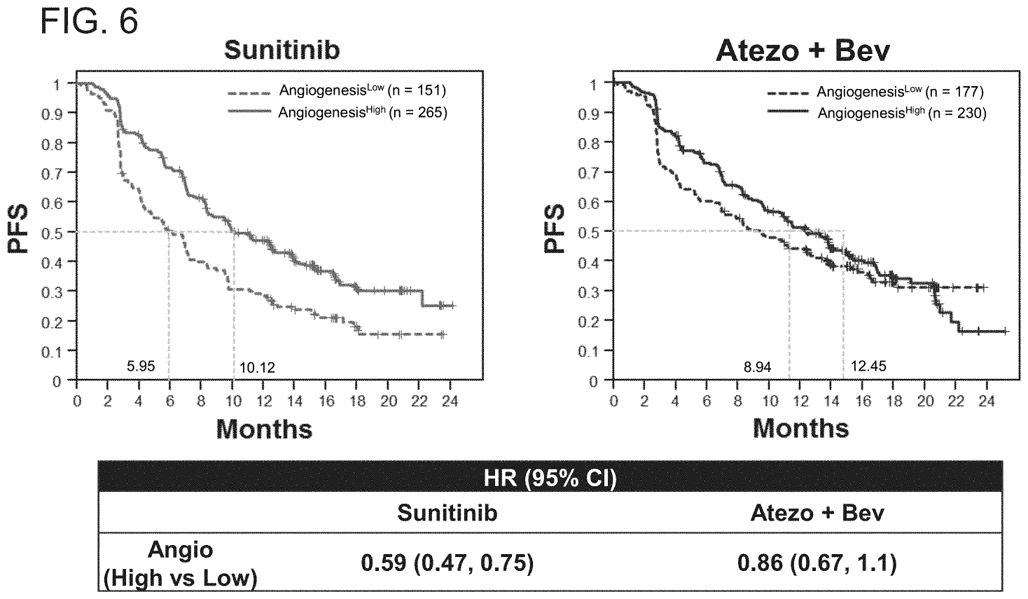

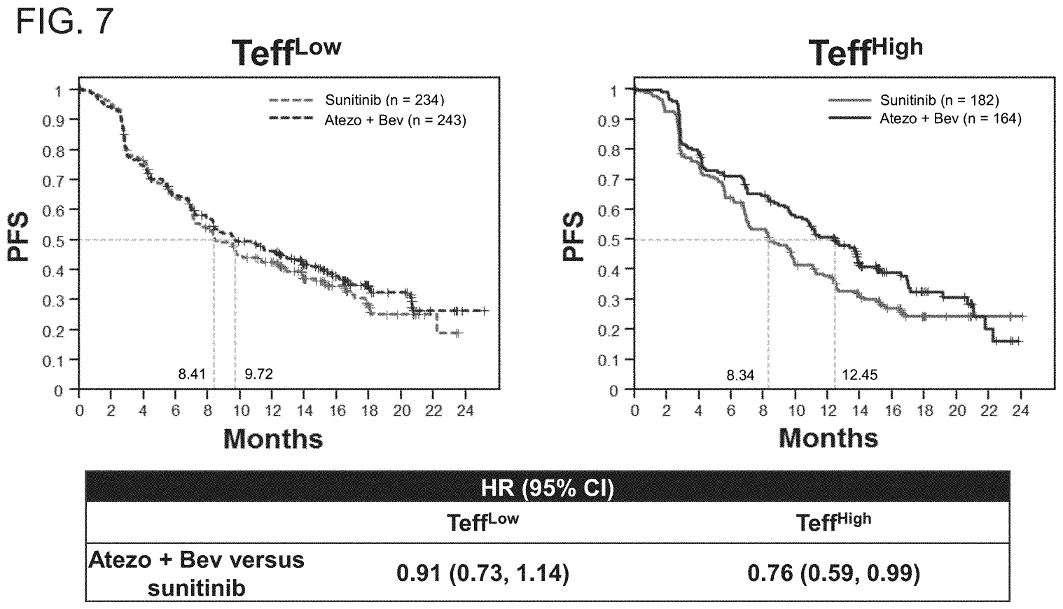

Atezo+Bev improved PFS versus sunitinib in the Angio^{Low} subgroup (FIG. 5). The HR (95% CI) for Atezo+Bev versus sunitinib in the Angio^{Low} subgroup was 0.68 (0.52, 0.88), and in the Angio^{High} subgroup was 0.95 (0.76, 1.19). Sunitinib demonstrated improved PFS in the Angio^{High} subgroup versus the Angio^{Low} subgroup (FIG. 6). The HR (95% CI) for Angiogenesis (High versus Low) was 0.59 (0.47, 0.75) for sunitinib, and 0.86 (0.67, 1.1) for Atezo+Bev. Atezo+Bev demonstrated improved PFS versus sunitinib in the Teff^{High} subgroup (FIG. 7). The HR (95% CI) for Atezo+Bev versus sunitinib in the Teff^{Low} subgroup was 0.91 (0.73, 1.14), and in the Teff^{High} subgroup was 0.76 (0.59, 0.99). The Teff gene signature did not differentiate PFS within the sunitinib or Atezo+Bev treatment arms. In summary, the pre-specified analyses in IMmotion151 validated the Angio and Teff gene signatures identified in IMmotion150. In particular, Atezo+Bev improved PFS versus sunitinib in Teff^{High} and in Angio^{Low} tumors, and within the sunitinib arm, patients with an Angio^{High} gene signature showed improved PFS versus the Angio^{Low} gene signature group.

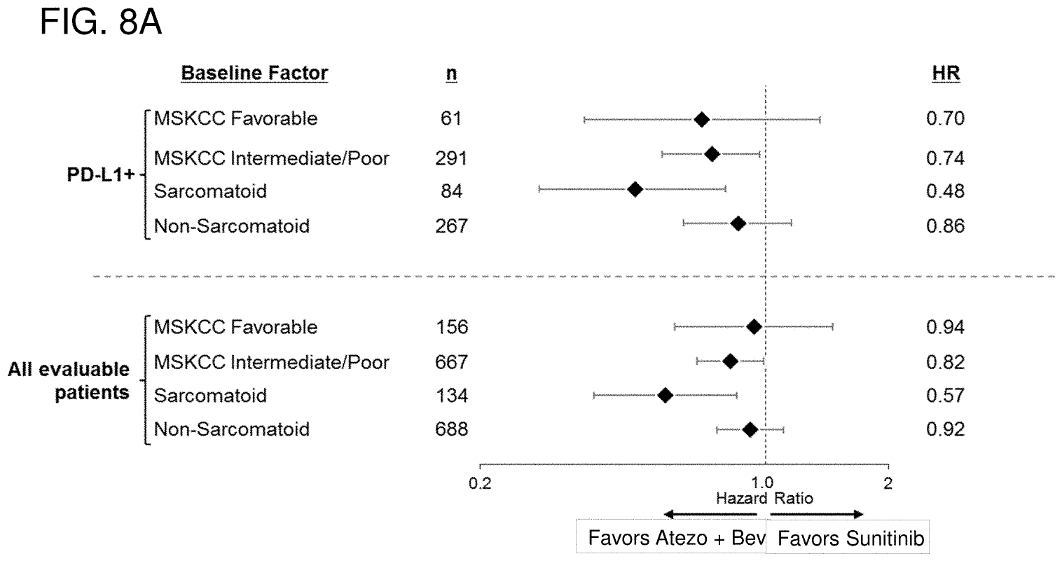

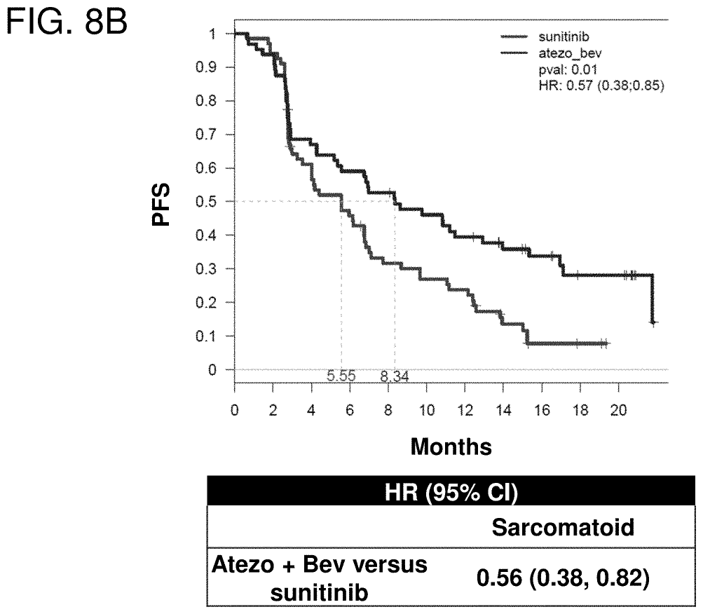

An analysis of subgroup PFS in the PD-L1+ and all evaluable patients (the biomarker-evaluable population) is shown in FIG. 8A. Sarcomatoid differentiation is an independent predictor of poor survival and poor response to VEGF inhibition (see, e.g., El Mouallem et al. Urol. Oncol. 36:265-271, 2018). In IMmotion151, 16% of patients showed sarcomatoid differentiation. The overall response rate (ORR) in patients with sarcomatoid differentiation was 56% in the atezolizumab+bevacizumab arm and 18% in the sunitinib arm. Atezo+Bev demonstrated improved PFS in sarcomatoid tumors (FIGS. 8A and 8B). In summary, these data demonstrate that the presence of sarcomatoid kidney cancer (e.g., sarcomatoid RCC) can be used to identify patients who are likely to benefit from treatment with an anti-cancer therapy that includes a VEGF antagonist (e.g., bevacizumab) and a PD-L1 axis binding antagonist (e.g., atezolizumab), as well as for patient selection and stratification.

Atezo+Bev also demonstrated improved PFS in patients with poor or intermediate MSKCC risk scores (FIG. 8A). This effect was observed both in PD-L1⁺ patients and in the biomarker-evaluable population (FIG. 8A). These data demonstrate that the MSKCC risk score, and in particular, the presence of a poor or intermediate MSKCC risk score, can be used to identify patients who are likely to benefit from treatment with an anti-cancer therapy that includes a VEGF antagonist (e.g., bevacizumab) and a PD-L1 axis binding antagonist (e.g., atezolizumab), as well as for patient selection and stratification.

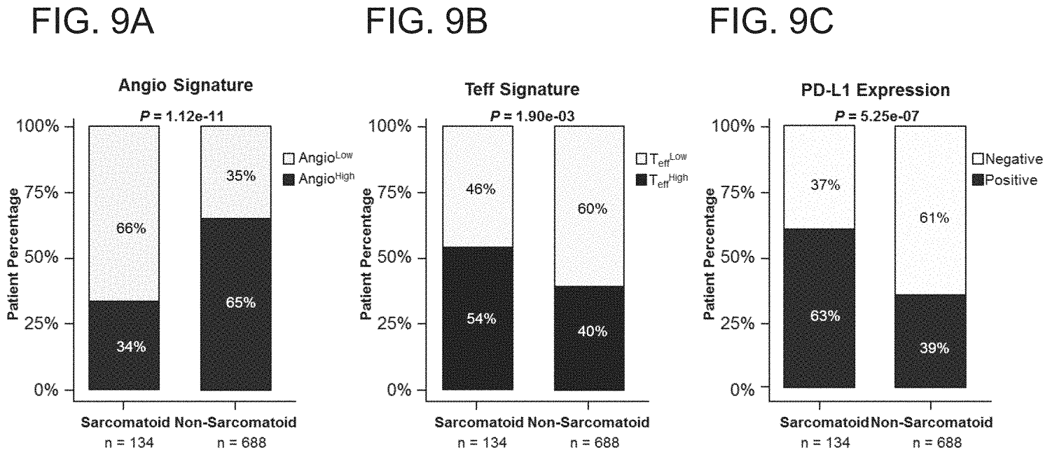

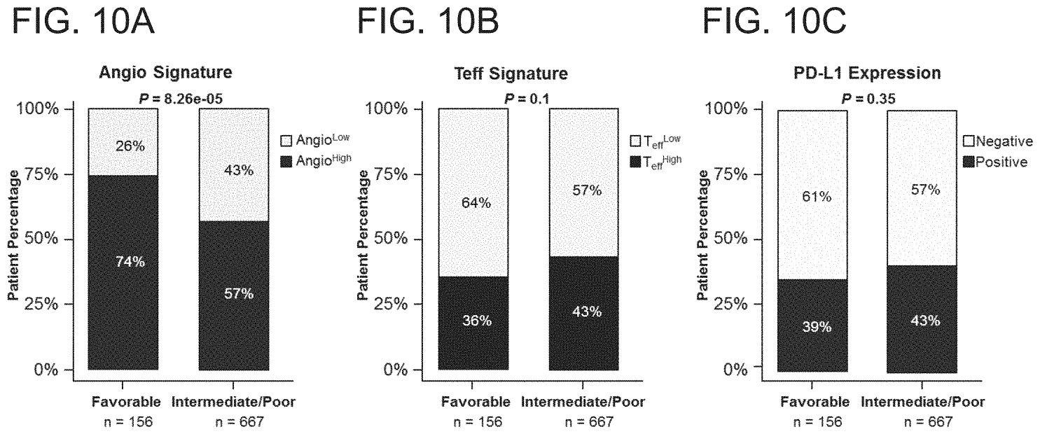

Expression of the Angio gene signature, the Teff gene signature, and PD-L1 was evaluated in the sarcomatoid versus non-sarcomatoid (FIGS. 9A-9C) and MSKCC risk score (FIGS. 10A-10C) subgroups. Angio gene signature expression was lower lower and PD-L1 expression was higher in sarcomatoid tumors compared to non-sarcomatoid tumors (FIGS. 9A and 9C). Angio gene signature expression was higher in the favorable MSKCC risk score subgroup compared to the intermediate/poor risk score subgroup (FIG. 10A). Overall, these data show that sarcomatoid RCC is characterized by higher PD-L1 expression and lower Angio gene signature expression compared to non-sarcomatoid tumors, while MSKCC favorable risk patients are characterized by a higher Angio gene signature expression as well as similar Teff gene signature and PD-L1 expression levels compared to MSKCC intermediate/poor risk score patients.

In summary, the data provided herein demonstrate that the presence of sarcomatoid kidney cancer or the presence of a poor or intermediate MSKCC risk score can be used to identify patients who are likely to benefit (e.g., in terms of PFS) from an anti-cancer therapy that includes a VEGF antagonist (e.g., bevacizumab) and a PD-L1 axis binding antagonist (e.g., atezolizumab). These data can be used for personalized therapy of patients having kidney cancer (e.g., RCC (e.g., mRCC)), for example, for treatment with an anti-cancer therapy that includes a VEGF antagonist (e.g., bevacizumab) and a PD-L1 axis binding antagonist (e.g., atezolizumab), as well as for patient selection and stratification for an optimized anti-cancer therapy.

### Example 2: Atezolizumab+Bevacizumab Versus Sunitinib in Patients with Untreated Metastatic Renal Cell Carcinoma and Sarcomatoid Histology: IMmotion151 Subgroup Analysis

As is described in Example 1, renal cell carcinoma (RCC) with sarcomatoid histology is characterized by the presence of spindle-shaped malignant epithelial cells. Sarcomatoid histology is associated with multiple histological subtypes of RCC and confers an aggressive phenotype. Patients who have metastatic RCC with sarcomatoid histology (approximately 10%-20% of patients with advanced disease) have a particularly poor prognosis and limited response to inhibition of the vascular endothelial growth factor pathway. Here we report results of a prespecified subgroup analysis in patients enrolled in IMmotion151 whose tumors had sarcomatoid histology in order to assess the effect of atezolizumab+bevacizumab relative to that of sunitinib and explore biological correlates of sarcomatoid versus non-sarcomatoid histologies.

Methods

IMmotion151 is a multicenter, randomized, open-label, Phase III study evaluating the efficacy and safety of atezolizumab+bevacizumab vs those of sunitinib in patients with previously untreated, inoperable, locally advanced or metastatic RCC (FIG. 1). The co-primary endpoints were (i) investigator (INV)-assessed progression-free survival (PFS) in patients with ≥1% IC expressing PD-L1 (PD-L1+) and (ii) interim overall survival (OS) in the intent-to-treat (ITT) population. Secondary endpoints inclue INV-assessed PFS and OS in patients with sarcomatoid histology and are reported here with INV-assessed objective response rate (ORR), safety, patient-reported outcomes (PROs) of symptoms and function and biomarker evaluation.

Patients were included in this subgroup analysis if their tumor had any component of sarcomatoid histology, referred to in this presentation as “All Sarc,” as reported by the investigator per the local pathology report. P values are for descriptive purposes only. Gene expression analyses of baseline tumor samples were performed as previously described and focused on T-effector and angiogenesis signatures (see, e.g., McDermott et al. Nat. Med. 24:749-757, 2018). The clinical cutoff for PFS, ORR, PRO and safety was Sep. 29, 2017, with a median follow-up of 13 months. The clinical cutoff for OS was Aug. 13, 2018, with a median follow-up of 17 months.

Results

Patients with sarcomatoid histology were more likely to have PD-L1+ disease and intermediate/poor risk than patietns in the ITT population (Table 18).

TABLE 18
Baseline Characteristics

| | All Sarc | | ITT | |
|---|---|---|---|---|
| | Atezo + Bev | Sunitinib | Atezo + Bev | Sunitinib |
| Characteristic | n = 68 | n = 74 | n = 454 | n = 461 |
| Median age | 59 (24-79) | 59 (28-81) | 62 (24-88) | 60 (18-84) |
| (range), years | | | | |
| Male, n (%) | 40 (59) | 55 (74) | 317 (70) | 352 (76) |
| KPS < 80, n (%) | 8 (12) | 4 (5) | 40 (9) | 35 (8) |
| Liver metastasis, | 14 (21) | 16 (22) | 78 (17) | 82 (18) |
| n (%) | | | | |
| Prior nephrectomy, | 55 (81) | 55 (74) | 334 (74) | 330 (72) |
| n (%) | | | | |
| Any sarcomatoid | 68 (100) | 74 (100) | 68 (15) | 74 (16) |
| component, n (%) | | | | |
| Component of | 27 (44) | 25 (40) | 27 (44) | 25 (40) |
| sarcomatoid > | | | | |
| 20%, n (%)* | | | | |
| PD-L1+, n (%) | 36 (53) | 50 (68) | 178 (39) | 184 (40) |
| MSKCC risk | | | | |
| category, n (%) | | | | |
| Favorable (0) | 3 (4) | 8 (11) | 89 (20) | 90 (20) |
| Intermediate (1 or 2) | 48 (71) | 56 (76) | 311 (69) | 318 (69) |
| Poor (≥3) | 17 (25) | 10 (14) | 54 (12) | 53 (12) |

Atezo, atezolizumab; Bev, bevacizumab; Sarc, sarcomatoid.
*Denominator is based on the number of > 20% component of sarcomatoid - evaluable patients (n = 62 for each treatment arm).

Efficacy was evaluated in patients with sarcomatoid histology, both overall and in patients with PD-L1+ tumors (Table 19). In patients with >20% sarcomatoid component (n=27 in the atezolizumab+bevacizumab arm and n=25 in the sunitinib arm, respectively), ORR was 44% versus 4% and CR rate was 7% versus 0%, respectively. PFS and ORR assessments by the investigator and independent review committee were consistent in the sarcomatoid populations.

TABLE 19
Efficacy Summary

| | All Sarc | | ITT¹ | | PD-Li + Sarc | | PD-L1 + ITT¹ | |
|---|---|---|---|---|---|---|---|---|
| | Atezo + | | Atezo + | | Atezo + | | Atezo + | |
| | Bev | Sunitinib | Bev | Sunitinib | Bev | Sunitinib | Bev | Sunitinib |
| | n = 68 | n = 74 | n = 454 | n = 461 | n = 36 | n = 50 | n = 178 | n = 184 |
| Median INV- | 8.3 | 5.3 | 11.2 | 8.4 | 8.6 | 5.6 | 11.2 | 7.7 |
| assessed PFS | (5.4, 12.9) | (3.3, 6.7) | (9.6, 13.3) | (7.5, 9.7) | (3.9, 15.3) | (3.3, 6.7) | (8.9, 15.0) | (6.8, 9.7) |
| (95% Cl), mo^{a} | | | | | | | | |
| Stratified HR | 0.52 | | 0.83 | | 0.45 | | 0.74 | |
| (95% Cl) | (0.34, 0.79) | | (0.70, 0.97) | | (0.26, 0.77) | | (0.57, 0.96) | |
| Confirmed INV- | 49 | 14 | 37 | 33 | 56 | 12 | 43 | 35 |
| assessed ORR | (36, 61) | (7, 23) | (32, 41) | (29, 38) | (38, 72) | (5, 24) | (35, 50) | (28, 42) |
| (95% CI), %^{a} | | | | | | | | |
| Ongoing | 17 | 3 | 107 | 90 | 9 | 0 | 49 | 34 |
| responses, | | | | | | | | |
| n | | | | | | | | |
| CR, % | 10 | 3 | 5 | 2 | 14 | 4 | 9 | 4 |
| Median OS | 21.7 | 15.4 | 33.6 | 34.9 | 19.3 | 15.0 | 34.0 | 32.7 |
| (95% Cl), mo^{b} | (15.3, NE) | (10.4, 19.5) | (29.0, NE) | (27.8, NE) | (14.8, NE) | (8.4, 19.5) | (28.6, NE) | (23.3, NE) |

| | | | | |
|---|---|---|---|---|
| Stratified HR | 0.64 | 0.93 | 0.61 | 0.84 |
| (95% Cl) | (0.41, 1.01) | (0.76, 1.14) | (0.35, 1.08) | (0.62, 1.15) |

CR, complete response; HR, hazard ratio; NE, not estimable.
^{a}Clinical cutoff: Sep. 29, 2017.
^{b}Clinical cutoff: Aug. 13, 2018.
¹Rini et al. pii:S0140-6736(10)30723-8 Lancet 2019 [epub ahead of print]; dx.doi.org/10.1016/S0140-6736(19)30723-8.

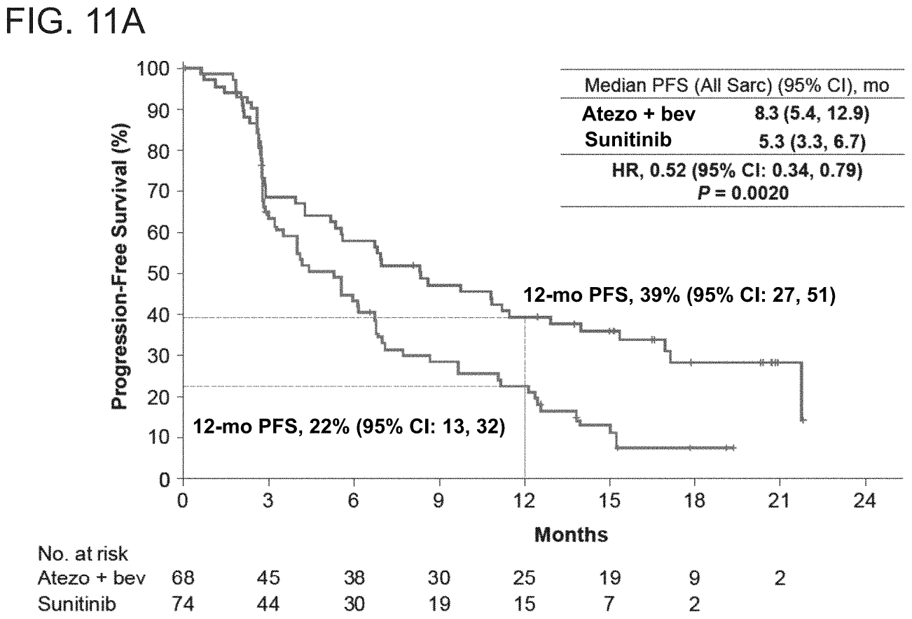

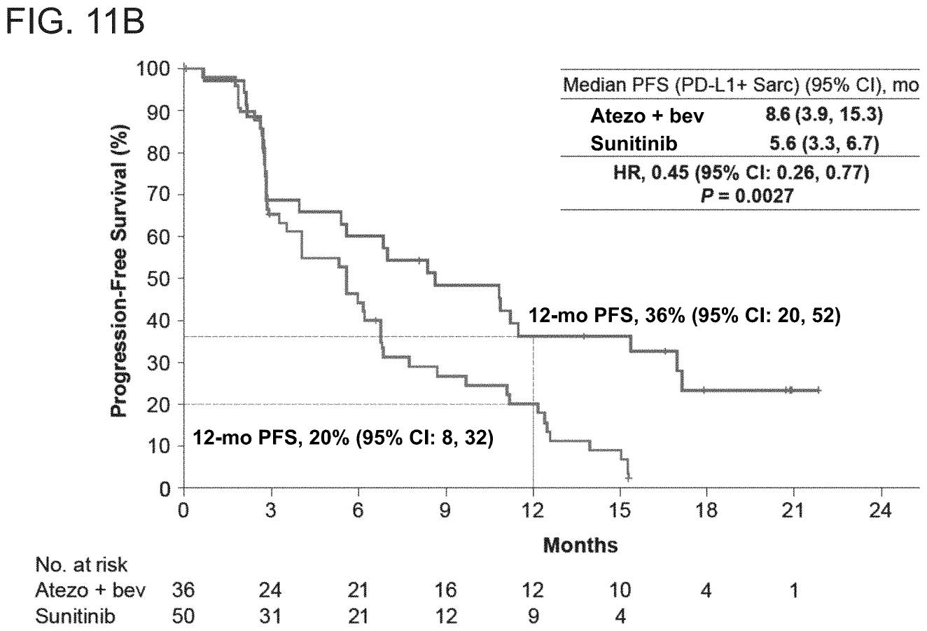

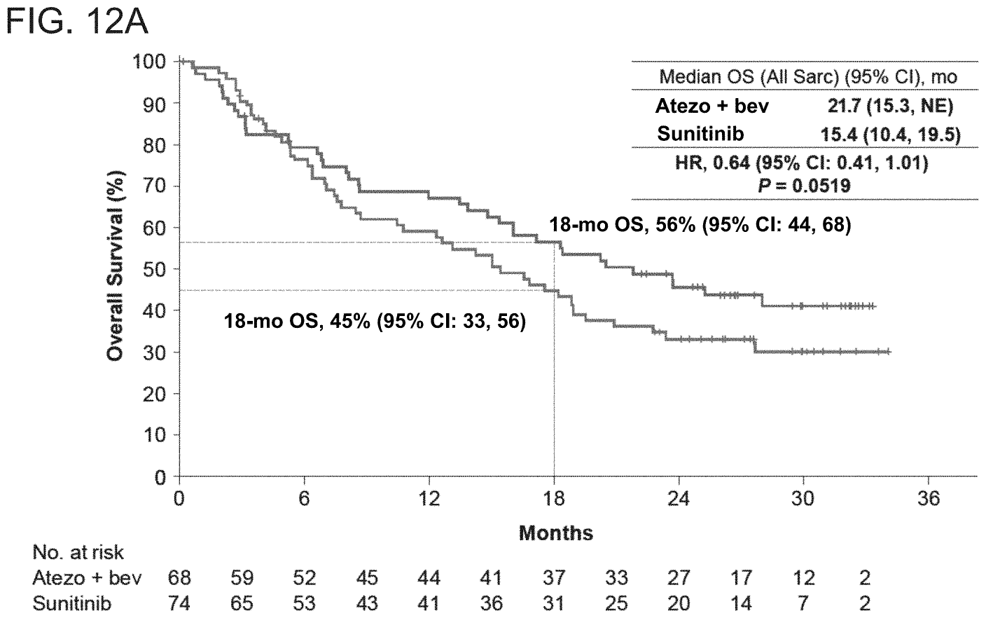

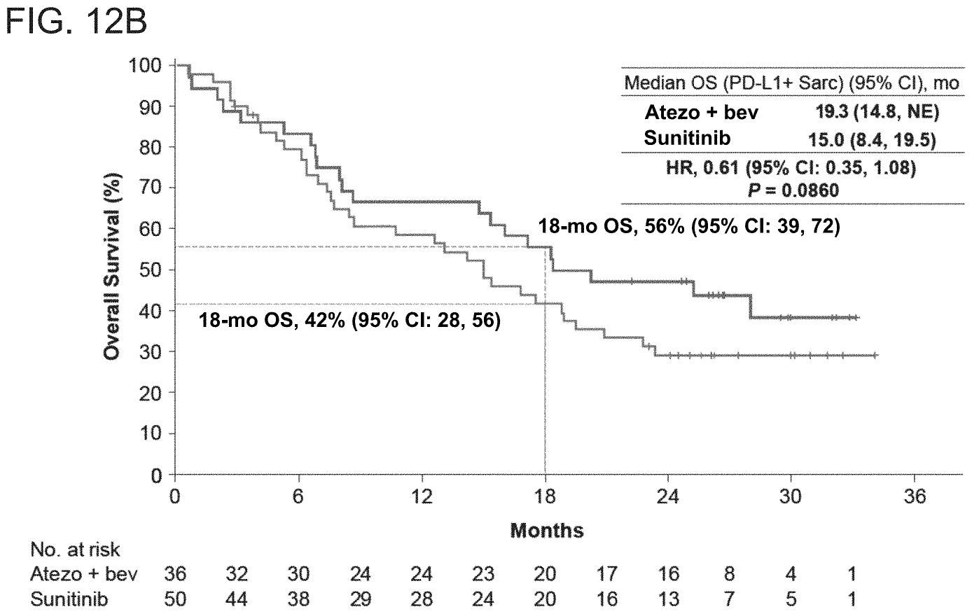

Patients with sarcomatoid histology in the atezolizumab+bevacizumab arm had a longer median PFS than those in the sunitinib arm, regardless of PD-L1⁺ status (FIGS. 11A and 11B). The difference in median PFS was more pronounced between treatment arms in the sarcomatoid than in the ITT population (Table 19). OS was increased in patients with sarcomatoid histology treated with atezolizumab+bevacizumab versus those treated with sunitinib, regardless of PD-L1⁺ status (FIGS. 12A and 12B). The difference in median OS was more pronounced between treatment arms in the sarcomatoid than in the ITT population (Table 19).

Safety in patients with sarcomatoid tumors in the atezolizumab+bevacizumab arm was generally consistent with the known safety profile of each treatment component and in line with that in the overall safety-evaluable population (Tables 20 and 21). Approximately 12% of patients (n=8) with sarcomatoid histology treated with atezolizumab+bevacizumab required systemic corticosteroid use (6% [n=4] required prednisone 40 mg per day) within 30 days of an AESI. No new safety signals were identified.

TABLE 20
Safety Summary

| | Sarc | | All | |
|---|---|---|---|---|
| | Safety Evaluable | | Safety Evaluable | |
| | Atezo + Bev | Sunitinib | Atezo + Bev | Sunitinib |
| | n = 67 | n = 70 | n = 451 | n = 446 |
| Median treatment | 7.7 | 4.1 | 12.0 | 9.2 |
| duration (range), mo | (0, 23.1) | (0.4, 21.3) | (0, 26.2) | (0, 26.6) |
| Treatment- | 90 | 94 | 91 | 96 |
| related AEs, % | | | | |
| Grade 3-4, % | 40 | 49 | 40 | 54 |
| Treatment-related | 3 | 3 | 5 | 8 |
| AEs leading to | | | | |
| discontinuation of | | | | |
| treatment regimen, % | | | | |
| Treatment-related | 6^{a} | 3 | 12^{b} | 8 |
| AEs leading to | | | | |
| discontinuation | | | | |
| of any treatment | | | | |
| component, % | | | | |
| Treatment-related | 1^{c} | 0 | 5^{d} | 1^{e} |
| deaths, n | | | | |

AE, adverse event.
Clinical cutoff: Sep. 29, 2017.
^{a}Atezo + bev, 3.0%; atezo only, 1.5%; bev only, 1.5%.
^{b}Atezo + bev, 5.3%; atezo only, 2.0%; bev only, 5.1%.
^{c}Sepsis.
^{d}Cerebral infarction, intracranial hemorrhage, adrenal insufficiency, multiple organ dysfunction syndrome, sepsis.
^{e}Cardiac arrest.

TABLE 21
Adverse Events of Special Interest (AESI) With Atezolizumab
(includes all treatment-emergent AEs)

| | Sarc | | | | All | | | |
|---|---|---|---|---|---|---|---|---|
| | Safety Evaluable | | | | Safety Evaluable | | | |
| | Atezo + Bev | | Sunitinib | | Atezo + Bev | | Sunitinib | |
| | n = 67 | | n = 70 | | n = 451 | | n = 446 | |
| Patients with ≥ | All | Grade | All | Grade | All | Grade | All | Grade |
| 1 event, % | Grades | 3-4 | Grades | 3-4 | Grades | 3-4 | Grades | 3-4 |
| Any AESI | 51 | 8 | 66 | 11 | 61 | 12 | 72 | 17 |
| Rash | 22 | 0 | 14 | 0 | 19 | <1 | 15 | <1 |
| Hypothyroidism | 12 | 0 | 16 | 0 | 22 | <1 | 26 | <1 |
| Hyperthyroidism | 6 | 0 | 1 | 0 | 7 | <1 | 3 | 0 |
| Adrenal | 2 | 0 | 0 | 0 | 2 | 0 | 0 | 0 |
| insufficiency | | | | | | | | |
| Liver function | 9 | 5 | 10 | 3 | 10 | 3 | 18 | 4 |
| test | | | | | | | | |
| abnormalities | | | | | | | | |
| Colitis | 0 | 0 | 1 | 0 | 2 | <1 | <1 | <1 |
| Pneumonitis | 3 | 0 | 0 | 0 | 3 | <1 | 0 | 0 |

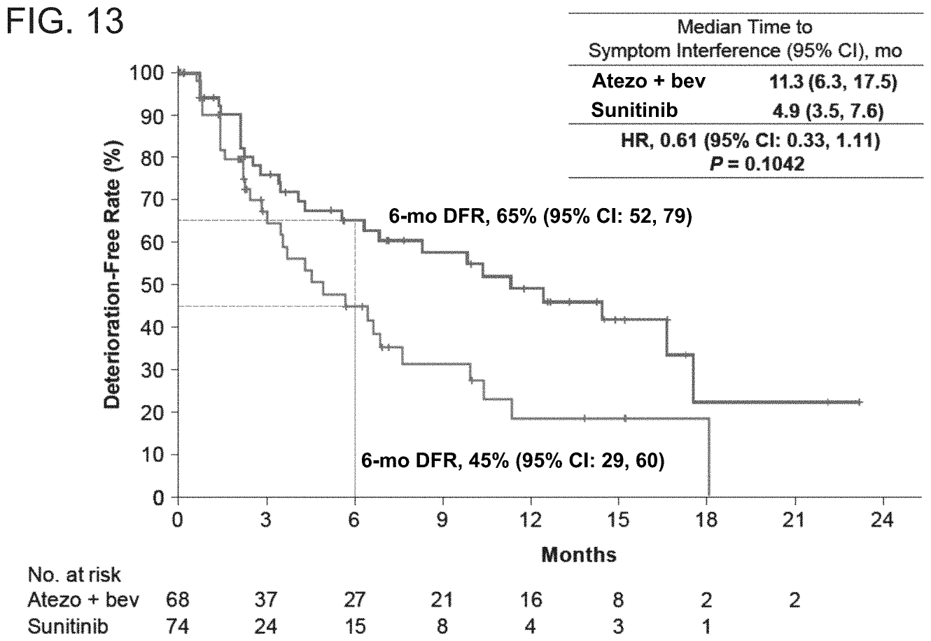

Patients with sarcomatoid histology treated with atezolizumab+bevacizumab reported a longer median time to deterioration of symptom interference with daily living than those treated with sunitinib (FIG. 13).

PD-L1 expression was higher in sarcomatoid vs non-sarcomatoid tumors (FIG. 9C). Prevalence of the Angiogenesis^{High} gene expression signature subset was lower and T-effector^{High} gene expression subset was higher in sarcomatoid versus non-sarcomatoid tumors (FIGS. 9A and 9B).

### CONCLUSIONS

Pre-specified analyses of patients with sarcomatoid histology enrolled in IMmotion151 suggest enhanced clinical efficacy of atezolizumab+bevacizumab versus sunitinib. Patients with sarcomatoid histology had longer PFS and OS and higher ORR, including complete responses, with atezolizumab+bevacizumab versus sunitinib regardless of PD-L1 status. The safety profile for the atezolizumab+bevacizumab combination was as expected for single agents and in line with that in the overall safety-evaluable population; no new safety signals were identified. Patients with sarcomatoid histology treated with atezolizumab+bevacizumab reported a longer median time to symptom interference with daily function than those treated with sunitinib. Biomarker data from tumors with sarcomatoid histology (Angiogenesis^{Low}; T-effector^{High}; PD-L1+) provide a biological correlate for the increased responsiveness to atezolizumab+bevacizumab in patients with metastatic RCC and a component of sarcomatoid histology. These data further demonstrate that patients with sarcomatoid differentiation are likely to benefit from immune checkpoint inhibitor therapy, e.g., therapy with a PD-L1 axis binding antagonist (e.g., an anti-PD-L1 antibody such as atezolizumab), including combination therapies that include a PD-L1 axis binding antagonist and a VEGF antagonist (e.g., an anti-VEGF antibody such as bevacizumab).

### VIII. Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method of treating an individual having a kidney cancer, the method comprising administering to the individual an effective amount of an anti-cancer therapy comprising a VEGF antagonist and a PD-L1 axis binding antagonist, wherein the individual has been identified as likely to benefit from the anti-cancer therapy based on having a sarcomatoid kidney cancer.

2. A method of treating an individual having a kidney cancer, the method comprising:
(a) determining whether the individual has a sarcomatoid kidney cancer, wherein the presence of a sarcomatoid kidney cancer indicates that the individual is likely to benefit from an anti-cancer therapy comprising a VEGF antagonist and a PD-L1 axis binding antagonist; and
(b) administering an effective amount of an anti-cancer therapy comprising a VEGF antagonist and a PD-L1 axis binding antagonist to the individual based on the presence of a sarcomatoid kidney cancer.

3. A method of identifying an individual having a kidney cancer who may benefit from treatment with an anti-cancer therapy comprising a VEGF antagonist and a PD-L1 axis binding antagonist, the method comprising determining whether the individual has a sarcomatoid kidney cancer, wherein the presence of a sarcomatoid kidney cancer identifies the individual as one who may benefit from treatment with an anti-cancer therapy comprising a VEGF antagonist and a PD-L1 axis binding antagonist.

4. A method for selecting a therapy for an individual having a kidney cancer, the method comprising
(a) determining whether the individual has a sarcomatoid kidney cancer, wherein the presence of a sarcomatoid kidney cancer identifies the individual as one who may benefit from treatment with an anti-cancer therapy comprising a VEGF antagonist and a PD-L1 axis binding antagonist; and
(b) selecting an anti-cancer therapy comprising a VEGF antagonist and a PD-L1 axis binding antagonist based on the presence of a sarcomatoid kidney cancer.

5. The method of claim 3 or 4, further comprising administering an effective amount of an anti-cancer therapy comprising a VEGF antagonist and a PD-L1 axis binding antagonist to the individual.

6. The method of any one of claims 1-5, wherein the presence of a sarcomatoid kidney cancer is assessed by histological analysis of a sample obtained from the individual.

7. The method of claim 6, wherein the kidney cancer is sarcomatoid if a tumor sample from the individual contains a focus or foci of high-grade malignant spindle cells of any component relative to the entire tumor area.

8. The method of claim 6 or 7, wherein the spindle cells show moderate to marked atypia and/or resemble any form of sarcoma.

9. The method of claim 7 or 8, wherein the spindle cells show evidence of epithelial differentiation as assessed by immunohistological positivity for keratin or epithelial membrane antigen (EMA).

10. The method of claim 7 or 8, wherein the kidney cancer is renal cell carcinoma, and the tumor sample has epithelial differentiation with concurrent areas of renal cell carcinoma.

11. The method of any one of claims 1-10, wherein the benefit is in terms of improved progression-free survival (PFS), overall survival (OS), overall response rate (ORR), complete response (CR) rate, or deterioration-free rate (DFR).

12. The method of claim 11, wherein the benefit is in terms of improved PFS.

13. The method of claim 11, wherein the benefit is in terms of improved OS.

14. The method of claim 11, wherein the benefit is in terms of improved ORR.

15. The method of claim 11, wherein the benefit is in terms of improved CR rate.

16. The method of claim 11, wherein the benefit is in terms of improved DFR.

17. The method of claim 16, wherein DFR is determined in terms of the time from onset of treatment to the individual's first increase of greater than or equal to 2 points above baseline on the MD Anderson Symptom Inventory (MDASI) interference scale.

18. The method of any one of claims 1-17, wherein the individual has a poor or intermediate Memorial Sloan Kettering Cancer Center (MSKCC) risk score.

19. A method of treating an individual having a kidney cancer, the method comprising administering to the individual an effective amount of an anti-cancer therapy comprising a VEGF antagonist and a PD-L1 axis binding antagonist, wherein the individual has been identified as likely to benefit from the anti-cancer therapy based on the individual having a poor or intermediate MSKCC risk score.

20. A method of treating an individual having a kidney cancer, the method comprising:
(a) determining the individual's MSKCC risk score, wherein a poor or intermediate MSKCC risk score indicates that the individual is likely to benefit from an anti-cancer therapy comprising a VEGF antagonist and a PD-L1 axis binding antagonist; and
(b) administering an effective amount of an anti-cancer therapy comprising a VEGF antagonist and a PD-L1 axis binding antagonist to the individual based on the individual having a poor or intermediate MSKCC risk score.

21. A method of identifying an individual having a kidney cancer who may benefit from treatment with an anti-cancer therapy comprising a VEGF antagonist and a PD-L1 axis binding antagonist, the method comprising determining the individual's MSKCC risk score, wherein a poor or intermediate MSKCC risk score identifies the individual as likely to benefit from an anti-cancer therapy comprising a VEGF antagonist and a PD-L1 axis binding antagonist.

22. A method for selecting a therapy for an individual having a kidney cancer, the method comprising
(a) determining the individual's MSKCC risk score, wherein a poor or intermediate MSKCC risk score identifies the individual as likely to benefit from an anti-cancer therapy comprising a VEGF antagonist and a PD-L1 axis binding antagonist; and
(b) selecting an anti-cancer therapy comprising a VEGF antagonist and a PD-L1 axis binding antagonist based on the individual having a poor or intermediate MSKCC risk score.

23. The method of any one of claims 18-22, wherein the individual has a poor MSKCC risk score if the individual has three or more of the following characteristics:
(i) a time from nephrectomy to systemic treatment of less than one year, a lack of a nephrectomy, or an initial diagnosis with metastatic disease;
(ii) a hemoglobin level less than the lower limit of normal (LLN), optionally wherein the normal range for hemoglobin is between 13.5 and 17.5 g/dL for men and between 12 and 15.5 g/dL for women;
(iii) a serum corrected calcium level greater than 10 mg/dL, optionally wherein the serum corrected calcium level is the serum calcium level (mg/dL)+0.8(4−serum albumin (g/dL));
(iv) a serum lactate dehydrogenase (LDH) level greater than 1.5 times the upper limit of normal (ULN), optionally wherein the ULN is 140 U/L; and/or
(v) a Karnofsky Performance Status (KPS) score of <80.

24. The method of any one of claims 18-22, wherein the individual has an intermediate MSKCC risk score if the individual has one or two of the following characteristics:
(i) a time from nephrectomy to systemic treatment of less than one year, a lack of a nephrectomy, or an initial diagnosis with metastatic disease;
(ii) a hemoglobin level less than the LLN, optionally wherein the normal range for hemoglobin is between 13.5 and 17.5 g/dL for men and between 12 and 15.5 g/dL for women;
(iii) a serum corrected calcium level greater than 10 mg/dL, optionally wherein the serum corrected calcium level is the serum calcium level (mg/dL)+0.8(4−serum albumin (g/dL));
(iv) a serum LDH level greater than 1.5 times the ULN, optionally wherein the ULN is 140 U/L; and/or
(v) a KPS score of <80.

25. The method of any one of claims 19-24, wherein the individual has a sarcomatoid kidney cancer.

26. The method of any one of claims 19-25, wherein the benefit is in terms of improved PFS, OS, ORR, CR rate, or DFR.

27. The method of claim 26, wherein the benefit is in terms of improved PFS.

28. The method of claim 26, wherein the benefit is in terms of improved OS.

29. The method of claim 26, wherein the benefit is in terms of improved ORR.

30. The method of claim 26, wherein the benefit is in terms of improved CR rate.

31. The method of claim 26, wherein the benefit is in terms of improved DFR.

32. The method of claim 31, wherein DFR is determined in terms of the time from onset of treatment to the individual's first increase of greater than or equal to 2 points above baseline on the MDASI interference scale.

33. The method of any one of claims 1-32, further comprising determining the expression level of one or more of the following genes in a sample from the individual:
CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2;
VEGFA, KDR, ESM1, PECAM1, FLT1, ANGPTL4, or CD34; or
IL6, CXCL1, CXCL2, CXCL3, CXCL8, or PTGS2.

34. The method of any one of claims 1-33, wherein:
(i) an expression level of one or more of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample that is at or above a reference expression level of the one or more genes; or
(ii) an expression level of one or more of VEGFA, KDR, ESM1, PECAM1, FLT1, ANGPTL4, or CD34; or IL6, CXCL1, CXCL2, CXCL3, CXCL8, or PTGS2 in the sample that is below a reference expression level of the one or more genes
identifies the individual as one who may benefit from treatment with an anti-cancer therapy comprising a VEGF antagonist and a PD-L1 axis binding antagonist.

35. The method of claim 33 or 34, wherein the expression level of one or more of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample is determined to be at or above a reference level of the one or more genes.

36. The method of claim 35, wherein the expression level of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or all twenty of CD8A, EOMES, GZMA, GZMB, PRF1, IFNG, PD-L1, CXCL9, CXCL10, CXCL11, CD27, FOXP3, PD-1, CTLA4, TIGIT, IDO1, PSMB8, PSMB9, TAP1, or TAP2 in the sample is determined to be at or above a reference level of the one or more genes.

37. The method of claim 35 or 36, wherein the expression level of one or more of CD8A, EOMES, PRF1, IFNG, or PD-L1 in the sample is determined to be at or above a reference level of the one or more genes.

38. The method of claim 37, wherein the expression level of CD8A, EOMES, PRF1, IFNG, and PD-L1 in the sample is determined to be at or above a reference level of CD8A, EOMES, PRF1, IFNG, and PD-L1.

39. The method of any one of claims 33-38, wherein the expression level of one or more of IL6, CXCL1, CXCL2, CXCL3, CXCL8, or PTGS2 in the sample is determined to be at or above a reference level of the one or more genes.

40. The method of claim 39, wherein the expression level of at least one, at least two, at least three, at least four, at least five, or all six of IL6, CXCL1, CXCL2, CXCL3, CXCL8, or PTGS2 in the sample is determined to be at or above a reference level of the one or more genes.

41. The method of claim 39 or 40, wherein the expression level of IL6, CXCL1, CXCL2, CXCL3, CXCL8, and PTGS2 in the sample is determined to be at or above a reference level of IL6, CXCL1, CXCL2, CXCL3, CXCL8, and PTGS2.

42. The method of any one of claims 33-41, wherein the expression level of PD-L1 in the sample is determined to be at or above a reference expression level of PD-L1, and the expression level of one or more additional genes selected from the group consisting of CD8A, EOMES, GZMA, GZMB, PRF1, IFNG, CXCL9, CXCL10, CXCL11, CD27, FOXP3, PD-1, CTLA4, TIGIT, IDO1, PSMB8, PSMB9, TAP1, or TAP2 in the sample is determined to be at or above a reference expression level of the one or more additional genes.

43. The method of claim 33 or 34, wherein the expression level of one or more of VEGFA, KDR, ESM1, PECAM1, FLT1, ANGPTL4, or CD34 in the sample is determined to be below a reference level of the one or more genes.

44. The method of claim 43, wherein the expression level of at least one, at least two, at least three, at least four, at least five, at least six, or all seven of VEGFA, KDR, ESM1, PECAM1, FLT1, ANGPTL4, or CD34 in the sample is determined to be below a reference level of the one or more genes.

45. The method of claim 43 or 44, wherein the expression level of one or more of VEGFA, KDR, ESM1, PECAM1, ANGPTL4, or CD34 in the sample is determined to be below a reference level of the one or more genes.

46. The method of claim 45, wherein the expression level of VEGFA, KDR, ESM1, PECAM1, ANGPTL4, and CD34 in the sample is determined to be below a reference level of VEGFA, KDR, ESM1, PECAM1, ANGPTL4, and CD34.

47. The method of claim 33 or 34, wherein the expression level of one or more of IL6, CXCL1, CXCL2, CXCL3, CXCL8, or PTGS2 in the sample is determined to be below a reference level of the one or more genes.

48. The method of claim 47, wherein the expression level of at least one, at least two, at least three, at least four, at least five, or all six of IL6, CXCL1, CXCL2, CXCL3, CXCL8, or PTGS2 in the sample is determined to be below a reference level of the one or more genes.

49. The method of claim 47 or 48, wherein the expression level of IL6, CXCL1, CXCL2, CXCL3, CXCL8, and PTGS2 in the sample is determined to be below a reference level of IL6, CXCL1, CXCL2, CXCL3, CXCL8, and PTGS2.

50. The method of any one of claims 34-49, wherein the reference level of the one or more genes is determined from a population of individuals having a kidney cancer.

51. The method of claim 50, wherein the reference level of the one or more genes is a median expression level determined in a population of patients having a kidney cancer.

52. The method of claim 51, wherein the reference level is a median of a Z-score of the normalized expression level of the one or more genes.

53. The method of any one of claims 33-52, wherein the expression level is a nucleic acid expression level.

54. The method of claim 53, wherein the nucleic acid expression level is an mRNA expression level.

55. The method of claim 54, wherein the mRNA expression level is determined by RNA-seq, RT-qPCR, qPCR, multiplex qPCR or RT-qPCR, microarray analysis, SAGE, MassARRAY technique, ISH, or a combination thereof.

56. The method of any one of claims 33-52, wherein the expression level is a protein expression level.

57. The method of claim 56, wherein the protein expression level is determined by immunohistochemistry (IHC), Western blot, enzyme-linked immunosorbent assay (ELISA), immunoprecipitation, immunofluorescence, radioimmunoassay, or mass spectrometry.

58. The method of any one of claim 6 or 33-57, wherein the sample is a tissue sample, a cell sample, a whole blood sample, a plasma sample, a serum sample, or a combination thereof.

59. The method of claim 58, wherein the tissue sample is a tumor tissue sample.

60. The method of claim 59, wherein the tumor tissue sample comprises tumor cells, tumor-infiltrating immune cells, stromal cells, or a combination thereof.

61. The method of claim 59 or 60, wherein the tumor tissue sample is a formalin-fixed and paraffin-embedded (FFPE) sample, an archival sample, a fresh sample, or a frozen sample.

62. The method of any one of claims 1-61, wherein the individual has not been previously treated for the kidney cancer.

63. The method of any one of claims 1-62, wherein the kidney cancer is renal cell carcinoma (RCC).

64. The method of claim 63, wherein the RCC is clear cell RCC.

65. The method of claim 63 or 64, wherein the RCC is locally advanced or metastatic RCC (mRCC).

66. The method of any one of claims 1-65, wherein a tumor sample obtained from the patient has been determined to have a detectable expression level of PD-L1 in tumor-infiltrating immune cells that comprise about 1% or more of the tumor sample.

67. The method of claim 66, wherein the tumor sample has been determined to have a detectable expression level of PD-L1 in tumor-infiltrating immune cells that comprise about 1% or more to less than 5% of the tumor sample.

68. The method of claim 66, wherein the tumor sample has been determined to have a detectable expression level of PD-L1 in tumor-infiltrating immune cells that comprise about 5% or more of the tumor sample.

69. The method of claim 68, wherein the tumor sample has been determined to have a detectable expression level of PD-L1 in tumor-infiltrating immune cells that comprise about 5% or more to less than 10% of the tumor sample.

70. The method of claim 66 or 68, wherein the tumor sample obtained from the patient has been determined to have a detectable expression level of PD-L1 in tumor-infiltrating immune cells that comprise about 10% or more of the tumor sample.

71. The method of any one of claims 1-65, wherein a tumor sample obtained from the patient has been determined to have a detectable expression level of PD-L1 in tumor-infiltrating immune cells that comprise less than 1% of the tumor sample.

72. The method of any one of claims 1-71, wherein the VEGF antagonist is an anti-VEGF antibody or a VEGF receptor (VEGFR) inhibitor.

73. The method of claim 72, wherein the VEGF antagonist is an anti-VEGF antibody.

74. The method of claim 73, wherein the anti-VEGF antibody is bevacizumab.

75. The method of claim 72, wherein the VEGF antagonist is a VEGFR inhibitor.

76. The method of claim 75, wherein the VEGFR inhibitor is a multi-targeted tyrosine kinase inhibitor.

77. The method of claim 76, wherein the multi-targeted tyrosine kinase inhibitor is sunitinib, axitinib, pazopanib, or cabozantinib.

78. The method of claim 77, wherein the multi-targeted tyrosine kinase inhibitor is sunitinib.

79. The method of any one of claims 1-78, wherein the PD-L1 axis binding antagonist is selected from the group consisting of a PD-L1 binding antagonist, a PD-1 binding antagonist, and a PD-L2 binding antagonist.

80. The method of claim 79, wherein the PD-L1 axis binding antagonist is a PD-L1 binding antagonist.

81. The method of claim 80, wherein the PD-L1 binding antagonist inhibits the binding of PD-L1 to one or more of its ligand binding partners.

82. The method of claim 81, wherein the PD-L1 binding antagonist inhibits the binding of PD-L1 to PD-1.

83. The method of claim 81, wherein the PD-L1 binding antagonist inhibits the binding of PD-L1 to B7-1.

84. The method of any one of claims 81-83, wherein the PD-L1 binding antagonist inhibits the binding of PD-L1 to both PD-1 and B7-1.

85. The method of any one of claims 80-84, wherein the PD-L1 binding antagonist is an anti-PD-L1 antibody.

86. The method of claim 85, wherein the anti-PD-L1 antibody is selected from the group consisting of:
MPDL3280A (atezolizumab), YW243.55.S70, MDX-1105, MED14736 (durvalumab), and MSB0010718C (avelumab).

87. The method of claim 85 or 86, wherein the anti-PD-L1 antibody comprises the following hypervariable regions (HVRs):
(a) an HVR-H1 sequence of
(SEQ ID NO: 63)
GFTFSDSWIH;
(b) an HVR-H2 sequence of
(SEQ ID NO: 64)
AWISPYGGSTYYADSVKG;
(c) an HVR-H3 sequence of
(SEQ ID NO: 65)
RHWPGGFDY;
(d) an HVR-L1 sequence of
(SEQ ID NO: 66)
RASQDVSTAVA;
(e) an HVR-L2 sequence of
(SEQ ID NO: 67)
SASFLYS;
and
(f) an HVR-L3 sequence of
(SEQ ID NO: 68)
QQYLYHPAT.

88. The method of any one of claims 85-87, wherein the anti-PD-L1 antibody comprises:
(a) a heavy chain variable (VH) domain comprising
an amino acid sequence having at least 90%
sequence identity to the amino acid sequence of
(SEQ ID NO: 69)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSS;
(b) a light chain variable (VL) domain comprising
an amino acid sequence having at least 90%
sequence identity to the amino acid sequence of
(SEQ ID NO: 70)
DIQMTQSPSSLSASVGDRVTITCRASQDVSTAVAWYQQKPGKAPKLLI
YSASFLYSGVPSRFSGSGSGTDFTLTISSLQPEDFATYYCQQYLYHPA
TFGQGTKVEIKR;
or
(c) a VH domain as in (a) and a VL domain as in (b).

89. The method of claim 88, wherein the anti-PD-L1 antibody comprises:
(a) a VH domain comprising an amino acid sequence having at least 95% sequence identity to the amino acid sequence of SEQ ID NO: 69;
(b) a VL domain comprising an amino acid sequence having at least 95% sequence identity to the amino acid sequence of SEQ ID NO: 70; or
(c) a VH domain as in (a) and a VL domain as in (b).

90. The method of claim 89, wherein the anti-PD-L1 antibody comprises:
(a) a VH domain comprising an amino acid sequence having at least 96% sequence identity to the amino acid sequence of SEQ ID NO: 69;
(b) a VL domain comprising an amino acid sequence having at least 96% sequence identity to the amino acid sequence of SEQ ID NO: 70; or
(c) a VH domain as in (a) and a VL domain as in (b).

91. The method of claim 90, wherein the anti-PD-L1 antibody comprises:
(a) a VH domain comprising an amino acid sequence having at least 97% sequence identity to the amino acid sequence of SEQ ID NO: 69;
(b) a VL domain comprising an amino acid sequence having at least 97% sequence identity to the amino acid sequence of SEQ ID NO: 70; or
(c) a VH domain as in (a) and a VL domain as in (b).

92. The method of claim 91, wherein the anti-PD-L1 antibody comprises:
(a) a VH domain comprising an amino acid sequence having at least 98% sequence identity to the amino acid sequence of SEQ ID NO: 69;
(b) a VL domain comprising an amino acid sequence having at least 98% sequence identity to the amino acid sequence of SEQ ID NO: 70; or
(c) a VH domain as in (a) and a VL domain as in (b).

93. The method of claim 92, wherein the anti-PD-L1 antibody comprises:
(a) a VH domain comprising an amino acid sequence having at least 99% sequence identity to the amino acid sequence of SEQ ID NO: 69;
(b) a VL domain comprising an amino acid sequence having at least 99% sequence identity to the amino acid sequence of SEQ ID NO: 70; or
(c) a VH domain as in (a) and a VL domain as in (b).

94. The method of claim 93, wherein the anti-PD-L1 antibody comprises:
(a) a VH domain comprising the amino acid sequence of SEQ ID NO: 69;
(b) a VL domain comprising the amino acid sequence of SEQ ID NO: 70; or
(c) a VH domain as in (a) and a VL domain as in (b).

95. The method of claim 94, wherein the anti-PD-L1 antibody comprises:
(a) a VH domain comprising the amino acid sequence of SEQ ID NO: 69; and
(b) a VL domain comprising the amino acid sequence of SEQ ID NO: 70.

96. The method of claim 95, wherein the anti-PD-L1 antibody is atezolizumab.

97. The method of any one of claims 1-96, wherein the PD-L1 axis binding antagonist is atezolizumab and the VEGF antagonist is bevacizumab.

98. The method of claim 97, wherein the atezolizumab is administered intravenously every three weeks at a dose of about 1200 mg.

99. The method of claim 97 or 98, wherein the bevacizumab is administered intravenously every three weeks at a dose of about 15 mg/kg.

100. The method of any one of claims 1-99, further comprising administering an additional therapeutic agent to the individual.

101. The method of claim 100, wherein the additional therapeutic agent is selected from the group consisting of an immunotherapy agent, a cytotoxic agent, a growth inhibitory agent, a radiation therapy agent, an anti-angiogenic agent, and combinations thereof.

102. The method of any one of claims 1-101, wherein the individual is a human.

103. A pharmaceutical composition comprising a PD-L1 axis binding antagonist for use in treatment of an individual having a kidney cancer, wherein the treatment comprises administration of the PD-L1 axis binding antagonist in combination with a VEGF antagonist, wherein the individual is identified as likely to benefit from the anti-cancer therapy based on having a sarcomatoid kidney cancer.

104. A pharmaceutical composition comprising a PD-L1 axis binding antagonist for use in treatment of an individual having a kidney cancer, wherein the treatment comprises administration of the PD-L1 axis binding antagonist in combination with a VEGF antagonist, wherein the individual is identified as likely to benefit from the anti-cancer therapy based on having a poor or intermediate MSKCC risk score.

105. Use of a PD-L1 axis binding antagonist in the manufacture of a medicament for treatment of an individual having a kidney cancer, wherein the treatment comprises administration of the PD-L1 axis binding antagonist in combination with a VEGF antagonist, wherein the individual is identified as likely to benefit from the anti-cancer therapy based on having a sarcomatoid kidney cancer.

106. Use of a PD-L1 axis binding antagonist in the manufacture of a medicament for treatment of an individual having a kidney cancer, wherein the treatment comprises administration of the PD-L1 axis binding antagonist in combination with a VEGF antagonist, wherein the individual is identified as likely to benefit from the anti-cancer therapy based on having a poor or intermediate MSKCC risk score.

107. The pharmaceutical composition for use of claim 103 or 104, or the use of claim 105 or 106, wherein the benefit is in terms of improved progression-free survival (PFS), overall survival (OS), overall response rate (ORR), complete response (CR) rate, or deterioration-free rate (DFR).

108. The pharmaceutical composition for use or the use of claim 107, wherein the benefit is in terms of improved PFS.

109. The pharmaceutical composition for use or the use of claim 107, wherein the benefit is in terms of improved OS.

110. The pharmaceutical composition for use or the use of claim 107, wherein the benefit is in terms of improved ORR.

111. The pharmaceutical composition for use or the use of claim 107, wherein the benefit is in terms of improved CR rate.

112. The pharmaceutical composition for use or the use of claim 107, wherein the benefit is in terms of improved DFR.

113. The pharmaceutical composition for use or the use of claim 112, wherein DFR is determined in terms of the time from onset of treatment to the individual's first increase of greater than or equal to 2 points above baseline on the MD Anderson Symptom Inventory (MDASI) interference scale.


SEQUENCE LISTING (82 sequences)

SEQ ID NO: 1 - RNA, 1975 nt, Homo sapiens
acuuuccccccucggcgccccaccggcucccgcgcgccuccccucgcgcccgagcuucgagccaagcagcguccuggggagcgcgucauggccuuaccagugaccgccuugcuccugccgcuggccuugcugcuccacgccgccaggccgagccaguuccgggugucgccgcuggaucggaccuggaaccugggcgagacaguggagcugaagugccaggugcugcuguccaacccgacgucgggcugcucguggcucuuccagccgcgcggcgccgccgccagucccaccuuccuccuauaccucucccaaaacaagcccaaggcggccgaggggcuggacacccagcgguucucgggcaagagguugggggacaccuucguccucacccugagcgacuuccgccgagagaacgagggcuacuauuucugcucggcccugagcaacuccaucauguacuucagccacuucgugccggucuuccugccagcgaagcccaccacgacgccagcgccgcgaccaccaacaccggcgcccaccaucgcgucgcagccccugucccugcgcccagaggcgugccggccagcggcggggggcgcagugcacacgagggggcuggacuucgccugugauaucuacaucugggcgccccuggccgggacuugugggguccuucuccugucacugguuaucacccuuuacugcaaccacaggaaccgaagacguguuugcaaauguccccggccuguggucaaaucgggagacaagcccagccuuucggcgagauacgucuaacccugugcaacagccacuacauuacuucaaacugagauccuuccuuuugagggagcaaguccuucccuuucauuuuuuccagucuuccucccuguguauucauuuucaugauuauuauuuuagugggggcggggugggaaagauuacuuuuucuuuauguguuugacgggaaacaaaacuagguaaaaucuacaguacaccacaagggucacaauacuguugugcgcacaucgcgguagggcguggaaaggggcaggccagagcuacccgcagaguucucagaaucaugcugagagagcuggaggcacccaugccaucucaaccucuuccccgcccguuuuacaaagggggaggcuaaagcccagagacagcuugaucaaaggcacacagcaagucaggguuggagcaguagcuggagggaccuugucucccagcucagggcucuuuccuccacaccauucaggucuuucuuuccgaggccccugucucagggugaggugcuugagucuccaacggcaagggaacaaguacuucuugauaccugggauacugugcccagagccucgaggagguaaugaauuaaagaagagaacugccuuuggcagaguucuauaauguaaacaauaucagacuuuuuuuuuuuauaaucaagccuaaaauuguauagaccuaaaauaaaaugaaguggugagcuuaacccuggaaaaugaaucccucuaucucuaaagaaaaucucugugaaaccccuacguggaggcggaauugcucucccagcccuugcauugcagaggggcccaugaaagaggacaggcuaccccuuuacaaauagaauuugagcaucagugagguuaaacuaaggcccucuugaaucucugaauuugagauacaaacauguuccugggaucacugaugacuuuuuauacuuuguaaagacaauuguuggagagccccucacacagcccuggccuccgcucaacuagcagauacagggaugaggcagaccugacucucuuaaggaggcugagagcccaaacugcugucccaaacaugcacuuccuugcuuaagguaugguacaagcaaugccugcccauuggagagaaaaaacuuaaguagauaaggaaauaagaaccacucauaauucuucaccuuaggaauaaucuccuguuaauaugguguaca

SEQ ID NO: 2 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 3 - RNA, 2061 nt, Homo sapiens
augcaguugggagagcagcucuuggugagcucagugaaccugccuggcgcgcacuucuacccgcuggagagugcgcgaggcggcagcggcgggagcgcuggccaccuccccagcgcggcccccucuccucagaaguuggacuuaggcaaagcguccaagaaguuuuccggcagucucuccugcgaggcggugagcggggagccugcagccgccagcgcaggggcccccgcggccaugcuuagugacaccgacgccggggacgcauuugccagcgcugcggcaguggccaagccggggcccccggacggccgcaagggcucccccugcggggaggaggagcugcccuccgccgcugcagccgccgccgccgccgccgccgcggcugcggccacugcgcgcuacuccauggacagccugagcuccgugcgguacuaccuccagucccccgguccucaggggucggagcuggcugcgcccugcucacucuucccguaccaggcggcggcuggggcgccccacggaccuguguacccggcuccuaacggggcgcgcuaccccuacggcuccaugcugccccccggcggcuuccccgcggcugcgugcccacccgggagggcgcaguucggcccaggagccggugcgggcaguggcgcgggcgguaucaacggcgggggcggcggcccgggcaccuaucaguacagccagggggcuccgcucuacgggccguacccuggagccgcagcggcgggaucuugcggaggacuggggggccuggggguuccaaguucuggcuuccgugcccacgucuaccugugcaaccggccucuguggcucaaauuccaccggcaccaaacugagaugaucaucaccaaacagggcaggcgcauguuuccuuucuugagcuucaacauaaacggacucaaccccaccgcccacuacaauguuuucguggaagugguucuggccgacccuaaccacuggcgcuuccaggggggcaaaugggugaccuguggcaaagccgacaauaacaugcagggcaacaaaauguauguucacccagagucuccuaauacugguucccacuggaugagacaggagauuucauucgggaaauuaaaacucaccaauaacaaaggcgcaaauaacaacaacacccagaugauagucuuacaauccuuacacaaauaccaaccccgacugcauauuguugaaguuacagaggauggcguggaggacuugaaugagcccucaaagacccagacuuuuaccuucucagaaacgcaauucauugcagugacugccuaccaaaacaccgauauuacucaacuaaagauugaucauaaccccuuugcaaaaggcuucagagacaacuaugauucaucccaucagauugucccuggaggucgguacggcguucaauccuucuucccggagcccuuugucaacacuuuaccucaagcccgcuauuauaauggcgagagaaccgugccacagaccaacggccuccuuucaccccaacagagcgaagagguggccaacccuccccagcgguggcuugucacgccuguccagcaaccugggaccaacaaacuagacaucaguuccuaugaaucugaauauacuucuagcacauugcucccauauggcauuaaauccuugccccuucagacaucccaugcccugggguauuacccagacccaaccuuuccugcaauggcaggguggggaggucgagguucuuaccagaggaagauggcagcuggacuaccauggaccuccagaacaagccccacuguguucucugaagaucagcucuccaaggagaaagugaaagaggaaauuggcucuucuuggauagagacacccccuuccaucaaaucucuagauuccaaugauucaggaguauacaccagugcuuguaagcgaaggcggcugucuccuagcaacuccaguaaugaaaauucacccuccauaaaguguggggacauuaaugcugaagaguauaguaaagacaccucaaaaggcaugggaggguauuaugcuuuuuacacaagucccuag

SEQ ID NO: 4 - protein, 686 aa, Homo sapiens
MQLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTP

SEQ ID NO: 5 - RNA, 1668 nt, Homo sapiens
auggcagcccgucugcuccuccugggcauccuucuccugcugcugccccugcccgucccugccccgugccacacagccgcacgcucagagugcaagcgcagccacaaguucgugccuggugcauggcuggccggggaggguguggacgugaccagccuccgccgcucgggcuccuucccaguggacacacaaagguuccugcggcccgacggcaccugcacccucugugaaaaugcccuacaggagggcacccuccagcgccugccucuggcgcucaccaacuggcgggcccagggcucuggcugccagcgccauguaaccagggccaaagucagcuccacugaagcuguggcccgggaugcggcucguagcauccgcaacgacuggaaggucgggcuggacgugacuccuaagcccaccagcaaugugcaugugucuguggccggcucacacucacaggcagccaacuuugcagcccagaagacccaccaggaccaguacagcuucagcacugacacgguggagugccgcuucuacaguuuccaugugguacacacucccccgcugcacccugacuucaagagggcccucggggaccugccccaccacuucaacgccuccacccagcccgccuaccucaggcuuaucuccaacuacggcacccacuucauccgggcuguggagcuggguggccgcauaucggcccucacugcccugcgcaccugcgagcuggcccuggaagggcucacggacaacgagguggaggacugccugacugucgaggcccaggucaacauaggcauccacggcagcaucucugccgaagccaaggccugugaggagaagaagaagaagcacaagaugacggccuccuuccaccaaaccuaccgggagcgccacucggaagugguuggcggccaucacaccuccauuaacgaccugcuguucgggauccaggccgggcccgagcaguacucagccuggguaaacuccgugcccggcagcccuggccugguggacuacacccuggaaccccugcacgugcugcuggacagccaggacccgcggcgggaggcacugaggagggcccugagucaguaccugacggacagggcucgcuggagggacugcagccggccgugcccaccagggcggcagaagagcccccgagacccaugccagugugugugccauggcucagcggucaccacccaggacugcugcccucggcagaggggccuggcccagcuggaggugaccuucauccaagcauggagccuguggggggacugguucacugccacggaugccuaugugaagcucuucuuugguggccaggagcugaggacgagcaccgugugggacaauaacaaccccaucuggucagugcggcuggauuuuggggaugugcuccuggccacaggggggccccugagguugcaggucugggaucaggacucuggcagggacgaugaccuccuuggcaccugugaucaggcucccaagucugguucccaugaggugagaugcaaccugaaucauggccaccuaaaauuccgcuaucaugccaggugcuugccccaccugggaggaggcaccugccuggacuaugucccccaaaugcuucugggggagccuccaggaaaccggaguggggccgugugguga

SEQ ID NO: 6 - protein, 555 aa, Homo sapiens
MAARLLLLGILLLLLPLPVPAPCHTAARSECKRSHKFVPGAWLAGEGVDVTSLRRSGSFPVDTQRFLRPDGTCTLCENALQEGTLQRLPLALTNWRAQGSGCQRHVTRAKVSSTEAVARDAARSIRNDWKVGLDVTPKPTSNVHVSVAGSHSQAANFAAQKTHQDQYSFSTDTVECRFYSFHVVHTPPLHPDFKRALGDLPHHFNASTQPAYLRLISNYGTHFIRAVELGGRISALTALRTCELALEGLTDNEVEDCLTVEAQVNIGIHGSISAEAKACEEKKKKHKMTASFHQTYRERHSEVVGGHHTSINDLLFGIQAGPEQYSAWVNSLPGSPGLVDYTLEPLHVLLDSQDPRREALRRALSQYLTDRARWRDCSRPCPPGRQKSPRDPCQCVCHGSAVTTQDCCPRQRGLAQLEVTFIQAWGLWGDWFTATDAYVKLFFGGQELRTSTVWDNNNPIWSVRLDFGDVLLATGGPLRLQVWDQDSGRDDDLLGTCDQAPKSGSHEVRCNLNHGHLKFRYHARCLPHLGGGTCLDYVPQMLLGEPPGNRSGAVW

SEQ ID NO: 7 - RNA, 1193 nt, Homo sapiens
ugaagaucagcuauuagaagagaaagaucaguuaaguccuuuggaccugaucagcuugauacaagaacuacugauuucaacuucuuuggcuuaauucucucggaaacgaugaaauauacaaguuauaucuuggcuuuucagcucugcaucguuuuggguucucuuggcuguuacugccaggacccauauguaaaagaagcagaaaaccuuaagaaauauuuuaaugcaggucauucagauguagcggauaauggaacucuuuucuuaggcauuuugaagaauuggaaagaggagagugacagaaaaauaaugcagagccaaauugucuccuuuuacuucaaacuuuuuaaaaacuuuaaagaugaccagagcauccaaaagaguguggagaccaucaaggaagacaugaaugucaaguuuuucaauagcaacaaaaagaaacgagaugacuucgaaaagcugacuaauuauucgguaacugacuugaauguccaacgcaaagcaauacaugaacucauccaagugauggcugaacugucgccagcagcuaaaacagggaagcgaaaaaggagucagaugcuguuucaaggucgaagagcaucccaguaaugguuguccugccugcaauauuugaauuuuaaaucuaaaucuauuuauuaauauuuaacauuauuuauauggggaauauauuuuuagacucaucaaucaaauaaguauuuauaauagcaacuuuuguguaaugaaaaugaauaucuauuaauauauguauuauuuauaauuccuauauccugugacugucucacuuaauccuuuguuuucugacuaauuaggcaaggcuaugugauuacaaggcuuuaucucaggggccaacuaggcagccaaccuaagcaagaucccauggguuguguguuuauuucacuugaugauacaaugaacacuuauaagugaagugauacuauccaguuacugccgguuugaaaauaugccugcaaucugagccagugcuuuaauggcaugucagacagaacuugaaugugucaggugacccugaugaaaacauagcaucucaggagauuucaugccuggugcuuccaaauauuguugacaacugugacuguacccaaauggaaaguaacucauuuguuaaaauuaucaauaucuaauauauaugaauaaaguguaaguucacaacu

SEQ ID NO: 8 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 9 - RNA, 5830 nt, Homo sapiens
acugagucccgggaccccgggagagcggucaguguguggucgcugcguuuccucugccugcgccgggcaucacuugcgcgccgcagaaaguccgucuggcagccuggauauccucuccuaccggcacccgcagacgccccugcagccgccggucggcgcccgggcucccuagcccugugcgcucaacuguccugcgcugcggggugccgcgaguuccaccuccgcgccuccuucucuagacaggcgcugggagaaagaaccggcucccgaguucugggcauuucgcccggcucgaggugcaggaugcagagcaaggugcugcuggccgucgcccuguggcucugcguggagacccgggccgccucuguggguuugccuaguguuucucuugaucugcccaggcucagcauacaaaaagacauacuuacaauuaaggcuaauacaacucuucaaauuacuugcaggggacagagggacuuggacuggcuuuggcccaauaaucagaguggcagugagcaaaggguggaggugacugagugcagcgauggccucuucuguaagacacucacaauuccaaaagugaucggaaaugacacuggagccuacaagugcuucuaccgggaaacugacuuggccucggucauuuaugucuauguucaagauuacagaucuccauuuauugcuucuguuagugaccaacauggagucguguacauuacugagaacaaaaacaaaacuguggugauuccaugucucggguccauuucaaaucucaacgugucacuuugugcaagauacccagaaaagagauuuguuccugaugguaacagaauuuccugggacagcaagaagggcuuuacuauucccagcuacaugaucagcuaugcuggcauggucuucugugaagcaaaaauuaaugaugaaaguuaccagucuauuauguacauaguugucguuguaggguauaggauuuaugaugugguucugaguccgucucauggaauugaacuaucuguuggagaaaagcuugucuuaaauuguacagcaagaacugaacuaaauguggggauugacuucaacugggaauacccuucuucgaagcaucagcauaagaaacuuguaaaccgagaccuaaaaacccagucugggagugagaugaagaaauuuuugagcaccuuaacuauagaugguguaacccggagugaccaaggauuguacaccugugcagcauccagugggcugaugaccaagaagaacagcacauuugucaggguccaugaaaaaccuuuuguugcuuuuggaaguggcauggaaucucugguggaagccacggugggggagcgugucagaaucccugcgaaguaccuugguuacccacccccagaaauaaaaugguauaaaaauggaauaccccuugaguccaaucacacaauuaaagcggggcauguacugacgauuauggaagugagugaaagagacacaggaaauuacacugucauccuuaccaaucccauuucaaaggagaagcagagccauguggucucucugguuguguaugucccaccccagauuggugagaaaucucuaaucucuccuguggauuccuaccaguacggcaccacucaaacgcugacauguacggucuaugccauuccucccccgcaucacauccacugguauuggcaguuggaggaagagugcgccaacgagcccagccaagcugucucagugacaaacccauacccuugugaagaauggagaaguguggaggacuuccagggaggaaauaaaauugaaguuaauaaaaaucaauuugcucuaauugaaggaaaaaacaaaacuguaaguacccuuguuauccaagcggcaaaugugucagcuuuguacaaaugugaagcggucaacaaagucgggagaggagagagggugaucuccuuccacgugaccagggguccugaaauuacuuugcaaccugacaugcagcccacugagcaggagagcgugucuuuguggugcacugcagacagaucuacguuugagaaccucacaugguacaagcuuggcccacagccucugccaauccaugugggagaguugcccacaccuguuugcaagaacuuggauacucuuuggaaauugaaugccaccauguucucuaauagcacaaaugacauuuugaucauggagcuuaagaaugcauccuugcaggaccaaggagacuaugucugccuugcucaagacaggaagaccaagaaaagacauugcguggucaggcagcucacaguccuagagcguguggcacccacgaucacaggaaaccuggagaaucagacgacaaguauuggggaaagcaucgaagucucaugcacggcaucugggaaucccccuccacagaucaugugguuuaaagauaaugagacccuuguagaagacucaggcauuguauugaaggaugggaaccggaaccucacuauccgcagagugaggaaggaggacgaaggccucuacaccugccaggcaugcaguguucuuggcugugcaaaaguggaggcauuuuucauaauagaaggugcccaggaaaagacgaacuuggaaaucauuauucuaguaggcacggcggugauugccauguucuucuggcuacuucuugucaucauccuacggaccguuaagcgggccaauggaggggaacugaagacaggcuacuuguccaucgucauggauccagaugaacucccauuggaugaacauugugaacgacugccuuaugaugccagcaaaugggaauuccccagagaccggcugaagcuagguaagccucuuggccguggugccuuuggccaagugauugaagcagaugccuuuggaauugacaagacagcaacuugcaggacaguagcagucaaaauguugaaagaaggagcaacacacagugagcaucgagcucucaugucugaacucaagauccucauucauauuggucaccaucucaauguggucaaccuucuaggugccuguaccaagccaggagggccacucauggugauuguggaauucugcaaauuuggaaaccuguccacuuaccugaggagcaagagaaaugaauuuguccccuacaagaccaaaggggcacgauuccgucaagggaaagacuacguuggagcaaucccuguggaucugaaacggcgcuuggacagcaucaccaguagccagagcucagccagcucuggauuuguggaggagaagucccucagugauguagaagaagaggaagcuccugaagaucuguauaaggacuuccugaccuuggagcaucucaucuguuacagcuuccaaguggcuaagggcauggaguucuuggcaucgcgaaaguguauccacagggaccuggcggcacgaaauauccucuuaucggagaagaacgugguuaaaaucugugacuuuggcuuggcccgggauauuuauaaagauccagauuaugucagaaaaggagaugcucgccucccuuugaaauggauggccccagaaacaauuuuugacagaguguacacaauccagagugacgucuggucuuuugguguuuugcugugggaaauauuuuccuuaggugcuucuccauauccugggguaaagauugaugaagaauuuuguaggcgauugaaagaaggaacuagaaugagggccccugauuauacuacaccagaaauguaccagaccaugcuggacugcuggcacggggagcccagucagagacccacguuuucagaguugguggaacauuugggaaaucucuugcaagcuaaugcucagcaggauggcaaagacuacauuguucuuccgauaucagagacuuugagcauggaagaggauucuggacucucucugccuaccucaccuguuuccuguauggaggaggaggaaguaugugaccccaaauuccauuaugacaacacagcaggaaucagucaguaucugcagaacaguaagcgaaagagccggccugugaguguaaaaacauuugaagauaucccguuagaagaaccagaaguaaaaguaaucccagaugacaaccagacggacagugguaugguucuugccucagaagagcugaaaacuuuggaagacagaaccaaauuaucuccaucuuuugguggaauggugcccagcaaaagcagggagucuguggcaucugaaggcucaaaccagacaagcggcuaccaguccggauaucacuccgaugacacagacaccaccguguacuccagugaggaagcagaacuuuuaaagcugauagagauuggagugcaaaccgguagcacagcccagauucuccagccugacucggggaccacacugagcucuccuccuguuuaaaaggaagcauccacaccccaacucccggacaucacaugagaggucugcucagauuuugaaguguuguucuuuccaccagcaggaaguagccgcauuugauuuucauuucgacaacagaaaaaggaccucggacugcagggagccagucuucuaggcauauccuggaagaggcuugugacccaagaaugugucugugucuucucccaguguugaccugauccucuuuuuucauucauuuaaaaagcauuaucaugccccugcugcgggucucaccauggguuuagaacaaagagcuucaagcaauggccccauccucaaagaaguagcaguaccuggggagcugacacuucuguaaaacuagaagauaaaccaggcaacguaaguguucgagguguugaagaugggaaggauuugcagggcugagucuauccaagaggcuuuguuuaggacgugggucccaagccaagccuuaaguguggaauucggauugauagaaaggaagacuaacguuaccuugcuuuggagaguacuggagccugcaaaugcauuguguuugcucugguggaggugggcauggggucuguucugaaauguaaaggguucagacgggguuucugguuuuagaagguugcguguucuucgaguugggcuaaaguagaguucguugugcuguuucugacuccuaaugagaguuccuuccagaccguuagcugucuccuugccaagccccaggaagaaaaugaugcagcucuggcuccuugucucccaggcugauccuuuauucagaauaccacaaagaaaggacauucagcucaaggcucccugccguguugaagaguucugacugcacaaaccagcuucugguuucuucuggaaugaauacccucauaucuguccugaugugauaugucugagacugaaugcgggagguucaaugugaagcuguguguggugucaaaguuucaggaaggauuuuacccuuuuguucuucccccuguccccaacccacucucaccccgcaacccaucaguauuuuaguuauuuggccucuacuccaguaaaccugauuggguuuguucacucucugaaugauuauuagccagacuucaaaauuauuuuauagcccaaauuauaacaucuauuguauuauuuagacuuuuaacauauagagcuauuucuacugauuuuugcccuuguucuguccuuuuuuucaaaaaagaaaauguguuuuuuguuugguaccauagugugaaaugcugggaacaaugacuauaagacaugcuauggcacauauauuuauagucuguuuauguagaaacaaauguaauauauuaaagccuuauauauaaugaacuuuguacuauucacauuuuguaucaguauuauguagcauaacaaaggucauaaugcuuucagcaauugaugucauuuuauuaaagaacauugaaaaacuuga

SEQ ID NO: 10 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 11 - RNA, 2089 nt, Homo sapiens
aguuuggacggcugcuucccaccagcaaagaccacgacuggagagccgagccggaggcagcugggaaacaugaagagcgucuugcugcugaccacgcuccucgugccugcacaccugguggccgccuggagcaauaauuaugcgguggacugcccucaacacugugacagcagugagugcaaaagcagcccgcgcugcaagaggacagugcucgacgacuguggcugcugccgagugugcgcugcagggcggggagaaacuugcuaccgcacagucucaggcauggauggcaugaaguguggcccggggcugaggugucagccuucuaauggggaggauccuuuuggugaagaguuugguaucugcaaagacugucccuacggcaccuucgggauggauugcagagagaccugcaacugccagucaggcaucugugacagggggacgggaaaaugccugaaauuccccuucuuccaauauucaguaaccaagucuuccaacagauuuguuucucucacggagcaugacauggcaucuggagauggcaauauugugagagaagaaguugugaaagagaaugcugccgggucucccguaaugaggaaaugguuaaauccacgcugaucccggcugugauuucugagagaaggcucuauuuucgugauuguucaacacacagccaacauuuuaggaacuuucuagauuauagcauaaggacauguaauuuuugaagaccaaaugugaugcaugguggauccagaaaacaaaaaguaggauacuuacaauccauaacauccauaugacugaacacuuguauguguuuguuaaauauucgaaugcauguagauuuguuaaauguguguguauaguaacacugaagaacuaaaaaugcaauuuagguaaucuuacguggagacaggucaaccaaagagggagcuaggcaaagcugaagaccgcagugagucaaauuaguucuuugacuuugauguacauuaauguugggauauggaaugaagacuuaagagcaggagaagauggggagggggugggagugggaaauaaaauauuuagcccuuccuugguagguagcuucucuagaauuuaauugugcuuuuuuuuuuuuuuuuggcuuugggaaaagucaaaauaaaacaaccagaaaaccccugaaggaaguaagauguuugaagcuuauggaaauuugaguaacaaacagcuuugaacugagagcaauuucaaaaggcugcugauguaguucccggguuaccuguaucugaaggacgguucuggggcauaggaaacacauacacuuccauaaauagcuuuaacguaugccaccucagagauaaaucuaagaaguauuuuacccacuggugguuuguguguguaugaagguaaauauuuauauauuuuuauaaauaaauguguuagugcaagucaucuucccuacccauauuuaucauccucuugaggaaagaaaucuaguauuauuuguugaaaaugguuagaauaaaacuaugacucuauaagguuuucaaacaucugaggcaugauaaauuuauuauccauaauuauaguaauaauaaccuuaauaagcauaagaaaaacagagucacucuggauuucaaaaaugucaaaaaaugagcaacagaggguccuuauuuaaacauaagugcugugacuuaggugaauuuucaauuuaagguagaaaauaaguuuuuaggagguuuguaaaagaagaaucaauuuucagcagaaaacaugucaacuuuaaaauauaguuuauuuucauauuuuuuucuuuuaaacuugguugauaaguggaauuaggaguauauuugaaagaaucuuagcacaaacaggacuguuguacuagauguucuuaggaaauaucucagaaguauuuuauuugaagugaagaacuuauuuaagaauuauuucaguauuuaccuguauuuuauucuugaaguuggccaacagaguugugaaugugugugggaaggccuuugaauguaaagcugcauaagcuguuagguuuuguuuuaaaaggacauguuuauuauuguucaauaaaaaagaacaagauaca

SEQ ID NO: 12 - protein, 184 aa, Homo sapiens
MKSVLLLTTLLVPAHLVAAWSNNYAVDCPQHCDSSECKSSPRCKRTVLDDCGCCRVCAAGRGETCYRTVSGMDGMKCGPGLRCQPSNGEDPFGEEFGICKDCPYGTFGMDCRETCNCQSGICDRGTGKCLKFPFFQYSVTKSSNRFVSLTEHDMASGDGNIVREEVVKENAAGSPVMRKWLNPR

SEQ ID NO: 13 - RNA, 6831 nt, Homo sapiens
ccaggccccauuguucccgguuuccagccauggcugccauuaccugaccagcgccacagccggucucucugcaggcgccgggagaagugaccagagcaauuucugcuuuucacagggcggguuucucaacggugacuugugggcagugccuucugcugagcgagucauggcccgaaggcagaacuaacugugccugcagucuucacucucaggaugcagccgaggugggcccaaggggccacgauguggcuuggaguccugcugacccuucugcucuguucaagccuugagggucaagaaaacucuuucacaaucaacaguguugacaugaagagccugccggacuggacggugcaaaaugggaagaaccugacccugcagugcuucgcggaugucagcaccaccucucacgucaagccucagcaccagaugcuguucuauaaggaugacgugcuguuuuacaacaucuccuccaugaagagcacagagaguuauuuuauuccugaaguccggaucuaugacucagggacauauaaauguacugugauugugaacaacaaagagaaaaccacugcagaguaccagguguugguggaaggagugcccagucccagggugacacuggacaagaaagaggccauccaaggugggaucgugagggucaacuguucugucccagaggaaaaggccccaauacacuucacaauugaaaaacuugaacuaaaugaaaaaauggucaagcugaaaagagagaagaauucucgagaccagaauuuugugauacuggaauuccccguugaggaacaggaccgcguuuuauccuuccgaugucaagcuaggaucauuucugggauccauaugcagaccucagaaucuaccaagagugaacuggucaccgugacggaauccuucucuacacccaaguuccacaucagccccaccggaaugaucauggaaggagcucagcuccacauuaagugcaccauucaagugacucaccuggcccaggaguuuccagaaaucauaauucagaaggacaaggcgauuguggcccacaacagacauggcaacaaggcuguguacucagucauggccaugguggagcacaguggcaacuacacgugcaaaguggaguccagccgcauauccaaggucagcagcaucguggucaacauaacagaacuauuuuccaagcccgaacuggaaucuuccuucacacaucuggaccaaggugaaagacugaaccuguccugcuccaucccaggagcaccuccagccaacuucaccauccagaaggaagauacgauugugucacagacucaagauuucaccaagauagccucaaagucggacagugggacguauaucugcacugcagguauugacaaaguggucaagaaaagcaacacaguccagauagucguaugugaaaugcucucccagcccaggauuucuuaugaugcccaguuugaggucauaaaaggacagaccaucgaaguccguugcgaaucgaucaguggaacuuugccuauuucuuaccaacuuuuaaaaacaaguaaaguuuuggagaauaguaccaagaacucaaaugauccugcgguauucaaagacaaccccacugaagacgucgaauaccaguguguugcagauaauugccauucccaugccaaaauguuaagugagguucugagggugaaggugauagccccgguggaugagguccagauuucuauccugucaaguaaggugguggagucuggagaggacauugugcugcaaugugcugugaaugaaggaucuggucccaucaccuauaaguuuuacagagaaaaagagggcaaacccuucuaucaaaugaccucaaaugccacccaggcauuuuggaccaagcagaaggcuagcaaggaacaggagggagaguauuacugcacagccuucaacagagccaaccacgccuccaguguccccagaagcaaaauacugacagucagagucauucuugccccauggaagaaaggacuuauugcagugguuaucaucggagugaucauugcucucuugaucauugcggccaaauguuauuuucugaggaaagccaaggccaagcagaugccaguggaaauguccaggccagcaguaccacuucugaacuccaacaacgagaaaaugucagaucccaauauggaagcuaacagucauuacggucacaaugacgaugucagaaaccaugcaaugaaaccaauaaaugauaauaaagagccucugaacucagacgugcaguacacggaaguucaaguguccucagcugagucucacaaagaucuaggaaagaaggacacagagacaguguacagugaaguccggaaagcugucccugaugccguggaaagcagauacucuagaacggaaggcucccuugauggaacuuagacagcaaggccagaugcacaucccuggaaggacauccauguuccgagaagaacagauaaucccuguauuucaagaccucugugcacuuauuuaugaaccugcccugcucccacagaacacagcaauuccucaggcuaagcugccgguucuuaaauccauccugcuaaguuaauguuggguagaaagagauacagaggggcuguugaauuucccacauacccuccuuccaccaaguuggaacauccuuggaaauuggaagagcacaagaggagauccagggcaaggccauugggauauucugaaacuugaauauuuuguuuugugcagagauaaagaccuuuuccaugcacccucauacacagaaaccaauuuucuuuuuuauacucaaucauuucuagcgcauggccugguuagaggcugguuuuuucucuuuuccuuugguccuucaaaggcuuguaguuuuggcuaguccuuguucuuuggaaauacacagugcugaccagacagccucccccuguccccucuaugaccucgcccuccacaaaugggaaaaccagacuacuugggagcaccgccugugaaauaccaaccugaagacaccguucauucaggcaacgcacaaaacagaaaaugaagguggaacaagcacagauguucuucaacuguuuuugucuacacucuuucucuuuuccucuaccaugcugaaggcugaaagacaggaagauggugccaucagcaaauauuauucuuaauugaaaacuugaaauguguauguuucuuacuaauuuuuaaaaauguauuccuugccagggcaggcaagguggcucacgccuguaaucccagcacuucaggaggcugaggugggcggaucaccugaggucaggaguuugagaccagccugaugaaacccugucucuacuaaaaauacaagaauuagccgggcgugguggcgcaugccuguaguaucagcuacucaagaggcugaggugagauuaucgcuugaacccaggaaacggagguuguagugagcggagaucgcgccacugcacuccagccugagugacagagugagaauccaucucaaaaaaaacaaaaaacaaaauugcuugcuaaagaaguggucuccugaggucuuaagacauuccugacagugucuugagugggugggagagaggcugcugucauugcgcuguggaauuucacagaugagaaccacgccuagccaaaaucacuuuuccuguuugccucagugacacagcugcagggacccucguggauguuguauuaaauaaauuugaccuuugcucuuugcagaucugugaaauguugucuucugaggggccacaugcaucuauagugcugaggacuccuugggccucugaagucacagagagaaccgagcaggucuauguuuuuguuuuguuguuuugagacggagauucgcucuuguugcccgggcuggacugcagcggcgcaaccucugcucacugcaaccuccgccuccuggguucaagcaguucuccugucucagccucccgaguagcugggauuacaggcacaugucaccacgccuggcuaauuuuuguauuuuuaguagagaugggguuucaccacguuggccaggcugaucucgaaugccugaccuuuggugaucugcccgccuuguccucaugugugcuccacaggccuuuggguugggauugcaggcgugagccaccaugcccagccuagacucuuuugacaauaugaugaaagcuguugguuccuuuccccaacacacacacaccgaguuguaucacgaaaaugucauacaauuuccagguuuucugaguggugggcucagauugaggucaaaggaucagacgaccucuaacgaccuucaugucucuguugaugaucuggggacagccagauccccuguguccagggaguuccuuagucccuugccaccaccagagaagggcaauugccacgggagcugcaaagacccuauuccuacuccuggugccuuacuuaugcagcacgacugaauuuuuuguuuuguuuuguuuuguugagacaggggcuugcucuguugcccaggcuggagugcaguggcacaacaauggcucaccgcagccucgaaccccugggcucaagcgauccucccaucucagcuuccuggguagcugggaccagaggcgugagccgccauagcuggcuaauuuuuaauuuuuuuuuugcagagaugagguuucaccauggugcccaggcuggucucgaacuucugggcucaagugauccucccuccuuggccucgcaaagugcugggauugcaggcaugagccaccgcccccggccuguggagcacacaugaguuuaaaauuacuuucccuucugccuauauuuccgaggaggaaacuucaugcgcagggaucuuucuuaguggauuuaauggcuaaaaggucugucugaauccaggacgcuggcuuuagccuuccucggcagcugccguaaccccggugucuaaaccugaagcaucccaggagcacccacuccaggaguuuucucggccgcggaacucauuaguuagagcgcccucuuguguucucaugugguaaucggucacugaaggacuuaaaaugguccuuagccaacacacaguaaaacuuuucccucuucugaccccaagaggucagccacccauuucaugagcauauacuggucgccccaucagcguucucugauuggcuaacugaacccacuccccgaccuagacucaagacaggcgaagugacgcuuaggucaacauucacucacuaaagcaacgacugucgggcgauuuugucucccgcugguuuuggaauggugucuggagacauuuuugguugucacagcugggugggugugcucccggcaucugguggguagaaaccaagcaugcuccuaaacauccuacaggcacagaaccgucucccacgaccaagcaugaucaagucccaaaugccaauaauggccagguugagaaacucugcacagaagcauccaguuauuugucuguuugcucaacaagcuugugcucaucaugcucuguguuccugacgcugugcuggguguuggcggugggaagauuacaagagucacauggcagcuguccuccuggaagguacaacccaguagagaugcagacuaacagagagccaauuacaaagcagugugacaagcgucaugguggaaaauuaaaagcucaaacaagggcacaugggaggggcuuccaacacagacuuugggggauccaggaaggucuaagaggaaagugggucucaccaaagccuugaccauaggcagaggguaccaguggaaaagguggggugaagaacauugaggacaaaaggaagaagugcaggaaggcccugaggcaagggaguggggggugcccuggagggauggcagcagggcagucugucagacccaaguggccuccagcccuagaagccaauuaguccuccucaaaaagcugucacuguccccuaagaauugcugccaggcucccacuggccugacucagucuuugagagucuuaaggaggaggucucugaaagguacacaccaagaacucuccccagcacagcuguuuuuaagacucuccaccagcgucauuggcguguugggaagaaacccucugccacagaggccagcuucagccuuugccuaacaccgcaagggcaaauggaaagguaaacgggaaggagaugucuccccagcaggcuauuugaggacagucuucccugcagaagaucucaaccugggguccacagaguggaaauguuagaguagggagcuaggcaaacaugagcaggacaggugagggcccccacaggaaugucaggcuaccaucaggugauggucaggugguuguuaaacugucucuguaaaauaauaauugguugcagccagcuccaagcaaggacagucucucaauagauacaaaacacccugaucuggugaucagccgcuucccgauaagaucucaggagcugggcaagcagccuggagcaugcgcaccaagaggcaaaauggcggaauuuaaccaguauaugaccuaccuuccucugggaacgcacgacugguaaggggaaaaaugccucaagugagcaugcgcgcaacuucaguaaucacacugugcaugcgaccccuuccaagugcuggcaggucaccacauacgcggacagccugcugcaagggaagaaucaggggagaugagacguaaaucccagaacuaugccaaauacauaaaaccccaaguuaagggucaggcagggcacuuagaucucucaaguugccugccugacccaaguguaguguacuuccuuuuguuccugcucuaaaacuuuuuaauaaacucucacuccugcucuaaaa

SEQ ID NO: 14 - protein, 738 aa, Homo sapiens
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQLLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 15 - RNA, 1350 nt, Homo sapiens
guucguugcaacaaauugaugagcaaugcuuuuuuauaaugccaacuuuguacaaaaaaguuggcaugagcggugcuccgacggccggggcagcccugaugcucugcgccgccaccgccgugcuacugagcgcucagggcggacccgugcaguccaagucgccgcgcuuugcguccugggacgagaugaauguccuggcgcacggacuccugcagcucggccaggggcugcgcgaacacgcggagcgcacccgcagucagcugagcgcgcuggagcggcgccugagcgcgugcggguccgccugucagggaaccgagggguccaccgaccucccguuagccccugagagccggguggacccugagguccuucacagccugcagacacaacucaaggcucagaacagcaggauccagcaacucuuccacaagguggcccagcagcagcggcaccuggagaagcagcaccugcgaauucagcaucugcaaagccaguuuggccuccuggaccacaagcaccuagaccaugagguggccaagccugcccgaagaaagaggcugcccgagauggcccagccaguugacccggcucacaaugucagccgccugcaccggcugcccagggauugccaggagcuguuccagguuggggagaggcagaguggacuauuugaaauccagccucaggggucuccgccauuuuuggugaacugcaagaugaccucagauggaggcuggacaguaauucagaggcgccacgauggcucaguggacuucaaccggcccugggaagccuacaaggcgggguuuggggauccccacggcgaguucuggcugggucuggagaaggugcauagcaucacgggggaccgcaacagccgccuggccgugcagcugcgggacugggauggcaacgccgaguugcugcaguucuccgugcaccuggguggcgaggacacggccuauagccugcagcucacugcacccguggccggccagcugggcgccaccaccgucccacccagcggccucuccguacccuucuccacuugggaccaggaucacgaccuccgcagggacaagaacugcgccaagagccucucuggaggcuggugguuuggcaccugcagccauuccaaccucaacagccaguacuuccgcuccaucccacagcagcggcagaagcuuaagaagggaaucuucuggaagaccuggcggggccgcuacuacccacugcaggccaccaccauguugauccagcccauggcagcagaggcagccuccuacccaacuuucuuguacaaaguuggcauuauaagaaagcauugcuuaucaauuuguugcaacgaac

SEQ ID NO: 16 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 17 - RNA, 2615 nt, Homo sapiens
ccuuuuuuggccucgacggcggcaacccagccucccuccuaacgcccuccgccuuugggaccaaccaggggagcucaaguuaguagcagccaaggagaggcgcugccuugccaagacuaaaaagggaggggagaagagaggaaaaaagcaagaaucccccaccccucucccgggcggagggggcgggaagagcgcguccuggccaagccgaguagugucuuccacucggugcgucucucuaggagccgcgcgggaaggaugcugguccgcaggggcgcgcgcgagggcccaggaugccgcggggcuggaccgcgcuuugcuugcugaguuugcugccuucuggguucaugagucuugacaacaacgguacugcuaccccagaguuaccuacccagggaacauuuucaaauguuucuacaaauguauccuaccaagaaacuacaacaccuaguacccuuggaaguaccagccugcacccugugucucaacauggcaaugaggccacaacaaacaucacagaaacgacagucaaauucacaucuaccucugugauaaccucaguuuauggaaacacaaacucuucuguccagucacagaccucuguaaucagcacaguguucaccaccccagccaacguuucaacuccagagacaaccuugaagccuagccugucaccuggaaauguuucagaccuuucaaccacuagcacuagccuugcaacaucucccacuaaacccuauacaucaucuucuccuauccuaagugacaucaaggcagaaaucaaauguucaggcaucagagaagugaaauugacucagggcaucugccuggagcaaaauaagaccuccagcugugcggaguuuaagaaggacaggggagagggccuggcccgagugcuguguggggaggagcaggcugaugcugaugcuggggcccagguaugcucccugcuccuugcccagucugaggugaggccucagugucuacugcuggucuuggccaacagaacagaaauuuccagcaaacuccaacuuaugaaaaagcaccaaucugaccugaaaaagcuggggauccuagauuucacugagcaagauguugcaagccaccagagcuauucccaaaagacccugauugcacuggucaccucgggagcccugcuggcugucuugggcaucacuggcuauuuccugaugaaucgccgcagcuggagccccacaggagaaaggcugggcgaagacccuuauuacacggaaaacgguggaggccagggcuauagcucaggaccugggaccuccccugaggcucagggaaaggccagugugaaccgaggggcucagaaaaacgggaccggccaggccaccuccagaaacggccauucagcaagacaacacgugguggcugauaccgaauugugacucggcuagguggggcaaggcugggcaguguccgagagagcaccccucucugcaucugaccacgugcuacccccaugcuggaggugacaucucuuacgcccaacccuuccccacugcacacaccucagaggcuguucuuggggcccuacaccuugaggagggggcagguaaacuccuguccuuuacacauucggcucccuggagccagacucuggucuucuuuggguaaacgugugacgggggaaagccaaggucuggagaagcucccaggaacaaucgauggccuugcagcacucacacaggacccccuuccccuacccccuccucucugccgcaauacaggaacccccaggggaaagaugagcuuuucuaggcuacaauuuucucccaggaagcuuugauuuuuaccguuucuucccuguauuuucuuucucuacuuugaggaaaccaaaguaaccuuuugcaccugcucucuuguaaugauauagccagaaaaacguguugccuugaaccacuucccucaucucuccuccaagacacuguggacuuggucaccagcuccucccuuguucucuaaguuccacugagcuccaugugcccccucuaccauuugcagaguccugcacaguuuucuggcuggagccuagaacaggccucccaaguuuuaggacaaacagcucaguucuagucucucuggggccacacagaaacucuuuuugggcuccuuuuucucccucuggaucaaaguaggcaggaccaugggaccaggucuuggagcugagccucucaccuguacucuuccgaaaaauccucuuccucugaggcuggauccuagccuuauccucugaucuccauggcuuccuccucccuccugccgacuccuggguugagcuguugccucagucccccaacagaugcuuuucugucucugccucccucacccugagccccuuccuugcucugcacccccauauggucauagcccagaucagcuccuaacccuuaucaccagcugccucuucugugggugacccagguccuuguuugcuguugauuucuuuccagagggguugagcagggauccugguuucaaugacgguuggaaauagaaauuuccagagaagagaguauuggguagauauuuuuucugaauacaaagugauguguuuaaauacugcaauuaaagugauacugaaacac

SEQ ID NO: 18 - protein, 385 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 19 - RNA, 1197 nt, Homo sapiens
gucucaauauuagagucucaacccccaauaaauauaggacuggagaugucugaggcucauucugcccucgagcccaccgggaacgaaagagaagcucuaucuccccuccaggagcccagcuaugaacuccuucuccacaagcgccuucgguccaguugccuucucccuggggcugcuccugguguugccugcugccuucccugccccaguacccccaggagaagauuccaaagauguagccgccccacacagacagccacucaccucuucagaacgaauugacaaacaaauucgguacauccucgacggcaucucagcccugagaaaggagacauguaacaagaguaacaugugugaaagcagcaaagaggcacuggcagaaaacaaccugaaccuuccaaagauggcugaaaaagauggaugcuuccaaucuggauucaaugaggagacuugccuggugaaaaucaucacuggucuuuuggaguuugagguauaccuagaguaccuccagaacagauuugagaguagugaggaacaagccagagcugugcagaugaguacaaaaguccugauccaguuccugcagaaaaaggcaaagaaucuagaugcaauaaccaccccugacccaaccacaaaugccagccugcugacgaagcugcaggcacagaaccaguggcugcaggacaugacaacucaucucauucugcgcagcuuuaaggaguuccugcaguccagccugagggcucuucggcaaauguagcaugggcaccucagauuguuguuguuaaugggcauuccuucuucuggucagaaaccuguccacugggcacagaacuuauguuguucucuauggagaacuaaaaguaugagcguuaggacacuauuuuaauuauuuuuaauuuauuaauauuuaaauaugugaagcugaguuaauuuauguaagucauauuuauauuuuuaagaaguaccacuugaaacauuuuauguauuaguuuugaaauaauaauggaaaguggcuaugcaguuugaauauccuuuguuucagagccagaucauuucuuggaaaguguaggcuuaccucaaauaaauggcuaacuuauacauauuuuuaaagaaauauuuauauuguauuuauauaauguauaaaugguuuuuauaccaauaaauggcauuuuaaaaaauucagcaaaaaaaaaa

SEQ ID NO: 20 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 21 - RNA, 1184 nt, Homo sapiens
cacagagcccgggccgcaggcaccuccucgccagcucuuccgcuccucucacagccgccagacccgccugcugagccccauggcccgcgcugcucucuccgccgcccccagcaauccccggcuccugcgaguggcacugcugcuccugcuccugguagccgcuggccggcgcgcagcaggagcguccguggccacugaacugcgcugccagugcuugcagacccugcagggaauucaccccaagaacauccaaagugugaacgugaagucccccggaccccacugcgcccaaaccgaagucauagccacacucaagaaugggcggaaagcuugccucaauccugcaucccccauaguuaagaaaaucaucgaaaagaugcugaacagugacaaauccaacugaccagaagggaggaggaagcucacugguggcuguuccugaaggaggcccugcccuuauaggaacagaagaggaaagagagacacagcugcagaggccaccuggauugugccuaauguguuugagcaucgcuuaggagaagucuucuauuuauuuauuuauucauuaguuuugaagauucuauguuaauauuuuagguguaaaauaauuaaggguaugauuaacucuaccugcacacuguccuauuauauucauucuuuuugaaaugucaaccccaaguuaguucaaucuggauucauauuuaauuugaagguagaauguuuucaaauguucuccagucauuauguuaauauuucugaggagccugcaacaugccagccacugugauagaggcuggcggauccaagcaaauggccaaugagaucauugugaaggcaggggaauguaugugcacaucuguuuuguaacuguuuagaugaaugucaguuguuauuuauugaaaugauuucacaguguguggucaacauuucucauguugaaacuuuaagaacuaaaauguucuaaauaucccuuggacauuuuaugucuuucuuguaaggcauacugccuuguuuaaugguaguuuuacaguguuucuggcuuagaacaaaggggcuuaauuauugauguuuucauagagaauauaaaaauaaagcacuuauagaaaaaacucguuugauuuuuggggggaaacaagggcuaccuuuacuggaaaaucuggugauuuauaaaaaaaaaaaaaaaa

SEQ ID NO: 22 - protein, 107 aa, Homo sapiens
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 23 - RNA, 1234 nt, Homo sapiens
gagcuccgggaauuucccuggcccgggacuccgggcuuuccagccccaaccaugcauaaaagggguucgccguucucggagagccacagagcccgggccacaggcagcuccuugccagcucuccuccucgcacagccgcucgaaccgccugcugagccccauggcccgcgccacgcucuccgccgcccccagcaauccccggcuccugcggguggcgcugcugcuccugcuccugguggccgccagccggcgcgcagcaggagcgccccuggccacugaacugcgcugccagugcuugcagacccugcagggaauucaccucaagaacauccaaagugugaaggugaagucccccggaccccacugcgcccaaaccgaagucauagccacacucaagaaugggcagaaagcuugucucaaccccgcaucgcccaugguuaagaaaaucaucgaaaagaugcugaaaaauggcaaauccaacugaccagaaggaaggaggaagcuuauugguggcuguuccugaaggaggcccugcccuuacaggaacagaagaggaaagagagacacagcugcagaggccaccuggauugcgccuaauguguuugagcaucacuuaggagaagucuucuauuuauuuauuuauuuauuuauuuguuuguuuuagaagauucuauguuaauauuuuauguguaaaauaagguuaugauugaaucuacuugcacacucucccauuauauuuauuguuuauuuuaggucaaacccaaguuaguucaauccugauucauauuuaauuugaagauagaagguuugcagauauucucuagucauuuguuaauauuucuucgugaugacauaucacaugucagccacugugauagaggcugaggaauccaagaaaauggccagugagaucaaugugacggcagggaaauguaugugugucuauuuuguaacuguaaagaugaaugucaguuguuauuuauugaaaugauuucacaguguguggucaacauuucucauguugaagcuuuaagaacuaaaauguucuaaauaucccuuggacauuuuaugucuuucuuguaaggcauacugccuuguuuaauguuaauuaugcaguguuucccucuguguuagagcagagagguuucgauauuuauugauguuuucacaaagaacaggaaaauaaaauauuuaaaaauauaaaaaaaaaaaaaaaaaaaaaaaaaaaaaaaaa

SEQ ID NO: 24 - protein, 107 aa, Homo sapiens
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 25 - RNA, 1166 nt, Homo sapiens
gcuccgggaauuucccuggcccggccgcuccgggcuuuccagucucaaccaugcauaaaaaggguucgccgaucuuggggagccacacagcccgggucgcaggcaccuccccgccagcucucccgcuucucgcacagcuucccgacgcgucugcugagccccauggcccacgccacgcucuccgccgcccccagcaauccccggcuccugcggguggcgcugcugcuccugcuccugguggccgccagccggcgcgcagcaggagcguccguggucacugaacugcgcugccagugcuugcagacacugcagggaauucaccucaagaacauccaaagugugaauguaaggucccccggaccccacugcgcccaaaccgaagucauagccacacucaagaaugggaagaaagcuugucucaaccccgcaucccccaugguucagaaaaucaucgaaaagauacugaacaaggggagcaccaacugacaggagagaaguaagaagcuuaucagcguaucauugacacuuccugcaggguggucccugcccuuaccagagcugaaaaugaaaaagagaacagcagcuuucuagggacagcuggaaaggacuuaauguguuugacuauuucuuacgaggguucuacuuauuuauguauuuauuuuugaaagcuuguauuuuaauauuuuacaugcuguuauuuaaagaugugaguguguuucaucaaacauagcucaguccugauuauuuaauuggaauaugauggguuuuaaaugugucauuaaacuaauauuuagugggagaccauaaugugucagccaccuugauaaaugacaggguggggaacuggaggguggggggauugaaaugcaagcaauuaguggaucacuguuaggguaagggaauguauguacacaucuauuuuuuauacuuuuuuuuuaaaaaaagaaugucaguuguuauuuauucaaauuaucucacauuauguguucaacauuuuuaugcugaaguuucccuuagacauuuuaugucuugcuuguagggcauaaugccuuguuuaauguccauucugcagcguuucucuuucccuuggaaaagagaauuuaucauuacuguuacauuuguacaaaugacaugauaauaaaaguuuuaugaaaaaaaaaaaaaaa

SEQ ID NO: 26 - protein, 107 aa, Homo sapiens
MAHATLSAAPSNPRLLRVALLLLLLVAASRRAAGASVVTELRCQCLQTLQGIHLKNIQSVNVRSPGPHCAQTEVIATLKNGKKACLNPASPMVQKIIEKILNKGSTN

SEQ ID NO: 27 - RNA, 1718 nt, Homo sapiens
gagggugcauaaguucucuaguagggugaugauauaaaaagccaccggagcacuccauaaggcacaaacuuucagagacagcagagcacacaagcuucuaggacaagagccaggaagaaaccaccggaaggaaccaucucacuguguguaaacaugacuuccaagcuggccguggcucucuuggcagccuuccugauuucugcagcucugugugaaggugcaguuuugccaaggagugcuaaagaacuuagaugucagugcauaaagacauacuccaaaccuuuccaccccaaauuuaucaaagaacugagagugauugagaguggaccacacugcgccaacacagaaauuauuguaaagcuuucugauggaagagagcucugucuggaccccaaggaaaacugggugcagaggguuguggagaaguuuuugaagagggcugagaauucauaaaaaaauucauucucugugguauccaagaaucagugaagaugccagugaaacuucaagcaaaucuacuucaacacuucauguauugugugggucuguuguaggguugccagaugcaauacaagauuccugguuaaauuugaauuucaguaaacaaugaauaguuuuucauuguaccaugaaauauccagaacauacuuauauguaaaguauuauuuauuugaaucuacaaaaaacaacaaauaauuuuuaaauauaaggauuuuccuagauauugcacgggagaauauacaaauagcaaaauugaggccaagggccaagagaauauccgaacuuuaauuucaggaauugaauggguuugcuagaaugugauauuugaagcaucacauaaaaaugaugggacaauaaauuuugccauaaagucaaauuuagcuggaaauccuggauuuuuuucuguuaaaucuggcaacccuagucugcuagccaggauccacaaguccuuguuccacugugccuugguuucuccuuuauuucuaaguggaaaaaguauuagccaccaucuuaccucacagugauguugugaggacauguggaagcacuuuaaguuuuuucaucauaacauaaauuauuuucaaguguaacuuauuaaccuauuuauuauuuauguauuuauuuaagcaucaaauauuugugcaagaauuuggaaaaauagaagaugaaucauugauugaauaguuauaaagauguuauaguaaauuuauuuuauuuuagauauuaaaugauguuuuauuagauaaauuucaaucaggguuuuuagauuaaacaaacaaacaauuggguacccaguuaaauuuucauuucagauaaacaacaaauaauuuuuuaguauaaguacauuauuguuuaucugaaauuuuaauugaacuaacaauccuaguuugauacucccagucuugucauugccagcuguguugguagugcuguguugaauuacggaauaaugaguuagaacuauuaaaacagccaaaacuccacagucaauauuaguaauuucuugcugguugaaacuuguuuauuauguacaaauagauucuuauaauauuauuuaaaugacugcauuuuuaaauacaaggcuuuauauuuuuaacuuuaagauguuuuuaugugcucuccaaauuuuuuuuacuguuucugauuguauggaaauauaaaaguaaauaugaaacauuuaaaauauaauuuguugucaaaguaaaaaaaaaaaaaaa

SEQ ID NO: 28 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 29 - RNA, 4507 nt, Homo sapiens
gaccaauugucauacgacuugcagugagcgucaggagcacguccaggaacuccucagcagcgccuccuucagcuccacagccagacgcccucagacagcaaagccuacccccgcgccgcgcccugcccgccgcugcgaugcucgcccgcgcccugcugcugugcgcgguccuggcgcucagccauacagcaaauccuugcuguucccacccaugucaaaaccgagguguauguaugagugugggauuugaccaguauaagugcgauuguacccggacaggauucuauggagaaaacugcucaacaccggaauuuuugacaagaauaaaauuauuucugaaacccacuccaaacacagugcacuacauacuuacccacuucaagggauuuuggaacguugugaauaacauucccuuccuucgaaaugcaauuaugaguuauguguugacauccagaucacauuugauugacaguccaccaacuuacaaugcugacuauggcuacaaaagcugggaagccuucucuaaccucuccuauuauacuagagcccuuccuccugugccugaugauugcccgacucccuugggugucaaagguaaaaagcagcuuccugauucaaaugagauuguggaaaaauugcuucuaagaagaaaguucaucccugauccccagggcucaaacaugauguuugcauucuuugcccagcacuucacgcaucaguuuuucaagacagaucauaagcgagggccagcuuucaccaacgggcugggccaugggguggacuuaaaucauauuuacggugaaacucuggcuagacagcguaaacugcgccuuuucaaggauggaaaaaugaaauaucagauaauugauggagagauguauccucccacagucaaagauacucaggcagagaugaucuacccuccucaagucccugagcaucuacgguuugcuguggggcaggaggucuuuggucuggugccuggucugaugauguaugccacaaucuggcugcgggaacacaacagaguaugcgaugugcuuaaacaggagcauccugaauggggugaugagcaguuguuccagacaagcaggcuaauacugauaggagagacuauuaagauugugauugaagauuaugugcaacacuugaguggcuaucacuucaaacugaaauuugacccagaacuacuuuucaacaaacaauuccaguaccaaaaucguauugcugcugaauuuaacacccucuaucacuggcauccccuucugccugacaccuuucaaauucaugaccagaaauacaacuaucaacaguuuaucuacaacaacucuauauugcuggaacauggaauuacccaguuuguugaaucauucaccaggcaaauugcuggcaggguugcuggugguaggaauguuccacccgcaguacagaaaguaucacaggcuuccauugaccagagcaggcagaugaaauaccagucuuuuaaugaguaccgcaaacgcuuuaugcugaagcccuaugaaucauuugaagaacuuacaggagaaaaggaaaugucugcagaguuggaagcacucuauggugacaucgaugcuguggagcuguauccugcccuucugguagaaaagccucggccagaugccaucuuuggugaaaccaugguagaaguuggagcaccauucuccuugaaaggacuuauggguaauguuauauguucuccugccuacuggaagccaagcacuuuugguggagaaguggguuuucaaaucaucaacacugccucaauucagucucucaucugcaauaacgugaagggcugucccuuuacuucauucaguguuccagauccagagcucauuaaaacagucaccaucaaugcaaguucuucccgcuccggacuagaugauaucaaucccacaguacuacuaaaagaacguucgacugaacuguagaagucuaaugaucauauuuauuuauuuauaugaaccaugucuauuaauuuaauuauuuaauaauauuuauauuaaacuccuuauguuacuuaacaucuucuguaacagaagucaguacuccuguugcggagaaaggagucauacuugugaagacuuuuaugucacuacucuaaagauuuugcuguugcuguuaaguuuggaaaacaguuuuuauucuguuuuauaaaccagagagaaaugaguuuugacgucuuuuuacuugaauuucaacuuauauuauaagaacgaaaguaaagauguuugaauacuuaaacacugucacaagauggcaaaaugcugaaaguuuuuacacugucgauguuuccaaugcaucuuccaugaugcauuagaaguaacuaauguuugaaauuuuaaaguacuuuugguuauuuuucugucaucaaacaaaaacagguaucagugcauuauuaaaugaauauuuaaauuagacauuaccaguaauuucaugucuacuuuuuaaaaucagcaaugaaacaauaauuugaaauuucuaaauucauaggguagaaucaccuguaaaagcuuguuugauuucuuaaaguuauuaaacuuguacauauaccaaaaagaagcugucuuggauuuaaaucuguaaaaucaguagaaauuuuacuacaauugcuuguuaaaauauuuuauaagugauguuccuuuuucaccaagaguauaaaccuuuuuagugugacuguuaaaacuuccuuuuaaaucaaaaugccaaauuuauuaaggugguggagccacugcaguguuaucuuaaaauaagaauauuuuguugagauauuccagaauuuguuuauauggcugguaacauguaaaaucuauaucagcaaaagggucuaccuuuaaaauaagcaauaacaaagaagaaaaccaaauuauuguucaaauuuagguuuaaacuuuugaagcaaacuuuuuuuuauccuugugcacugcaggccugguacucagauuuugcuaugagguuaaugaaguaccaagcugugcuugaauaaugauauguuuucucagauuuucuguuguacaguuuaauuuagcaguccauaucacauugcaaaaguagcaaugaccucauaaaauaccucuucaaaaugcuuaaauucauuucacacauuaauuuuaucucagucuugaagccaauucaguaggugcauuggaaucaagccuggcuaccugcaugcuguuccuuuucuuuucuucuuuuagccauuuugcuaagagacacagucuucucaucacuucguuucuccuauuuuguuuuacuaguuuuaagaucagaguucacuuucuuuggacucugccuauauuuucuuaccugaacuuuugcaaguuuucagguaaaccucagcucaggacugcuauuuagcuccucuuaagaagauuaaaagagaaaaaaaaaggcccuuuuaaaaauaguauacacuuauuuuaagugaaaagcagagaauuuuauuuauagcuaauuuuagcuaucuguaaccaagauggaugcaaagaggcuagugccucagagagaacuguacgggguuugugacuggaaaaaguuacguucccauucuaauuaaugcccuuucuuauuuaaaaacaaaaccaaaugauaucuaaguaguucucagcaauaauaauaaugacgauaauacuucuuuuccacaucucauugucacugacauuuaaugguacuguauauuacuuaauuuauugaagauuauuauuuaugucuuauuaggacacuaugguuauaaacuguguuuaagccuacaaucauugauuuuuuuuuguuaugucacaaucaguauauuuucuuugggguuaccucucugaauauuauguaaacaauccaaagaaaugauuguauuaagauuugugaauaaauuuuuagaaaucugauuggcauauugagauauuuaagguugaauguuuguccuuaggauaggccuaugugcuagcccacaaagaauauugucucauuagccugaaugugccauaagacugaccuuuuaaaauguuuugagggaucuguggaugcuucguuaauuuguucagccacaauuuauugagaaaauauucugugucaagcacuguggguuuuaauauuuuuaaaucaaacgcugauuacagauaauaguauuuauauaaauaauugaaaaaaauuuucuuuugggaagagggagaaaaugaaauaaauaucauuaaagauaacucaggagaaucuucuuuacaauuuuacguuuagaauguuuaagguuaagaaagaaauagucaauaugcuuguauaaaacacuguucacuguuuuuuuuaaaaaaaaaacuugauuuguuauuaacauugaucugcugacaaaaccugggaauuuggguuguguaugcgaauguuucagugccucagacaaauguguauuuaacuuauguaaaagauaagucuggaaauaaaugucuguuuauuuuuguacuauuuaaaaauugacagaucuuuucugaagaaaaaaaaaaaaaaa

SEQ ID NO: 30 - protein, 604 aa, Homo sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 31 - RNA, 1204 nt, Homo sapiens
ggggugcaaagaagagacagcagcgcccagcuuggaggugcuaacuccagaggccagcaucagcaacugggcacagaaaggagccgccugggcagggaccauggcacggccacaucccugguggcugugcguucuggggacccugguggggcucucagcuacuccagcccccaagagcugcccagagaggcacuacugggcucagggaaagcugugcugccagaugugugagccaggaacauuccucgugaaggacugugaccagcauagaaaggcugcucagugugauccuugcauaccgggggucuccuucucuccugaccaccacacccggccccacugugagagcugucggcacuguaacucuggucuucucguucgcaacugcaccaucacugccaaugcugagugugccugucgcaauggcuggcagugcagggacaaggagugcaccgagugugauccucuuccaaacccuucgcugaccgcucggucgucucaggcccugagcccacacccucagcccacccacuuaccuuaugucagugagaugcuggaggccaggacagcugggcacaugcagacucuggcugacuucaggcagcugccugcccggacucucucuacccacuggccaccccaaagaucccugugcagcuccgauuuuauucgcauccuugugaucuucucuggaauguuccuuguuuucacccuggccggggcccuguuccuccaucaacgaaggaaauauagaucaaacaaaggagaaaguccuguggagccugcagagccuugucguuacagcugccccagggaggaggagggcagcaccauccccauccaggaggauuaccgaaaaccggagccugccugcucccccugagccagcaccugcgguagcugcacuacagcccuggccuccacccccaccccgccgaccauccaagggagagugagaccuggcagccacaacugcagucccauccucuugucagggcccuuuccuguguacacgugacagagugccuuuucgagacuggcagggacgaggacaaauauggaugagguggagagugggaagcaggagcccagccagcugcgccugcgcugcaggagggcgggggcucugguuguaaaacacacuuccugcugcgaaagacccacaugcuacaagacgggcaaaauaaagugacagaugaccacc

SEQ ID NO: 32 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 33 - RNA, 2397 nt, Homo sapiens
gcacacacucaucgaaaaaaauuuggauuauuagaagagagaggucugcggcuuccacaccguacagcgugguuuuucuucucgguauaaaagcaaaguuguuuuugauacgugacaguuucccacaagccaggcugauccuuuucugucaguccacuucaccaagccugcccuuggacaaggacccgaugcccaaccccaggccuggcaagcccucggccccuuccuuggcccuuggcccauccccaggagccucgcccagcuggagggcugcacccaaagccucagaccugcugggggcccggggcccagggggaaccuuccagggccgagaucuucgaggcggggcccaugccuccucuucuuccuugaaccccaugccaccaucgcagcugcagcugcccacacugccccuagucaugguggcacccuccggggcacggcugggccccuugccccacuuacaggcacuccuccaggacaggccacauuucaugcaccagcucucaacgguggaugcccacgcccggaccccugugcugcaggugcacccccuggagagcccagccaugaucagccucacaccacccaccaccgccacuggggucuucucccucaaggcccggccuggccucccaccugggaucaacguggccagccuggaauggguguccagggagccggcacugcucugcaccuucccaaaucccagugcacccaggaaggacagcacccuuucggcugugccccagagcuccuacccacugcuggcaaauggugucugcaaguggcccggaugugagaaggucuucgaagagccagaggacuuccucaagcacugccaggcggaccaucuucuggaugagaagggcagggcacaaugucuccuccagagagagaugguacagucucuggagcagcagcuggugcuggagaaggagaagcugagugccaugcaggcccaccuggcugggaaaauggcacugaccaaggcuucaucuguggcaucauccgacaagggcuccugcugcaucguagcugcuggcagccaaggcccugucgucccagccuggucuggcccccgggaggccccugacagccuguuugcuguccggaggcaccugugggguagccauggaaacagcacauucccagaguuccuccacaacauggacuacuucaaguuccacaacaugcgacccccuuucaccuacgccacgcucauccgcugggccauccuggaggcuccagagaagcagcggacacucaaugagaucuaccacugguucacacgcauguuugccuucuucagaaaccauccugccaccuggaagaacgccauccgccacaaccugagucugcacaagugcuuugugcggguggagagcgagaagggggcuguguggaccguggaugagcuggaguuccgcaagaaacggagccagaggcccagcagguguuccaacccuacaccuggccccugaccucaagaucaaggaaaggaggauggacgaacaggggccaaacuggugggaggcagagguggugggggcagggaugauaggcccuggaugugcccacagggaccaagaagugagguuuccacugucuugccugccagggccccuguucccccgcuggcagccacccccucccccaucauauccuuugccccaaggcugcucagaggggccccgguccuggccccagcccccaccuccgccccagacacaccccccagucgagcccugcagccaaacagagccuucacaaccagccacacagagccugccucagcugcucgcacagauuacuucagggcuggaaaagucacacagacacacaaaaugucacaauccugucccucacucaacacaaaccccaaaacacagagagccugccucaguacacucaaacaaccucaaagcugcaucaucacacaaucacacacaagcacagcccugacaacccacacaccccaaggcacgcacccacagccagccucagggcccacaggggcacugucaacacaggggugugcccagaggccuacacagaagcagcgucaguacccucaggaucugaggucccaacacgugcucgcucacacacacggccuguuagaauucaccuguguaucucacgcauaugcacacgcacagccccccagugggucucuugagucccgugcagacacacacagccacacacacugccuugccaaaaauaccccgugucuccccugccacucaccucacucccauucccugagcccugauccaugccucagcuuagacugcagaggaacuacucauuuauuugggauccaaggcccccaacccacaguaccguccccaauaaacugcagccgagcuccccacaaaaaaaaaaaaaaaa

SEQ ID NO: 34 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 35 - RNA, 2115 nt, Homo sapiens
aguuucccuuccgcucaccuccgccugagcaguggagaaggcggcacucugguggggcugcuccaggcaugcagaucccacaggcgcccuggccagucgucugggcggugcuacaacugggcuggcggccaggaugguucuuagacuccccagacaggcccuggaacccccccaccuucuccccagcccugcucguggugaccgaaggggacaacgccaccuucaccugcagcuucuccaacacaucggagagcuucgugcuaaacugguaccgcaugagccccagcaaccagacggacaagcuggccgccuuccccgaggaccgcagccagcccggccaggacugccgcuuccgugucacacaacugcccaacgggcgugacuuccacaugagcguggucagggcccggcgcaaugacagcggcaccuaccucuguggggccaucucccuggcccccaaggcgcagaucaaagagagccugcgggcagagcucagggugacagagagaagggcagaagugcccacagcccaccccagccccucacccaggccagccggccaguuccaaacccuggugguuggugucgugggcggccugcugggcagccuggugcugcuagucuggguccuggccgucaucugcucccgggccgcacgagggacaauaggagccaggcgcaccggccagccccugaaggaggaccccucagccgugccuguguucucuguggacuauggggagcuggauuuccaguggcgagagaagaccccggagccccccgugcccugugucccugagcagacggaguaugccaccauugucuuuccuagcggaaugggcaccucaucccccgcccgcaggggcucagcugacggcccucggagugcccagccacugaggccugaggauggacacugcucuuggccccucugaccggcuuccuuggccaccaguguucugcagacccuccaccaugagcccgggucagcgcauuuccucaggagaagcaggcagggugcaggccauugcaggccguccaggggcugagcugccugggggcgaccggggcuccagccugcaccugcaccaggcacagccccaccacaggacucaugucucaaugcccacagugagcccaggcagcaggugucaccguccccuacagggagggccagaugcagucacugcuucagguccugccagcacagagcugccugcguccagcucccugaaucucugcugcugcugcugcugcugcugcugcugccugcggcccggggcugaaggcgccguggcccugccugacgccccggagccuccugccugaacuugggggcugguuggagauggccuuggagcagccaaggugccccuggcaguggcaucccgaaacgcccuggacgcagggcccaagacugggcacaggagugggagguacauggggcuggggacuccccaggaguuaucugcucccugcaggccuagagaaguuucagggaaggucagaagagcuccuggcuguggugggcagggcaggaaaccccuccaccuuuacacaugcccaggcagcaccucaggcccuuuguggggcagggaagcugaggcaguaagcgggcaggcagagcuggaggccuuucaggcccagccagcacucuggccuccugccgccgcauuccaccccagccccucacaccacucgggagagggacauccuacggucccaaggucaggagggcagggcugggguugacucaggccccucccagcuguggccaccuggguguugggagggcagaagugcaggcaccuagggccccccaugugcccacccugggagcucuccuuggaacccauuccugaaauuauuuaaagggguuggccgggcucccaccagggccugggugggaagguacaggcguucccccggggccuaguacccccgccguggccuauccacuccucacauccacacacugcacccccacuccuggggcagggccaccagcauccaggcggccagcaggcaccugaguggcugggacaagggaucccccuucccugugguucuauuauauuauaauuauaauuaaauaugagagcaugcuaaggaaaa

SEQ ID NO: 36 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 37 - RNA, 2033 nt, Homo sapiens
cuucugugugugcacauguguaauacauaucugggaucaaagcuaucuauauaaaguccuugauucuguguggguucaaacacauuucaaagcuucaggauccugaaagguuuugcucuacuuccugaagaccugaacaccgcucccauaaagccauggcuugccuuggauuucagcggcacaaggcucagcugaaccuggcuaccaggaccuggcccugcacucuccuguuuuuucuucucuucaucccugucuucugcaaagcaaugcacguggcccagccugcugugguacuggccagcagccgaggcaucgccagcuuugugugugaguaugcaucuccaggcaaagccacugagguccgggugacagugcuucggcaggcugacagccaggugacugaagucugugcggcaaccuacaugauggggaaugaguugaccuuccuagaugauuccaucugcacgggcaccuccaguggaaaucaagugaaccucacuauccaaggacugagggccauggacacgggacucuacaucugcaagguggagcucauguacccaccgccauacuaccugggcauaggcaacggaacccagauuuauguaauugauccagaaccgugcccagauucugacuuccuccucuggauccuugcagcaguuaguucgggguuguuuuuuuauagcuuucuccucacagcuguuucuuugagcaaaaugcuaaagaaaagaagcccucuuacaacaggggucuaugugaaaaugcccccaacagagccagaaugugaaaagcaauuucagccuuauuuuauucccaucaauugagaaaccauuaugaagaagagaguccauauuucaauuuccaagagcugaggcaauucuaacuuuuuugcuauccagcuauuuuuauuuguuugugcauuuggggggaauucaucucucuuuaauauaaaguuggaugcggaacccaaauuacguguacuacaauuuaaagcaaaggaguagaaagacagagcugggauguuucugucacaucagcuccacuuucagugaaagcaucacuugggauuaauauggggaugcagcauuaugaugugggucaaggaauuaaguuagggaauggcacagcccaaagaaggaaaaggcagggagcgagggagaagacuauauuguacacaccuuauauuuacguaugagacguuuauagccgaaaugaucuuuucaaguuaaauuuuaugccuuuuauuucuuaaacaaauguaugauuacaucaaggcuucaaaaauacucacauggcuauguuuuagccagugaugcuaaagguuguauugcauauauacauauauauauauauauauauauauauauauauauauauauauauauauauauauauuuuaauuugauaguauugugcauagagccacguauguuuuuguguauuuguuaaugguuugaauauaaacacuauauggcagugucuuuccaccuugggucccagggaaguuuuguggaggagcucaggacacuaauacaccagguagaacacaaggucauuugcuaacuagcuuggaaacuggaugaggucauagcagugcuugauugcguggaauugugcugaguugguguugacaugugcuuuggggcuuuuacaccaguuccuuucaaugguuugcaaggaagccacagcuggugguaucugaguugacuugacagaacacugucuugaagacaauggcuuacuccaggagacccacagguaugaccuucuaggaagcuccaguucgaugggcccaauucuuacaaacaugugguuaaugccauggacagaagaaggcagcagguggcagaauggggugcaugaagguuucugaaaauuaacacugcuuguguuuuuaacucaauauuuuccaugaaaaugcaacaacauguauaauauuuuuaauuaaauaaaaaucugugguggucguuuuaaaaaaaaaaaaaaaaaaaaaaaaaaaaaaaaaaaaaaaaaaaaaaaaaaaaaaaaaa

SEQ ID NO: 38 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 39 - RNA, 2978 nt, Homo sapiens
cguccuaucugcagucggcuacuuucaguggcagaagaggccacaucugcuuccuguaggcccucugggcagaagcaugcgcuggugucuccuccugaucugggcccaggggcugaggcaggcuccccucgccucaggaaugaugacaggcacaauagaaacaacggggaacauuucugcagagaaagguggcucuaucaucuuacaaugucaccucuccuccaccacggcacaagugacccaggucaacugggagcagcaggaccagcuucuggccauuuguaaugcugacuugggguggcacaucuccccauccuucaaggaucgaguggccccaggucccggccugggccucacccuccagucgcugaccgugaacgauacaggggaguacuucugcaucuaucacaccuacccugaugggacguacacugggagaaucuuccuggagguccuagaaagcucaguggcugagcacggugccagguuccagauuccauugcuuggagccauggccgcgacgcugguggucaucugcacagcagucaucgugguggucgcguugacuagaaagaagaaagcccucagaauccauucuguggaaggugaccucaggagaaaaucagcuggacaggaggaauggagccccagugcucccucacccccaggaagcuguguccaggcagaagcugcaccugcugggcucuguggagagcagcggggagaggacugugccgagcugcaugacuacuucaauguccugaguuacagaagccuggguaacugcagcuucuucacagagacugguuagcaaccagaggcaucuucuggaagauacacuuuugucuuugcuauuauagaugaauauauaagcagcuguacucuccaucagugcugcguguguguguguguguguauguguguguguguucaguugagugaauaaaugucauccucuucuccaucuucauuuccuuggccuuuucguucuauuccauuuugcauuauggcaggccuagggugaguaacguggaucuugaucauaaaugcaaaauuaaaaaauaucuugaccugguuuuaaaucuggcaguuugagcagauccuaugucucugagagacacauuccucauaauggccagcauuuugggcuacaagguuuugugguugaugaugaggauggcaugacugcagagccauccucaucucauuuuuucacgucauuuucaguaacuuucacucauucaaaggcagguuauaaguaaguccugguagcagccucuauggggagauuugagagugacuaaaucuugguaucugcccucaagaacuuacaguuaaauggggagacaauguugucaugaaaagguauuauaguaaggagagaaggagacauacacaggccuucaggaagagacgacaguuuggggugagguaguuggcauaggcuuaucugugaugaaguggccugggagcaccaaggggauguugaggcuagucugggaggagcaggaguuuugucuagggaacuuguaggaaauucuuggagcugaaagucccacaaagaaggcccuggcaccaagggagucagcaaacuucagauuuuauucucugggcaggcauuucaaguuuccuuuugcugugacauacucauccauuagacagccugauacaggccuguagccucuuccggccgugugugcuggggaagccccaggaaacgcacaugcccacacagggagccaagucguagcauuugggccuugaucuaccuuuucugcaucaauacacucuugagccuuugaaaaaagaacguuucccacuaaaaagaaaauguggauuuuuaaaauagggacucuuccuaggggaaaaaggggggcugggagugauagaggguuuaaaaaauaaacaccuucaaacuaacuucuucgaacccuuuuauucacucccugacgacuuugugcugggguugggguaacugaaccgcuuauuucuguuuaauugcauucaggcuggaucuuagaagacuuuuauccuuccaccaucucucucagaggaaugagcggggagguuggauuuacuggugacugauuuucuuucaugggccaaggaacugaaagagaaugugaagcaagguugugucuugcgcaugguuaaaaauaaagcauuguccugcuuccuaagacuuagacugggguugacaauuguuuuagcaacaagacaauucaacuauuucuccuaggauuuuuauuauuauuauuuuuucacuuuucuaccaaauggguuacauaggaagaaugaacugaaaucuguccagagcuccaaguccuuuggaagaaagauuagaugaacguaaaaauguuguuguuugcuguggcaguuuacagcauuuuucuugcaaaauuagugcaaaucuguuggaaauagaacacaauucacaaauuggaagugaacuaaaauguaaugacgaaaagggaguaguguuuugauuuggaggagguguauauucggcagagguuggacugagaguuggguguuauuuaacauaauuaugguaauugggaaacauuuauaaacacuauugggauggugauaaaauacaaaagggccuauagauguuagaaaugggucagguuacugaaaugggauucaauuugaaaaaaauuuuuuuaaauagaacucacugaacuagauucuccucugagaaccagagaagaccauuucauaguuggauuccuggagacaugcgcuauccaccacguagccacuuuccacauguggccaucaaccacuuaagaugggguuaguuuaaaucaagaugugcuguuauaauugguauaagcauaaaaucacacuagauucuggagauuuaauaugaauaauaagaauacuauuucaguaguuuugguauauugugugucaaaaaugauaauauuuuggauguauugggugaaauaaaauauuaacauuaaaaaaaaaaa

SEQ ID NO: 40 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 41 - RNA, 1944 nt, Homo sapiens
aauuucucacugccccugugauaaacuguggucacuggcuguggcagcaacuauuauaagaugcucugaaaacucuucagacacugaggggcaccagaggagcagacuacaagaauggcacacgcuauggaaaacuccuggacaaucaguaaagaguaccauauugaugaagaagugggcuuugcucugccaaauccacaggaaaaucuaccugauuuuuauaaugacuggauguucauugcuaaacaucugccugaucucauagagucuggccagcuucgagaaagaguugagaaguuaaacaugcucagcauugaucaucucacagaccacaagucacagcgccuugcacgucuaguucugggaugcaucaccauggcauauguguggggcaaaggucauggagauguccguaaggucuugccaagaaauauugcuguuccuuacugccaacucuccaagaaacuggaacugccuccuauuuugguuuaugcagacugugucuuggcaaacuggaagaaaaaggauccuaauaagccccugacuuaugagaacauggacguuuuguucucauuucgugauggagacugcaguaaaggauucuuccuggucucucuauugguggaaauagcagcugcuucugcaaucaaaguaauuccuacuguauucaaggcaaugcaaaugcaagaacgggacacuuugcuaaaggcgcuguuggaaauagcuucuugcuuggagaaagcccuucaaguguuucaccaaauccacgaucaugugaacccaaaagcauuuuucaguguucuucgcauauauuugucuggcuggaaaggcaacccccagcuaucagacggucugguguaugaaggguucugggaagacccaaaggaguuugcagggggcagugcaggccaaagcagcgucuuucagugcuuugacguccugcugggcauccagcagacugcugguggaggacaugcugcucaguuccuccaggacaugagaagauauaugccaccagcucacaggaacuuccugugcucauuagagucaaaucccucaguccgugaguuuguccuuucaaaaggugaugcuggccugcgggaagcuuaugacgccugugugaaagcucuggucucccugaggagcuaccaucugcaaaucgugacuaaguacauccugauuccugcaagccagcagccaaaggagaauaagaccucugaagacccuucaaaacuggaagccaaaggaacuggaggcacugauuuaaugaauuuccugaagacuguaagaaguacaacugagaaaucccuuuugaaggaagguuaauguaacccaacaagagcacauuuuaucauagcagagacaucuguaugcauuccugucauuacccauuguaacagagccacaaacuaauacuaugcaauguuuuaccaauaaugcaauacaaaagaccucaaaauaccugugcauuucuuguaggaaaacaacaaaagguaauuauguguaauuauacuagaaguuuuguaaucuguaucuuaucauuggaauaaaaugacauucaauaaauaaaaaugcauaagauauauucugucggcugggcgcgguggcucacgccuguaaucccagcacuuugggaggccgaggcgggcggaucacaaggucaggagaucgagaccaucuuggcuaacacggugaaaccccgucucuacuaaaaauacaaaaaauuagccgggcgcgguggcgggcaccuguagucccagcuacucgggaggcugaggcaggagaauggcgugaaccugggaggcggagcuugcagugagccaagauugugccacugcaauccggccugggcuaaagagcgggacuccgucucaaaaaaaaaaaaaaaaagauauauucugucauaauaaauaaaaaugcauaagauauaaaaaaaaaaaaaaa

SEQ ID NO: 42 - protein, 403 aa, Homo sapiens [sequence omitted]

SEQ ID NO: 43 - RNA, 1135 nt, Homo sapiens
uuccuccuccgagagcggacagaucucugggugcugggcggucauggcgcuacuagauguaugcggagccccccgagggcagcggccggaaucggcucucccgguugcgggaagcgggcgucgcucggacccaggacacuacaguuucucuaugcgaucuccagagcucgcuuuaccccggggaaugcagcccacagaauucuuccagucccuggguggggacggagaaaggaacguucagauugagauggcccauggcaccaccacgcucgccuucaaguuccagcauggagugauugcagcaguggauucucgggccucagcuggguccuacauuagugccuuacgggugaacaaggugauugagauuaacccuuaccugcuuggcaccaugucuggcugugcagcagacugucaguacugggagcgccugcuggccaaggaaugcaggcuguacuaucugcgaaauggagaacguauuucagugucggcagccuccaagcugcuguccaacaugaugugccaguaccggggcaugggccucucuaugggcaguaugaucuguggcugggauaagaaggguccuggacucuacuacguggaugaacaugggacucggcucucaggaaauauguucuccacggguagugggaacacuuaugccuacggggucauggacaguggcuaucggccuaaucuuagcccugaagaggccuaugaccuuggccgcagggcuauugcuuaugccacucacagagacagcuauucuggaggcguugucaauauguaccacaugaaggaagaugguugggugaaaguagaaaguacagaugucagugaccugcugcaccaguaccgggaagccaaucaauaaugguggugguggcagcugggcaggucuccucugggaggucuuggccgacucagggaccuaagccacguuaaguccaaggagaagaagaggccuagccugagccaaagagagaguacgggcucagcagccagaggaggccggugaagugcaucuucugcguguucucuauuugaacaagcauuucccccagggaaguuucugggugccccacuaaguagaauaaagaaaaacgguuauaaauaaaaaaaaaaaaaaaaaaaa

SEQ ID NO: 44 - protein, 276 aa, Homo sapiens
MALLDVCGAPRGQRPESALPVAGSGRRSDPGHYSFSMRSPELALPRGMQPTEFFQSLGGDGERNVQIEMAHGTTTLAFKFQHGVIAAVDSRASAGSYISALRVNKVIEINPYLLGTMSGCAADCQYWERLLAKECRLYYLRNGERISVSAASKLLSNMMCQYRGMGLSMGSMICGWDKKGPGLYYVDEHGTRLSGNMFSTGSGNTYAYGVMDSGYRPNLSPEEAYDLGRRAIAYATHRDSYSGGVVNMYHMKEDGWVKVESTDVSDLLHQYREANQ

SEQ ID NO: 45 - RNA, 1048 nt, Homo sapiens
gcgcguugugcgcugucccagguuggaaaccagugccccaggcggcgaggagagcggugccuugcagggaugcugcgggcgggagcaccaaccggggacuuaccccgggcgggagaaguccacaccgggaccaccaucauggcaguggaguuugacgggggcguugugauggguucugauucccgagugucugcaggcgaggcgguggugaaccgaguguuugacaagcuguccccgcugcacgagcgcaucuacugugcacucucugguucagcugcugaugcccaagccguggccgacauggccgccuaccagcuggagcuccaugggauagaacuggaggaaccuccacuuguuuuggcugcugcaaauguggugagaaauaucagcuauaaauaucgagaggacuugucugcacaucucaugguagcuggcugggaccaacgugaaggaggucagguauauggaacccugggaggaaugcugacucgacagccuuuugccauugguggcuccggcagcaccuuuaucuaugguuauguggaugcagcauauaagccaggcaugucucccgaggagugcaggcgcuucaccacagacgcuauugcucuggccaugagccgggauggcucaagcgggggugucaucuaccuggucacuauuacagcugccgguguggaccaucgagucaucuugggcaaugaacugccaaaauucuaugaugagugaaccuuccccagacuucucuuucuuauuuuguaauaaacucucuagggccaaaaccugguauggucauugggaaaugagugcucagggagauggagcuuaggggaggugggugcuucccuccuagaugucagcauacacucuuucuucuuuugucccaggucuaaaacaucuuuccuagagaaaacaaaagggacuaaacuagaaauauaaagagcccuauacaugacaggugaucacguacugaaugauuuugaaguaguacaaacaauaaaaauucucauuccgcaucaucaugcgguccaugaugaugaggccgcaa

SEQ ID NO: 46 - protein, 219 aa, Homo sapiens
MLRAGAPTGDLPRAGEVHTGTTIMAVEFDGGVVMGSDSRVSAGEAVVNRVFDKLSPLHERIYCALSGSAADAQAVADMAAYQLELHGIELEEPPLVLAAANVVRNISYKYREDLSAHLMVAGWDQREGGQVYGTLGGMLTRQPFAIGGSGSTFIYGYVDAAYKPGMSPEECRRFTTDAIALAMSRDGSSGGVIYLVTITAAGVDHRVILGNELPKFYDE

SEQ ID NO: 47 - RNA, 2974 nt, Homo sapiens
gugugcgugauggagaaaauugggcaccagggcugcucccgagauucucagaucugauuuccacgcuugcuaccaaaauagucugggcaggccacuuuuggaaguaggcguuaucuagugagcaggcggccgcuuucgauuucgcuuuccccuaaauggcugagcuucucgccagcgcaggaucagccuguuccugggacuuuccgagagccccgcccucguucccucccccagccgccaguaggggaggacucggcgguacccggagcuucaggccccaccggggcgcggagagucccaggcccggccgggaccgggacggcguccgagugccaauggcuagcucuaggugucccgcuccccgcgggugccgcugccuccccggagcuucucucgcauggcuggggacaguacugcuacuucucgccgacugggugcugcuccggaccgcgcugccccgcauauucucccugcuggugcccaccgcgcugccacugcuccgggucugggcggugggccugagccgcugggccgugcucuggcugggggccugcgggguccucagggcaacgguuggcuccaagagcgaaaacgcaggugcccagggcuggcuggcugcuuugaagccauuagcugcggcacugggcuuggcccugccgggacuugccuuguuccgagagcugaucucauggggagcccccggguccgcggauagcaccaggcuacugcacuggggaagucacccuaccgccuucguugucaguuaugcagcggcacugcccgcagcagcccuguggcacaaacucgggagccucugggugcccggcggucagggcggcucuggaaacccugugcgucggcuucuaggcugccugggcucggagacgcgccgccucucgcuguuccugguccuggugguccucuccucucuuggggagauggccauuccauucuuuacgggccgccucacugacuggauucuacaagauggcucagccgauaccuucacucgaaacuuaacucucauguccauucucaccauagccagugcagugcuggaguucgugggugacgggaucuauaacaacaccaugggccacgugcacagccacuugcagggagagguguuuggggcuguccugcgccaggagacggaguuuuuccaacagaaccagacagguaacaucaugucucggguaacagaggacacguccacccugagugauucucugagugagaaucugagcuuauuucugugguaccuggugcgaggccuaugucucuuggggaucaugcucuggggaucagugucccucaccauggucacccugaucacccugccucugcuuuuccuucugcccaagaaggugggaaaaugguaccaguugcuggaagugcaggugcgggaaucucuggcaaaguccagccagguggccauugaggcucugucggccaugccuacaguucgaagcuuugccaacgaggagggcgaagcccagaaguuuagggaaaagcugcaagaaauaaagacacucaaccagaaggaggcuguggccuaugcagucaacuccuggaccacuaguauuucagguaugcugcugaaagugggaauccucuacauuggugggcagcuggugaccaguggggcuguaagcagugggaaccuugucacauuuguucucuaccagaugcaguucacccaggcuguggagguacugcucuccaucuaccccagaguacagaaggcugugggcuccucagagaaaauauuugaguaccuggaccgcaccccucgcugcccacccaguggucuguugacucccuuacacuuggagggccuuguccaguuccaagaugucuccuuugccuacccaaaccgcccagaugucuuagugcuacaggggcugacauucacccuacgcccuggcgaggugacggcgcuggugggacccaaugggucugggaagagcacaguggcugcccugcugcagaaucuguaccagcccaccgggggacagcugcuguuggaugggaagccccuuccccaauaugagcaccgcuaccugcacaggcagguggcugcagugggacaagagccacagguauuuggaagaagucuucaagaaaauauugccuauggccugacccagaagccaacuauggaggaaaucacagcugcugcaguaaagucuggggcccauaguuucaucucuggacucccucagggcuaugacacagagguagacgaggcugggagccagcugucagggggucagcgacaggcaguggcguuggcccgagcauugauccggaaaccguguguacuuauccuggaugaugccaccagugcccuggaugcaaacagccaguuacagguggagcagcuccuguacgaaagcccugagcgguacucccgcucagugcuucucaucacccagcaccucagccugguggagcaggcugaccacauccucuuucuggaaggaggcgcuauccgggaggggggaacccaccagcagcucauggagaaaaaggggugcuacugggccauggugcaggcuccugcagaugcuccagaaugaaagccuucucagaccugcgcacuccaucucccucccuuuucuucucucugugguggagaaccacagcugcagaguaggcagcugccuccaggaugaguuacuugaaauuugccuugaguguguuaccuccuuuccaagcuccucgugauaaugcagacuuccuggaguacaaacacaggauuuguaauuccuuacuguaacggaguuuagagccagggcugaugcuuugguguggccagcacucugaaacugagaaauguucagaauguacggaaagaugaucagcuauuuucaacauaacugaaggcauaugcuggcccauaaacacccuguagguucuugauauuuauaauaaaauugguguuuuguaaaaaaaaaaaaaaaaaaaaaaa

SEQ ID NO: 48 - protein, 808 aa, Homo sapiens
MAELLASAGSACSWDFPRAPPSFPPPAASRGGLGGTRSFRPHRGAESPRPGRDRDGVRVPMASSRCPAPRGCRCLPGASLAWLGTVLLLLADWVLLRTALPRIFSLLVPTALPLLRVWAVGLSRWAVLWLGACGVLRATVGSKSENAGAQGWLAALKPLAAALGLALPGLALFRELISWGAPGSADSTRLLHWGSHPTAFVVSYAAALPAAALWHKLGSLWVPGGQGGSGNPVRRLLGCLGSETRRLSLFLVLVVLSSLGEMAIPFFTGRLTDWILQDGSADTFTRNLTLMSILTIASAVLEFVGDGIYNNTMGHVHSHLQGEVFGAVLRQETEFFQQNQTGNIMSRVTEDTSTLSDSLSENLSLFLWYLVRGLCLLGIMLWGSVSLTMVTLITLPLLFLLPKKVGKWYQLLEVQVRESLAKSSQVAIEALSAMPTVRSFANEEGEAQKFREKLQEIKTLNQKEAVAYAVNSWTTSISGMLLKVGILYIGGQLVTSGAVSSGNLVTFVLYQMQFTQAVEVLLSIYPRVQKAVGSSEKIFEYLDRTPRCPPSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPCVLILDDATSALDANSQLQVEQLLYESPERYSRSVLLITQHLSLVEQADHILFLEGGAIREGGTHQQLMEKKGCYWAMVQAPADAPE

SEQ ID NO: 49 - RNA, 5732 nt, Homo sapiens
agaaugaaggccuuggcuggggaagcgaaagcgaaagcugcccgagcccugacgcccgcccuggccgagcguagcuggcggaccagagccgguagcgagguugggagagacggagcggaccucagcgcugaagcagaaguccccggagcugcggucuccccgccgcggcugagccaugcggcucccugaccugagacccuggaccucccugcugcugguggacgcggcuuuacuguggcugcuucagggcccucuggggacuuugcuuccucaagggcugccaggacuauggcuggaggggacccugcggcugggagggcugugggggcugcuaaagcuaagagggcugcugggauuuguggggacacugcugcucccgcucugucuggccaccccccugacugucucccugagagcccuggucgcgggggccucacgugcucccccagccagagucgcuucagccccuuggagcuggcugcuggugggguacggggcugcggggcucagcuggucacugugggcuguucugagcccuccuggagcccaggagaaggagcaggaccaggugaacaacaaagucuugauguggaggcugcugaagcucuccaggccggaccugccucuccucguugccgccuucuucuuccuuguccuugcuguuuugggugagacauuaaucccucacuauucuggucgugugauugacauccugggaggugauuuugacccccaugccuuugccagugccaucuucuucaugugccucuucuccuuuggcagcucacugucugcaggcugccgaggaggcugcuucaccuacaccaugucucgaaucaacuugcggauccgggagcagcuuuucuccucccugcugcgccaggaccucgguuucuuccaggagacuaagacaggggagcugaacucacggcugagcucggauaccacccugaugaguaacuggcuuccuuuaaaugccaaugugcucuugcgaagccuggugaaagugguggggcuguauggcuucaugcucagcauaucgccucgacucacccuccuuucucugcugcacaugcccuucacaauagcagcggagaagguguacaacacccgccaucaggaagugcuucgggagauccaggaugcaguggccagggcggggcagguggugcgggaagccguuggagggcugcagaccguucgcaguuuuggggccgaggagcaugaagucugucgcuauaaagaggcccuugaacaaugucggcagcuguauuggcggagagaccuggaacgcgccuuguaccugcucguaaggagggugcugcacuugggggugcagaugcugaugcugagcugugggcugcagcagaugcaggauggggagcucacccagggcagccugcuuuccuuuaugaucuaccaggagagcguggggagcuaugugcagacccugguauacauauauggggauaugcucagcaacgugggagcugcagagaagguuuucuccuacauggaccgacagccaaaucugccuucaccuggcacgcuugcccccaccacucugcaggggguugugaaauuccaagacgucuccuuugcauaucccaaucgcccugacaggccugugcucaaggggcugacguuuacccuacguccuggugaggugacggcgcuggugggacccaaugggucugggaagagcacaguggcugcccugcugcagaaucuguaccagcccacagggggacaggugcugcuggaugaaaagcccaucucacaguaugaacacugcuaccugcacagccaggugguuucaguugggcaggagccugugcuguucuccgguucugugaggaacaacauugcuuaugggcugcagagcugcgaagaugauaaggugauggcggcugcccaggcugcccacgcagaugacuucauccaggaaauggagcauggaauauacacagauguaggggagaagggaagccagcuggcugcgggacagaaacaacgucuggccauugcccgggcccuuguacgagacccgcggguccucauccuggaugaggcuacuagugcccuagaugugcagugcgagcaggcccugcaggacuggaauucccguggggaucgcacagugcuggugauugcucacaggcugcagacaguucagcgcgcccaccagauccuggugcuccaggagggcaagcugcagaagcuugcccagcucuaggagggacaggaccucuauucccgccuggugcagcagcggcugauggacugaggccccagggauacugggcccucuucucaggggcgucuccaggacccagagcuguuccugcuuugaguuucccuagagcugugcggccagauagcuguuccugaguugcaggcacgauggagauuuggacacugugugcuuuuggugggguagagagguggggugggguggggugggggcugucuguguccaggaaacuuaauucccuggugacuagagcuuugccuggugaugaggaguauuuuguggcauaauacauauauuuuaaaauauuuuccuucuuacaugaacuguauacauucauauagaaaauuuagacaauauaaaaaaguacaaagaagaaaaguaaaaguacccauuguuucacuuccuggagauaaccauaguugcuauuuugcugccugucccaucagucguuuaucuguuguuugagauagaaauuaaccaaaaaugacauaaauauucaugagauugccuuccuauauccuuccuuguuccuaccagugucugcuauuuugaagaagcuagggucuggagggacagagaacaguucccugauuaacaguauuaauagcgacauugguaacagcuaccauuuauagaguuuuaaugggaguaggagcuaugcuaaguguuuuucauguauuaucguuuuuaaucauuauccccaacccuaugagguugguuauuauccccauuuuacagaugaggaaacugaagcucaaagaggcucaaugacuuucccaagguggucguagugguggaguuggaguuugaacacaggccugacccuagaguccacacccugacccaaucaauuauauugcaucuuggguccauaaacccuaauccauaaucccaucaagaaaagcucugcugcucuuagcucuaaauaauucagaaucuauucucuucucuccagucccguuguuauagucuucacucauagacuuaagaugaucccaucaccagagagguuucucuaccauuagcuucccucuuccggccauucuucacaaagucauuuuucuaaauucugugucacauacgaugauggcauuucuggaaauuccuucaggugcucucaagcccugcugcagagauccuuuucagagcacacacuguuccagcccaucugucucacccucuccuguuguauccagcuccacgacaaacuucugccuuccccaacaccuuugugccuuugcauaugguguuuucuugcccauuuucugcucgacucgccccugauuuucaaguucaagacuuaacucaggguucaggucuuccaggaggccuuacuuaugucgucagucuggggaacucuccaugugcuucuaucacugugcgguuaccucuuucacagcccuuuuaaaguucuaucuucccuuucccaccuuuuuugaccuuccacuagaccaugagcaccugggcggaaagccauauaucuuauuaagcuuuauaucugcuaccuggccgagggccuaauucauaguggagaauaaauagucaauugaauaaaugaauaaauaucuccaccaucguacuaaucuuaauccucccugcccacucccaccacugaaaaugcaacauuguacacaucacugguuguugggagggacuuaccuuggaaaguugcuauucuaggaaagagaaaccuucauauuccuggaaacagcagguaguuuccagugcuggcaaugaauuccccagaacugcuguuuuggauuuuuucuugccuggcagcuguugggagcagggugcagugaggauggggugagagugggcaguuucuugugcagauuugccuuucuuucauccuggggcugacuugcagcuccacacccauccaucucucaaauuucacagaggguaaaauaggcauuuggagagaaagaacucuggccugauuccuuucucucccacaaauguccuuuauucauaaaacaggaauaauaauuccuguaucucccaacuacauggaagcugcagcccucacagaagaagaugaucugagaaauucuuugauuuccucaguacaguuauacccaugcaucauaauacuuuaagccuggaaggcaucuuaaaaauaaugcaacagucaaaccuaauuuuacagagaaacugacaugaaaucacgcagcuaaucaugauaaagcuggguggaaaacuuaucuugaugggcaguacaggaagaugcaguagaccuuaagauguccugaaaguuucuuaucucaggggaaacucccagguaggcuuuaugucagggacacagaaaaaugcucccugaaagucaaaauauucgggcuagacagacaaauuccuguaagugugguuugucugggaaccacagaugucacuaauccugguuugcuccagaguucuuuuuguucacuccuaccccccaucaccauuugauugaucuccuuacccuguaauuuccccuucuugucgcuuaccugcaguaucuuucccacccaggcaugccuuauucuuucuaaaggaaaguaugaauggagaggggaaagcuugggaaacugauagauuuccuuggaugccaaaacaccuccauagccugucugcccggcccuauguggaaacagcauugaguuucaaguccuuuaugccuccacccagggauagccacuuguaauccacauggcaauugugaaacaagcaggaaaugcguaauugucagaauuuuguggggaaaggacuagggaauaaggaaaacaaagaucuuccuuguguuuuagagcugucagcuagaggagcaccugcuugagucugaugccaucuaauggucccagaagaaacuggguuuugaaccuagaguuccauggacucuuaggaauuagacuacuacuacuacuaagcauucacuggugcuuacuaugugcuauugcugugccaaguaucugaaaccugucuucuuaccuuauuuuucaagauaauucuauguggcagguauuacuaucucaauucuaagagugagaaaauggaguuuuagaaacauuuacuaacuugccugggucacauagcuaaggaagagguggacuugcccagcuuugcauaaaacuccucaaaagaguugccuauacucccugacuccacuuaucuuccuacuauccucuuuuuaaaauauauuauuuauuuauuuaaauaagcaauauaugaaugugguuugaaauucaaaagacacaaagaaguauacagaggaaagccucacucucaauccuucucaagguuugcuaauuccucuugcauaggcaauccguucuuccagcuuuguguuuaucuuuccagagaaguuuacuguguauuaagcaaauauguauaucuuuauucuugcucaguauuuucgcaaacagcagcugucuaaguucacuguucugaacuuuauuuuuuaaauuaaaaauauauggcuauguaguauucuauuuua

SEQ ID NO: 50 - protein, 686 aa, Homo sapiens
MRLPDLRPWTSLLLVDAALLWLLQGPLGTLLPQGLPGLWLEGTLRLGGLWGLLKLRGLLGFVGTLLLPLCLATPLTVSLRALVAGASRAPPARVASAPWSWLLVGYGAAGLSWSLWAVLSPPGAQEKEQDQVNNKVLMWRLLKLSRPDLPLLVAAFFFLVLAVLGETLIPHYSGRVIDILGGDFDPHAFASAIFFMCLFSFGSSLSAGCRGGCFTYTMSRINLRIREQLFSSLLRQDLGFFQETKTGELNSRLSSDTTLMSNWLPLNANVLLRSLVKVVGLYGFMLSISPRLTLLSLLHMPFTIAAEKVYNTRHQEVLREIQDAVARAGQVVREAVGGLQTVRSFGAEEHEVCRYKEALEQCRQLYWRRDLERALYLLVRRVLHLGVQMLMLSCGLQQMQDGELTQGSLLSFMIYQESVGSYVQTLVYIYGDMLSNVGAAEKVFSYMDRQPNLPSPGTLAPTTLQGVVKFQDVSFAYPNRPDRPVLKGLTFTLRPGEVTALVGPNGSGKSTVAALLQNLYQPTGGQVLLDEKPISQYEHCYLHSQVVSVGQEPVLFSGSVRNNIAYGLQSCEDDKVMAAAQAAHADDFIQEMEHGIYTDVGEKGSQLAAGQKQRLAIARALVRDPRVLILDEATSALDVQCEQALQDWNSRGDRTVLVIAHRLQTVQRAHQILVLQEGKLQKLAQL

SEQ ID NO: 51 - RNA, 849 nt, Homo sapiens
augaggaacuccuauagauuucuggcauccucucucucaguugucguuucucuccugcuaauuccugaagaugucugugaaaaaauuauuggaggaaaugaaguaacuccucauucaagacccuacaugguccuacuuagucuugacagaaaaaccaucugugcuggggcuuugauugcaaaagacuggguguugacugcagcucacuguaacuugaacaaaaggucccaggucauucuuggggcucacucaauaaccagggaagagccaacaaaacagauaaugcuuguuaagaaagaguuucccuauccaugcuaugacccagccacacgcgaaggugaccuuaaacuuuuacagcugacggaaaaagcaaaaauuaacaaauaugugacuauccuucaucuaccuaaaaagggggaugaugugaaaccaggaaccaugugccaaguugcagggugggggaggacucacaauagugcaucuugguccgauacucugagagaagucaauaucaccaucauagacagaaaagucugcaaugaucgaaaucacuauaauuuuaacccugugauuggaaugaauaugguuugugcuggaagccuccgagguggaagagacucgugcaauggagauucuggaagcccuuuguugugcgaggguguuuuccgaggggucacuuccuuuggccuugaaaauaaaugcggagacccucgugggccuggugucuauauucuucucucaaagaaacaccucaacuggauaauuaugacuaucaagggagcaguuuaaauaaccguuuccuuucauuuacuguggcuucuuaaucuuuucacaaauaaaaucaauuug

SEQ ID NO: 52 - protein, 262 aa, Homo sapiens
MRNSYRFLASSLSVVVSLLLIPEDVCEKIIGGNEVTPHSRPYMVLLSLDRKTICAGALIAKDWVLTAAHCNLNKRSQVILGAHSITREEPTKQIMLVKKEFPYPCYDPATREGDLKLLQLMEKAKINKYVTILHLPKKGDDVKPGTMCQVAGWGRTHNSASWSDTLREVNITIIDRKVCNDRNHYNFNPVIGMNMVCAGSLRGGRDSCNGDSGSPLLCEGVFRGVTSFGLENKCGDPRGPGVYILLSKKHLNWIIMTIKGAV

SEQ ID NO: 53 - RNA, 934 nt, Homo sapiens
ugagaagaugcaaccaauccugcuucugcuggccuuccuccugcugcccagggcagaugcaggggagaucaucgggggacaugaggccaagccccacucccgccccuacauggcuuaucuuaugaucugggaucagaagucucugaagaggugcgguggcuuccugauacaagacgacuucgugcugacagcugcucacuguuggggaagcuccauaaaugucaccuugggggcccacaauaucaaagaacaggagccgacccagcaguuuaucccugugaaaagacccaucccccauccagccuauaauccuaagaacuucuccaacgacaucaugcuacugcagcuggagagaaaggccaagcggaccagagcugugcagccccucaggcuaccuagcaacaaggcccaggugaagccagggcagacaugcaguguggccggcugggggcagacggccccccugggaaaacacucacacacacuacaagaggugaagaugacagugcaggaagaucgaaagugcgaaucugacuuacgccauuauuacgacaguaccauugaguugugcgugggggacccagagauuaaaaagacuuccuuuaagggggacucuggaggcccucuuguguguaacaagguggcccagggcauugucuccuauggacgaaacaauggcaugccuccacgagccugcaccaaagucucaagcuuuguacacuggauaaagaaaaccaugaaacgcuacuaacuacaggaagcaaacuaagcccccgcuguaaugaaacaccuucucuggagccaaguccagauuuacacugggagaggugccagcaacugaauaaauaccucucccaguguaaaucuggagccaaguccagauuuacacugggagaggugccagcaacugaauaaauaccucuuagcugagugg

SEQ ID NO: 54 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 55 - RNA, 7123 nt, Homo sapiens
aucgagguccgcgggaggcucggagcgcgccaggcggacacuccucucggcuccuccccggcagcggcggcggcucggagcgggcuccggggcucgggugcagcggccagcgggcgccuggcggcgaggauuacccggggaagugguugucuccuggcuggagccgcgagacgggcgcucagggcgcggggccggcggcggcgaacgagaggacggacucuggcggccgggucguuggccgcggggagcgcgggcaccgggcgagcaggccgcgucgcgcucaccauggucagcuacugggacaccgggguccugcugugcgcgcugcucagcugucugcuucucacaggaucuaguucagguucaaaauuaaaagauccugaacugaguuuaaaaggcacccagcacaucaugcaagcaggccagacacugcaucuccaaugcaggggggaagcagcccauaaauggucuuugccugaaauggugaguaaggaaagcgaaaggcugagcauaacuaaaucugccuguggaagaaauggcaaacaauucugcaguacuuuaaccuugaacacagcucaagcaaaccacacuggcuucuacagcugcaaauaucuagcuguaccuacuucaaagaagaaggaaacagaaucugcaaucuauauauuuauuagugauacagguagaccuuucguagagauguacagugaaauccccgaaauuauacacaugacugaaggaagggagcucgucauucccugccggguuacgucaccuaacaucacuguuacuuuaaaaaaguuuccacuugacacuuugaucccugauggaaaacgcauaaucugggacaguagaaagggcuucaucauaucaaaugcaacguacaaagaaauagggcuucugaccugugaagcaacagucaaugggcauuuguauaagacaaacuaucucacacaucgacaaaccaauacaaucauagauguccaaauaagcacaccacgcccagucaaauuacuuagaggccauacucuuguccucaauuguacugcuaccacucccuugaacacgagaguucaaaugaccuggaguuacccugaugaaaaaaauaagagagcuuccguaaggcgacgaauugaccaaagcaauucccaugccaacauauucuacaguguucuuacuauugacaaaaugcagaacaaagacaaaggacuuuauacuugucguguaaggaguggaccaucauucaaaucuguuaacaccucagugcauauauaugauaaagcauucaucacugugaaacaucgaaaacagcaggugcuugaaaccguagcuggcaagcggucuuaccggcucucuaugaaagugaaggcauuucccucgccggaaguuguaugguuaaaagauggguuaccugcgacugagaaaucugcucgcuauuugacucguggcuacucguuaauuaucaaggacguaacugaagaggaugcagggaauuauacaaucuugcugagcauaaaacagucaaauguguuuaaaaaccucacugccacucuaauugucaaugugaaaccccagauuuacgaaaaggccgugucaucguuuccagacccggcucucuacccacugggcagcagacaaauccugacuuguaccgcauaugguaucccucaaccuacaaucaagugguucuggcaccccuguaaccauaaucauuccgaagcaaggugugacuuuuguuccaauaaugaagaguccuuuauccuggaugcugacagcaacaugggaaacagaauugagagcaucacucagcgcauggcaauaauagaaggaaagaauaagauggcuagcaccuugguuguggcugacucuagaauuucuggaaucuacauuugcauagcuuccaauaaaguugggacugugggaagaaacauaagcuuuuauaucacagaugugccaaauggguuucauguuaacuuggaaaaaaugccgacggaaggagaggaccugaaacugucuugcacaguuaacaaguucuuauacagagacguuacuuggauuuuacugcggacaguuaauaacagaacaaugcacuacaguauuagcaagcaaaaaauggccaucacuaaggagcacuccaucacucuuaaucuuaccaucaugaauguuucccugcaagauucaggcaccuaugccugcagagccaggaauguauacacaggggaagaaauccuccagaagaaagaaauuacaaucagagaucaggaagcaccauaccuccugcgaaaccucagugaucacacaguggccaucagcaguuccaccacuuuagacugucaugcuaaugguguccccgagccucagaucacuugguuuaaaaacaaccacaaaauacaacaagagccuggaauuauuuuaggaccaggaagcagcacgcuguuuauugaaagagucacagaagaggaugaaggugucuaucacugcaaagccaccaaccagaagggcucuguggaaaguucagcauaccucacuguucaaggaaccucggacaagucuaaucuggagcugaucacucuaacaugcaccuguguggcugcgacucucuucuggcuccuauuaacccucuuuauccgaaaaaugaaaaggucuucuucugaaauaaagacugacuaccuaucaauuauaauggacccagaugaaguuccuuuggaugagcagugugagcggcucccuuaugaugccagcaagugggaguuugcccgggagagacuuaaacugggcaaaucacuuggaagaggggcuuuuggaaaagugguucaagcaucagcauuuggcauuaagaaaucaccuacgugccggacuguggcugugaaaaugcugaaagagggggccacggccagcgaguacaaagcucugaugacugagcuaaaaaucuugacccacauuggccaccaucugaacgugguuaaccugcugggagccugcaccaagcaaggagggccucugauggugauuguugaauacugcaaauauggaaaucucuccaacuaccucaagagcaaacgugacuuauuuuuucucaacaaggaugcagcacuacacauggagccuaagaaagaaaaaauggagccaggccuggaacaaggcaagaaaccaagacuagauagcgucaccagcagcgaaagcuuugcgagcuccggcuuucaggaagauaaaagucugagugauguugaggaagaggaggauucugacgguuucuacaaggagcccaucacuauggaagaucugauuucuuacaguuuucaaguggccagaggcauggaguuccugucuuccagaaagugcauucaucgggaccuggcagcgagaaacauucuuuuaucugagaacaacguggugaagauuugugauuuuggccuugcccgggauauuuauaagaaccccgauuaugugagaaaaggagauacucgacuuccucugaaauggauggcuccugaaucuaucuuugacaaaaucuacagcaccaagagcgacguguggucuuacggaguauugcugugggaaaucuucuccuuaggugggucuccauacccaggaguacaaauggaugaggacuuuugcagucgccugagggaaggcaugaggaugagagcuccugaguacucuacuccugaaaucuaucagaucaugcuggacugcuggcacagagacccaaaagaaaggccaagauuugcagaacuuguggaaaaacuaggugauuugcuucaagcaaauguacaacaggaugguaaagacuacaucccaaucaaugccauacugacaggaaauaguggguuuacauacucaacuccugccuucucugaggacuucuucaaggaaaguauuucagcuccgaaguuuaauucaggaagcucugaugaugucagauacguaaaugcuuucaaguucaugagccuggaaagaaucaaaaccuuugaagaacuuuuaccgaaugccaccuccauguuugaugacuaccagggcgacagcagcacucuguuggccucucccaugcugaagcgcuucaccuggacugacagcaaacccaaggccucgcucaagauugacuugagaguaaccaguaaaaguaaggagucggggcugucugaugucagcaggcccaguuucugccauuccagcugugggcacgucagcgaaggcaagcgcagguucaccuacgaccacgcugagcuggaaaggaaaaucgcgugcugcuccccgcccccagacuacaacucggugguccuguacuccaccccacccaucuagaguuugacacgaagccuuauuucuagaagcacauguguauuuauacccccaggaaacuagcuuuugccaguauuaugcauauauaaguuuacaccuuuaucuuuccaugggagccagcugcuuuuugugauuuuuuuaauagugcuuuuuuuuuuuugacuaacaagaauguaacuccagauagagaaauagugacaagugaagaacacuacugcuaaauccucauguuacucaguguuagagaaauccuuccuaaacccaaugacuucccugcuccaacccccgccaccucagggcacgcaggaccaguuugauugaggagcugcacugaucacccaaugcaucacguaccccacugggccagcccugcagcccaaaacccagggcaacaagcccguuagccccagggaucacuggcuggccugagcaacaucucgggaguccucuagcaggccuaagacaugugaggaggaaaaggaaaaaaagcaaaaagcaagggagaaaagagaaaccgggagaaggcaugagaaagaauuugagacgcaccaugugggcacggagggggacggggcucagcaaugccauuucaguggcuucccagcucugacccuucuacauuugagggcccagccaggagcagauggacagcgaugaggggacauuuucuggauucugggaggcaagaaaaggacaaauaucuuuuuuggaacuaaagcaaauuuuagaacuuuaccuauggaagugguucuauguccauucucauucguggcauguuuugauuuguagcacugaggguggcacucaacucugagcccauacuuuuggcuccucuaguaagaugcacugaaaacuuagccagaguuagguugucuccaggccaugauggccuuacacugaaaaugucacauucuauuuuggguauuaauauauaguccagacacuuaacucaauuucuugguauuauucuguuuugcacaguuaguugugaaagaaagcugagaagaaugaaaaugcaguccugaggagaggaguuuucuccauaucaaaacgagggcugauggaggaaaaaggucaauaaggucaagggaaaaccccgucucuauaccaaccaaaccaauucaccaacacaguugggacccaaaacacaggaagucagucacguuuccuuuucauuuaauggggauuccacuaucucacacuaaucugaaaggauguggaagagcauuagcuggcgcauauuaagcacuuuaagcuccuugaguaaaaaggugguauguaauuuaugcaagguauuucuccaguugggacucaggauauuaguuaaugagccaucacuagaagaaaagcccauuuucaacugcuuugaaacuugccuggggucugagcaugaugggaauagggagacaggguaggaaagggcgccuacucuucagggucuaaagaucaagugggccuuggaucgcuaagcuggcucuguuugaugcuauuuaugcaaguuagggucuauguauuuaugaugucugcaccuucugcagccagucagaagcuggagaggcaacaguggauugcugcuucuuggggagaagaguaugcuuccuuuuauccauguaauuuaacuguagaaccugagcucuaaguaaccgaagaauguaugccucuguucuuaugugccacauccuuguuuaaaggcucucuguaugaagagaugggaccgucaucagcacauucccuagugagccuacuggcuccuggcagcggcuuuuguggaagacucacuagccagaagagaggagugggacaguccucuccaccaagaucuaaauccaaacaaaagcaggcuagagccagaagagaggacaaaucuuuguucuuccucuucuuuacauacgcaaaccaccugugacagcuggcaauuuuauaaaucagguaacuggaaggagguuaaacacagaaaaaagaagaccucagucaauucucuacuuuuuuuuuuuuuuccaaaucagauaauagcccagcaaauagugauaacaaauaaaaccuuagcuauucaugucuugauuucaauaauuaauucuuaaucauuaagagaccauaauaaauacuccuuuucaagagaaaagcaaaaccauuagaauuguuacucagcuccuucaaacucagguuuguagcauacaugaguccauccaucagucaaagaaugguuccaucuggagucuuaauguagaaagaaaaauggagacuuguaauaaugagcuaguuacaaagugcuuguucauuaaaauagcacugaaaauugaaacaugaauuaacugauaauauuccaaucauuugccauuuaugacaaaaaugguuggcacuaacaaagaacgagcacuuccuuucagaguuucugagauaauguacguggaacagucuggguggaauggggcugaaaccaugugcaagucugugucuugucaguccaagaagugacaccgagauguuaauuuuagggacccgugccuuguuuccuagcccacaagaaugcaaacaucaaacagauacucgcuagccucauuuaaauugauuaaaggaggagugcaucuuuggccgacagugguguaacuguauguguguguguguguguguguguguguguguguguggguguauguguguuuugugcauaacuauuuaaggaaacuggaauuuuaaaguuacuuuuauacaaaccaagaauauaugcuacagauauaagacagacaugguuugguccuauauuucuagucaugaugaauguauuuuguauaccaucuucauauaauaaacuuccaaaaacaca

SEQ ID NO: 56 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 57 - RNA, 2545 nt, Homo sapiens
auccaauacaggagugacuuggaacuccauucuaucacuaugaagaaaagugguguucuuuuccucuugggcaucaucuugcugguucugauuggagugcaaggaaccccaguagugagaaagggucgcuguuccugcaucagcaccaaccaagggacuauccaccuacaauccuugaaagaccuuaaacaauuugccccaagcccuuccugcgagaaaauugaaaucauugcuacacugaagaauggaguucaaacaugucuaaacccagauucagcagaugugaaggaacugauuaaaaagugggagaaacaggucagccaaaagaaaaagcaaaagaaugggaaaaaacaucaaaaaaagaaaguucugaaaguucgaaaaucucaacguucucgucaaaagaagacuacauaagagaccacuucaccaauaaguauucuguguuaaaaauguucuauuuuaauuauaccgcuaucauuccaaaggaggauggcauauaauacaaaggcuuauuaauuugacuagaaaauuuaaaacauuacucugaaauuguaacuaaaguuagaaaguugauuuuaagaauccaaacguuaagaauuguuaaaggcuaugauugucuuuguucuucuaccacccaccaguugaauuucaucaugcuuaaggccaugauuuuagcaauacccaugucuacacagauguucacccaaccacaucccacucacaacagcugccuggaagagcagcccuaggcuuccacguacugcagccuccagagaguaucugaggcacaugucagcaaguccuaagccuguuagcaugcuggugagccaagcaguuugaaauugagcuggaccucaccaagcugcuguggccaucaaccucuguauuugaaucagccuacaggccucacacacaaugugucugagagauucaugcugauuguuauuggguaucaccacuggagaucaccaguguguggcuuucagagccuccuuucuggcuuuggaagccaugugauuccaucuugcccgcucaggcugaccacuuuauuucuuuuuguuccccuuugcuucauucaagucagcucuucuccauccuaccacaaugcagugccuuucuucucuccagugcaccugucauaugcucugauuuaucugagucaacuccuuucucaucuuguccccaacaccccacagaagugcuuucuucucccaauucauccucacucaguccagcuuaguucaaguccugccucuuaaauaaaccuuuuuggacacacaaauuaucuuaaaacuccuguuucacuugguucaguaccacaugggugaacacucaaugguuaacuaauucuuggguguuuauccuaucucuccaaccagauugucagcuccuugagggcaagagccacaguauauuucccuguuucuuccacagugccuaauaauacuguggaacuagguuuuaauaauuuuuuaauugauguuguuaugggcaggauggcaaccagaccauugucucagagcaggugcuggcucuuuccuggcuacuccauguuggcuagccucugguaaccucuuacuuauuaucuucaggacacucacuacagggaccagggaugaugcaacauccuugucuuuuuaugacaggauguuugcucagcuucuccaacaauaagaagcacgugguaaaacacuugcggauauucuggacuguuuuuaaaaaauauacaguuuaccgaaaaucauauaaucuuacaaugaaaaggacuuuauagaucagccagugaccaaccuuuucccaaccauacaaaaauuccuuuucccgaaggaaaagggcuuucucaauaagccucagcuuucuaagaucuaacaagauagccaccgagauccuuaucgaaacucauuuuaggcaaauaugaguuuuauuguccguuuacuuguuucagaguuuguauugugauuaucaauuaccacaccaucucccaugaagaaagggaacggugaaguacuaagcgcuagaggaagcagccaagucgguuaguggaagcaugauuggugcccaguuagccucugcaggauguggaaaccuccuuccaggggagguucagugaauuguguaggagagguugucuguggccagaauuuaaaccuauacucacuuucccaaauugaaucacugcucacacugcugaugauuuagagugcuguccgguggagaucccacccgaacgucuuaucuaaucaugaaacucccuaguuccuucauguaacuucccugaaaaaucuaaguguuucauaaauuugagagucugugacccacuuaccuugcaucucacagguagacaguauauaacuaacaaccaaagacuacauauugucacugacacacacguuauaaucauuuaucauauauauacauacaugcauacacucucaaagcaaauaauuuuucacuucaaaacaguauugacuuguauaccuuguaauuugaaauauuuucuuuguuaaaauagaaugguaucaauaaauagaccauuaaucag

SEQ ID NO: 58 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 59 - RNA, 1172 nt, Homo sapiens
gagacauuccucaauugcuuagacauauucugagccuacagcagaggaaccuccagucucagcaccaugaaucaaacugcgauucugauuugcugccuuaucuuucugacucuaaguggcauucaaggaguaccucucucuagaaccguacgcuguaccugcaucagcauuaguaaucaaccuguuaauccaaggucuuuagaaaaacuugaaauuauuccugcaagccaauuuuguccacguguugagaucauugcuacaaugaaaaagaagggugagaagagaugucugaauccagaaucgaaggccaucaagaauuuacugaaagcaguuagcaaggaaaugucuaaaagaucuccuuaaaaccagaggggagcaaaaucgaugcagugcuuccaaggauggaccacacagaggcugccucucccaucacuucccuacauggaguauaugucaagccauaauuguucuuaguuugcaguuacacuaaaaggugaccaaugauggucaccaaaucagcugcuacuacuccuguaggaagguuaauguucaucauccuaagcuauucaguaauaacucuacccuggcacuauaauguaagcucuacugaggugcuauguucuuaguggauguucugacccugcuucaaauauuucccucaccuuucccaucuuccaaggguacuaaggaaucuuucugcuuugggguuuaucagaauucucagaaucucaaauaacuaaaagguaugcaaucaaaucugcuuuuuaaagaaugcucuuuacuucauggacuuccacugccauccucccaaggggcccaaauucuuucaguggcuaccuacauacaauuccaaacacauacaggaagguagaaauaucugaaaauguauguguaaguauucuuauuuaaugaaagacuguacaaaguauaagucuuagauguauauauuuccuauauuguuuucaguguacauggaauaacauguaauuaaguacuauguaucaaugaguaacaggaaaauuuuaaaaauacagauagauauaugcucugcauguuacauaagauaaaugugcugaaugguuuucaaauaaaaaugagguacucuccuggaaauauuaagaaagacuaucuaaauguugaaagaucaaaagguuaauaaaguaauuauaacu

SEQ ID NO: 60 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 61 - RNA, 995 nt, Homo sapiens
gaauucggccaaagaggccuacuuccaagaagagcagcaaagcugaaguagcagcaacagcaccagcagcaacagcaaaaaacaaacaugagugugaagggcauggcuauagccuuggcugugauauugugugcuacaguuguucaaggcuuccccauguucaaaagaggacgcugucuuugcauaggcccugggguaaaagcagugaaaguggcagauauugagaaagccuccauaauguacccaaguaacaacugugacaaaauagaagugauuauuacccugaaagaaaauaaaggacaacgaugccuaaaucccaaaucgaagcaagcaaggcuuauaaucaaaaaaguugaaagaaagaauuuuuaaaaauaucaaaacauaugaaguccuggaaaagggcaucugaaaaaccuagaacaaguuuaacugugacuacugaaaugacaagaauucuacaguaggaaacugagacuuuucuaugguuuugugacuuucaacuuuuguacaguuaugugaaggaugaaaggugggugaaaggaccaaaaacagaaauacagucuuccugaaugaaugacaaucagaauuccacugcccaaaggaguccaacaauuaaauggauuucuaggaaaagcuaccuuaagaaaggcugguuaccaucggaguuuacaaagugcuuucacguucuuacuuguuguauuauacauucaugcauuucuaggcuagagaaccuucuagauuugaugcuuacaacuauucuguugugacuaugagaacauuucugucucuagaaguuaucugucuguauugaucuuuaugcuauauuacuaucugugguuacaguggagacauugacauuauuacuggagucaagcccuuauaagucaaaagcaccuaugugucguaaagcauuccucaaacauuuaaaaaaaaaaaaaaaaaaaaaaaaaaaaaaaaaaaaaaaaaaaaaaaaaaaaaaaaaaagcggccgc

SEQ ID NO: 62 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 63 - protein, 10 aa, Artificial Sequence
  Synthetic Construct
GFTFSDSWIH

SEQ ID NO: 64 - protein, 18 aa, Artificial Sequence
  Synthetic Construct
AWISPYGGSTYYADSVKG

SEQ ID NO: 65 - protein, 9 aa, Artificial Sequence
  Synthetic Construct
RHWPGGFDY

SEQ ID NO: 66 - protein, 11 aa, Artificial Sequence
  Synthetic Construct
RASQDVSTAVA

SEQ ID NO: 67 - protein, 7 aa, Artificial Sequence
  Synthetic Construct
SASFLYS

SEQ ID NO: 68 - protein, 9 aa, Artificial Sequence
  Synthetic Construct
QQYLYHPAT

SEQ ID NO: 69 - protein, 118 aa, Artificial Sequence [sequence omitted]

SEQ ID NO: 70 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 71 - protein, 447 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 440 aa, Artificial Sequence
  Synthetic Construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 214 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - RNA, 2502 nt, Homo sapiens
uauucaucaagugcccucuagcuguuaagucacucugaucucugacugcagcuccuacuguuggacacaccuggccggugcuucaguuagaucaaaccauugcugaaacugaagaggacaugucaaauauuacagauccacagaugugggauuuugaugaucuaaauuucacuggcaugccaccugcagaugaagauuacagccccuguaugcuagaaacugagacacucaacaaguauguugugaucaucgccuaugcccuaguguuccugcugagccugcugggaaacucccuggugaugcuggucaucuuauacagcagggucggccgcuccgucacugaugucuaccugcugaaccuggccuuggccgaccuacucuuugcccugaccuugcccaucugggccgccuccaaggugaauggcuggauuuuuggcacauuccugugcaagguggucucacuccugaaggaagucaacuucuacaguggcauccugcuguuggccugcaucaguguggaccguuaccuggccauuguccaugccacacgcacacugacccagaagcgucacuuggucaaguuuguuugucuuggcugcuggggacugucuaugaaucugucccugcccuucuuccuuuuccgccaggcuuaccauccaaacaauuccaguccaguuugcuaugagguccugggaaaugacacagcaaaauggcggaugguguugcggauccugccucacaccuuuggcuucaucgugccgcuguuugucaugcuguucugcuauggauucacccugcguacacuguuuaaggcccacauggggcagaagcaccgagccaugagggucaucuuugcugucguccucaucuuccugcuuugcuggcugcccuacaaccugguccugcuggcagacacccucaugaggacccaggugauccaggagagcugugagcgccgcaacaacaucggccgggcccuggaugccacugagauucugggauuucuccauagcugccucaaccccaucaucuacgccuucaucggccaaaauuuucgccauggauuccucaagauccuggcuaugcauggccuggucagcaaggaguucuuggcacgucaucguguuaccuccuacacuucuucgucugucaaugucucuuccaaccucugaaaaccaucgaugaaggaauaucucuucucagaaggaaagaauaaccaacacccugagguuguguguggaaggugaucuggcucuggacaggcacuaucuggguuuuggggggacgcuauaggauguggggaaguuaggaacuggugucuucaggggccacaccaaccuucugaggagcuguugagguaccuccaaggaccggccuuugcaccuccauggaaacgaagcaccaucauucccguugaacgucacaucuuuaacccacuaacuggcuaauuagcauggccacaucugagccccgaaucugacauuagaugagagaacagggcugaagcuguguccucaugagggcuggaugcucucguugacccucacaggagcaucuccucaacucugaguguuaagcguugagccaccaagcugguggcucugugugcucugauccgagcucaggggggugguuuucccaucucagguguguugcagugucugcuggagacauugaggcaggcacugccaaaacaucaaccugccagcuggccuugugaggagcuggaaacacauguuccccuugggggugguggaugaacaaagagaaagaggguuuggaagccagaucuaugccacaagaacccccuuuacccccaugaccaacaucgcagacacaugugcuggccaccugcugagccccaaguggaacgagacaagcagcccuuagcccuuccccucugcagcuuccaggcuggcgugcagcaucagcaucccuagaaagccaugugcagccaccaguccauugggcaggcagauguuccuaauaaagcuucuguuccgugcuugucccuguggaaguaucuugguugugacagagucaagggugugugcagcauuguuggcuguuccugcaguagaaugggggcagcaccuccuaagaaggcaccucucuggguugaagggcaguguucccuggggcuuuaacuccugcuagaacagucucuugaggcacagaaacuccuguucaugcccauaccccuggccaaggaagaucccuuuguccacaaguaaaaggaaaugcuccuccagggagucucagcuucacccugaggugagcaucaucuucuggguuaggccuugccuaggcauagcccugccucaagcuaugugagcucaccagucccuccccaaaugcuuuccaugaguugcaguuuuuuccuagucuguuuucccuccuuggagacagggcccugucgguuuauucacuguauguccuuggugccuggagccuacuaaaugcucaauaaauaaugaucacaggaaaaaaaaaaaaaaaaaa

SEQ ID NO: 76 - protein, 350 aa, Homo sapiens
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 77 - RNA, 2880 nt, Homo sapiens
agguucaaaacauucagagacagaagguggauagacaaaucuccaccuucagacugguaggcuccuccagaagccaucagacaggaagaugugaaaauccccagcacucaucccagaaucacuaaguggcaccuguccugggccaaagucccaggacagaccucauuguuccucugugggaauaccuccccaggagggcauccuggauuucccccuugcaacccaggucagaaguuucaucgucaagguuguuucaucuuuuuuuuccugucuaacagcucugacuaccacccaaccuugaggcacagugaagacaucgguggccacuccaauaacagcaggucacagcugcucuucuggagguguccuacaggugaaaagcccagcgacccagucaggauuuaaguuuaccucaaaaauggaagauuuuaacauggagagugacagcuuugaagauuucuggaaaggugaagaucuuaguaauuacaguuacagcucuacccugcccccuuuucuacuagaugccgccccaugugaaccagaaucccuggaaaucaacaaguauuuuguggucauuaucuaugcccugguauuccugcugagccugcugggaaacucccucgugaugcuggucaucuuauacagcagggucggccgcuccgucacugaugucuaccugcugaaccuagccuuggccgaccuacucuuugcccugaccuugcccaucugggccgccuccaaggugaauggcuggauuuuuggcacauuccugugcaagguggucucacuccugaaggaagucaacuucuauaguggcauccugcuacuggccugcaucaguguggaccguuaccuggccauuguccaugccacacgcacacugacccagaagcgcuacuuggucaaauucauaugucucagcaucuggggucuguccuugcuccuggcccugccugucuuacuuuuccgaaggaccgucuacucauccaauguuagcccagccugcuaugaggacaugggcaacaauacagcaaacuggcggaugcuguuacggauccugccccaguccuuuggcuucaucgugccacugcugaucaugcuguucugcuacggauucacccugcguacgcuguuuaaggcccacauggggcagaagcaccgggccaugcgggucaucuuugcugucguccucaucuuccugcucugcuggcugcccuacaaccugguccugcuggcagacacccucaugaggacccaggugauccaggagaccugugagcgccgcaaucacaucgaccgggcucuggaugccaccgagauucugggcauccuucacagcugccucaacccccucaucuacgccuucauuggccagaaguuucgccauggacuccucaagauucuagcuauacauggcuugaucagcaaggacucccugcccaaagacagcaggccuuccuuuguuggcucuucuucagggcacacuuccacuacucucuaagaccuccugccuaagugcagccccgugggguuccucccuucucuucacagucacauuccaagccucauguccacugguucuucuuggucucagugucaaugcagcccccauuguggucacaggaaguagaggaggccacguucuuacuaguuucccuugcaugguuuagaaagcuugcccuggugccucaccccuugccauaauuacuaugucauuugcuggagcucugcccauccugccccugagcccauggcacucuauguucuaagaagugaaaaucuacacuccagugagacagcucugcauacucauuaggauggcuaguaucaaaagaaagaaaaucaggcuggccaacggggugaaacccugucucuacuaaaaauacaaaaaaaaaaaaaaauuagccgggcgugguggugagugccuguaaucacagcuacuugggaggcugagaugggagaaucacuugaacccgggaggcagagguugcagugagccgagauugugccccugcacuccagccugagcgacagugagacucugucucaguccaugaagauguagaggagaaacuggaacucucgagcguugcugggggggauuguaaaauggugugaccacugcagaagacaguauggcagcuuuccucaaaacuucagacauagaauuaacacaugauccugcaauuccacuuauaggaauugacccacaagaaaugaaagcagggacuugaacccauauuuguacaccaauauucauagcagcuuauucacaagacccaaaaggcagaagcaacccaaauguucaucaaugaaugaaugaauggcuaagcaaaaugugauauguaccuaacgaaguauccuucagccugaaagaggaaugaaguacucauacauguuacaacacggacgaaccuugaaaacuuuaugcuaagugaaauaagccagacaucaacagauaaauaguuuaugauuccaccuacaugagguacugagagugaacaaauuuacagagacagaaagcagaacagugauuaccagggacugaggggaggggagcaugggaagugacgguuuaaugggcacaggguuuauguuuaggauguugaaaaaguucugcagauaaacaguagugauaguuguaccgcaaugugacuuaaugccacuaaauugacacuuaaaaaugguuuaaauggucaauuuuguuauguauauuuuauaucaauuuaaaaaaaaaccugagccccaaaagguauuuuaaucaccaaggcugauuaaaccaaggcuagaaccaccugccuauauuuuuuguuaaaugauuucauucaauaucuuuuuuuuaauaaaccauuuuuacuuggguguuuauaaaaaaaaaaa

SEQ ID NO: 78 - protein, 360 aa, Homo sapiens
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 79 - RNA, 532 nt, Homo sapiens
gagaaaccagagacuguagcaacucuggcagggagaagcugucucugauggccugaagcugugggcagcuggccaagccuaaccgcuauaaaaaggagcugccucucagcccugcaugucucuugucagcugucuuucagaagaccugguggggcaaguccgugggcaucauguugaccgagcuggagaaagccuugaacucuaucaucgacgucuaccacaaguacucccugauaaaggggaauuuccaugccgucuacagggaugaccugaagaaauugcuagagaccgaguguccucaguauaucaggaaaaagggugcagacgucugguucaaagaguuggauaucaacacugauggugcaguuaacuuccaggaguuccucauucuggugauaaagaugggcguggcagcccacaaaaaaagccaugaagaaagccacaaagaguagcugaguuacugggcccagaggcugggccccuggacauguaccugcagaauaauaaagucaucaauaccucaaaaaaaaaa

SEQ ID NO: 80 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 81 - RNA, 586 nt, Homo sapiens
aaacacucuguguggcuccucggcuuugacagagugcaagacgaugacuugcaaaaugucgcagcuggaacgcaacauagagaccaucaucaacaccuuccaccaauacucugugaagcuggggcacccagacacccugaaccagggggaauucaaagagcuggugcgaaaagaucugcaaaauuuucucaagaaggagaauaagaaugaaaaggucauagaacacaucauggaggaccuggacacaaaugcagacaagcagcugagcuucgaggaguucaucaugcugauggcgaggcuaaccugggccucccacgagaagaugcacgagggugacgagggcccuggccaccaccauaagccaggccucggggagggcacccccuaagaccacaguggccaagaucacaguggccacggccacggccacagucaugguggccacggccacagccacuaaucaggaggccaggccacccugccucuacccaaccagggccccggggccuguuaugucaaacugucuuggcuguggggcuaggggcuggggccaaauaaagucucuuccuccaagucaaaaaaaaaa

SEQ ID NO: 82 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP